(19) United States
(12) Patent Application Publication
(10) Pub. No.: US 2025/0326785 A1
(43) Pub. Date: Oct. 23, 2025

(54) SODIUM SALT AND ETHANOLAMINE SALT OF 23-HYDROXYTORMENTIC ACID

(71) Applicant: HUYABIO International, LLC, San Diego, CA (US)

(72) Inventors: Gary T. Elliott, Evergreen, CO (US); Ren Ke Li, Toronto (CA); Hayley Reece, Dalkeith (GB); Min-Woo Kim, Monteville, NJ (US); James Carl Keith, JR., Andover, MA (US); James E. Vath, Lynnvield, MA (US); Victoria Pollard, Bathgate (GB); Jun Wu, Toronto (CA); Farbod Shojaei, San Diego, CA (US); Mireille Gillings, San Diego, CA (US)

(73) Assignee: HUYABIO International, LLC, San Diego, CA (US)

(21) Appl. No.: 19/185,132

(22) Filed: Apr. 21, 2025

Related U.S. Application Data
(60) Provisional application No. 63/637,136, filed on Apr. 22, 2024.

(51) Int. Cl. C07J 63/00; A61K 31/56; A61P 9/10; C12N 5/077

(52) U.S. Cl. CPC C07J 63/008; A61K 31/56; A61P 9/10; C12N 5/0657; C12N 2501/39; C12N 2506/02

(57) ABSTRACT

Provided herein are sodium and ethanolamine salts of HBI-3808. Methods of making and using the salts are also disclosed. Pharmaceutical compositions comprising the sodium and ethanolamine salts of HBI-3808 are disclosed, as are methods of making and using the sodium and ethanolamine salts of HBI-3808.

### CROSS-REFERENCE

This application claims the benefit of U.S. Provisional Application No. 63/637,136 filed Apr. 22, 2024, the contents of which are incorporated herein by reference in their entirety.

### BACKGROUND

One of the greatest unmet needs for preventing and/or treating heart failure is regeneration of the heart muscle tissue. Regenerating the myocardium requires a replacement of the dead cardiomyocytes and endothelial cells/capillary beds in order to restore the structural and functional integrity of the heart. Many different approaches to regenerating heart muscle tissue are currently under study.

23-Hydroxytormentic acid (also referred to herein as HBI-3808) can be used to treat patients with injured or damaged heart muscles caused by an ischemic disease. Following an ischemic event, such as heart attack, natural repair processes for replacing injured or damaged heart muscles with new cardiomyocytes are greatly reduced or may stop altogether. Scar tissues may replace the necrosed myocardium, causing further deterioration in cardiac function. 23-Hydroxytormentic acid can regenerate cardiomyocytes, increase capillary density, reduce infarct scar size and thereby repair injured or damaged heart muscles.

There is a need for one or more solid forms of 23-hydroxytormentic acid that increase bioavailability, and improve the degree of stability and other chemical and physical properties of the compound. The disclosed salts and polymorphs are designed to address these needs and provide additional advantages as well.

### SUMMARY

In an aspect of the present disclosure a sodium salt of 23-hydroxytormentic acid (HBI-3808) is provided. HBI-3808 has the following chemical formula.

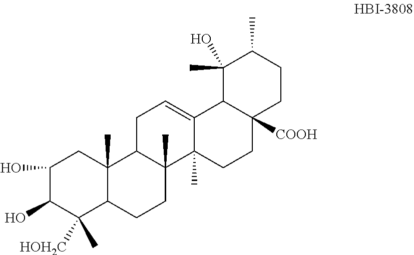

In certain embodiments, the sodium salt of HBI-3808 is a monosodium salt. In certain embodiments, the sodium salt of HBI-3808 comprises, consists essentially of, or consists of HBI-3808 monosodium salt.

The sodium salt of HBI-3808 can have an x-ray power diffraction (XRPD) diffractogram of Pattern 1, 2, 3, 4, 5, 6, 7, 8, 9, 10, 11, 12, 13, 14, 15, or 16.

In certain embodiments, the sodium salt of HBI-3808, has an XRPD diffractogram of Pattern 1, wherein the XRPD diffractogram comprises 1 to 8, 1 to 6, or 1 to 3 peaks of, or substantially matches, FIG. 1A. In certain embodiments, the sodium salt of HBI-3808, has an XRPD diffractogram of Pattern 1, wherein the XRPD diffractogram comprises 1 to 8, 1 to 6, 1 to 3, or all peaks (±0.2 °2θ) listed in Table 1. In certain embodiments, the sodium salt of HBI-3808, has an XRPD diffractogram of Pattern 1, wherein the XRPD diffractogram comprises 1 to 8, 1 to 6, or 1 to 3 peaks of, or substantially matches, FIG. 1A, and 1 to 8, 1 to 6, 1 to 3, or all peaks (±0.2°2θ) listed in Table 1.

In certain embodiments, the sodium salt of HBI-3808, has an XRPD diffractogram of Pattern 2, wherein the XRPD diffractogram comprises 1 to 9, 1 to 7, 1 to 5, 1 to 3, or 1 to 2 peaks of, or substantially matches, FIG. 2A. In certain embodiments, the sodium salt of HBI-3808, has an XRPD diffractogram of Pattern 2, wherein the XRPD diffractogram comprises 1 to 35, 1 to 30, 1 to 25, 1 to 20, 1 to 15, 1 to 10, 1 to 5, or all peaks (±0.2 °2θ) listed in Table 2A. In certain embodiments, the sodium salt of HBI-3808, has an XRPD diffractogram of Pattern 2, wherein the XRPD diffractogram comprises 1 to 9, 1 to 7, 1 to 5, 1 to 3, or 1 to 2 peaks of, or substantially matches, FIG. 2A, and 1 to 35, 1 to 30, 1 to 25, 1 to 20, 1 to 15, 1 to 10, 1 to 5, or all peaks (±0.2 °2θ) listed in Table 2A.

In certain embodiments, the sodium salt of HBI-3808, has an XRPD diffractogram of Pattern 3, wherein the XRPD diffractogram comprises 1 to 8, 1 to 6, or 1 to 3 peaks of, or substantially matches, FIG. 3A. In certain embodiments, the sodium salt of HBI-3808, has an XRPD diffractogram of Pattern 3, wherein the XRPD diffractogram comprises 1 to 11, 1 to 8, 1 to 5, 1 to 3, or all peaks (±0.2 °2θ) listed in Table 3. In certain embodiments, the sodium salt of HBI-3808, has an XRPD diffractogram of Pattern 3, wherein the XRPD diffractogram comprises 1 to 8, 1 to 6, or 1 to 3 peaks of, or substantially matches, FIG. 3A, and 1 to 11, 1 to 8, 1 to 5, 1 to 3, or all peaks (±0.2 °2θ) listed in Table 3.

In certain embodiments, the sodium salt of HBI-3808, has an XRPD diffractogram of Pattern 4, wherein the XRPD diffractogram comprises 1 to 8, 1 to 6, or 1 to 3 peaks of, or substantially matches, FIG. 4A. In certain embodiments, the sodium salt of HBI-3808, has an XRPD diffractogram of Pattern 4, wherein the XRPD diffractogram comprises 1 to 28, 1 to 20, 1 to 15, 1 to 10, 1 to 5, or all peaks (±0.2 °2θ) listed in Table 4. In certain embodiments, the sodium salt of HBI-3808, has an XRPD diffractogram of Pattern 4, wherein the XRPD diffractogram comprises 1 to 8, 1 to 6, or 1 to 3 peaks of, or substantially matches, FIG. 4A, and 1 to 28, 1 to 20, 1 to 15, 1 to 10, 1 to 5, or all peaks (±0.2°2θ) listed in Table 4.

In certain embodiments, the sodium salt of HBI-3808, has an XRPD diffractogram of Pattern 5, wherein the XRPD diffractogram comprises 1 to 10, 1 to 8, 1 to 6, or 1 to 3 peaks of, or substantially matches, FIG. 5A. In certain embodiments, the sodium salt of HBI-3808, has an XRPD diffractogram of Pattern 5, wherein the XRPD diffractogram comprises 1 to 35, 1 to 30, 1 to 25, 1 to 20, 1 to 15, 1 to 10, 1 to 5, or all peaks (±0.2 °2θ) listed in Table 5A. In certain embodiments, the sodium salt of HBI-3808, has an XRPD diffractogram of Pattern 5, wherein the XRPD diffractogram comprises 1 to 10, 1 to 8, 1 to 6, or 1 to 3 peaks of, or substantially matches, FIG. 5A, and 1 to 35, 1 to 30, 1 to 25, 1 to 20, 1 to 15, 1 to 10, 1 to 5, or all peaks (0.2 °2θ) listed in Table 5A. In certain embodiments, the sodium salt of HBI-3808 having XRPD diffractogram of Pattern 5, is a mono sodium mono-hydrate salt.

In certain embodiments, the sodium salt of HBI-3808, has an XRPD diffractogram of Pattern 6, wherein the XRPD diffractogram comprises 1 to 10, 1 to 8, 1 to 6, or 1 to 3 peaks of, or substantially matches, FIG. 6A. In certain embodiments, the sodium salt of HBI-3808, has an XRPD diffractogram of Pattern 6, wherein the XRPD diffractogram comprises 1 to 35, 1 to 30, 1 to 25, 1 to 20, 1 to 15, 1 to 10, 1 to 5, or all peaks (±0.2 °2θ) listed in Table 6. In certain embodiments, the sodium salt of HBI-3808, has an XRPD diffractogram of Pattern 6, wherein the XRPD diffractogram comprises 1 to 10, 1 to 8, 1 to 6, or 1 to 3 peaks of, or substantially matches, FIG. 6A, and 1 to 35, 1 to 30, 1 to 25, 1 to 20, 1 to 15, 1 to 10, 1 to 5, or all peaks (10.2 °2θ) listed in Table 6. In certain embodiments, the sodium salt of HBI-3808 having XRPD diffractogram of Pattern 6, is a mono sodium anhydrous salt.

In certain embodiments, the sodium salt of HBI-3808, has an XRPD diffractogram of Pattern 7, wherein the XRPD diffractogram comprises 1 to 10, 1 to 8, 1 to 6, or 1 to 3 peaks of, or substantially matches, FIG. 7A. In certain embodiments, the sodium salt of HBI-3808, has an XRPD diffractogram of Pattern 7, wherein the XRPD diffractogram comprises 1 to 28, 1 to 25, 1 to 20, 1 to 15, 1 to 10, 1 to 5, or all peaks (±0.2 °2θ) listed in Table 7. In certain embodiments, the sodium salt of HBI-3808, has an XRPD diffractogram of Pattern 7, wherein the XRPD diffractogram comprises 1 to 10, 1 to 8, 1 to 6, or 1 to 3 peaks of, or substantially matches, FIG. 7A, and 1 to 28, 1 to 25, 1 to 20, 1 to 15, 1 to 10, 1 to 5, or all peaks (±0.2 °2θ) listed in Table 7.

In certain embodiments, the sodium salt of HBI-3808, has an XRPD diffractogram of Pattern 8, wherein the XRPD diffractogram comprises 1 to 15, 1 to 10, 1 to 8, 1 to 6, or 1 to 3 peaks of, or substantially matches, FIG. 8A. In certain embodiments, the sodium salt of HBI-3808, has an XRPD diffractogram of Pattern 8, wherein the XRPD diffractogram comprises 1 to 34, 1 to 30, 1 to 25, 1 to 20, 1 to 15, 1 to 10, 1 to 5, or all peaks (±0.2°2θ) listed in Table 8. In certain embodiments, the sodium salt of HBI-3808, has an XRPD diffractogram of Pattern 8, wherein the XRPD diffractogram comprises 1 to 15, 1 to 10, 1 to 8, 1 to 6, or 1 to 3 peaks of, or substantially matches, FIG. 8A, and 1 to 34, 1 to 30, 1 to 25, 1 to 20, 1 to 15, 1 to 10, 1 to 5, or all peaks (±0.2 °2θ) listed in Table 8.

In certain embodiments, the sodium salt of HBI-3808, has an XRPD diffractogram of Pattern 9, wherein the XRPD diffractogram comprises 1 to 15, 1 to 10, 1 to 8, 1 to 6, or 1 to 3 peaks of, or substantially matches, FIG. 9A. In certain embodiments, the sodium salt of HBI-3808, has an XRPD diffractogram of Pattern 9, wherein the XRPD diffractogram comprises 1 to 35, 1 to 30, 1 to 25, 1 to 20, 1 to 15, 1 to 10, 1 to 5, or all peaks (±0.2 °2θ) listed in Table 9. In certain embodiments, the sodium salt of HBI-3808, has an XRPD diffractogram of Pattern 9, wherein the XRPD diffractogram comprises 1 to 15, 1 to 10, 1 to 8, 1 to 6, or 1 to 3 peaks of, or substantially matches, FIG. 9A, and 1 to 35, 1 to 30, 1 to 25, 1 to 20, 1 to 15, 1 to 10, 1 to 5, or all peaks (±0.2 °2θ) listed in Table 9.

In certain embodiments, the sodium salt of HBI-3808, has an XRPD diffractogram of Pattern 10, wherein the XRPD diffractogram comprises 1 to 20, 1 to 15, 1 to 10, 1 to 8, 1 to 6, or 1 to 3 peaks of, or substantially matches, FIG. 10A. In certain embodiments, the sodium salt of HBI-3808, has an XRPD diffractogram of Pattern 10, wherein the XRPD diffractogram comprises 1 to 30, 1 to 25, 1 to 20, 1 to 15, 1 to 10, 1 to 5, or all peaks (0.2 °2θ) listed in Table 10. In certain embodiments, the sodium salt of HBI-3808, has an XRPD diffractogram of Pattern 10, wherein the XRPD diffractogram comprises 1 to 20, 1 to 15, 1 to 10, 1 to 8, 1 to 6, or 1 to 3 peaks of, or substantially matches, FIG. 10A, and 1 to 30, 1 to 25, 1 to 20, 1 to 15, 1 to 10, 1 to 5, or all peaks (±0.2°2θ) listed in Table 10.

In certain embodiments, the sodium salt of HBI-3808, has an XRPD diffractogram of Pattern 11, wherein the XRPD diffractogram comprises 1 to 15, 1 to 10, 1 to 8, 1 to 6, or 1 to 3 peaks of, or substantially matches, FIG. 11A. In certain embodiments, the sodium salt of HBI-3808, has an XRPD diffractogram of Pattern 11, wherein the XRPD diffractogram comprises 1 to 38, 1 to 35, 1 to 30, 1 to 25, 1 to 20, 1 to 15, 1 to 10, 1 to 5, or all peaks (0.2 °2θ) listed in Table 11. In certain embodiments, the sodium salt of HBI-3808, has an XRPD diffractogram of Pattern 11, wherein the XRPD diffractogram comprises 1 to 15, 1 to 10, 1 to 8, 1 to 6, or 1 to 3 peaks of, or substantially matches, FIG. 11A, and 1 to 38, 1 to 35, 1 to 30, 1 to 25, 1 to 20, 1 to 15, 1 to 10, 1 to 5, or all peaks (±0.2°2θ) listed in Table 11.

In certain embodiments, the sodium salt of HBI-3808, has an XRPD diffractogram of Pattern 12, wherein the XRPD diffractogram comprises 1 to 15, 1 to 10, 1 to 8, 1 to 6, or 1 to 3 peaks of, or substantially matches, FIG. 12A. In certain embodiments, the sodium salt of HBI-3808, has an XRPD diffractogram of Pattern 12, wherein the XRPD diffractogram comprises 1 to 38, 1 to 35, 1 to 30, 1 to 25, 1 to 20, 1 to 15, 1 to 10, 1 to 5, or all peaks (0.2 °2θ) listed in Table 12. In certain embodiments, the sodium salt of HBI-3808, has an XRPD diffractogram of Pattern 12, wherein the XRPD diffractogram comprises 1 to 15, 1 to 10, 1 to 8, 1 to 6, or 1 to 3 peaks of, or substantially matches, FIG. 12A, and 1 to 38, 1 to 35, 1 to 30, 1 to 25, 1 to 20, 1 to 15, 1 to 10, 1 to 5, or all peaks (±0.2 °2θ) listed in Table 12.

In certain embodiments, the sodium salt of HBI-3808, has an XRPD diffractogram of Pattern 13, wherein the XRPD diffractogram comprises 1 to 20, 1 to 15, 1 to 10, 1 to 8, 1 to 6, or 1 to 3 peaks of, or substantially matches, FIG. 13A. In certain embodiments, the sodium salt of HBI-3808, has an XRPD diffractogram of Pattern 13, wherein the XRPD diffractogram comprises 1 to 28, 1 to 25, 1 to 20, 1 to 15, 1 to 10, 1 to 5, or all peaks (0.2 °2θ) listed in Table 13. In certain embodiments, the sodium salt of HBI-3808, has an XRPD diffractogram of Pattern 13, wherein the XRPD diffractogram comprises 1 to 20, 1 to 15, 1 to 10, 1 to 8, 1 to 6, or 1 to 3 peaks of, or substantially matches, FIG. 13A, and 1 to 28, 1 to 25, 1 to 20, 1 to 15, 1 to 10, 1 to 5, or all peaks (±0.2°2θ) listed in Table 13.

In certain embodiments, the sodium salt of HBI-3808, has an XRPD diffractogram of Pattern 14, wherein the XRPD diffractogram comprises 1 to 15, 1 to 10, 1 to 8, 1 to 6, or 1 to 3 peaks of, or substantially matches, FIG. 14A. In certain embodiments, the sodium salt of HBI-3808, has an XRPD diffractogram of Pattern 14, wherein the XRPD diffractogram comprises 1 to 28, 1 to 25, 1 to 20, 1 to 15, 1 to 10, 1 to 5, or all peaks (±0.2 °2θ) listed in Table 14. In certain embodiments, the sodium salt of HBI-3808, has an XRPD diffractogram of Pattern 14, wherein the XRPD diffractogram comprises 1 to 15, 1 to 10, 1 to 8, 1 to 6, or 1 to 3 peaks of, or substantially matches, FIG. 14A, and 1 to 28, 1 to 25, 1 to 20, 1 to 15, 1 to 10, 1 to 5, or all peaks (±0.2°2θ) listed in Table 14.

In certain embodiments, the sodium salt of HBI-3808 is a monohydrate mono acetone solvate, having an XRPD diffractogram of Pattern 15, wherein the XRPD diffractogram comprises 1 to 15, 1 to 10, 1 to 8, 1 to 6, or 1 to 3 peaks of, or substantially matches, FIG. 15A. In certain embodiments, the sodium salt of HBI-3808, has an XRPD diffractogram of Pattern 15, wherein the XRPD diffractogram comprises 1 to 40, 1 to 35, 1 to 30, 1 to 25, 1 to 20, 1 to 15, 1 to 10, 1 to 5, or all peaks (±0.2 °2θ) listed in Table 15. In certain embodiments, the sodium salt of HBI-3808, has an XRPD diffractogram of Pattern 15, wherein the XRPD diffractogram comprises 1 to 15, 1 to 10, 1 to 8, 1 to 6, or 1 to 3 peaks of, or substantially matches, FIG. 15A, and 1 to 40, 1 to 35, 1 to 30, 1 to 25, 1 to 20, 1 to 15, 1 to 10, 1 to 5, or all peaks (±0.2 °2θ) listed in Table 15. In certain embodiments, the sodium salt of HBI-3808 having XRPD diffractogram of Pattern 15, is a mono sodium, mono-hydrate, mono acetone solvate salt.

In certain embodiments, the sodium salt of HBI-3808, has an XRPD diffractogram of Pattern 16, wherein the XRPD diffractogram comprises 1 to 20, 1 to 15, 1 to 10, 1 to 8, 1 to 6, or 1 to 3 peaks of, or substantially matches, FIG. 16A. In certain embodiments, the sodium salt of HBI-3808, has an XRPD diffractogram of Pattern 16, wherein the XRPD diffractogram comprises 1 to 70, 1 to 60, 1 to 50, 1 to 40, 1 to 35, 1 to 30, 1 to 25, 1 to 20, 1 to 15, 1 to 10, 1 to 5, or all peaks (±0.2 °2θ) listed in Table 16. In certain embodiments, the sodium salt of HBI-3808, has an XRPD diffractogram of Pattern 16, wherein the XRPD diffractogram comprises 1 to 20, 1 to 15, 1 to 10, 1 to 8, 1 to 6, or 1 to 3 peaks of, or substantially matches, FIG. 16A, and 1 to 70, 1 to 60, 1 to 50, 1 to 40, 1 to 35, 1 to 30, 1 to 25, 1 to 20, 1 to 15, 1 to 10, 1 to 5, or all peaks (±0.2 °2θ) listed in Table 16.

In certain embodiments, the sodium salt of HBI-3808, has any one of, any combination of, or all of i) an XRPD diffractogram of Pattern 2, wherein the XRPD diffractogram comprises 1 to 9, 1 to 7, 1 to 5, 1 to 3, or 1 to 2 peaks of, or substantially matches, FIG. 2A, or 1 to 35, 1 to 30, 1 to 25, 1 to 20, 1 to 15, 1 to 10, 1 to 5, or all peaks (±0.2 °2θ) listed in Table 2A, or both; ii) a thermogravimetry (TG)—differential scanning calorimetry (DSC) thermogram substantially matching that of FIG. 2G; iii) a DSC thermogram substantially matching that of FIG. 2H; iv) a DVS mass plot substantially matching that of FIG. 2K; v) a dynamic vapor sorption (DVS) isotherm plot substantially matching that of FIG. 2L; vi) a FT-IR spectrum substantially matching the FT-IR spectrum of FIG. 2I; and vii) a Raman spectrum substantially matching the Raman spectrum of FIG. 2J.

In certain embodiments, the sodium salt of HBI-3808, has any one of, any combination of, or all of i) an XRPD diffractogram of Pattern 5, wherein the XRPD diffractogram comprises 1 to 10, 1 to 8, 1 to 6, or 1 to 3 peaks of, or substantially matches, FIG. 5A, or 1 to 35, 1 to 30, 1 to 25, 1 to 20, 1 to 15, 1 to 10, 1 to 5, or all peaks (±0.2°2θ) listed in Table 5A, or both; ii) a TG-DSC thermogram substantially matching that of FIG. 5G; iii) a DSC thermogram substantially matching that of FIG. 5H; iv) a DVS mass plot substantially matching that of FIG. 5I; and v) a DVS isotherm plot substantially matching the that of FIG. 5J.

In an aspect of the present disclosure a method of making a sodium salt of HBI-3808, is provided. In certain embodiments, the method is method A, which includes any one of, any combination of, or all of steps (a) to (f), of which: step (a) can include contacting HBI-3808 with a first solvent to form a first slurry; step (b) can include dissolving sodium hydroxide in a second solvent to form a first solution; step (c) can include contacting the first slurry of step (a) with the first solution to form a mixture at a first temperature; step (d) can include contacting the mixture of step (c) with an anti-solvent to form a second slurry; step (e) can include cooling the second slurry to a second temperature, wherein the second temperature is lower than the first temperature; step (f) can include isolating solids from the second slurry. In certain embodiments, the first solvent of method A comprises, consists essentially of, or consists of methanol. In certain embodiments, the second solvent of method A comprises, consists essentially of, or consists of water. In certain embodiments, the anti-solvent of method A comprises, consists essentially of, or consists of acetone and/or tert-butyl methyl ether (TBME). The first temperature of method A can be about 20° C. to 60° C., about 25° C. to 55° C., about 30° C. to 55° C., about 35° C. to 45° C., or about 40° C.

In certain embodiments, the first temperature of method A is about 40° C. The second temperature of method A can be about 0° C. to 20° C., about 2° C. to 15° C., about 3° C. to 10° C., or about 5° C.

In certain embodiments, the second temperature of method A is about 5° C. In certain embodiments, the method A, further includes washing the solids (e.g., isolated in step f) with a rinse solution. In certain embodiments, the rinse solution of method A contains methanol, water, and/or acetone. In certain embodiments, the rinse solution of method A comprises, consists essentially of, or consists of a solution of methanol, water, and acetone. In certain embodiments, the rinse solution of method A comprises, consists essentially of, or consists of a solution of methanol, water, and acetone in a ratio of about 9:1:90% v:% v:% v. In certain embodiments, the method A, further comprises drying the separated solids. In certain embodiments, the separated solids are dried under vacuum.

In certain embodiments, the method of making the sodium salt of HBI-3808, is method B, wherein the method B includes any one of, any combination of, or all of steps (a′) to (d′), of which: step (a′) can include contacting HBI-3808 with a first solvent at a first temperature to form a slurry; step (b′) can include contacting the slurry of step (a′) with sodium hydroxide and optionally an additional solvent; step (c′) can include temperature cycling the slurry (e.g., formed in step (b′)) between the first temperature and a second temperature, wherein the second temperature is lower than the first temperature. Step (d′) can include isolating solids from the slurry (e.g., formed during and/or after the temperature cycling). The first solvent of method B can be a lower alcohol, e.g., a Cp alcohol. In certain embodiments, the lower alcohol is 2-propanol. In certain embodiments, the method B can further include drying the solids isolated in step (d′). In certain embodiments, the solids are dried under vacuum. The first temperature of method B can be about 20° C. to 60° C., about 25° C. to 55° C., about 30° C. to 55° C., about 35° C. to 45° C., or about 40° C. In certain embodiments, the first temperature of method B is about 40° C. The second temperature of method B can be about 0° C. to 20° C., about 2° C. to 15° C., about 3° C. to 10° C., or about 5° C. In certain embodiments, the second temperature of method B is about 5° C.

Certain aspects are directed to a method of making a sodium salt of HBI-3808 polymorph. In certain embodiments, the method of making sodium salt of HBI-3808 polymorph, is method C, wherein the method C can include any one of, or any combination of, or all of steps (a″) to (d″). Step (a″) can include dissolving a sodium salt of HBI-3808, in a solvent to form a solution at a first temperature. Step (b″) can include contacting the solution of step (a″) with a counter solvent. Step (c″) can include optionally, cycling the solution (e.g., formed in step b″) between the first temperature and a second temperature, where the second temperature is lower than the first temperature. Step d″ can include isolating solids from the solution (e.g., formed in step (b″) and/or (c″)). In certain embodiments, the solvent of method C is or comprises water. In certain embodiments, the counter solvent of the method C is tetrahydrofuran (THF). In certain embodiments, the method C further includes drying the isolated solids (e.g., isolated in step d″).

In certain embodiments, the isolated solids can be dried under vacuum. The first temperature of the method C can be about 20° C. to 60° C., about 25° C. to 55° C., about 30° C. to 55° C., about 35° C. to 45° C., or about 40° C. In certain embodiments, the first temperature of the method C is about 40° C. The second temperature of the method C can be about 0° C. to 20° C., about 2° C. to 15° C., about 3° C. to 10° C., or about 5° C. In certain embodiments, the second temperature of the method C is about 5° C.

Certain aspects are directed to a method of making a sodium salt of HBI-3808 having of XRPD of Pattern 5. The method includes providing a sample of a sodium salt of HBI-3808 having XRPD of Pattern 2, and/or exposing the Pattern 2 sodium salt (e.g., sodium salt of HBI-3808 having XRPD of Pattern 2) to conditions including a combination of temperature and humidity for a time sufficient to convert the Pattern 2 sodium salt to a Pattern 5 sodium salt. In certain embodiments, the Pattern 2 sodium salt is exposed to the temperature of at least about 20° C., at least about 30° C., at least about 40° C., at least about 50° C., at least about 60° C., or 20° C. to 150° C. In certain embodiments, the Pattern 2 sodium salt is exposed to the temperature of at least about 40° C. In certain embodiments, the Pattern 2 sodium salt is exposed to the humidity of at least about 30% RH, 40% RH, 50% RH, 60% RH, 70% RH, 80% RH, 90% RH, or 30% RH to 90 % RH. In certain embodiments, the Pattern 2 sodium salt is exposed to the humidity of at least about 40% RH. In certain embodiments, the Pattern 2 sodium salt is exposed to the suitable temperature and/or humidity, of about 24 hours to about eight (8) weeks.

Certain aspects are directed to a method of using a sodium salt of HBI-3808 described herein.

In an aspect of this disclosure, an ethanolamine salt of HBI-3808 is provided. The ethanolamine salt of HBI-3808, can be a monoethanolamine salt. In certain embodiments, the ethanolamine salt of HBI-3808 comprises, consists essentially of, or consists of HBI-3808 monoethanolamine salt.

In certain embodiments, the ethanolamine salt of HBI-3808, has an XRPD diffractogram comprising 1 to 19, 1 to 11, 1 to 8, 1 to 6, or 1 to 3 peaks of, or substantially matches, FIG. 17A.

In certain embodiments, the ethanolamine salt of HBI-3808, has an XRPD diffractogram comprising 1 to 40, 1 to 35, 1 to 30, 1 to 25, 1 to 20, 1 to 15, 1 to 10, 1 to 5, or all peaks (±0.2 °2θ) listed in Table 17A. In certain embodiments, the ethanolamine salt of HBI-3808, has an XRPD diffractogram comprising 1 to 19, 1 to 11, 1 to 8, 1 to 6, or 1 to 3 peaks of, or substantially matches, FIG. 17A, and 1 to 40, 1 to 35, 1 to 30, 1 to 25, 1 to 20, 1 to 15, 1 to 10, 1 to 5, or all peaks (0.2 °2θ) listed in Table 17A.

In certain embodiments, the ethanolamine salt of HBI-3808, has any one of, any combination of, or all of i) an XRPD diffractogram comprising 1 to 19, 1 to 11, 1 to 8, 1 to 6, or 1 to 3 peaks of, or substantially matches, FIG. 17A, or 1 to 40, 1 to 35, 1 to 30, 1 to 25, 1 to 20, 1 to 15, 1 to 10, 1 to 5, or all peaks (±0.2 °2θ) listed in Table 17A, or both; ii) a TG-DSC thermogram substantially matching to that of FIG. 17H; iii) a DSC thermogram substantially matching to that of FIG. 17I; iv) a FT-IR spectrum substantially matching to that of FIG. 17J; v) a Raman spectrum substantially matching to that of FIG. 17K; vi) a DVS mass plot substantially matching to that of FIG. 17L; and vii) a DVS isotherm plot substantially matching to that of FIG. 17M.

Certain aspects are directed to a method of making an ethanolamine salt of HBI-3808. The method of making an ethanolamine salt of HBI-3808 can include any one of, any combination of, or all of steps (v), (w), (x), (y) and (z). Step (v) can include contacting HBI-3808, with a first solvent to form a slurry. Step (w) can include contacting the slurry of step (v) with an ethanolamine solution at a first temperature to form a solution. In certain embodiments, step (w) includes adding a volume of the ethanolamine solution, the volume being sufficient to form the solution of step (w). The ethanolamine solution contains ethanolamine and a second solvent. Step (x) can include adding a counter solvent to the solution formed in step (w). Step (y) can include cooling the solution (e.g., formed in step (w)) to a second temperature for a time sufficient to precipitate solids from the solution, wherein the second temperature is lower than the first temperature. Step (z) can include isolating the solids from the solution. In certain embodiments, the first solvent (e.g., of step (v)) contains water and methanol. In certain embodiments, the second solvent (e.g., of step (w) contains water and methanol. In certain embodiments, the first solvent (e.g., of step (v)), and the second solvent (e.g., of step (w) contains water and methanol. In certain embodiments, the first solvent (e.g., of step (v)), and the second solvent (e.g., of step (w)) independently contains water and methanol in a ratio of about 80:20 (% v:% v) to 99:1 (% v:% v), about 85:15 (% v:% v) to 95:5 (% v: % v), or about 90:10 (% v: % v). In certain embodiments, the first solvent (e.g., of step (v)), and the second solvent (e.g., of step (w)) contains water and methanol in a ratio of about 90:10 (% v:% v). In certain embodiments, the first temperature (e.g., of step (w)) is about 20° C. to 60° C., about 25° C. to 55° C., about 30° C. to 55° C., about 35° C. to 45° C., or about 40° C. In certain embodiments, the first temperature (e.g., of step (w)) is about 40° C. In certain embodiments, the counter solvent contains TBME. In certain embodiments, the second temperature (e.g., of step (y)) is about 0° C. to 20° C., about 2° C. to 15° C., about 3° C. to 10° C., or about 5° C. In certain embodiments, the second temperature (e.g., of step (y)) is about 5° C. In certain aspects, the cooling (e.g., of step (y)) is carried out at a rate of about 0.01 to 3° C./min, about 0.01 to 2° C./min, about 0.01 to 1° C./min, about 0.05 to 0.5° C./min, or about 0.1° C./min. In certain embodiments, the cooling (e.g., of step (y)) is carried out at a rate of about 0.1° C./min. In certain embodiments, the time (e.g., of step (y)) sufficient to precipitate solids from the solution is about 1 to about 100 hr.

In certain embodiments, in step (z), the solids can be isolated by pressure filtration, centrifuge filtration, and/or Buchner funnel filtration. In certain embodiments, isolating the solids from the solution includes drying the solids. The isolated solids can have a purity of at least about 95%, about 95% to 99.5%, about 95%, about 95% to 99.9%, about 95% to 99%. In some embodiments, the isolated solids can contain a monoethanolamine salt of HBI-3808.

Methods of using an ethanolamine salt of HBI-3808 are also described herein.

Various terms and phrases used throughout this specification are defined in the following paragraphs, as indicated by the use of quotation marks (“ ”) around the defined terms and phrases.

The terms “about” or “approximately” are defined as being close to, as understood by one of ordinary skill in the art. In non-limiting embodiments, the terms indicate a value within 10%, preferably within 5%, more preferably within 1%, and most preferably within 0.5% of a stated value.

The terms “wt. %,” “vol. %,” or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. %f component.

The term “substantially” and its variations are defined to include ranges within 10%, within 5%, within 1%, or within 0.5% of a stated value, unless otherwise defined within the context in which the term appears.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or an intended result, e.g., to improve or ameliorate a disease sign or symptom.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one,” unless a more restrictive meaning is clearly indicated by the context in which the term appears.

The phrase “and/or” can include “and” or “or.” To illustrate, A, B, and/or C can include: A alone, B alone, C alone, a combination of A and B, a combination of A and C, a combination of B and C, or a combination of A, B, and C.

The terms “sodium salt of HBI-3808 of XRPD Pattern X”, “sodium salt of HBI-3808, having an XRPD diffractogram of Pattern X”, and “sodium salt of HBI-3808, has an XRPD diffractogram of Pattern X”, may be used interchangeably throughout this disclosure to refer to the same salt, wherein X is an integer selected from 1, 2, 3, 4, 5, 6, 7, 8, 9, 10, 11, 12, 13, 14, 15 and 16, which indicates the Pattern number.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the aspects of the present disclosure will become apparent from the following figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present disclosure may become apparent to those skilled in the art with the benefit of the following detailed description and upon reference to the accompanying drawings.

FIG. 1A: XRPD diffractogram of the sodium salt of HBI-3808 of XRPD Pattern 1.

FIG. 1B: Peak fitting of the XRPD diffractogram of the sodium salt of HBI-3808 of XRPD Pattern 1 (FIG. 1A).

FIG. 2A: XRPD diffractogram of the sodium salt of HBI-3808 of XRPD Pattern 2.

FIG. 2B: Peak fitting of the XRPD diffractogram of the sodium salt of HBI-3808 of XRPD Pattern 2 (FIG. 2A).

FIG. 2C: XRPD spectra of sodium salt of HBI-3808 of XRPD Pattern 2 (top), damp isolated solid as prepared in Example 2 (second from top), dried solids as prepared in Example 2 (third from top), and HBI-3808 acid of Pattern 1 (bottom).

FIG. 2D: HPLC chromatogram of the isolated solids, as described in Example 2.

FIG. 2E: ¹H NMR spectrum of the isolated solids as described in Example 2, in d⁶-DMSO.

FIG. 2F: PLM image of the isolated solids, as described in Example 2. Objective: ×20. Scale bar 100 μm.

FIG. 2G: TG-DSC thermogram of the isolated solids, as described in Example 2.

FIG. 2H: DSC thermogram of the isolated solids, as described in Example 2.

FIG. 2I: FT-IR spectrum of the isolated solids, as described in Example 2.

FIG. 2J: Raman spectrum of the isolated solids, as described in Example 2.

FIG. 2K: DVS mass plot of the isolated solids, as described in Example 2.

FIG. 2L: DVS isotherm plot of the isolated solids, as described in Example 2.

FIG. 2M: XRPD diffractogram of recovered material from DVS analysis of the isolated solids, as described in Example 2. Input, sodium salt of HBI-3808 of XRPD Pattern 2 (top), and material post DVS (bottom).

FIG. 2N: TG-DSC thermogram of recovered material from DVS analysis of the isolated solids, as described in Example 2.

FIG. 20: Hot stage microscopy of the isolated solids, as described in Example 2.

Objective: ×10.

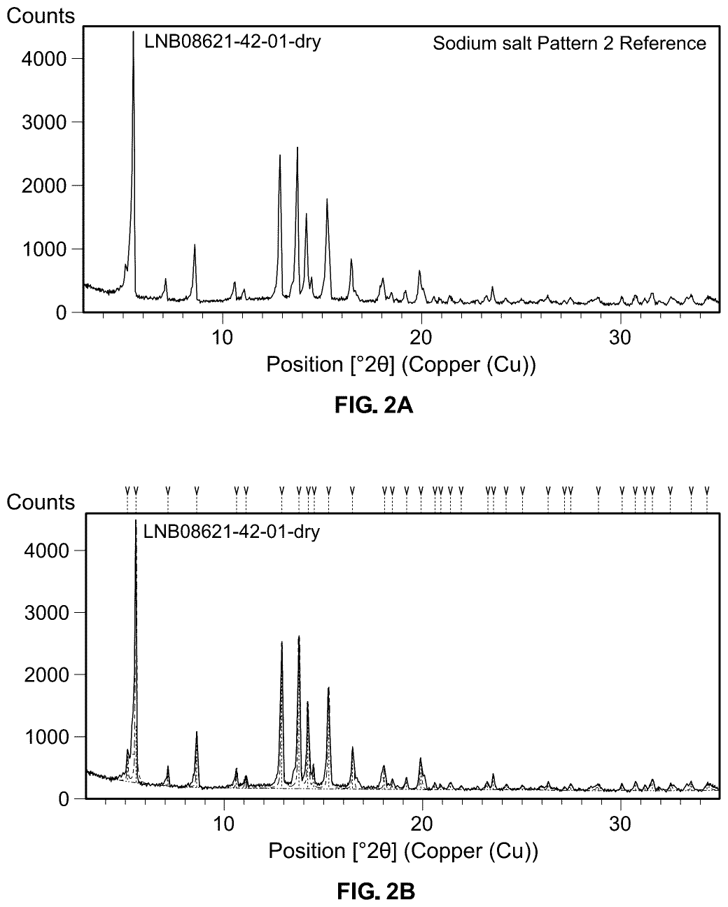

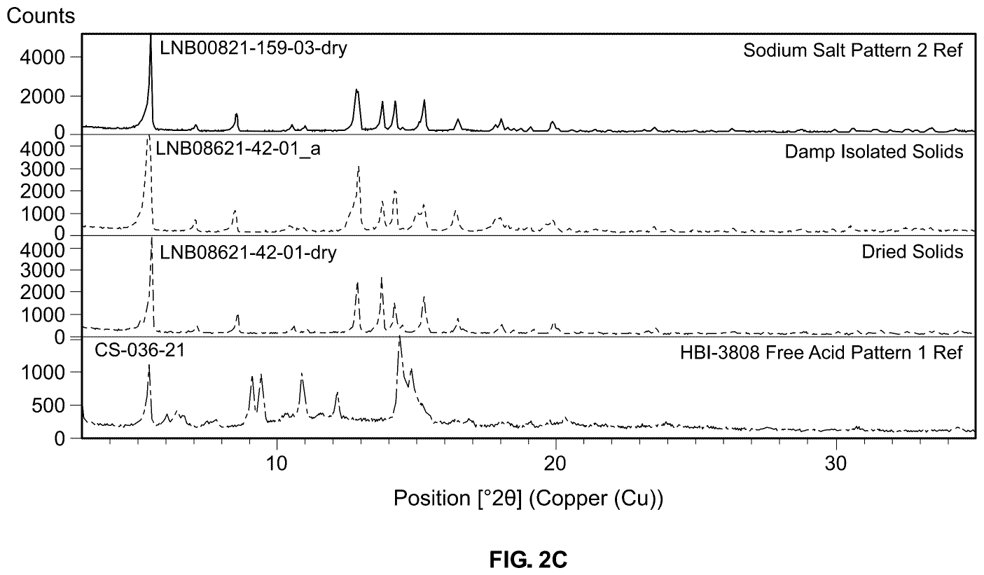

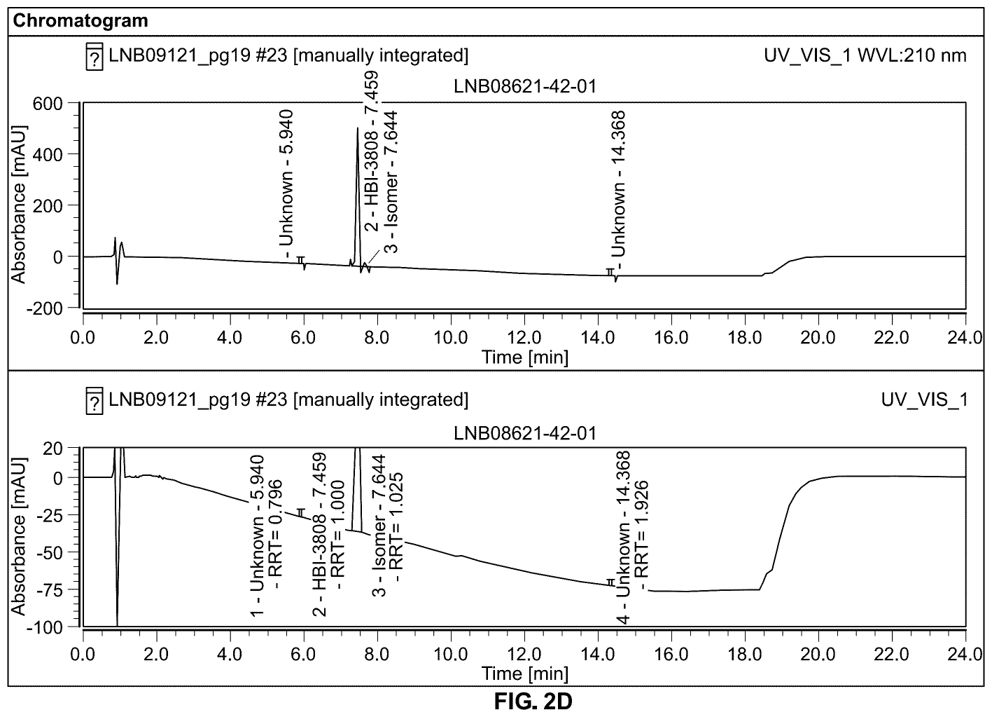

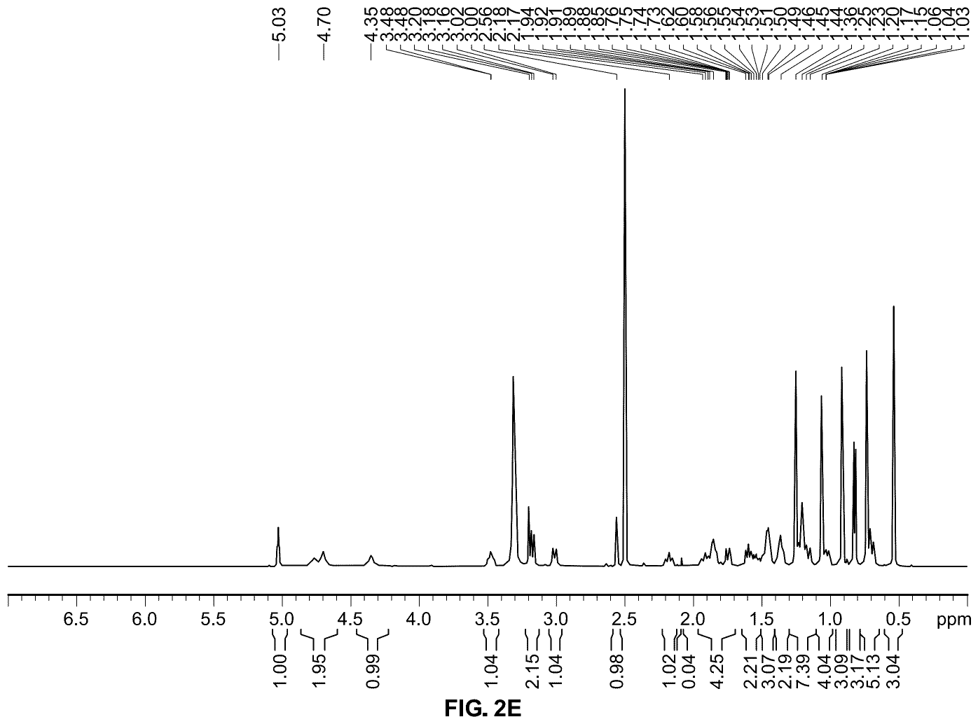

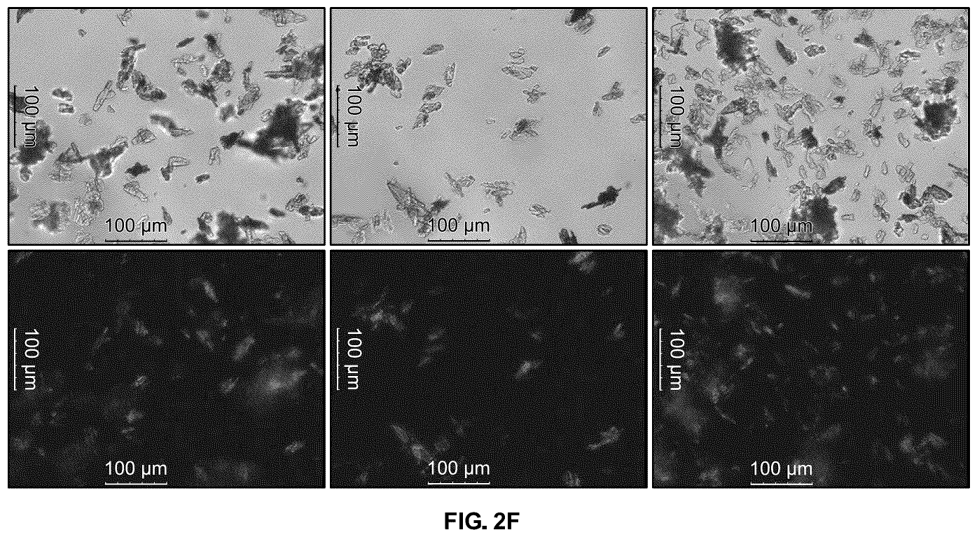

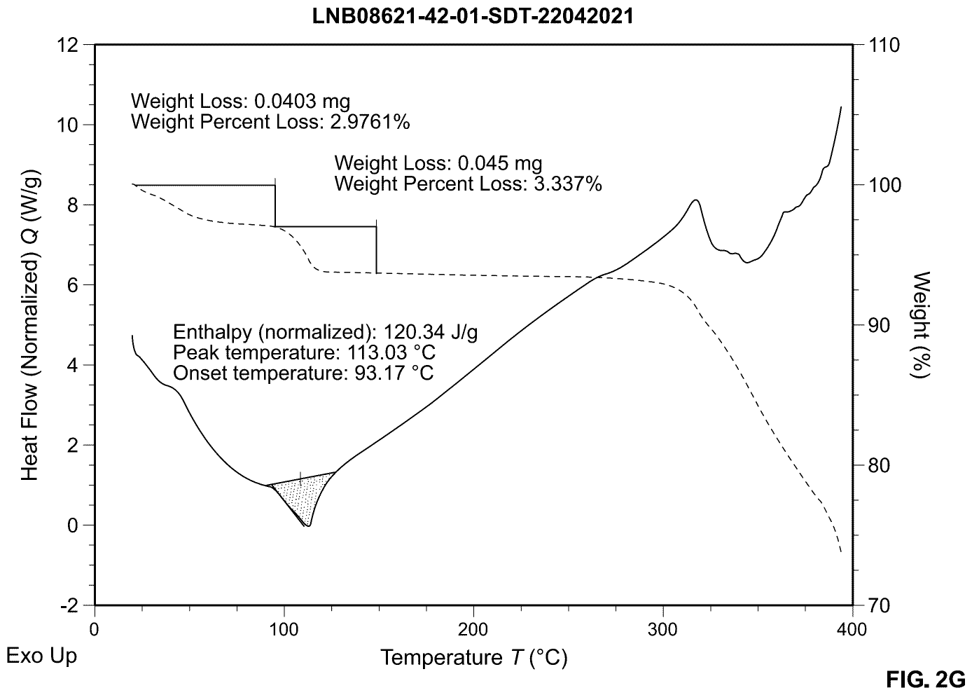

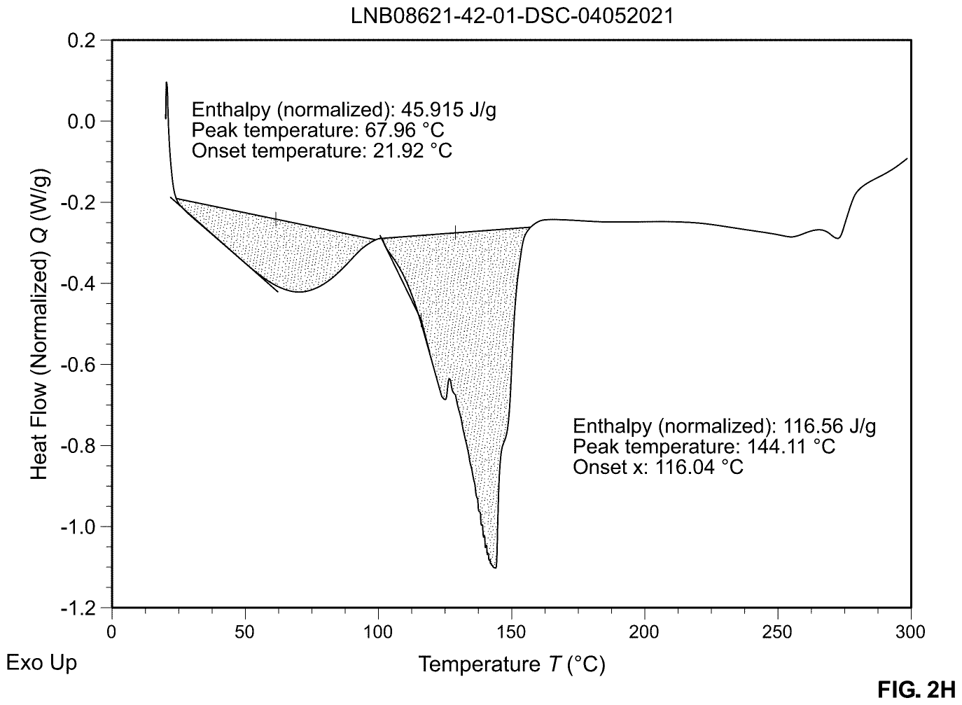

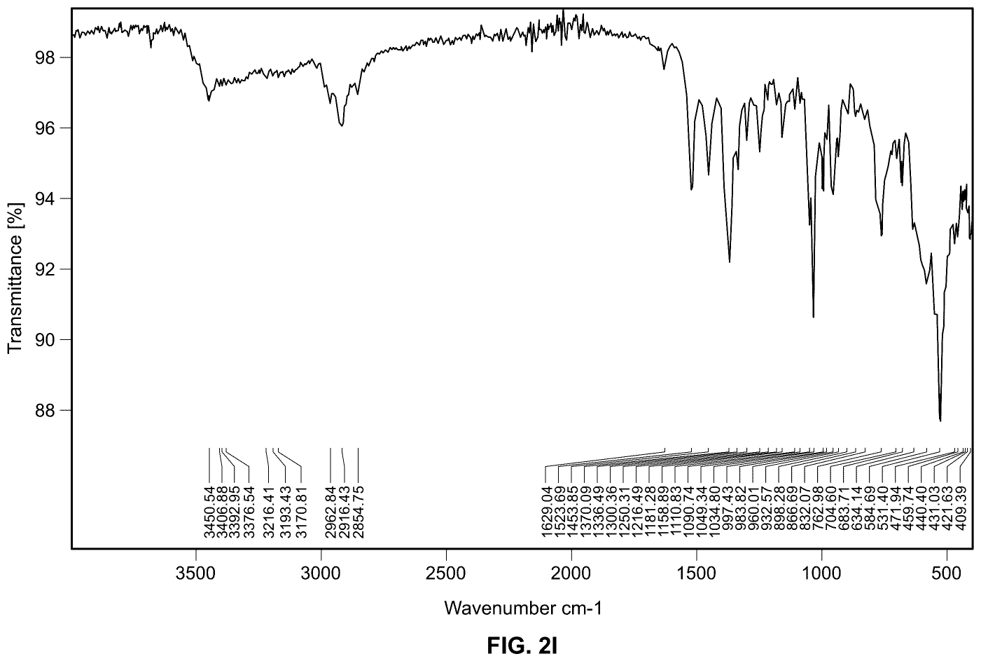

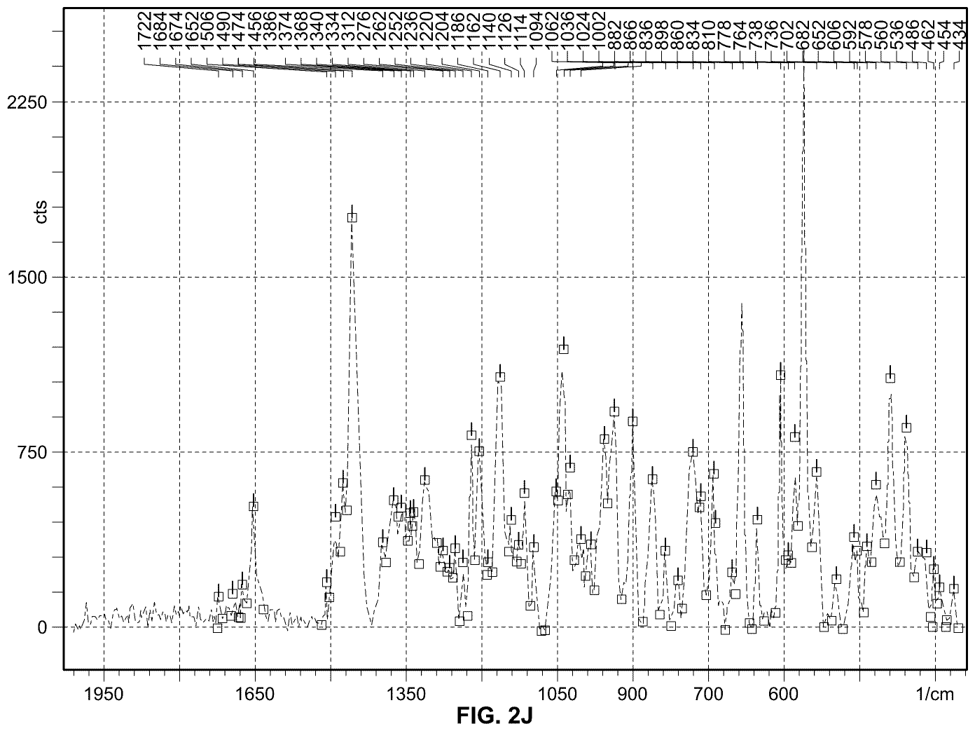

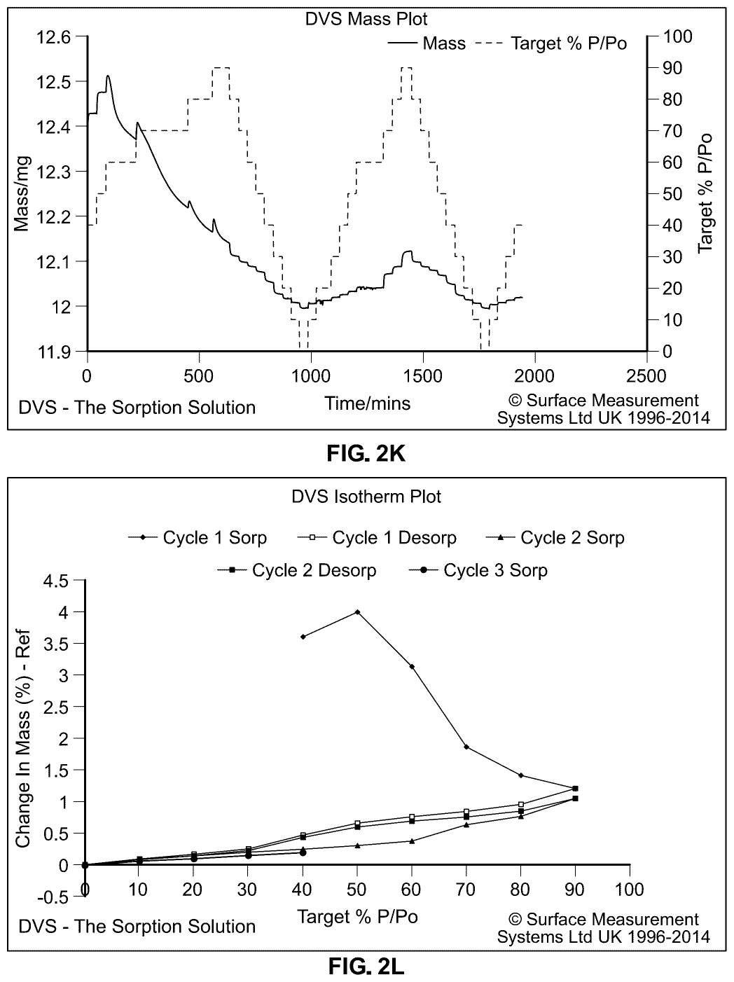

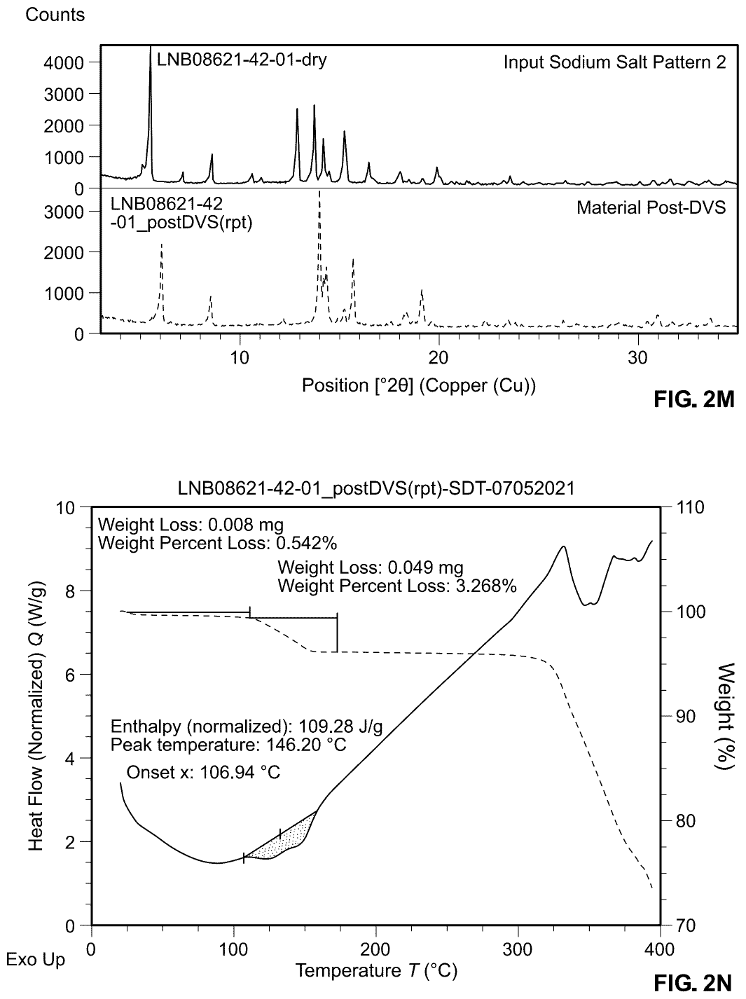

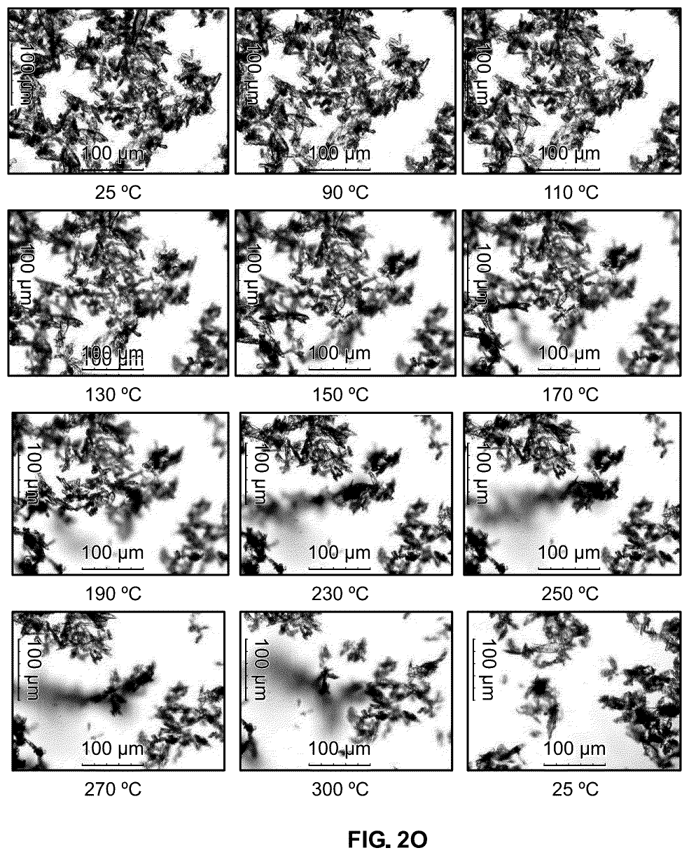

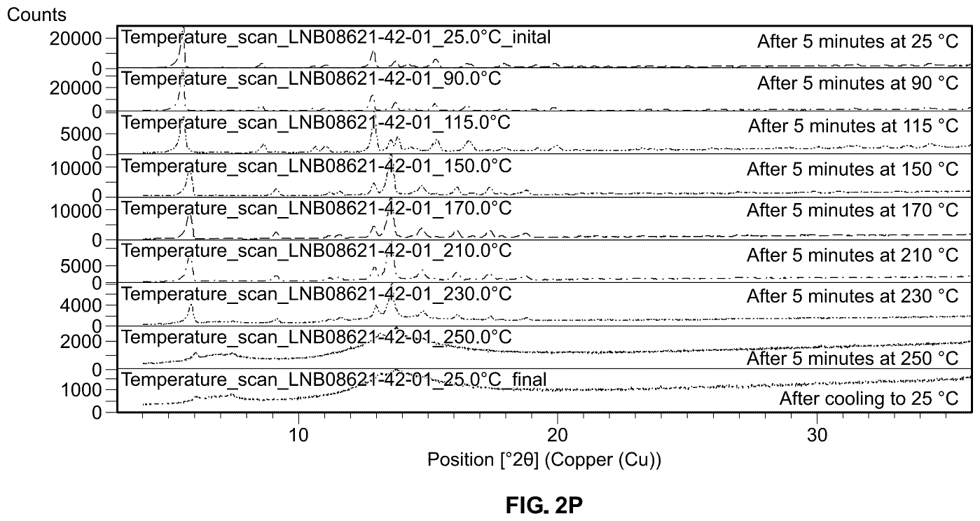

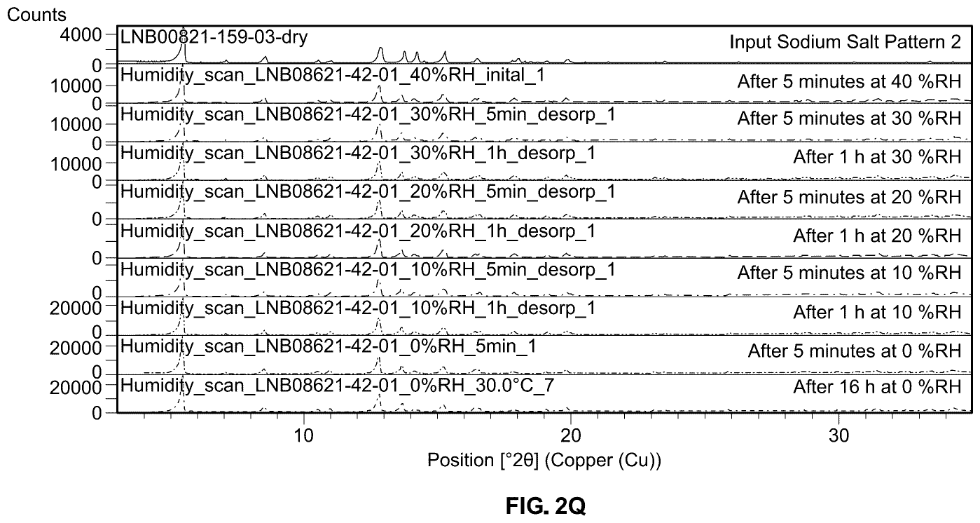

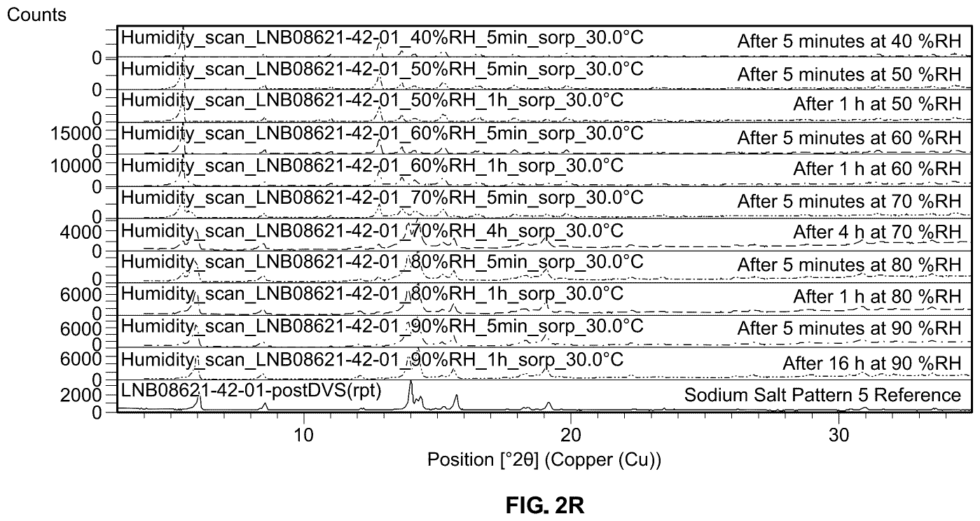

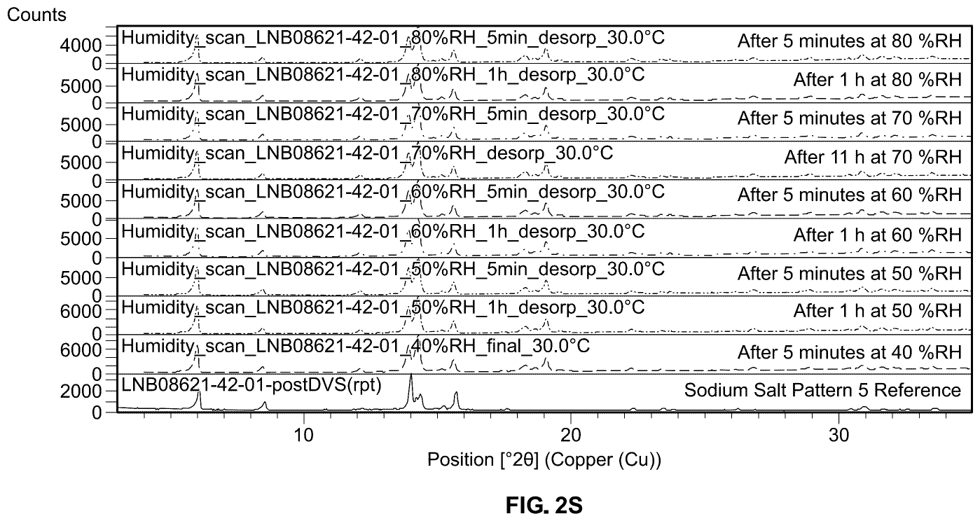

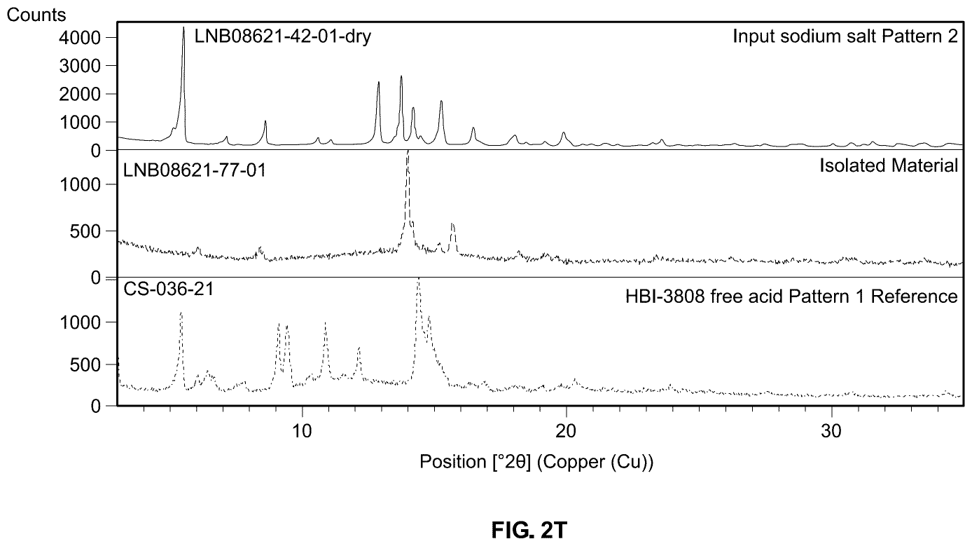

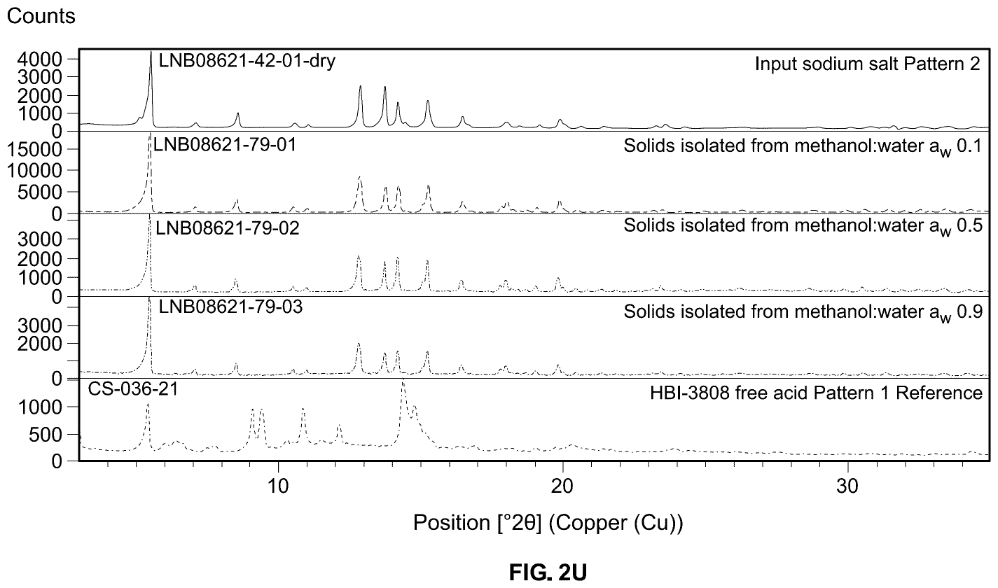

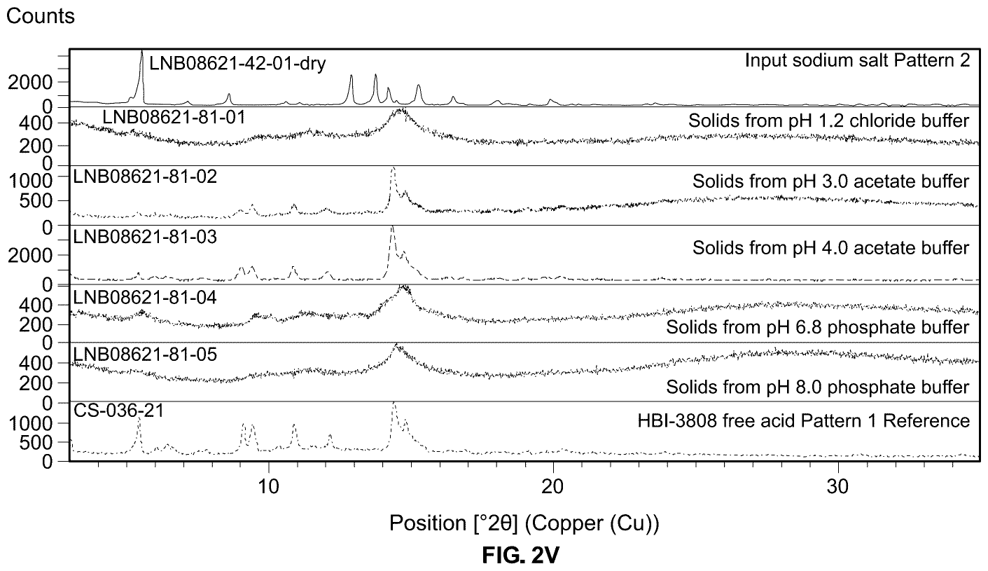

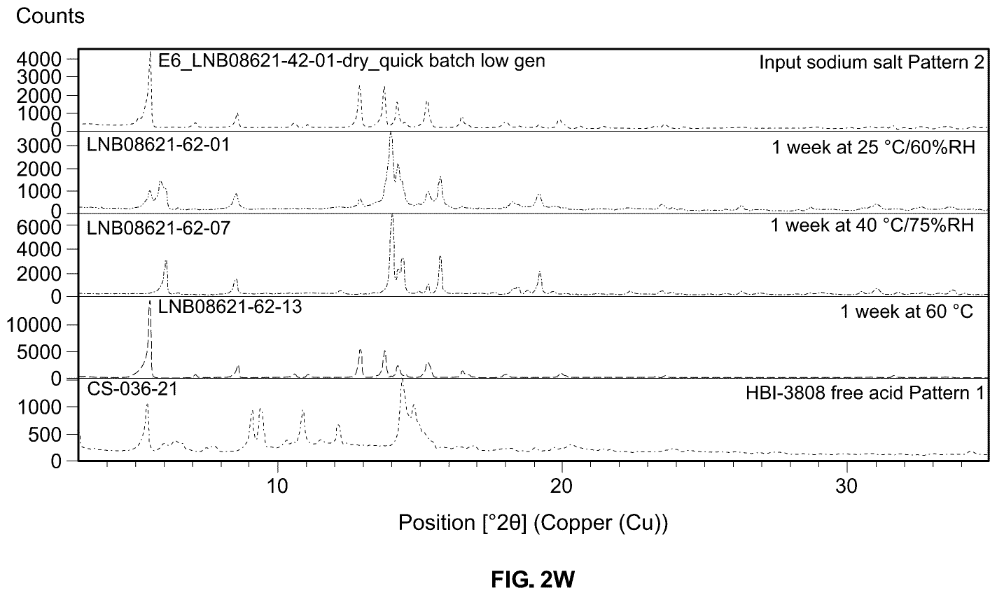

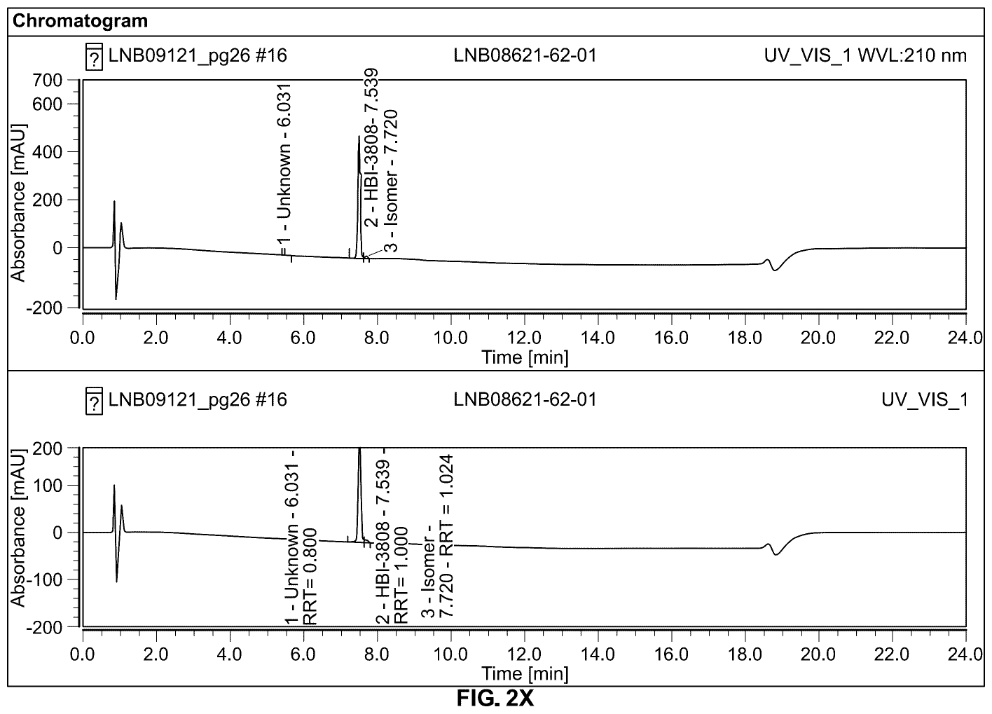

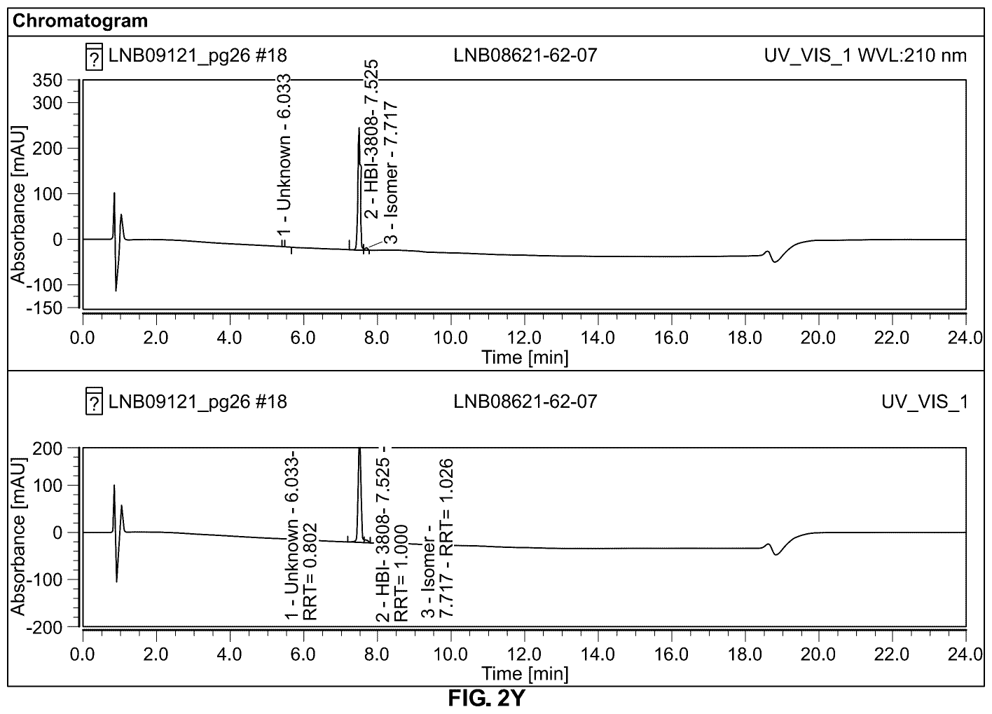

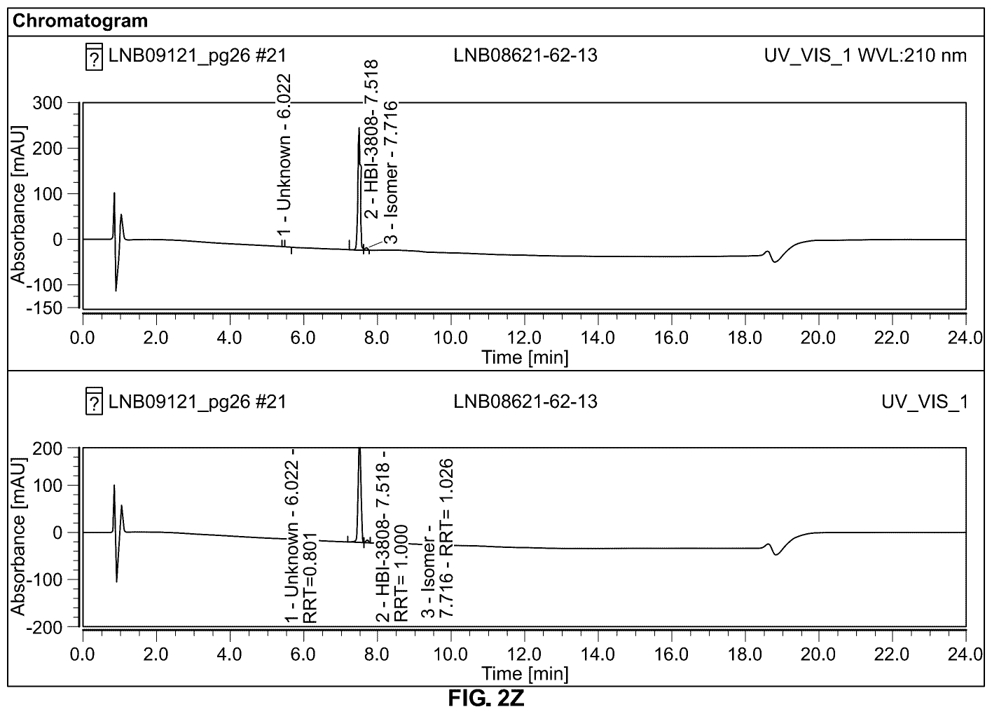

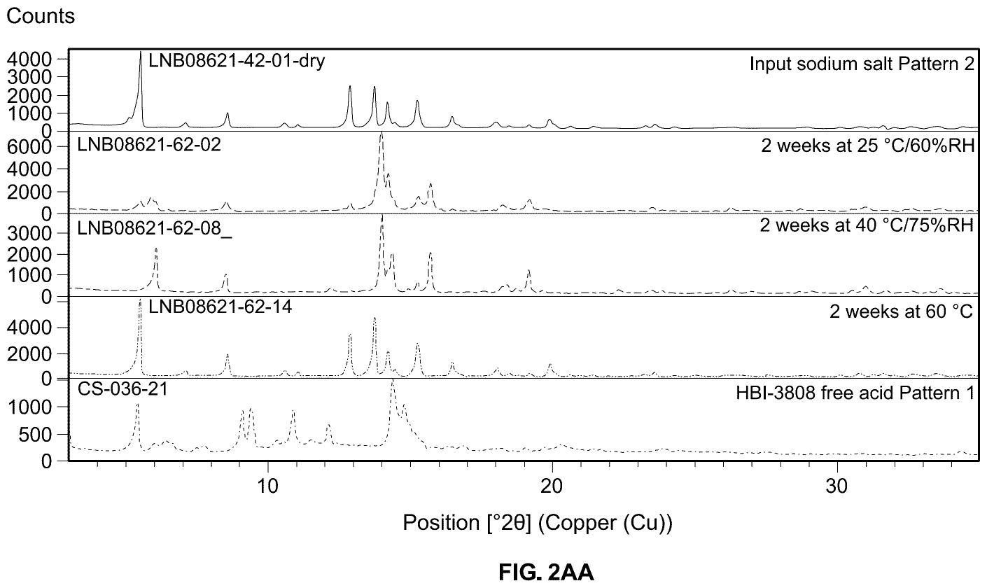

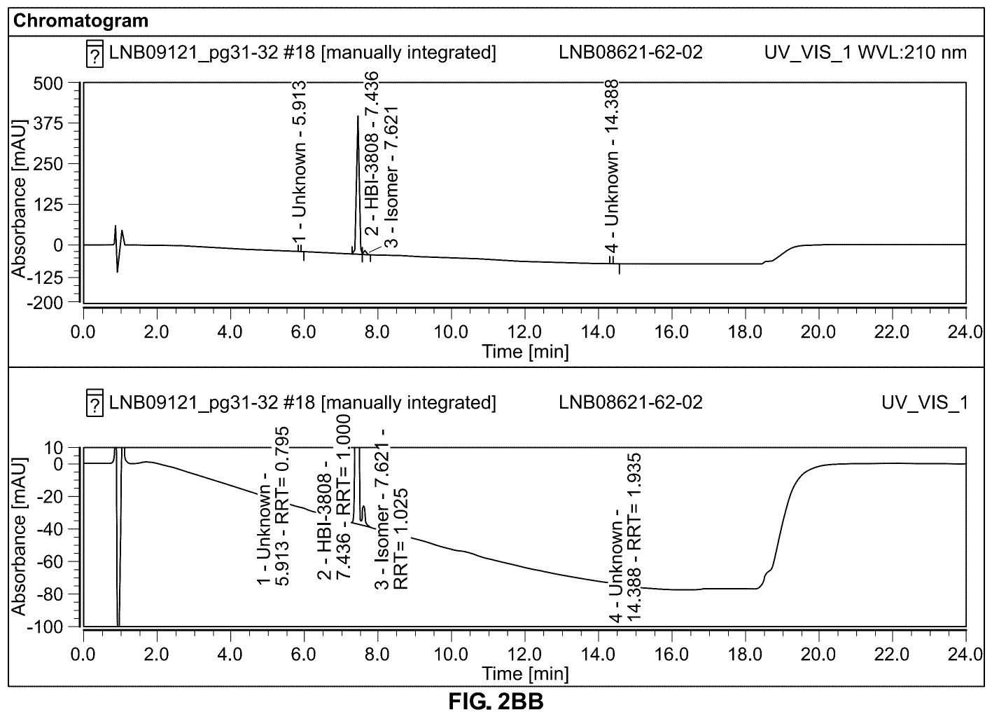

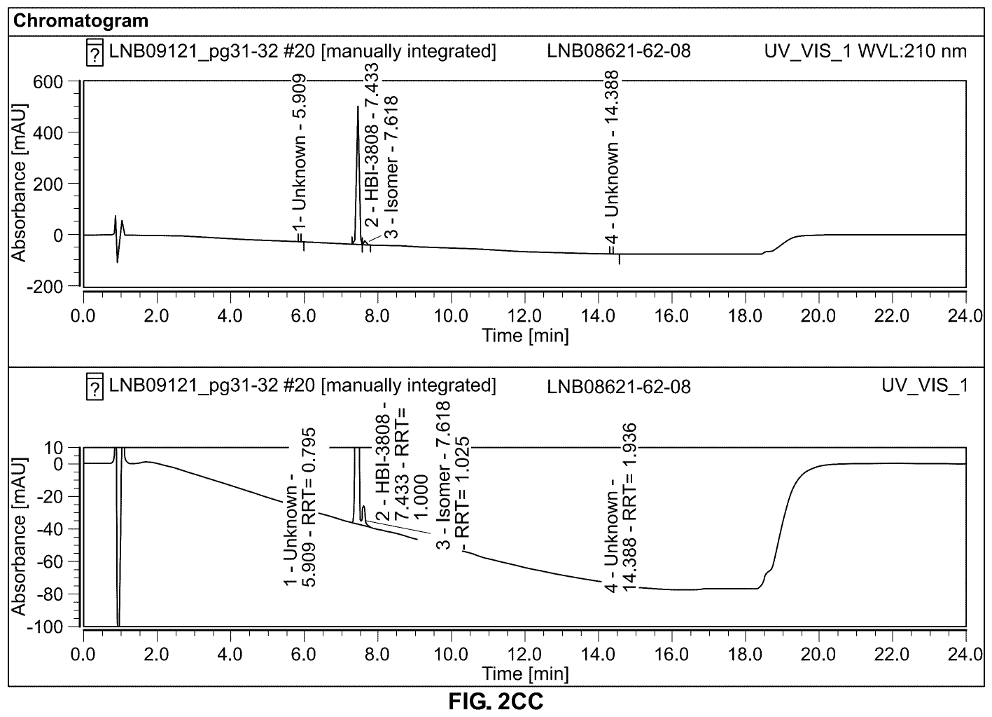

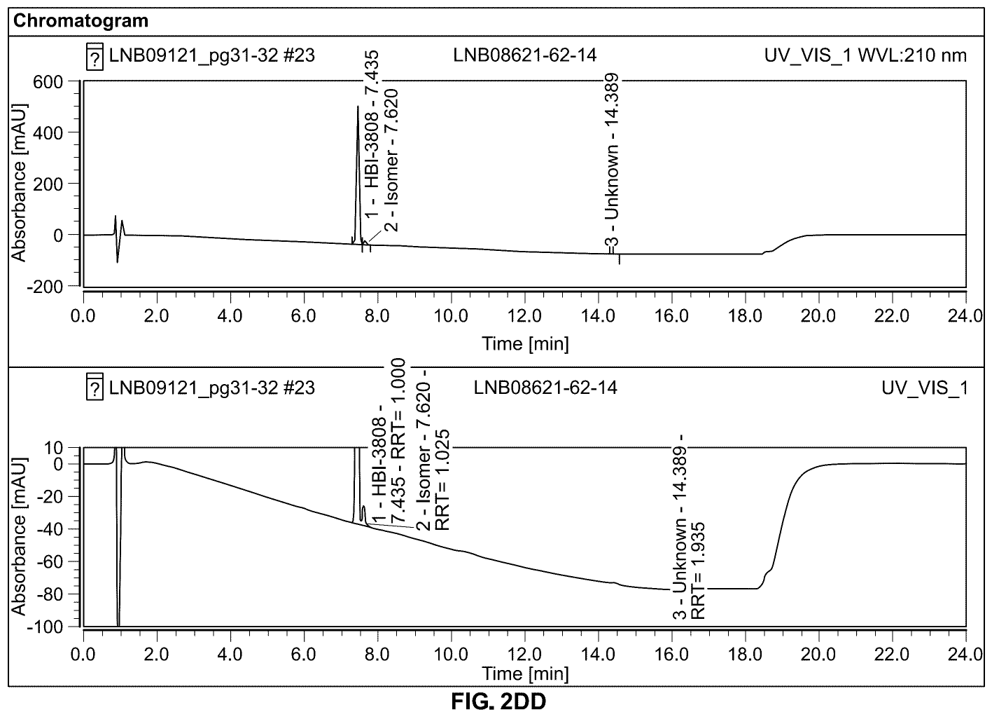

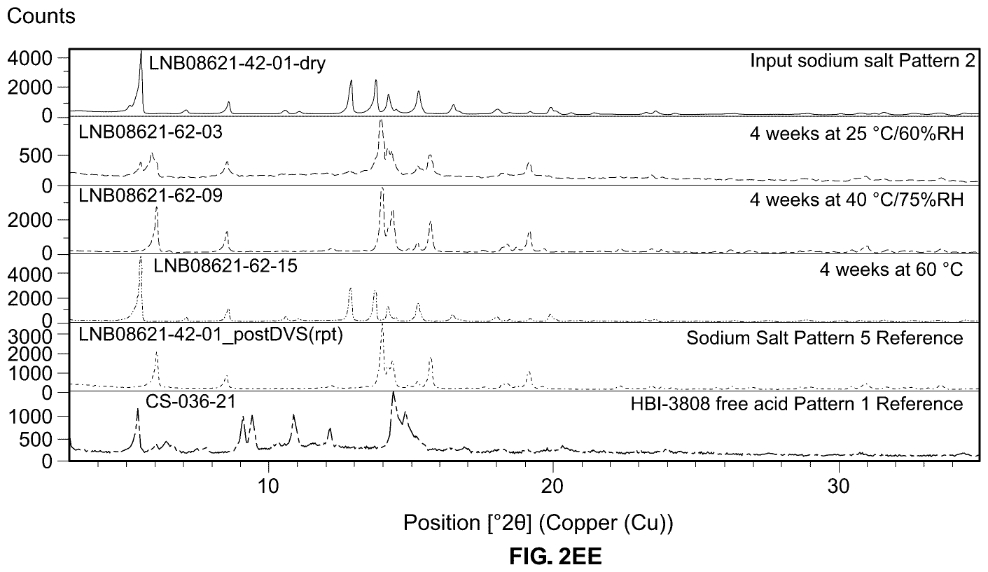

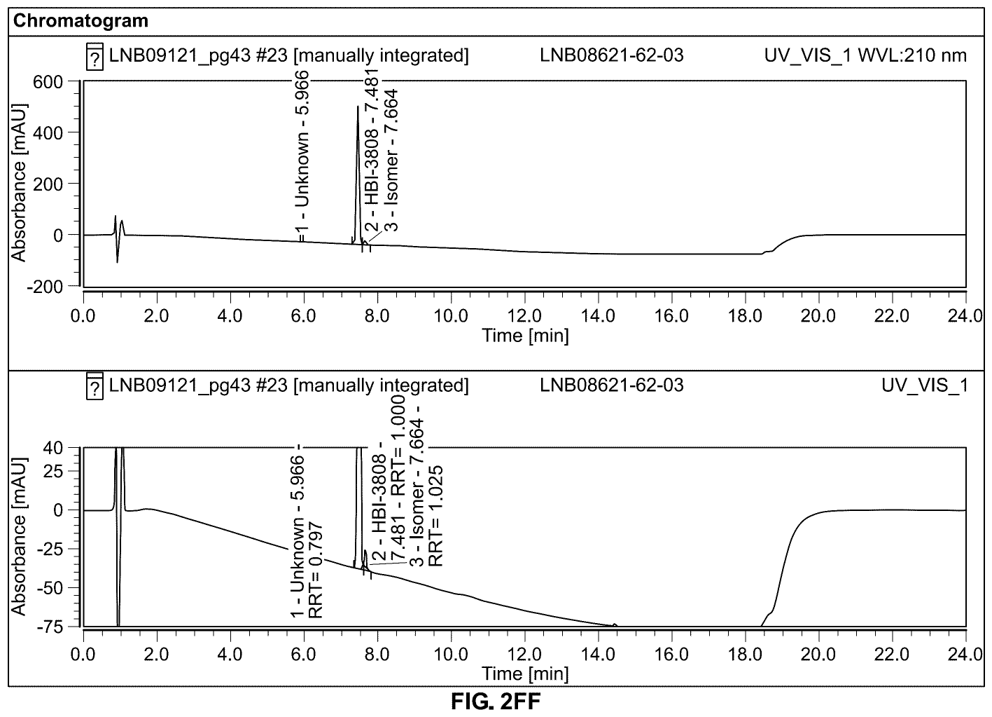

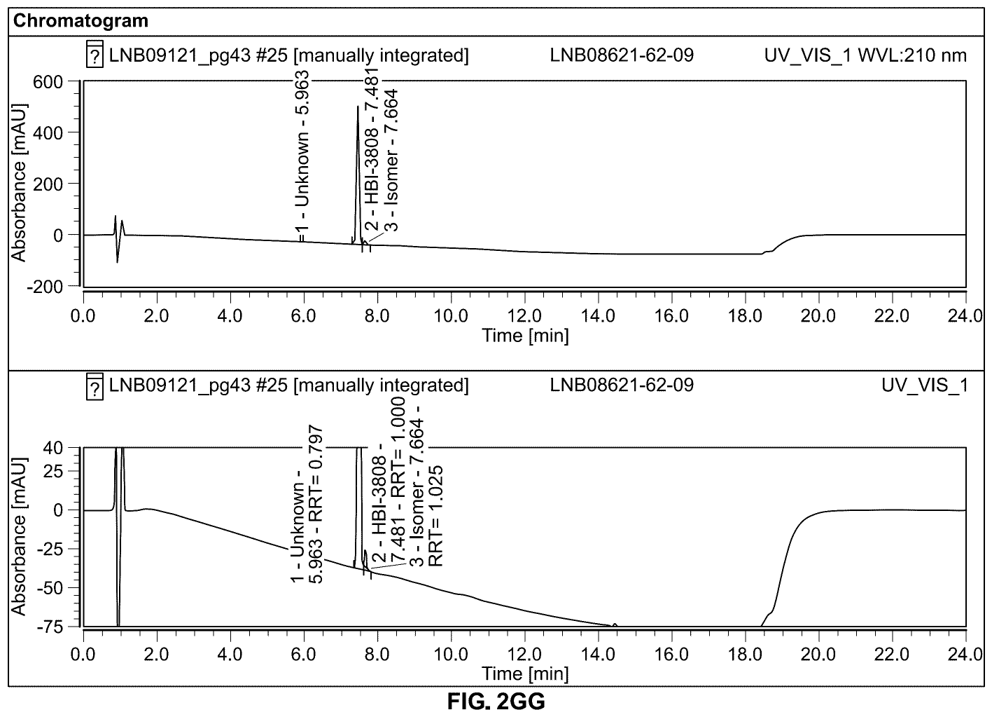

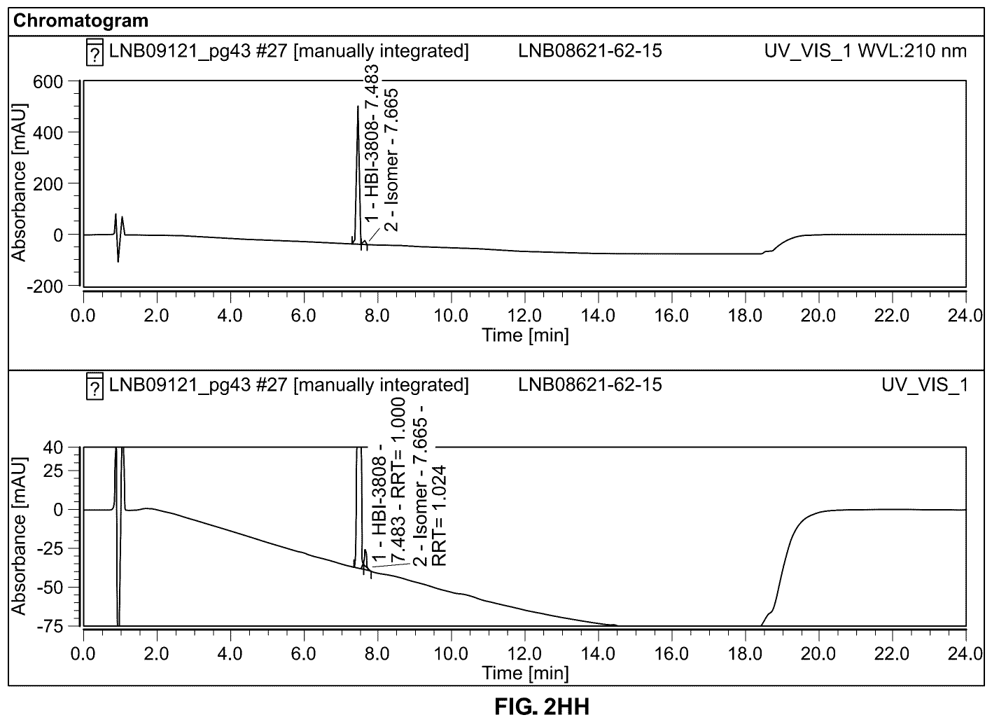

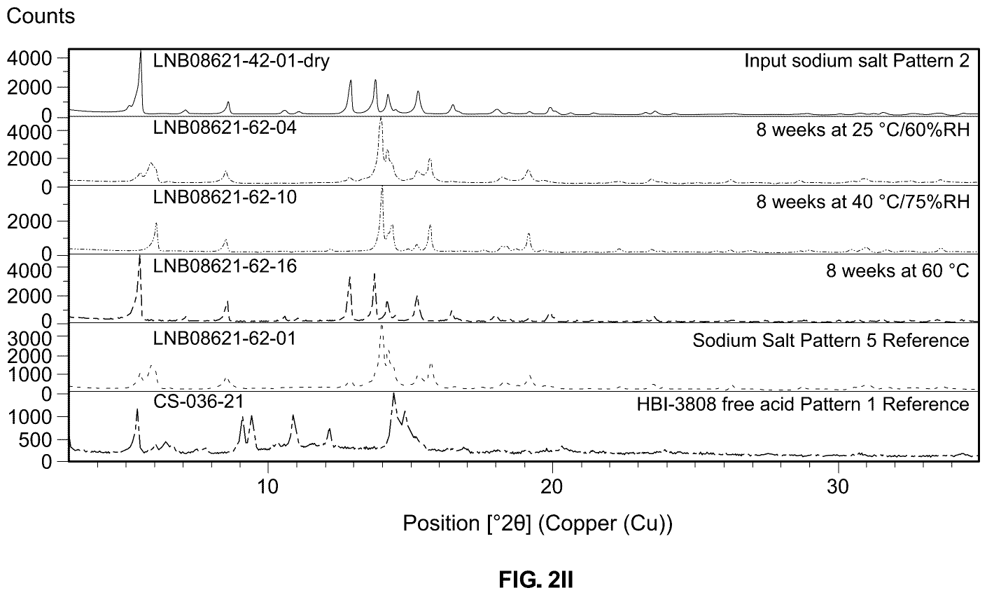

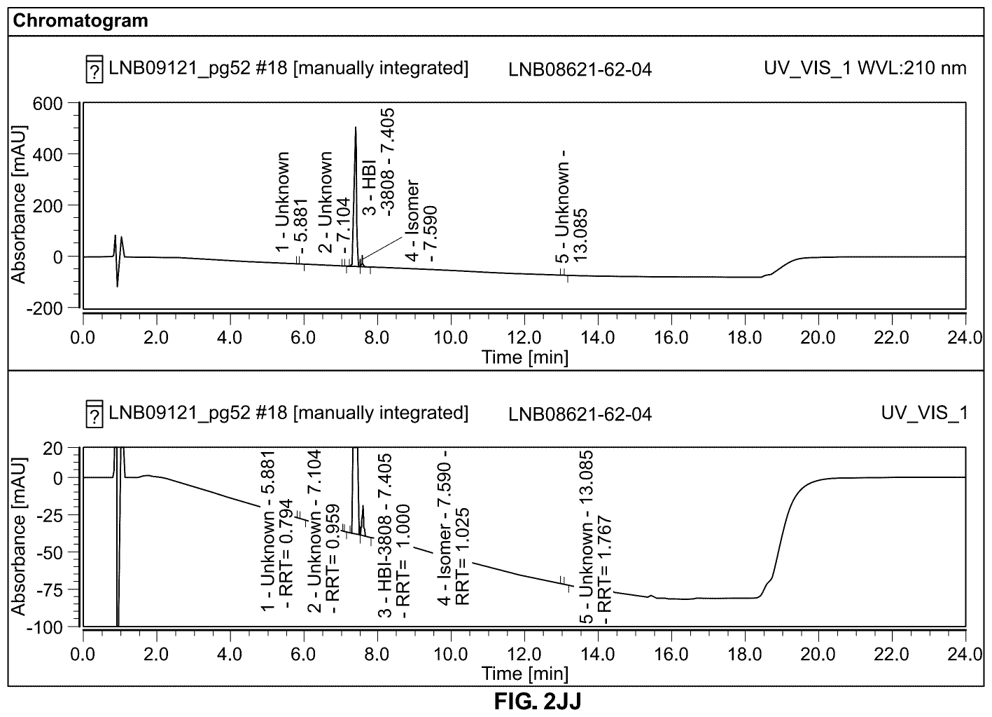

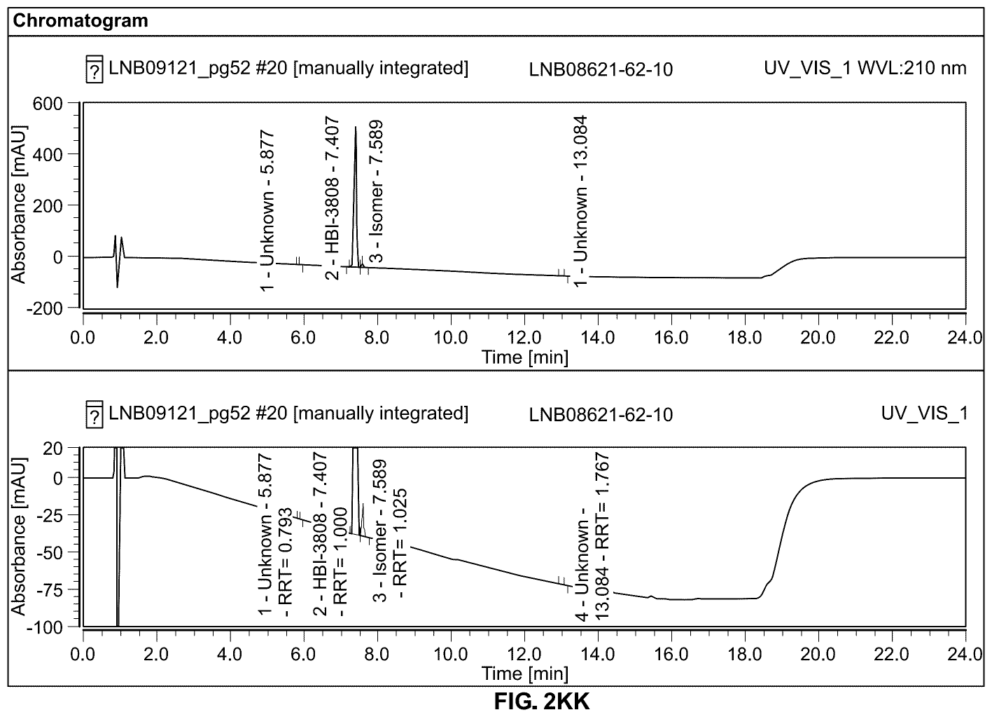

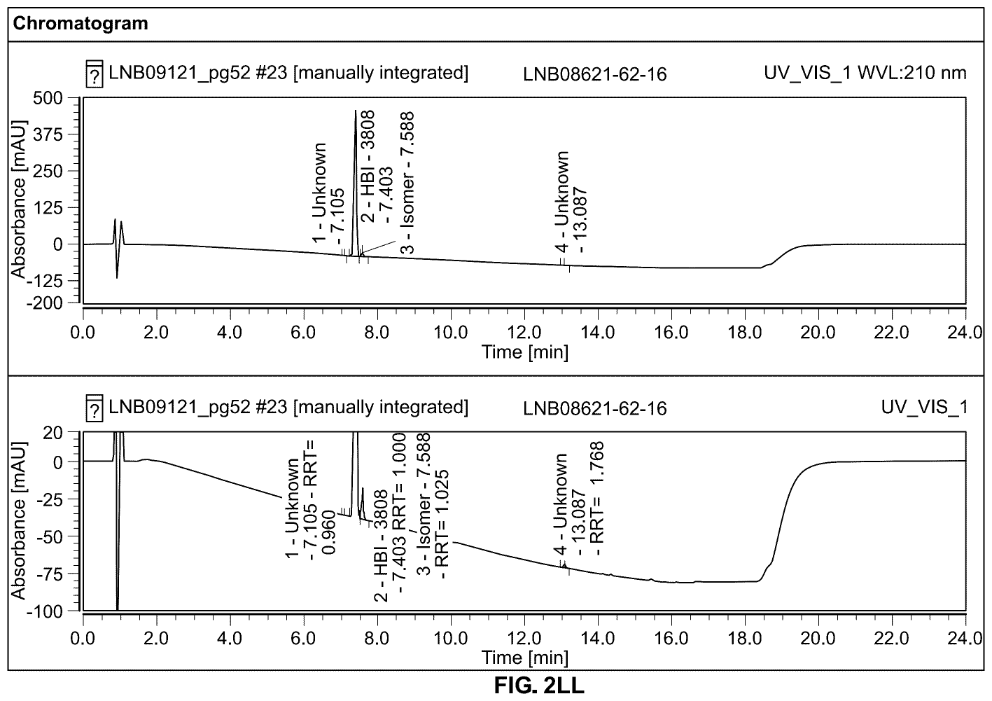

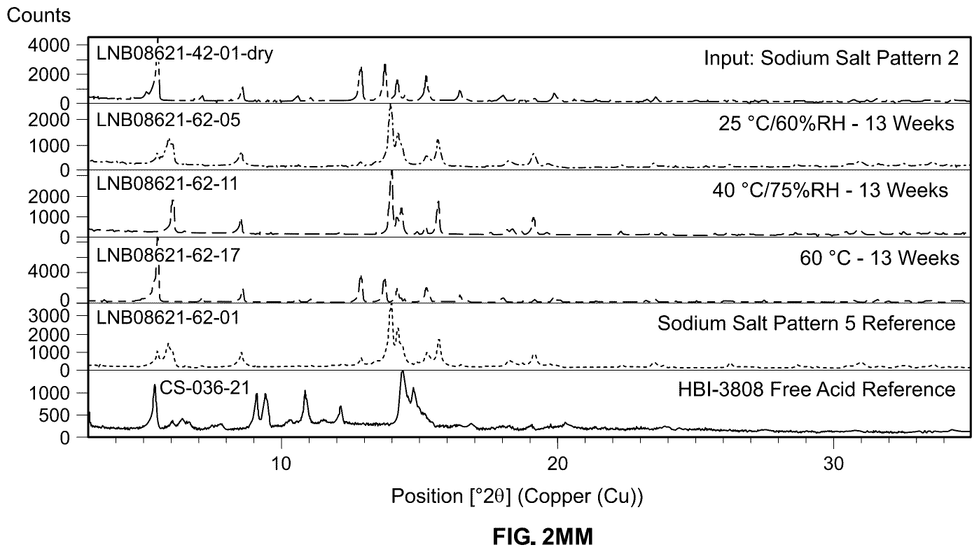

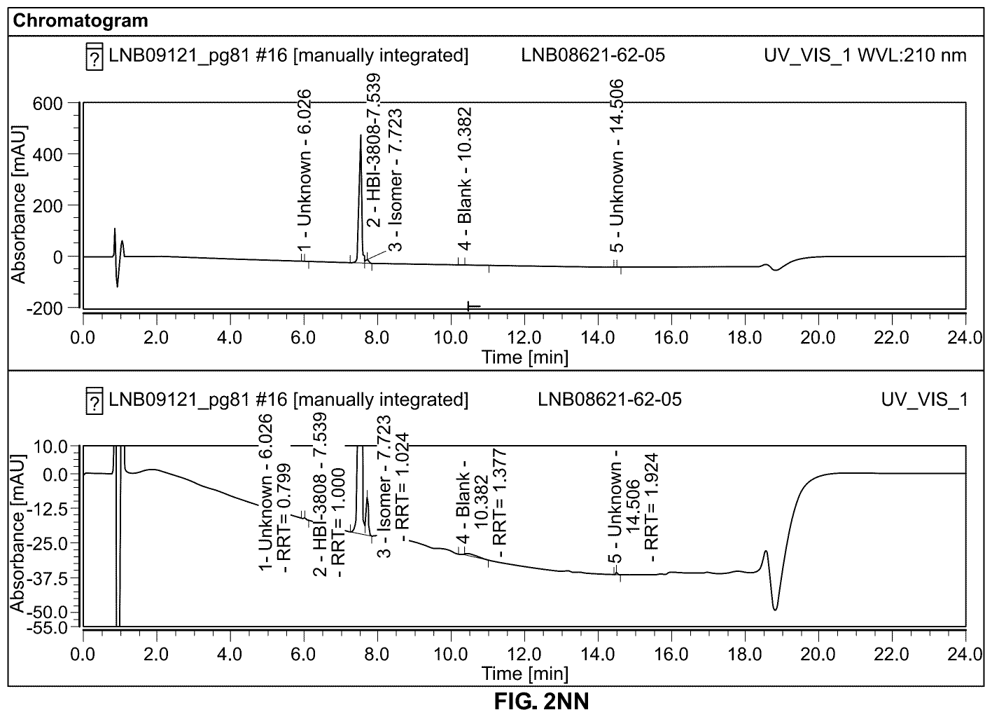

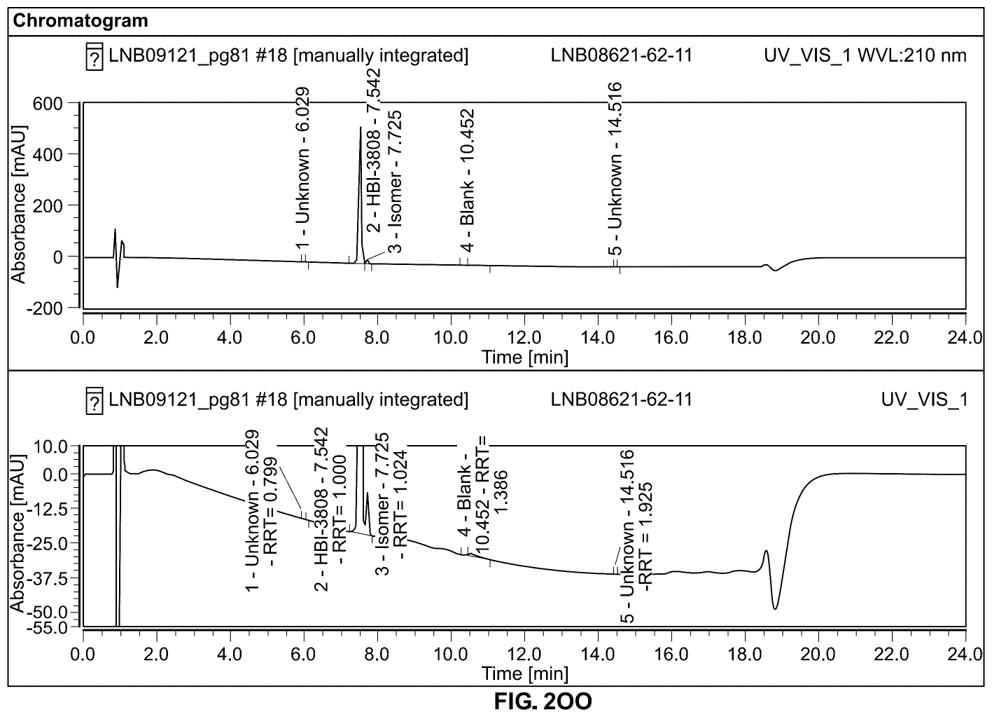

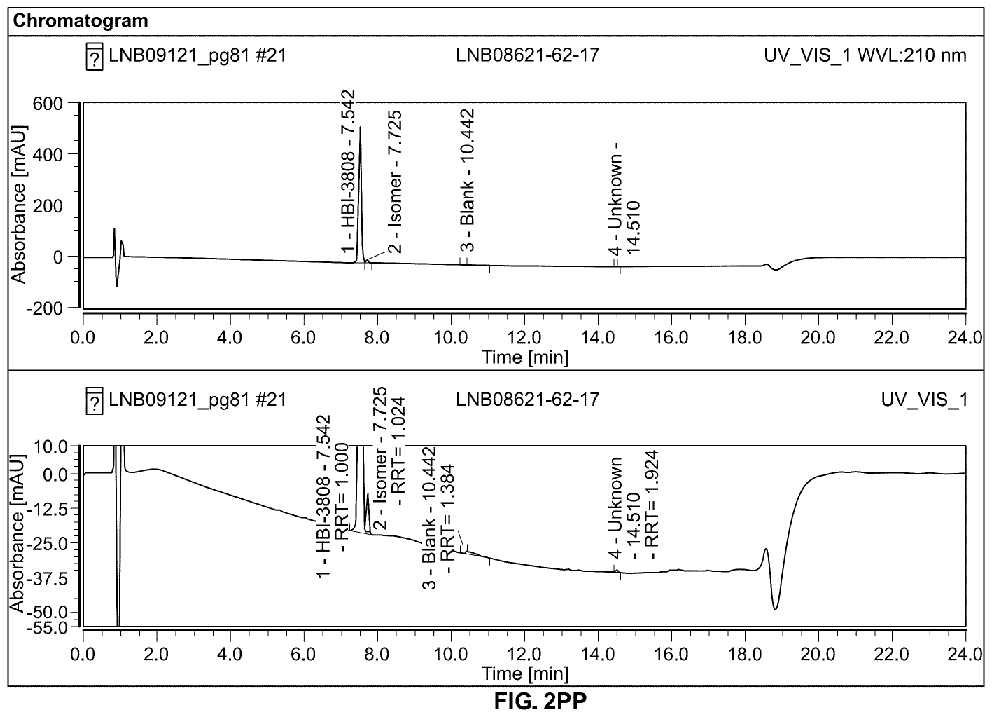

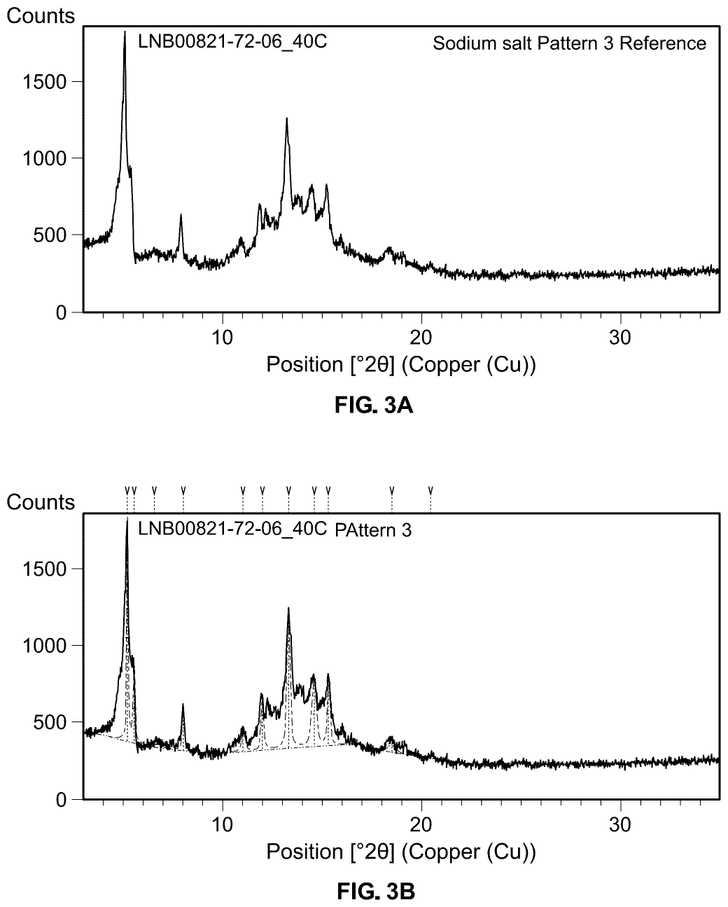

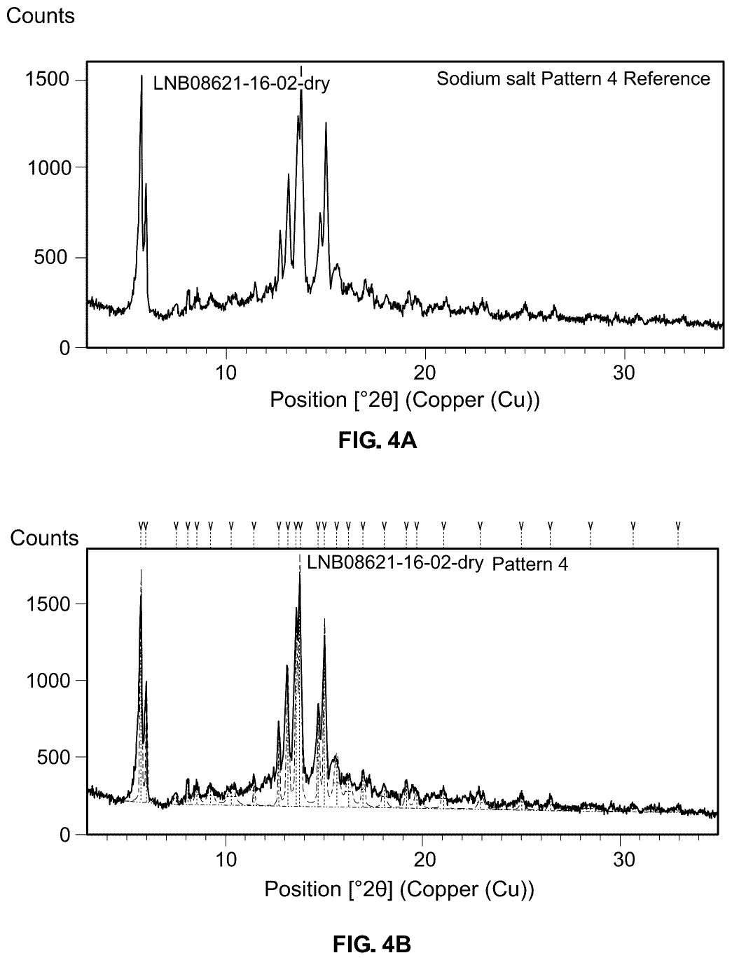

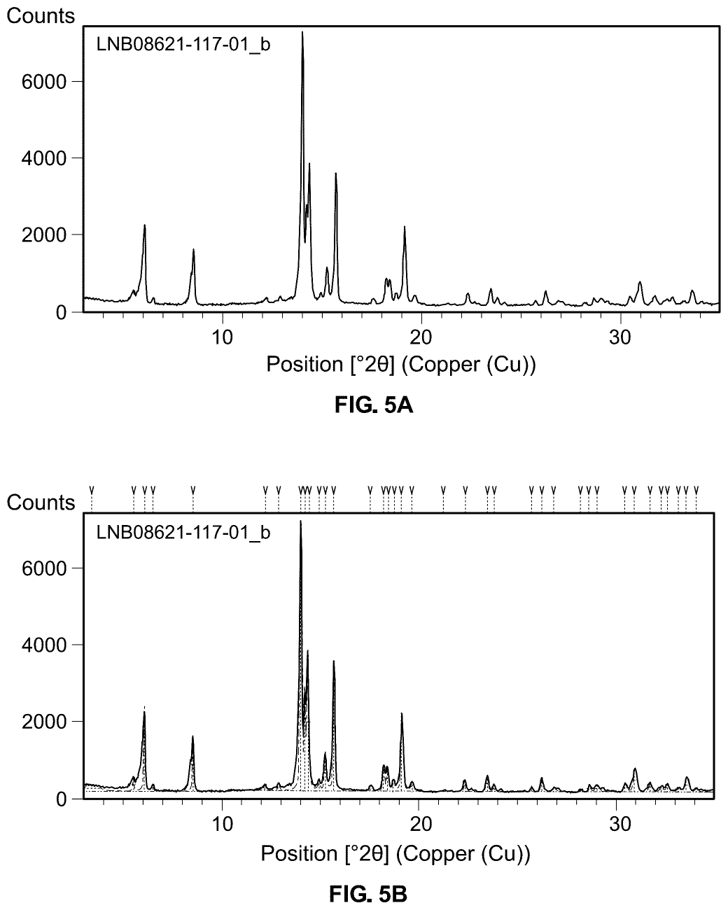

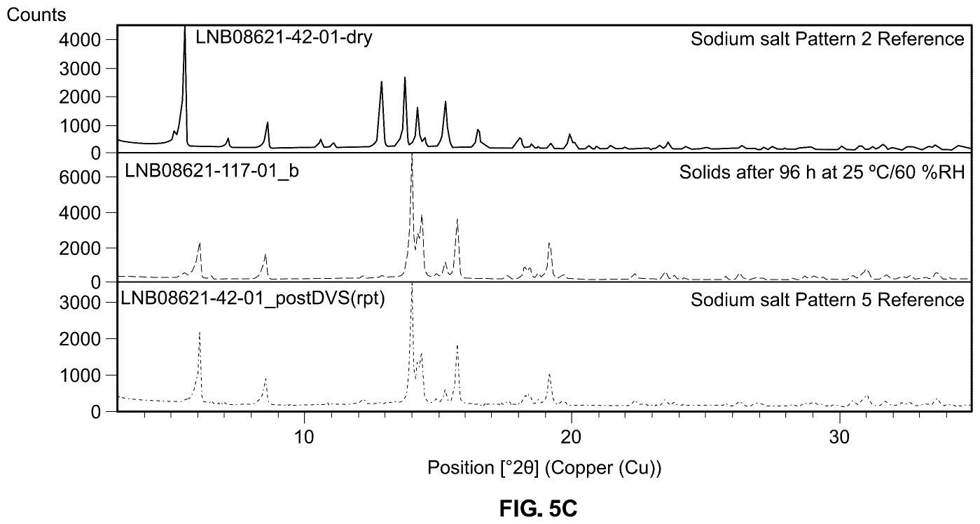

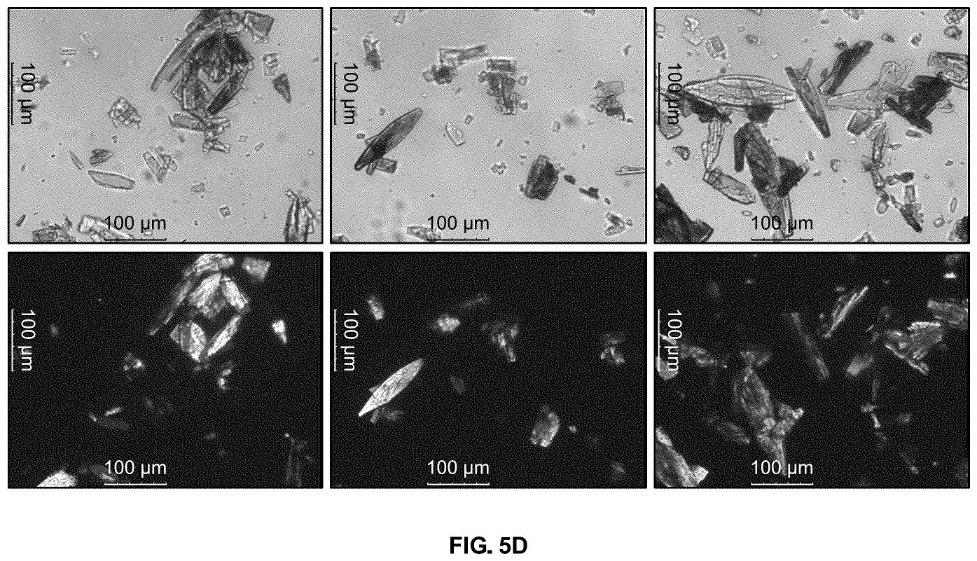

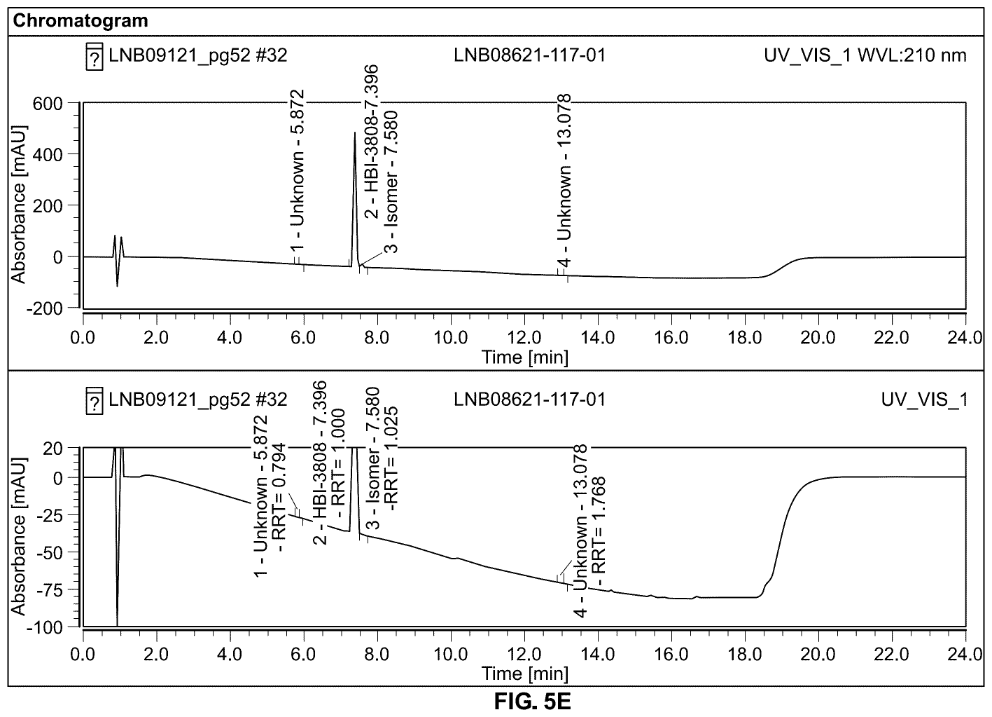

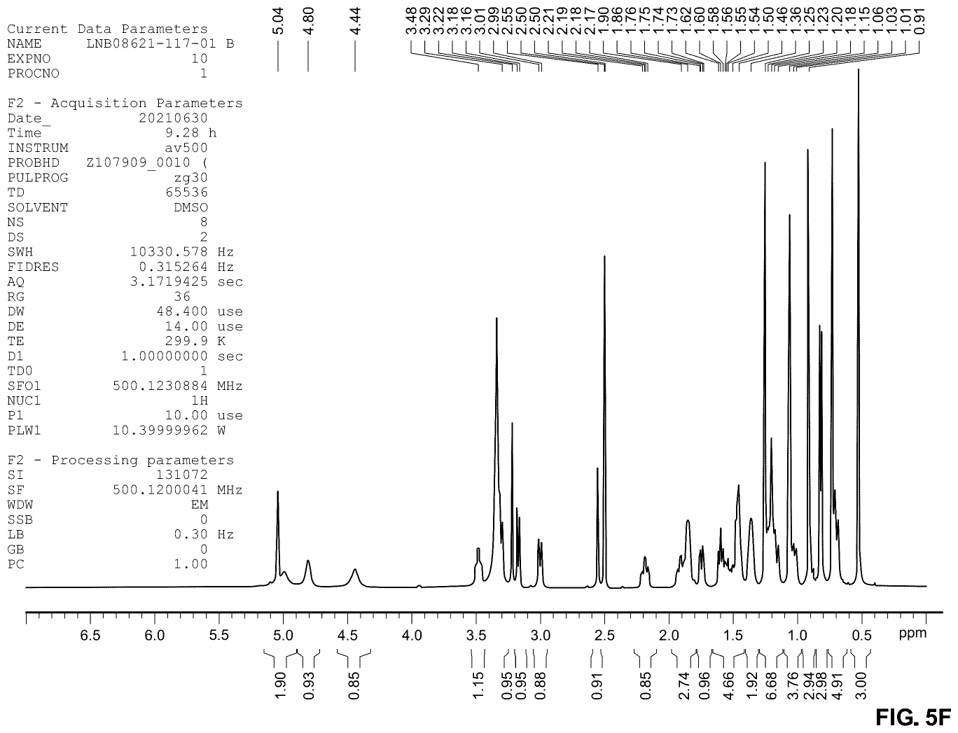

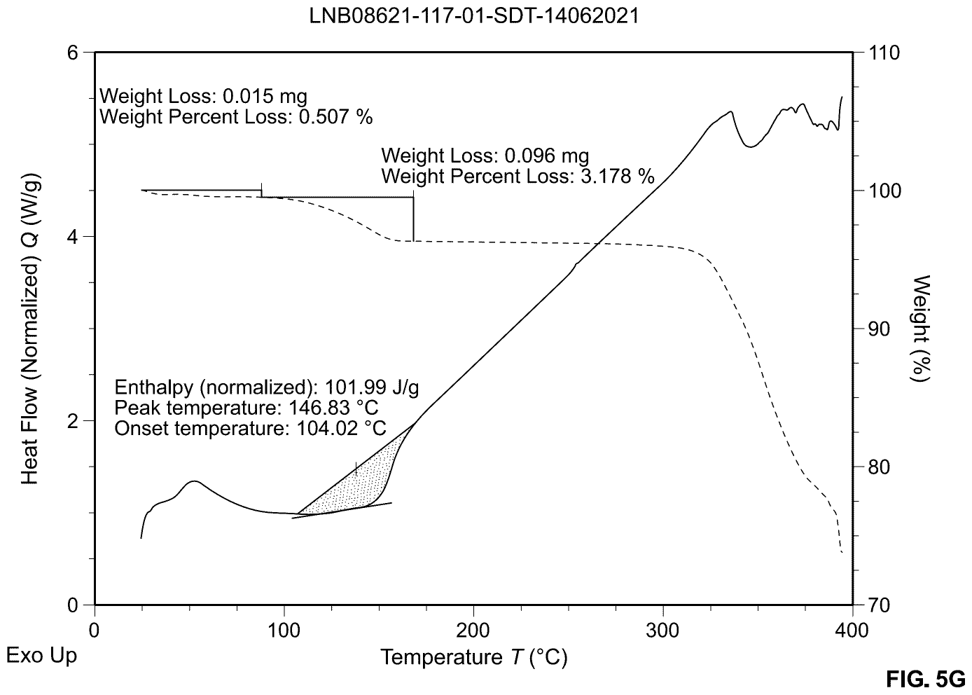

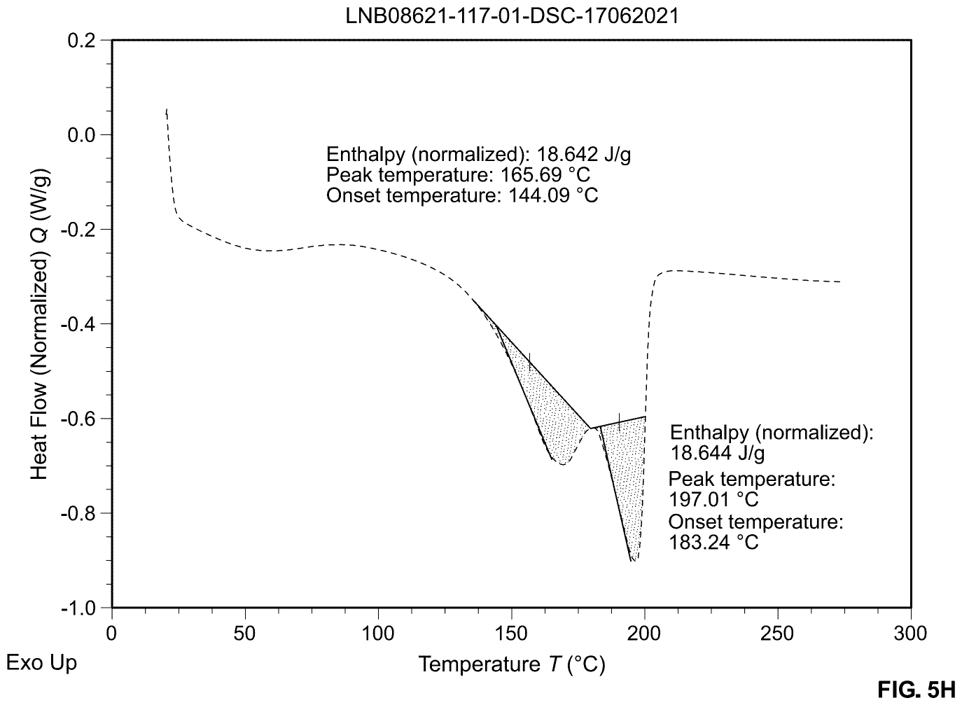

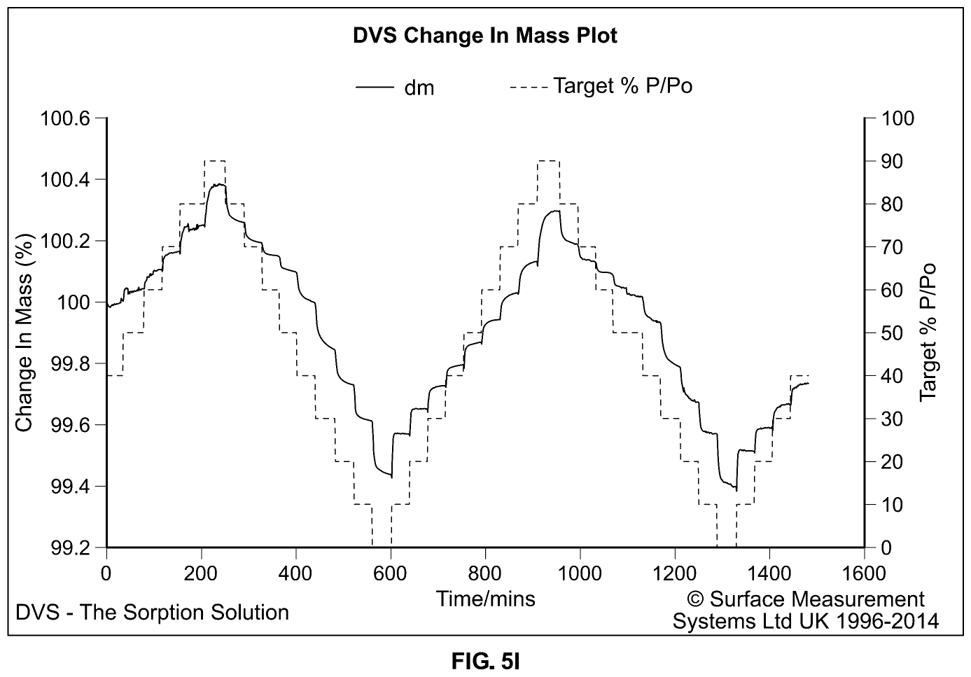

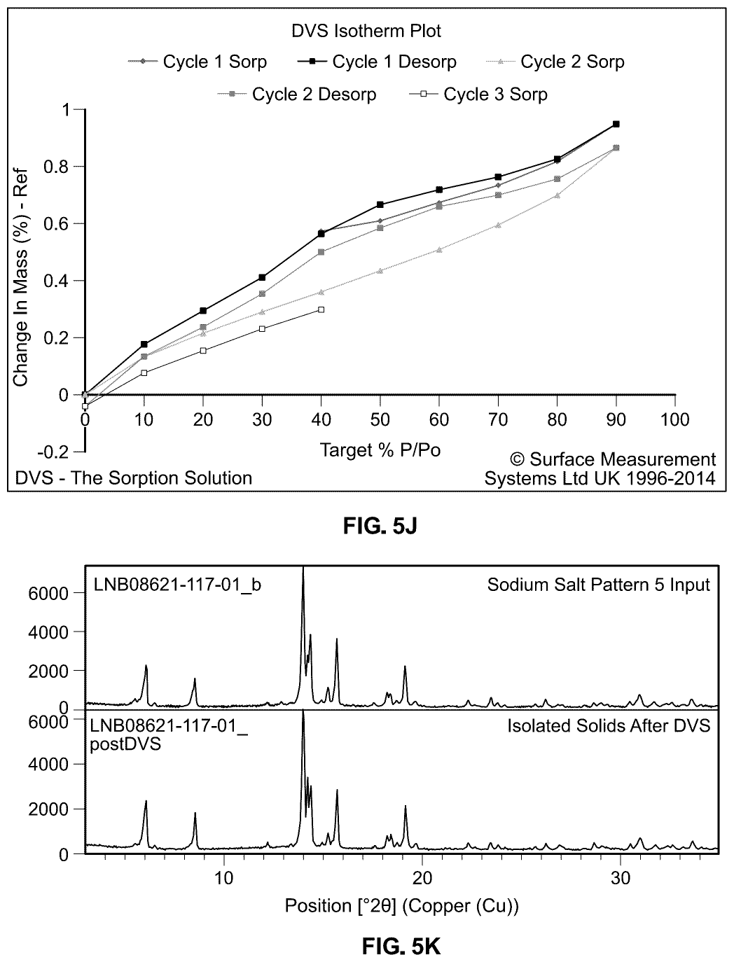

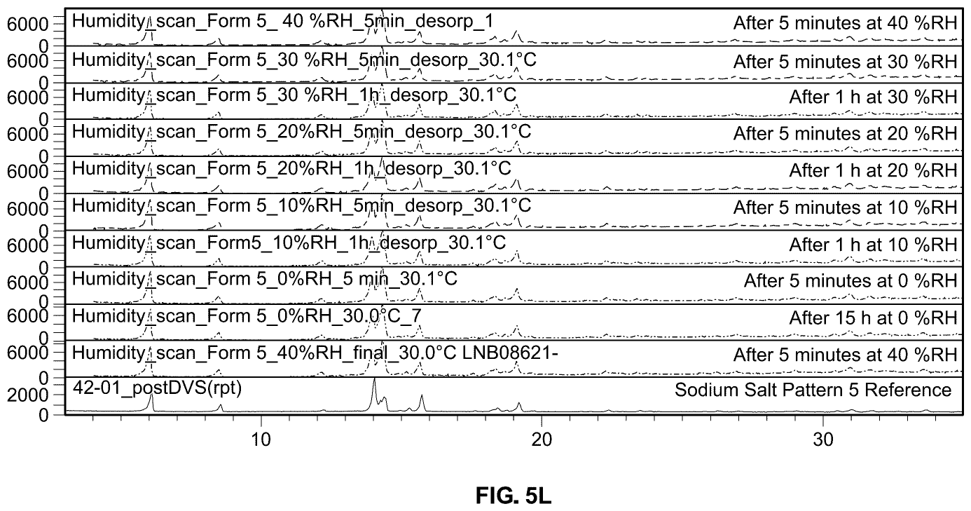

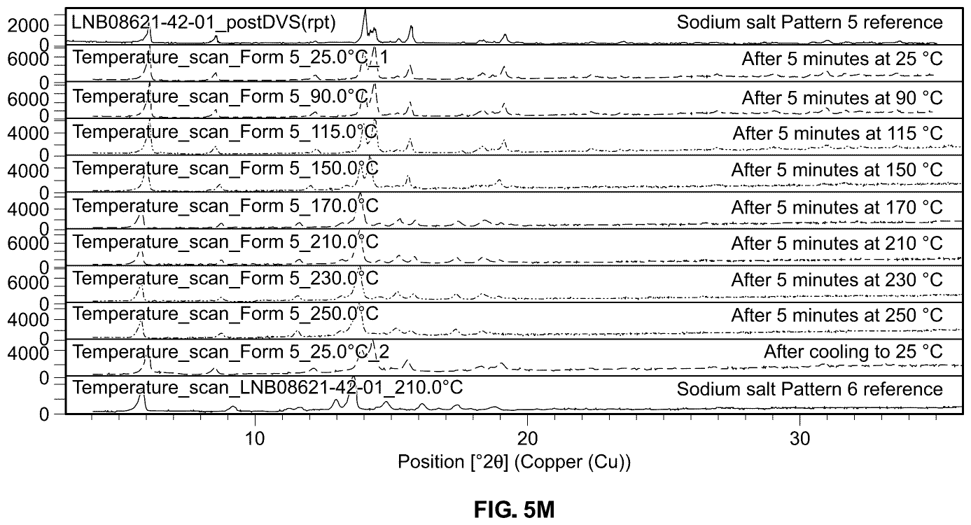

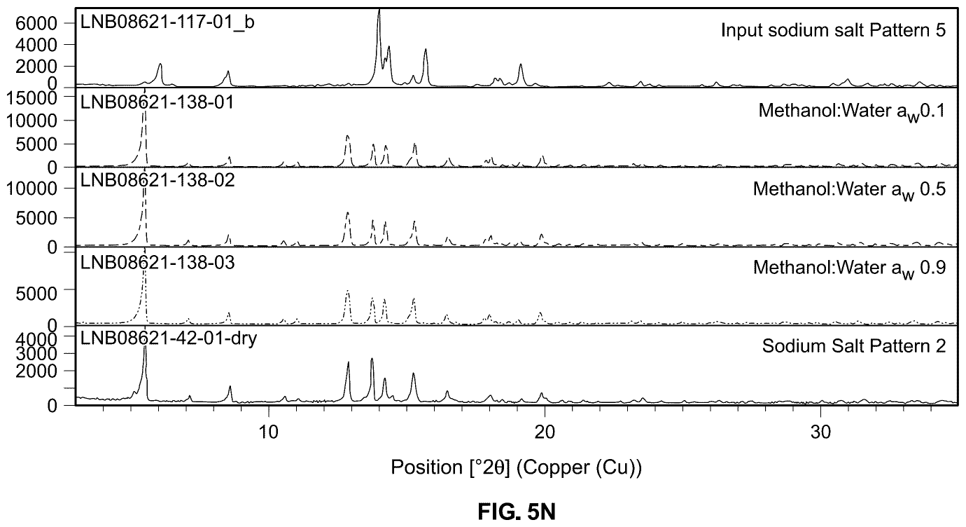

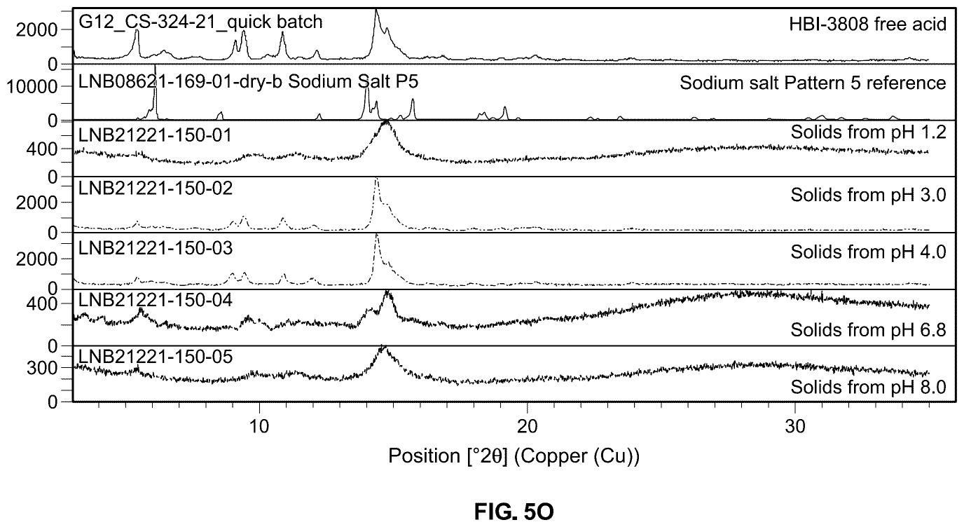

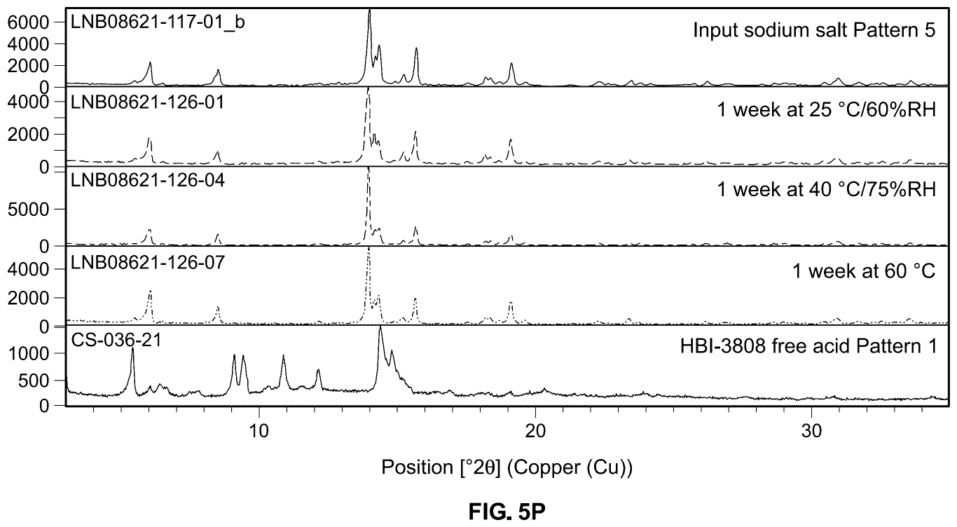

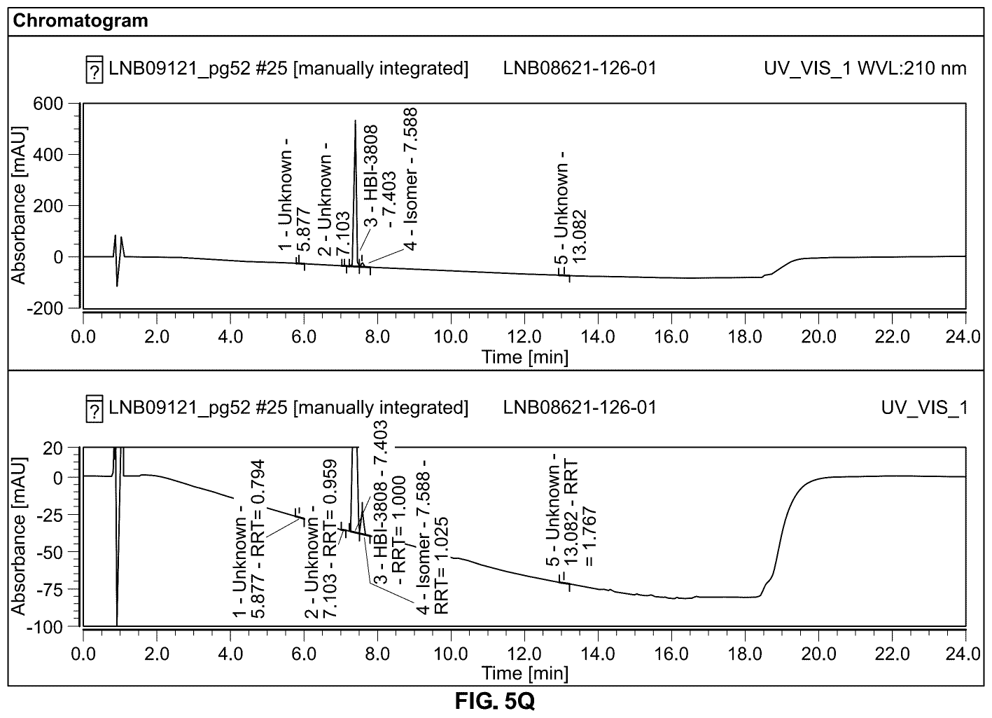

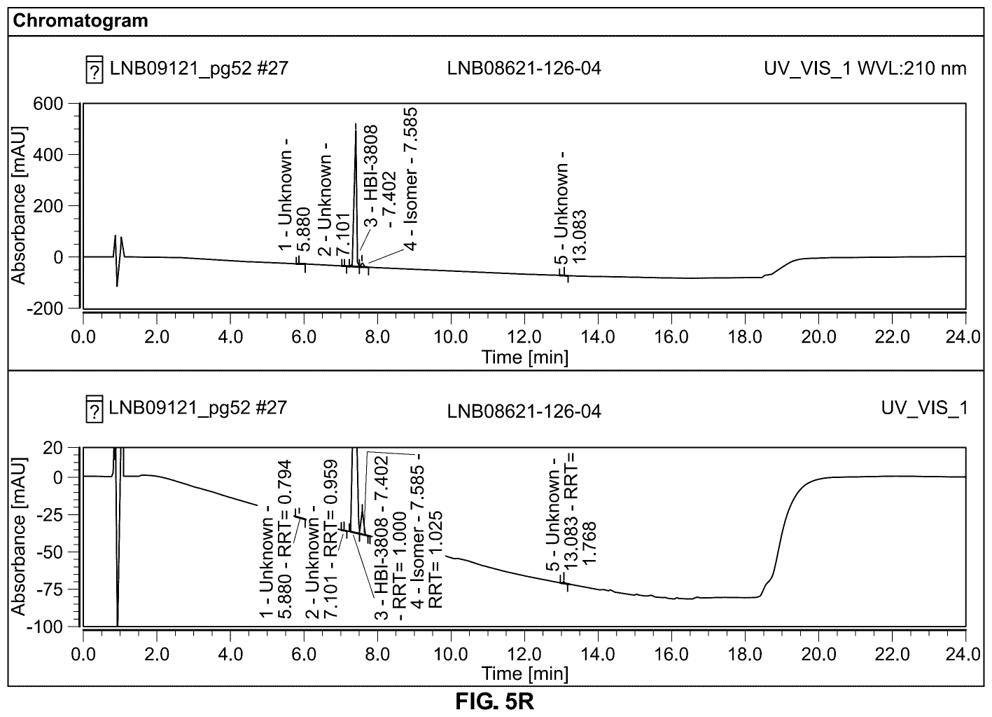

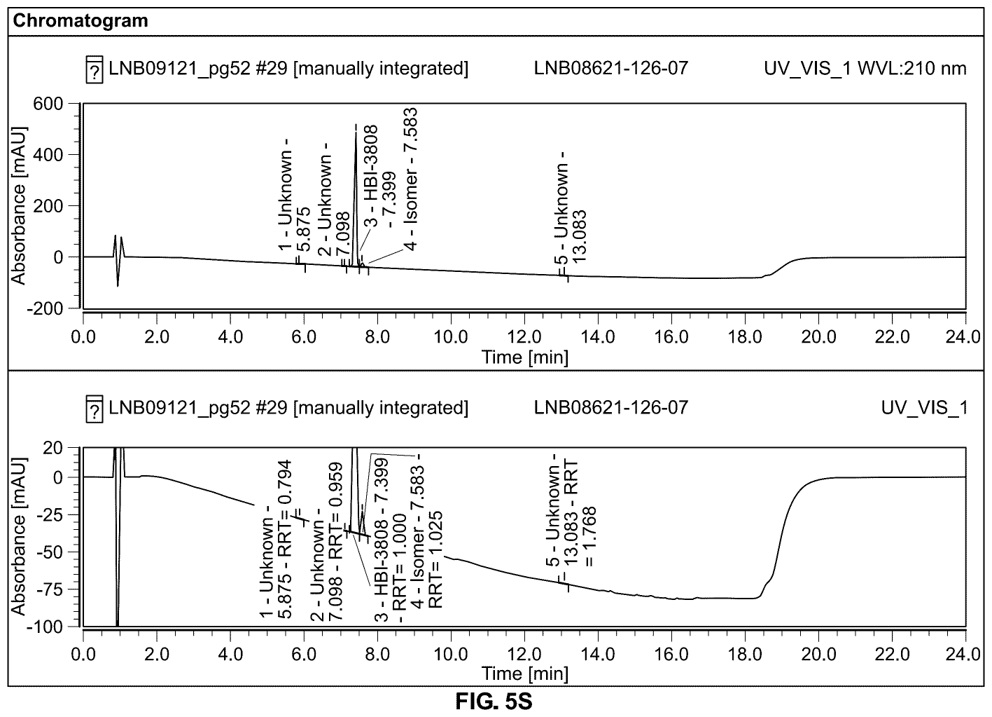

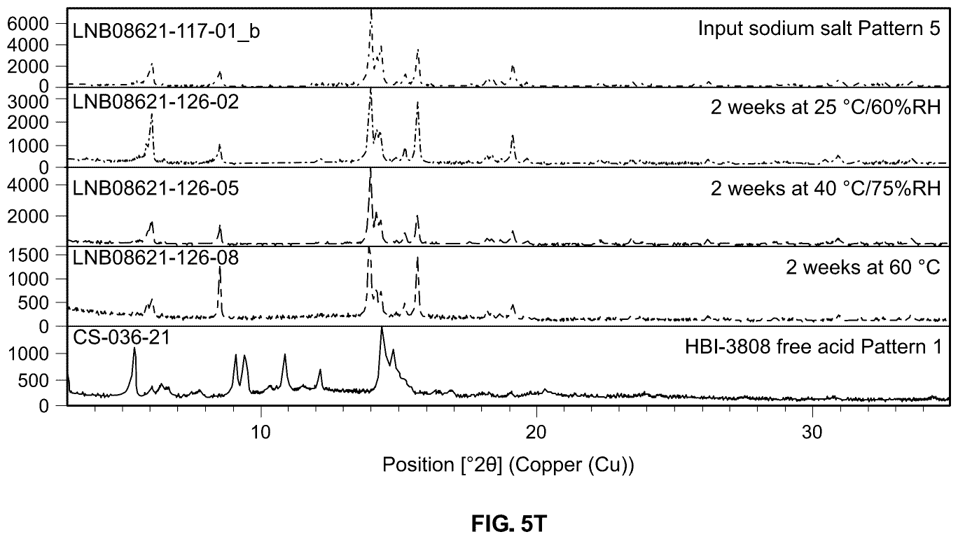

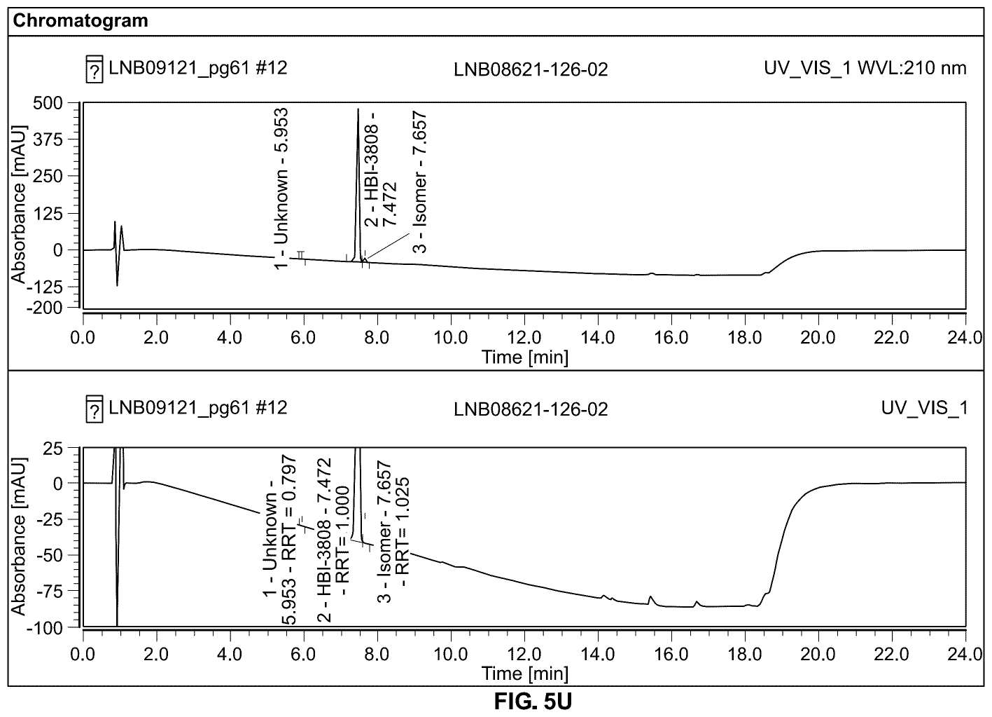

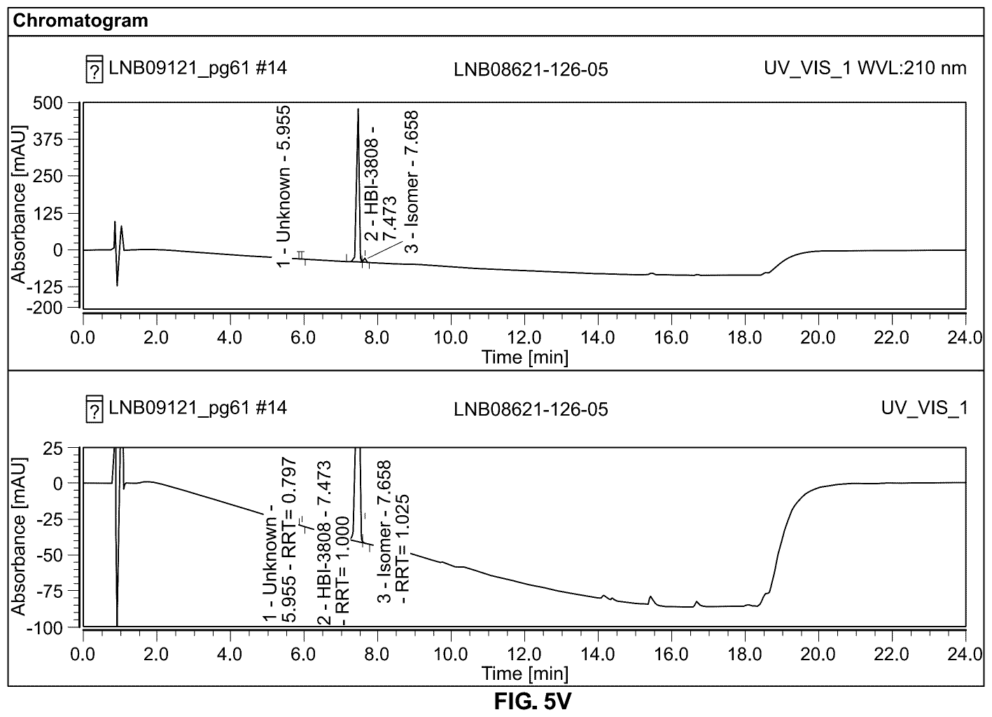

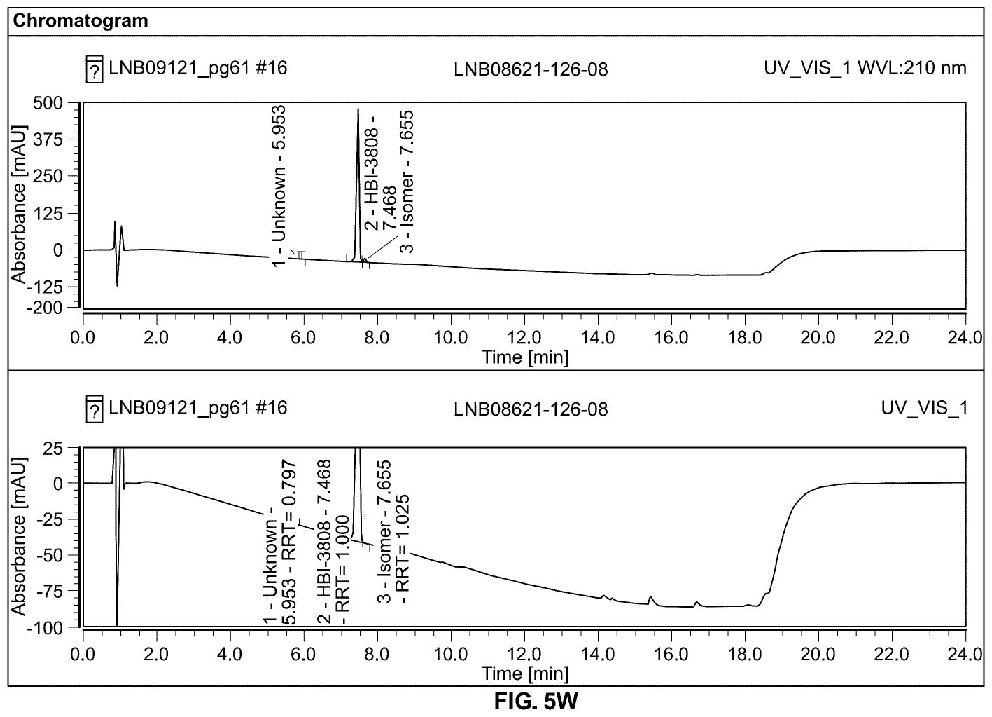

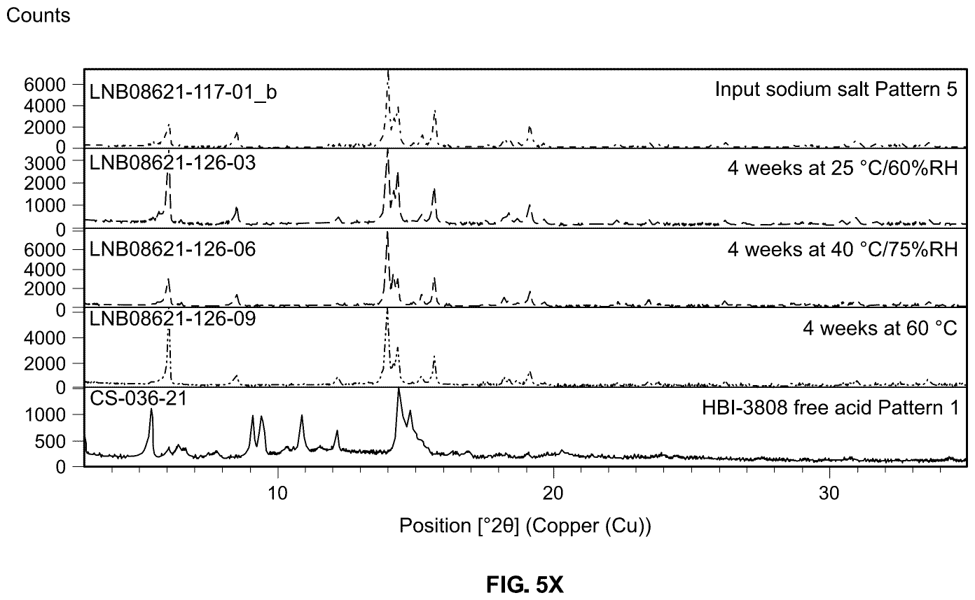

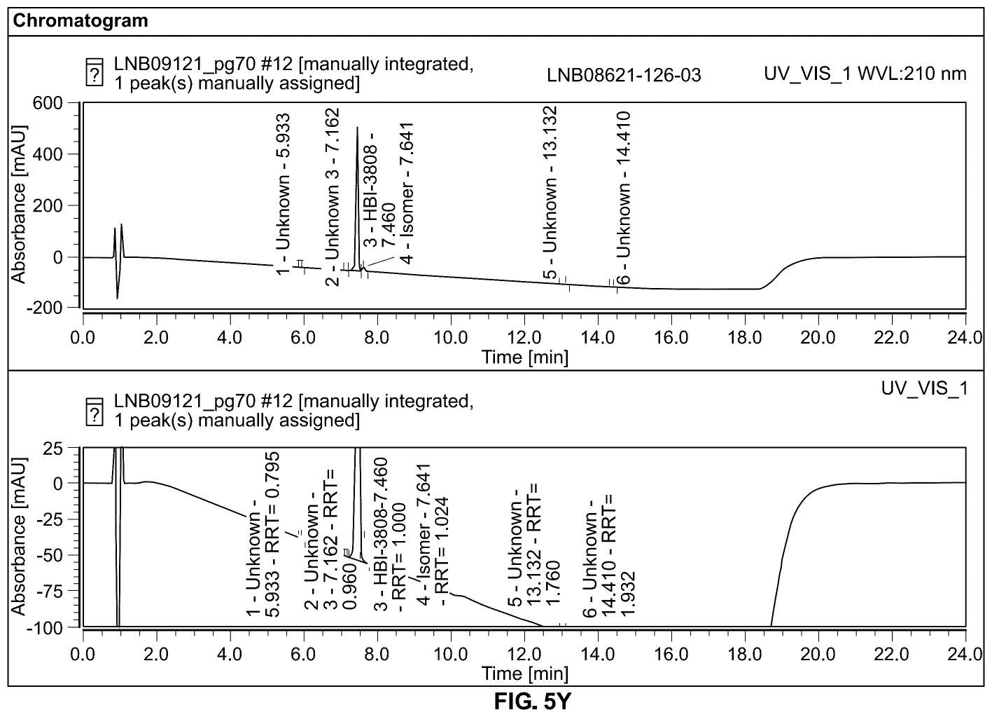

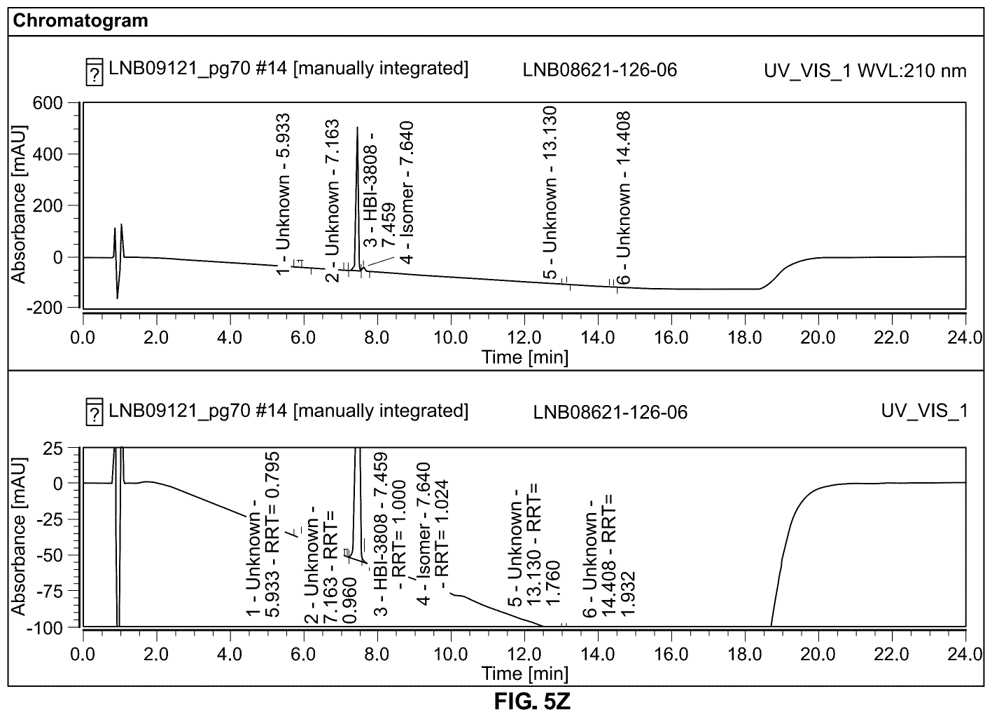

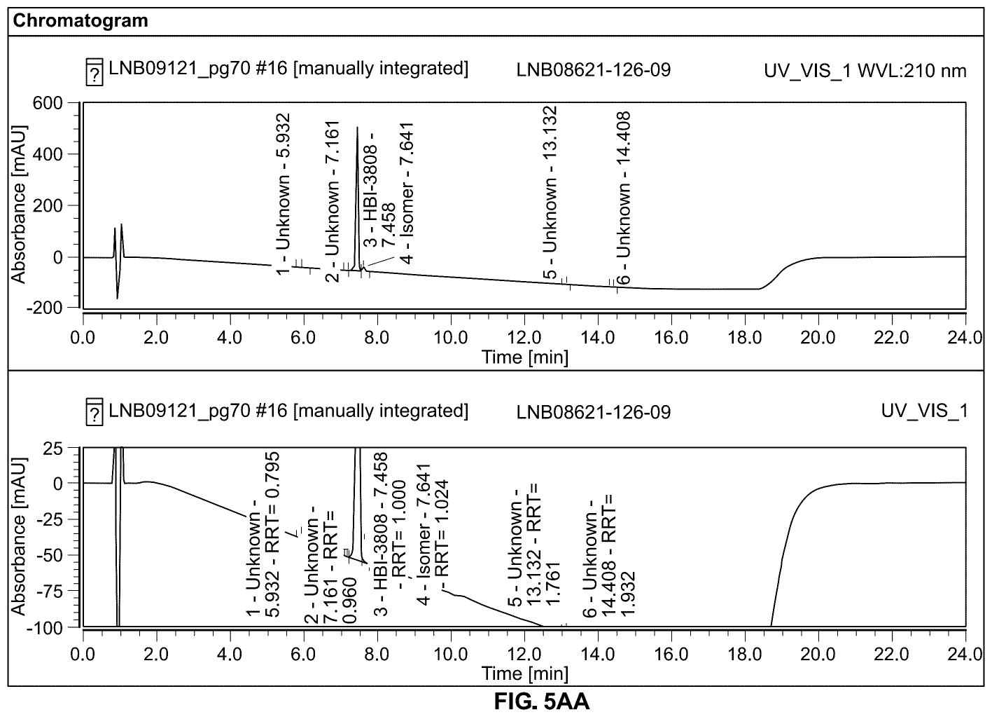

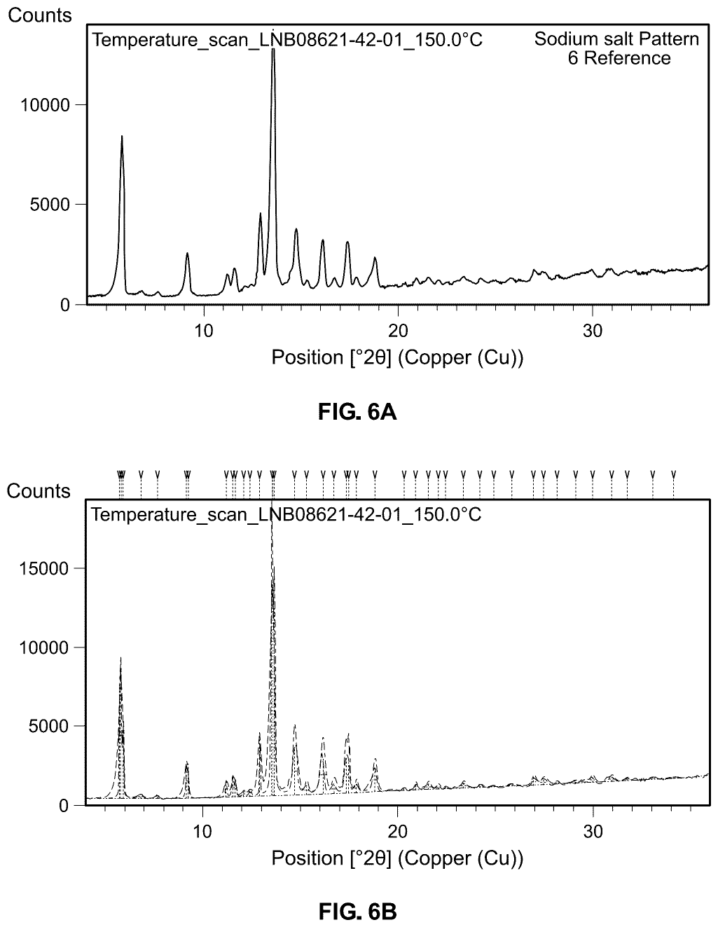

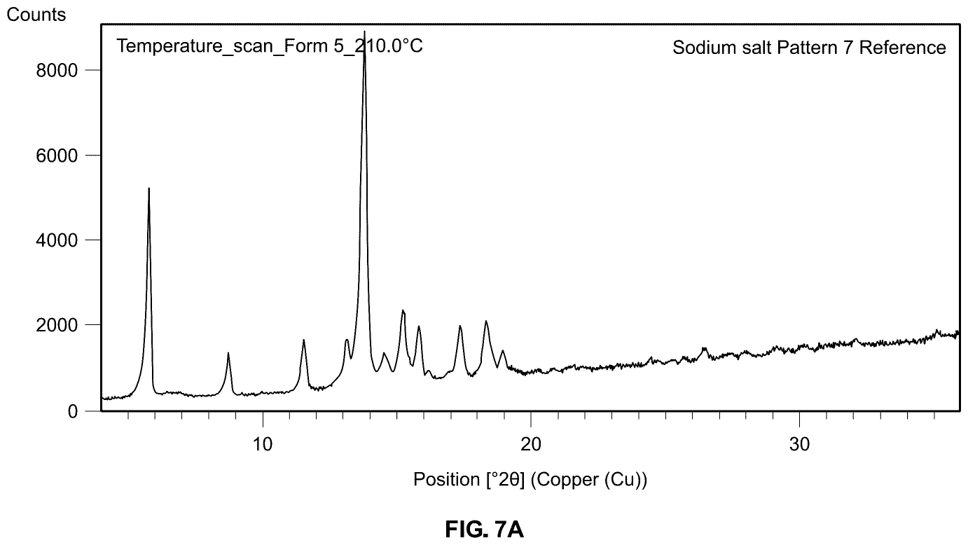

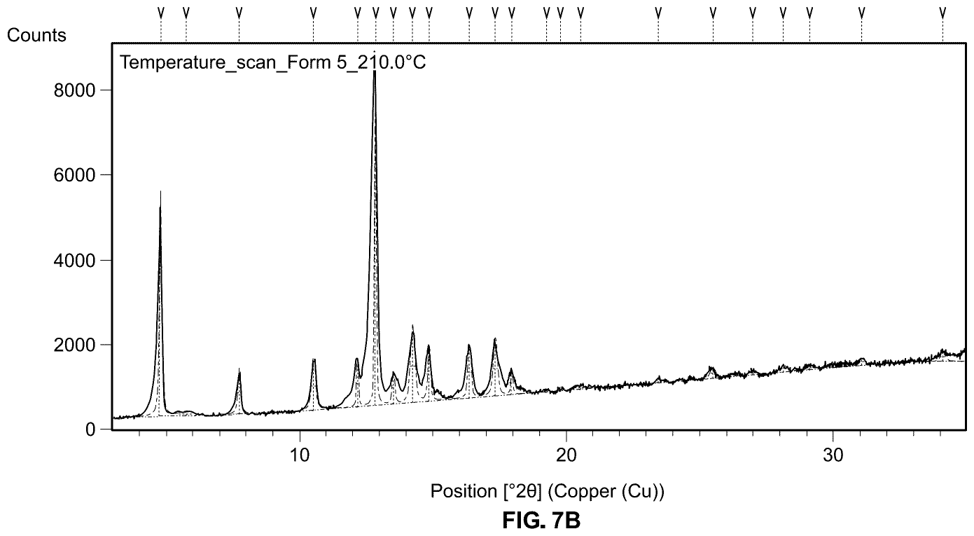

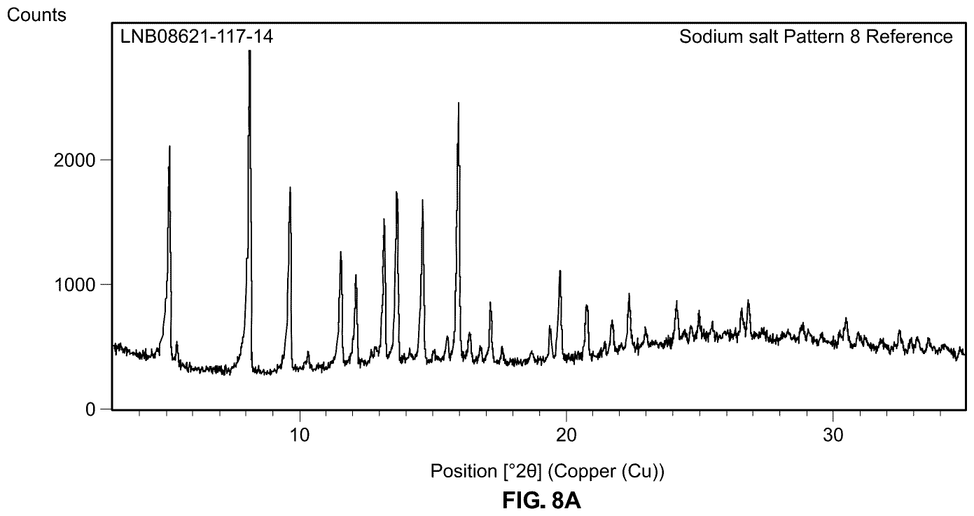

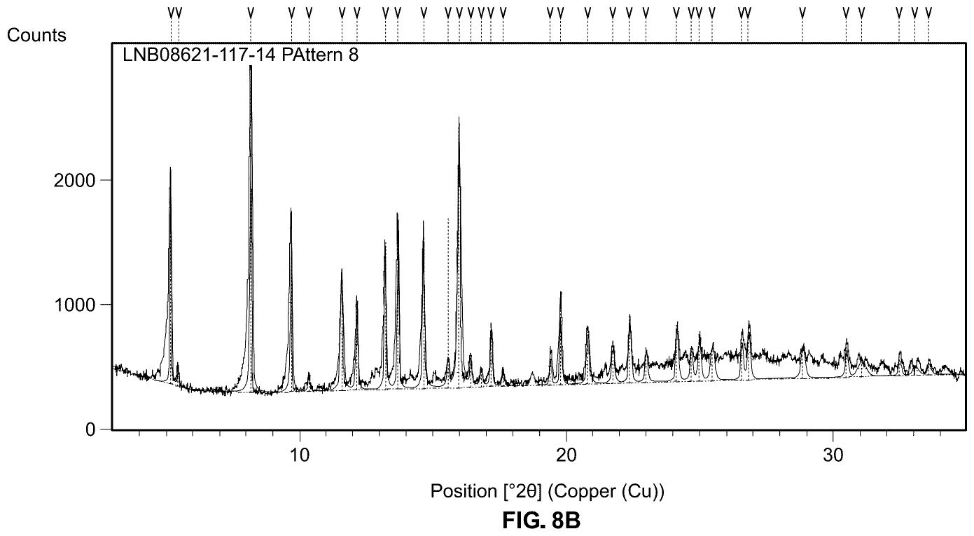

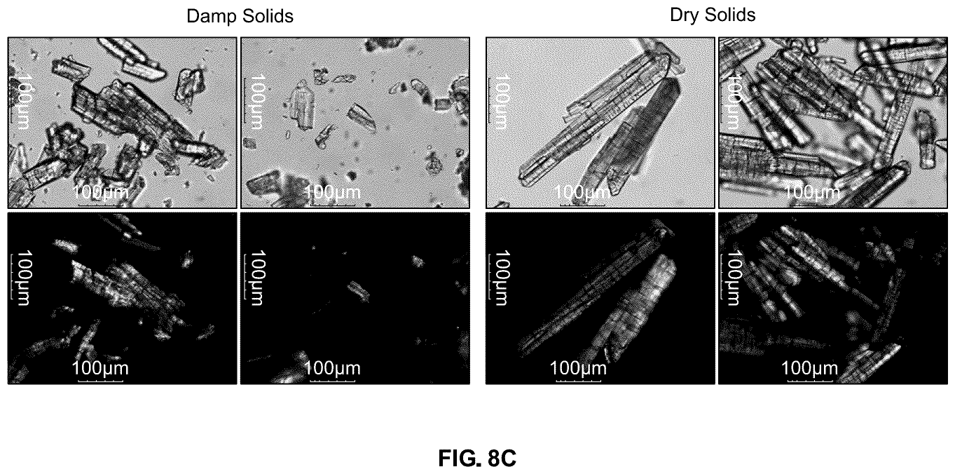

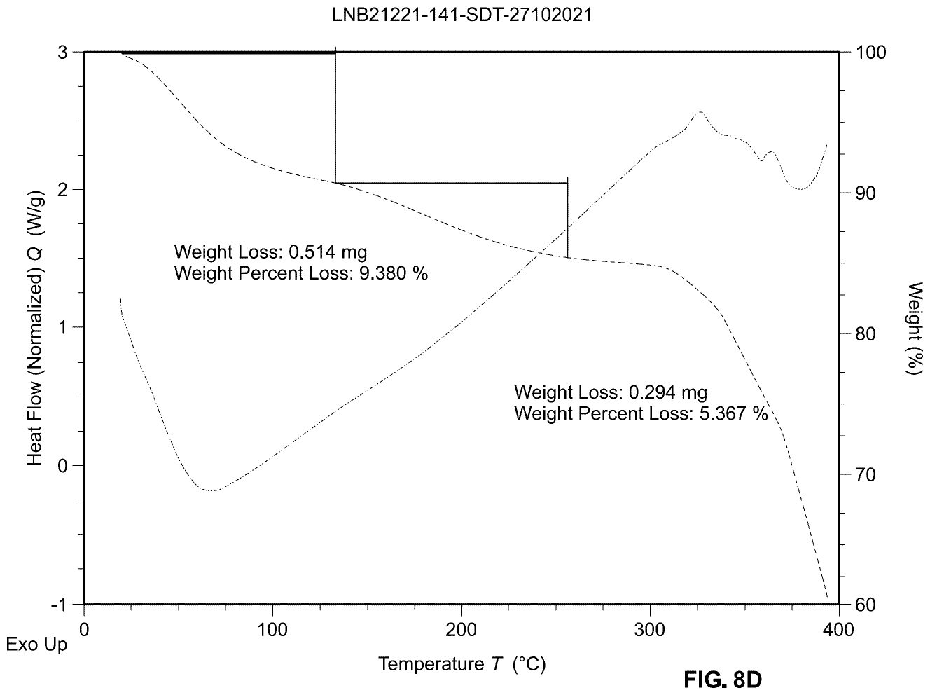

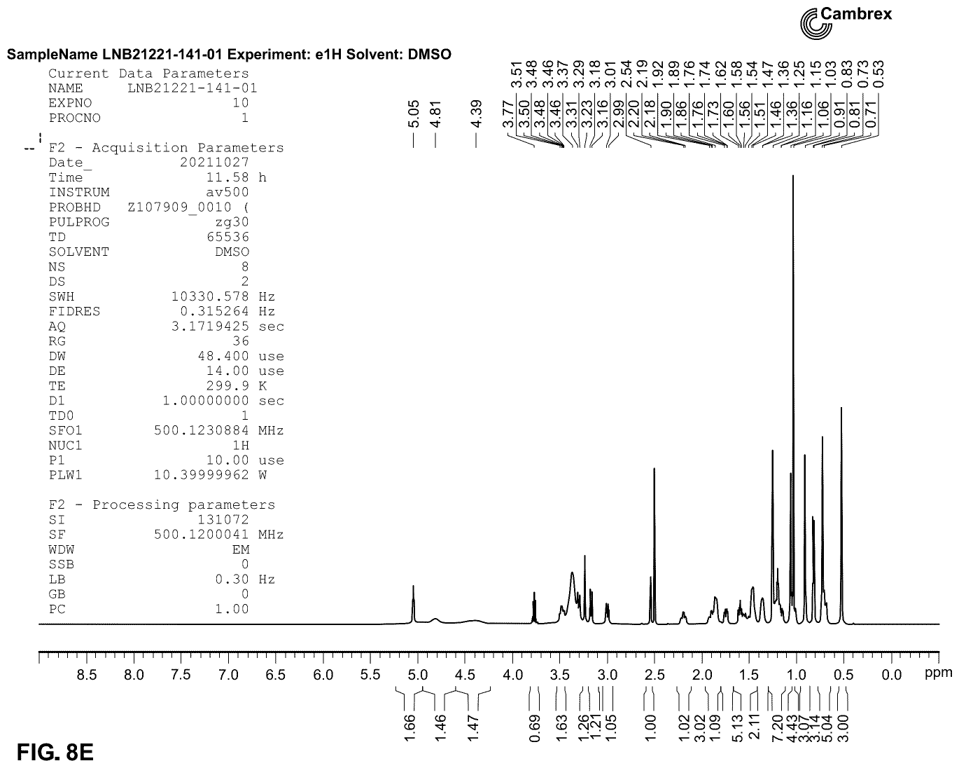

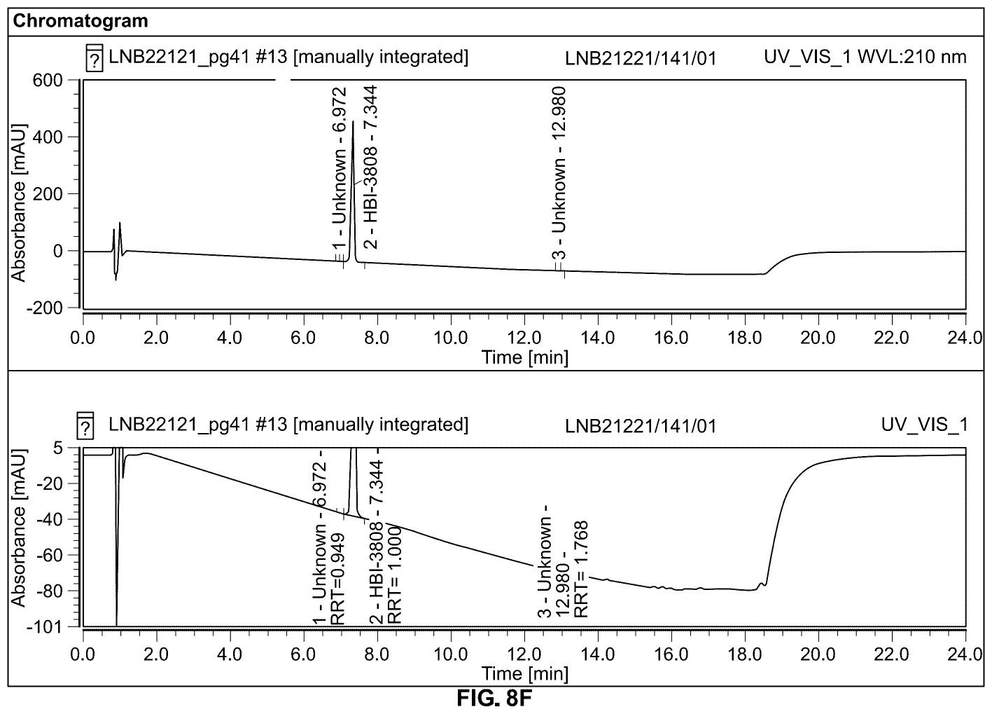

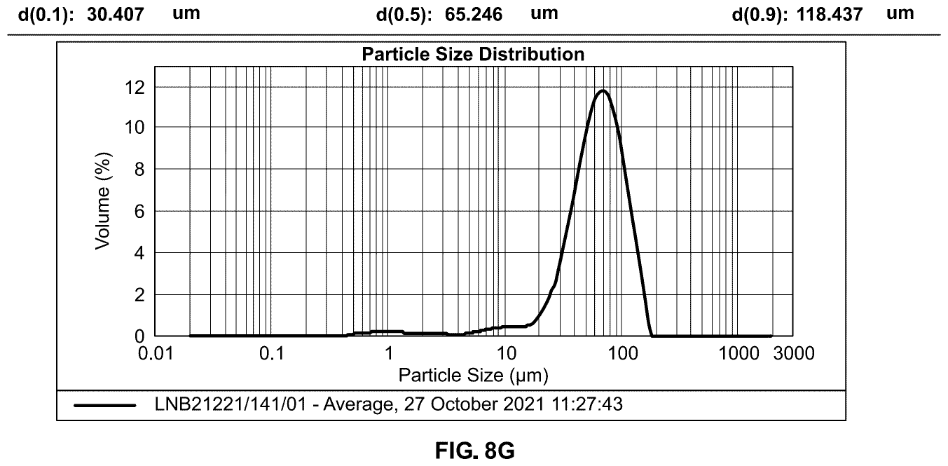

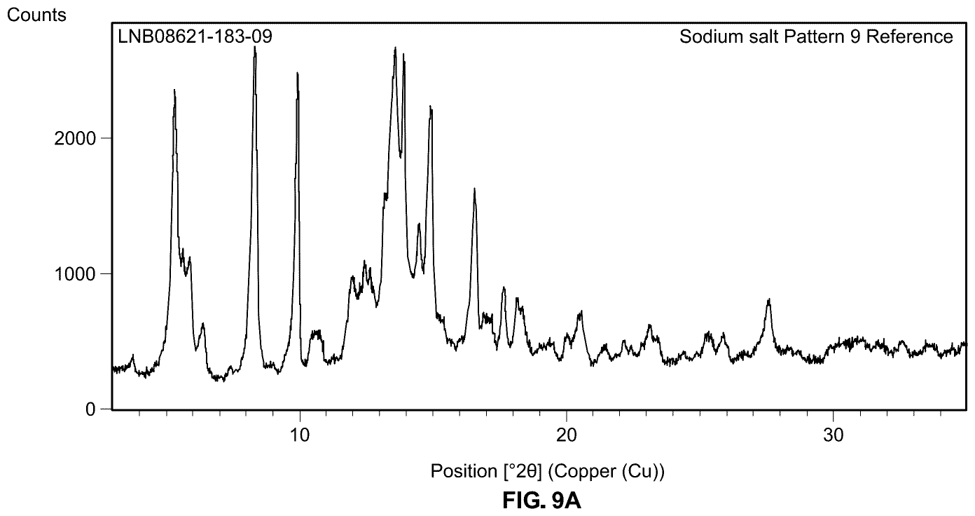

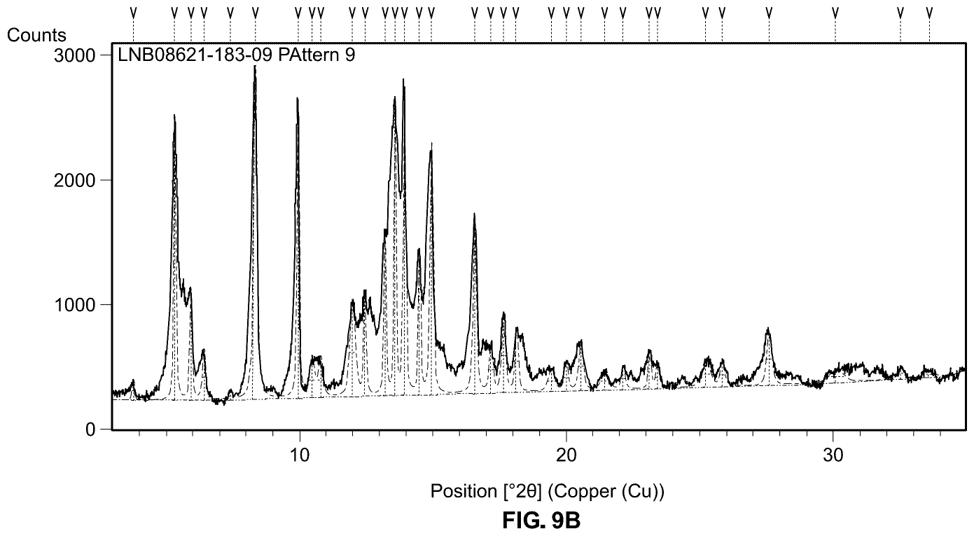

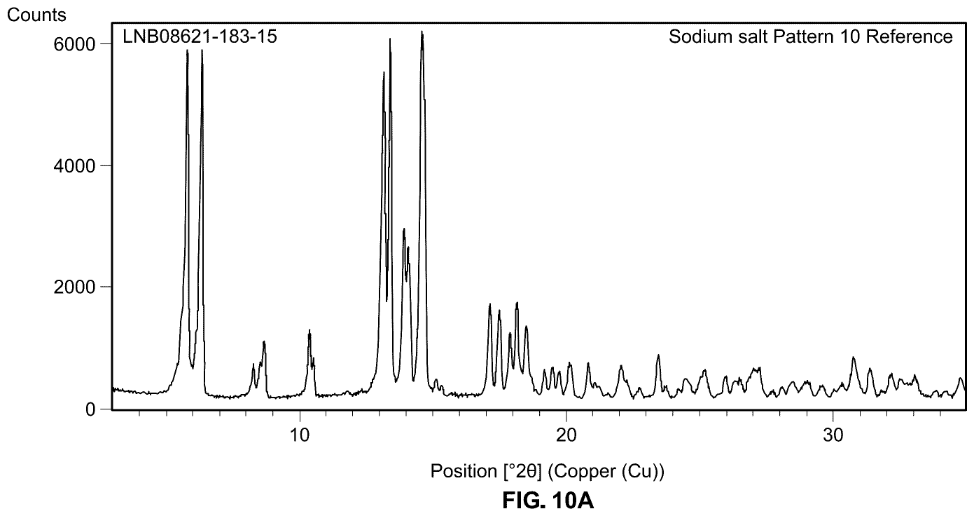

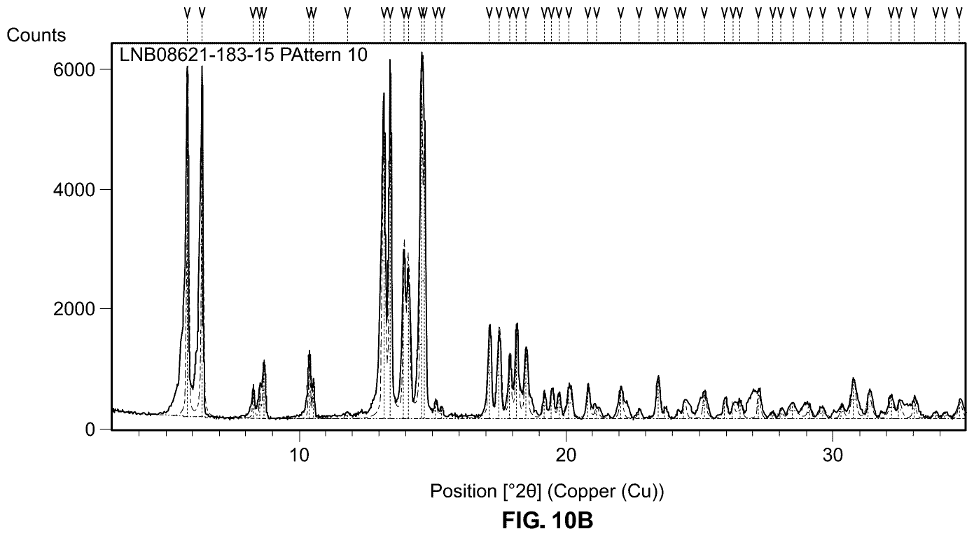

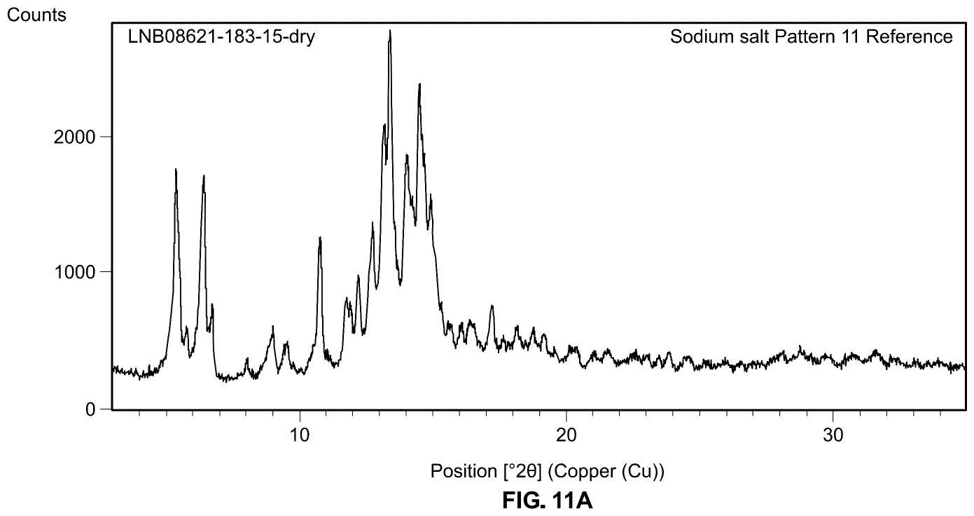

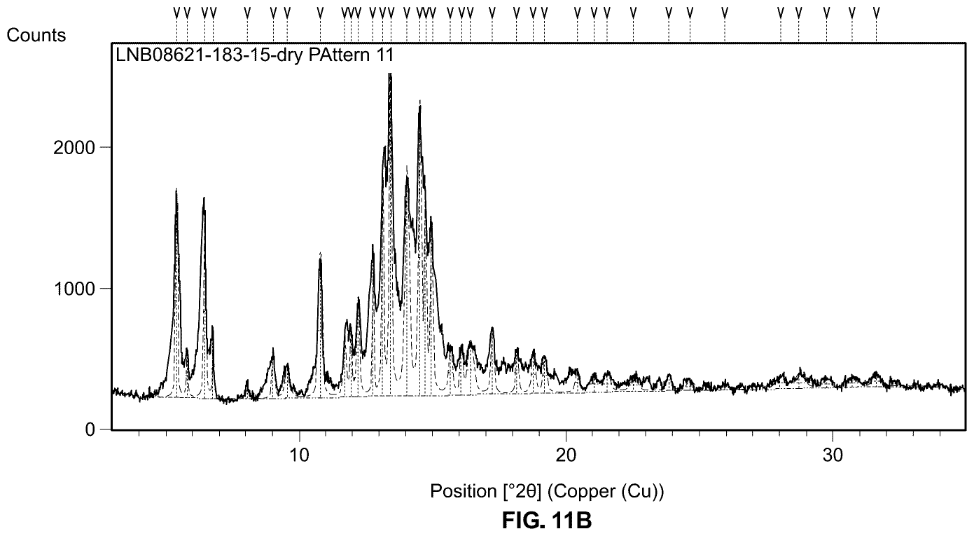

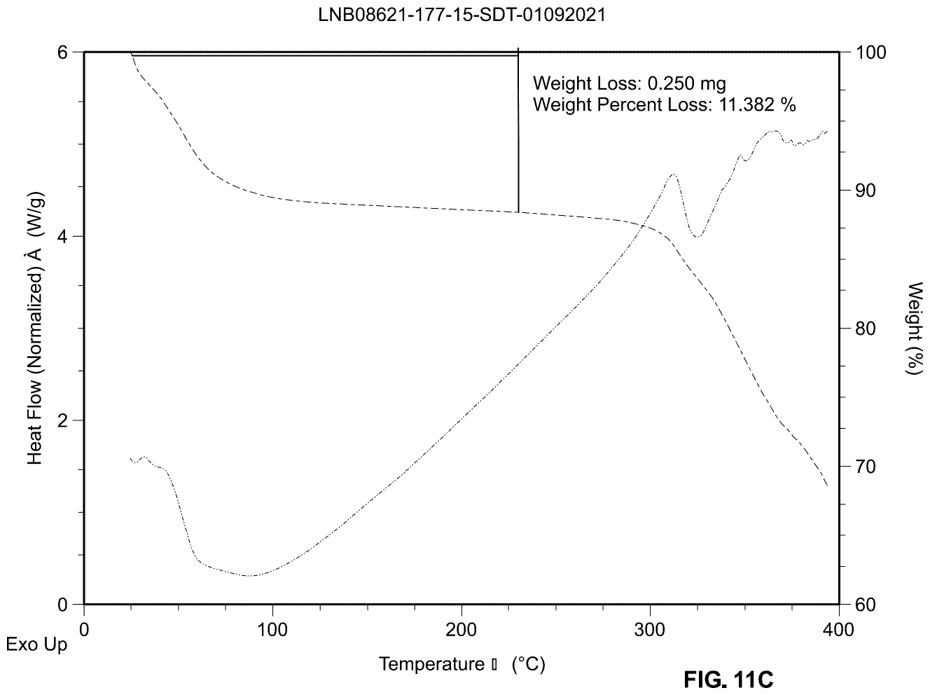

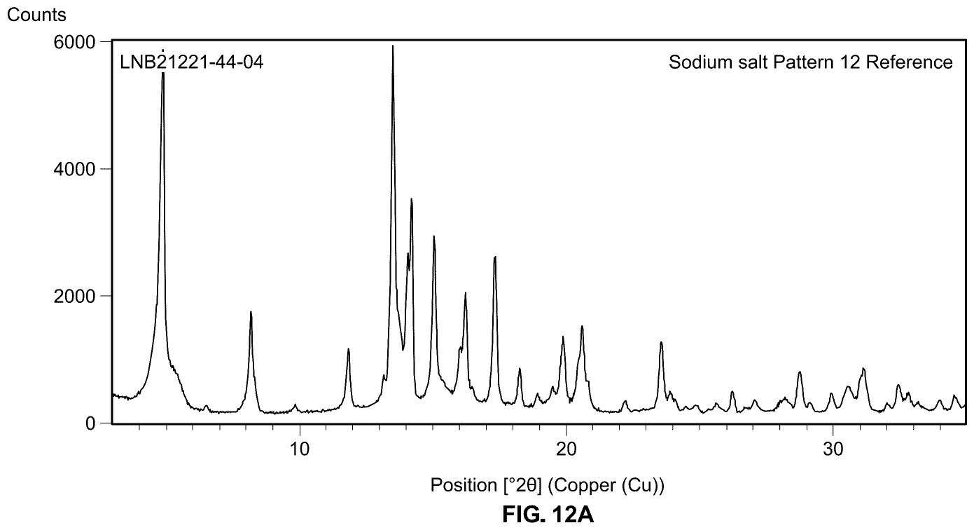

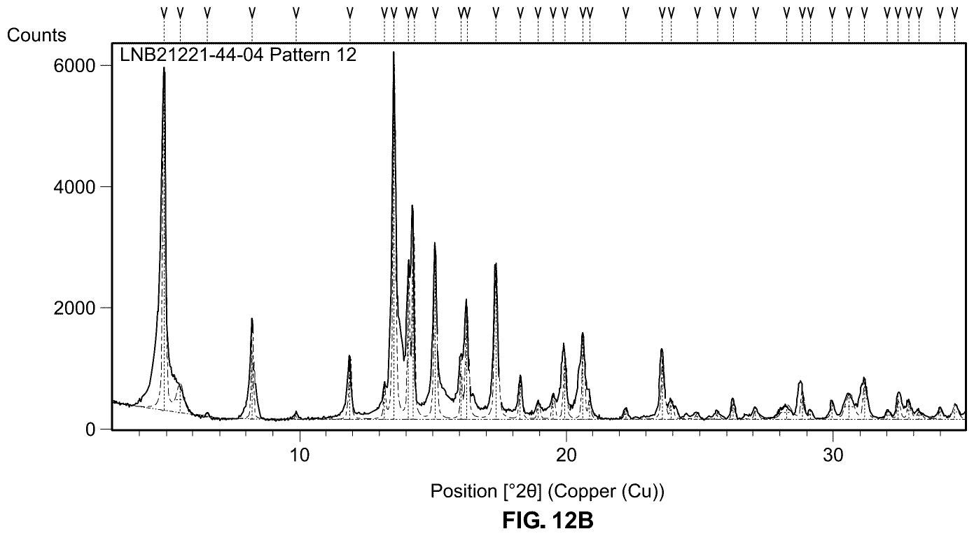

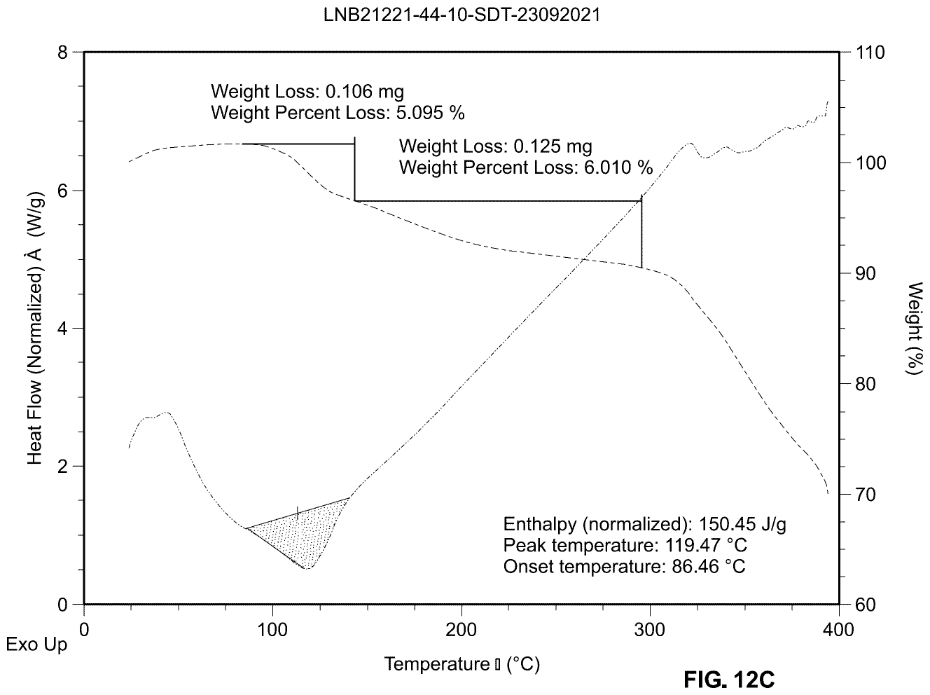

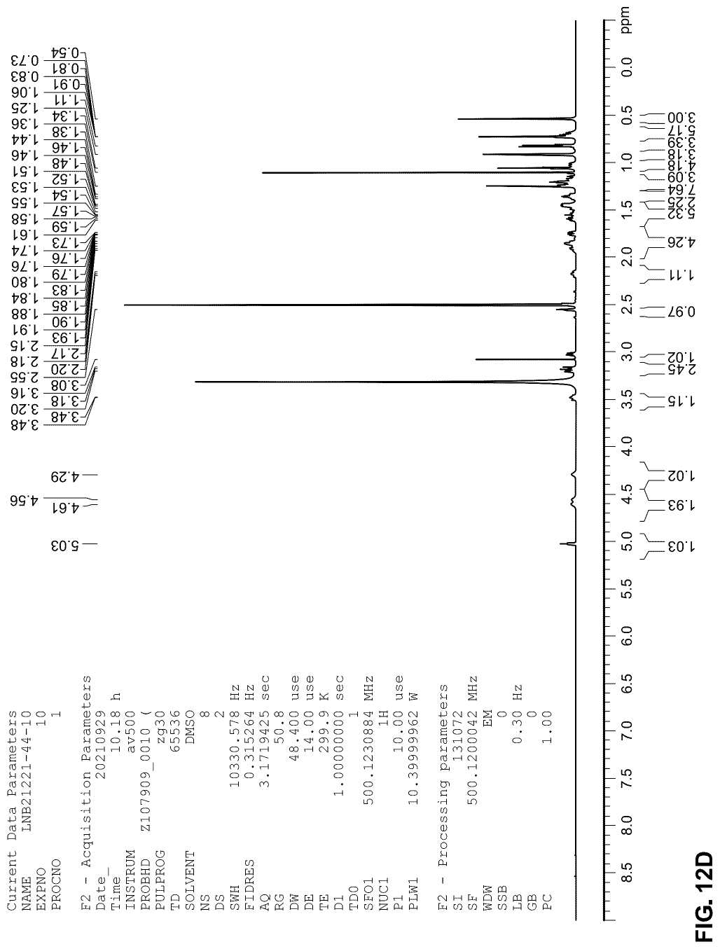

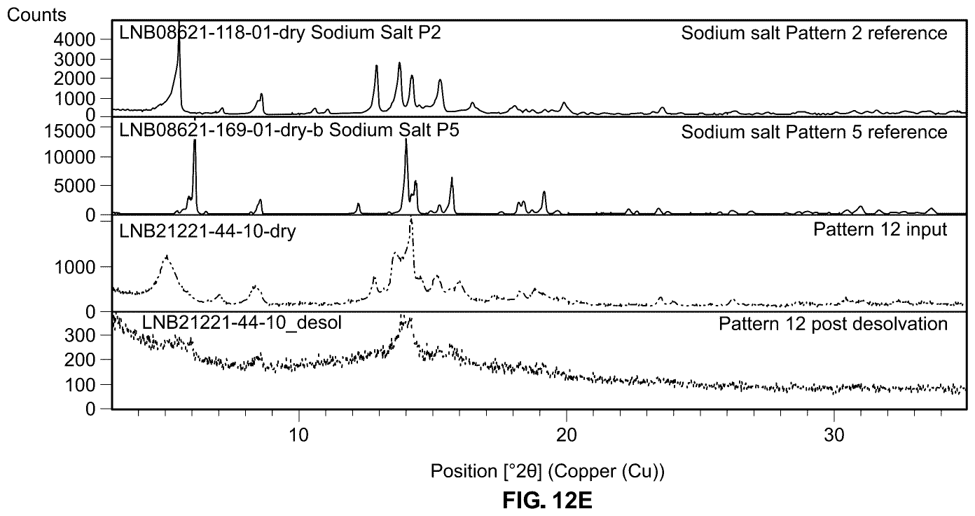

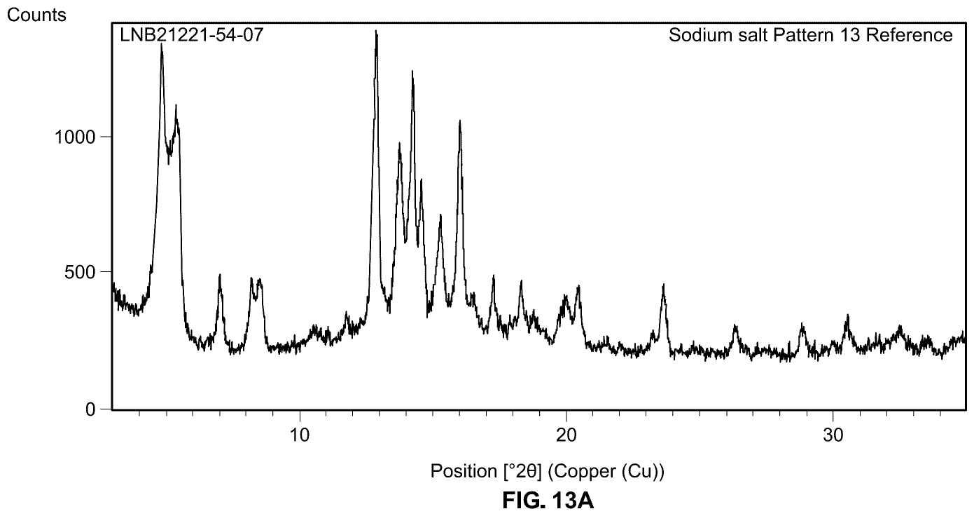

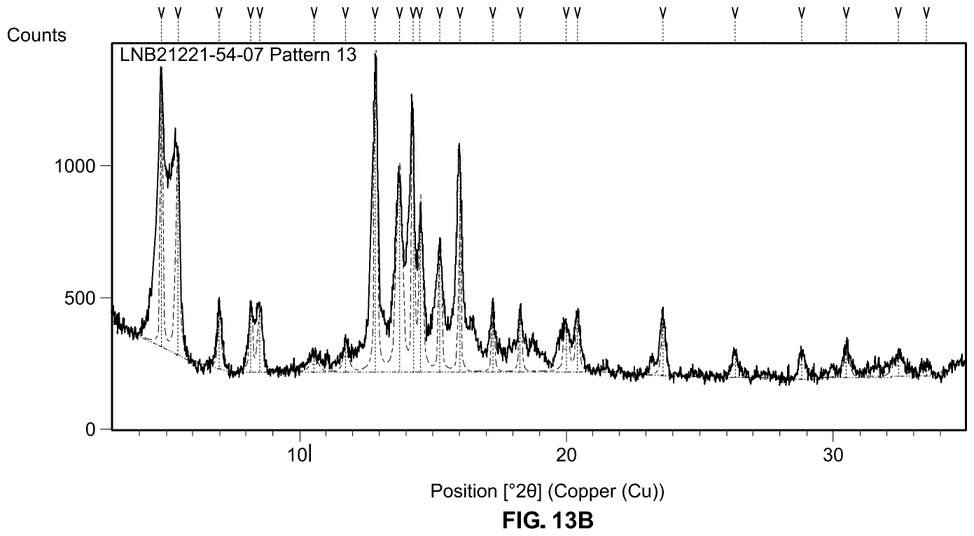

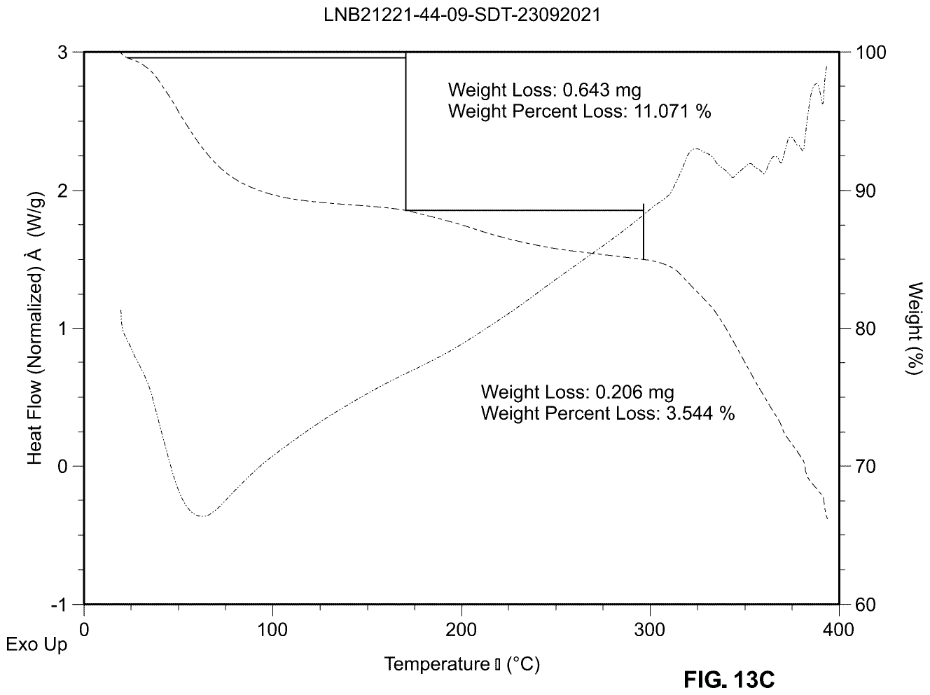

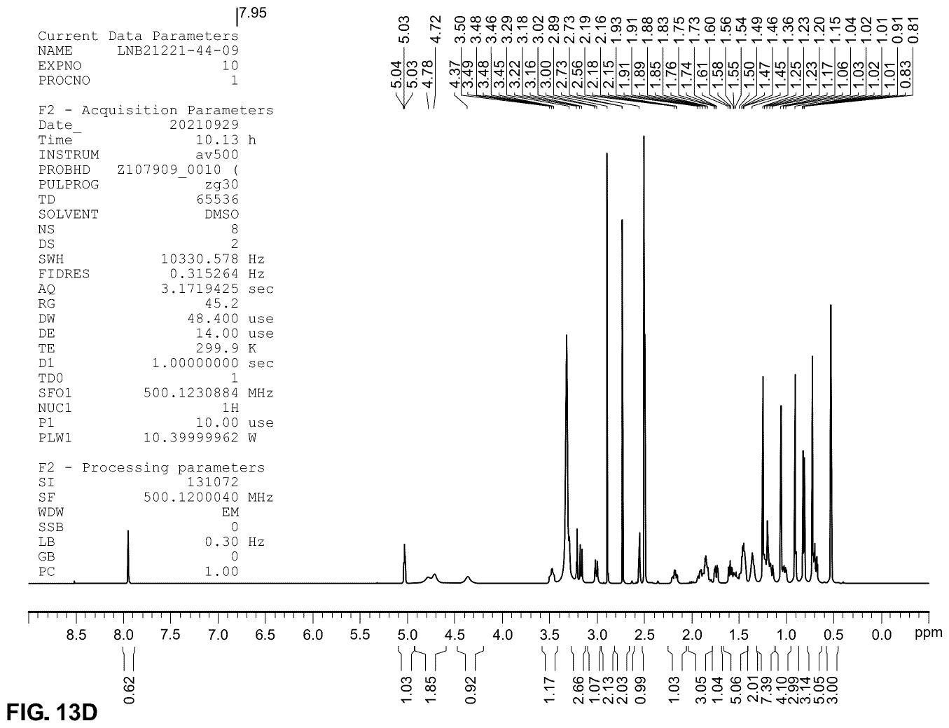

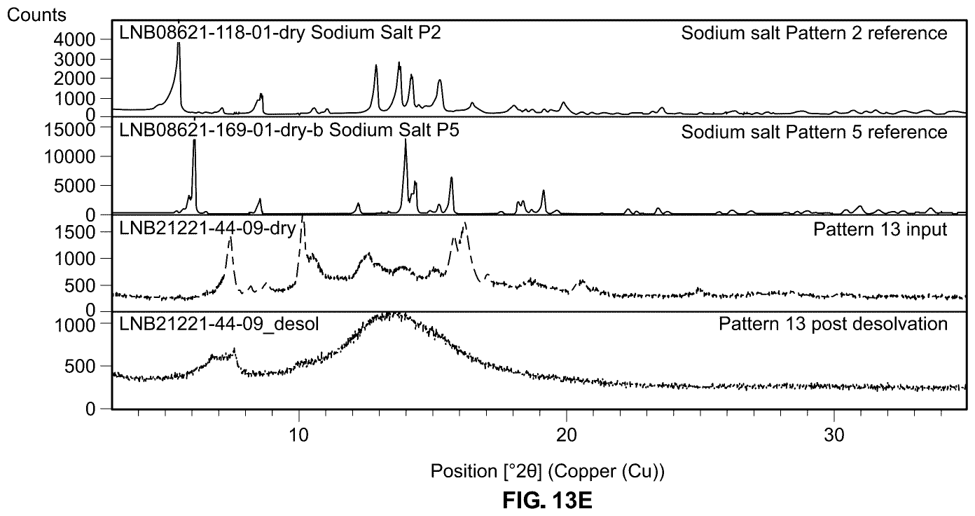

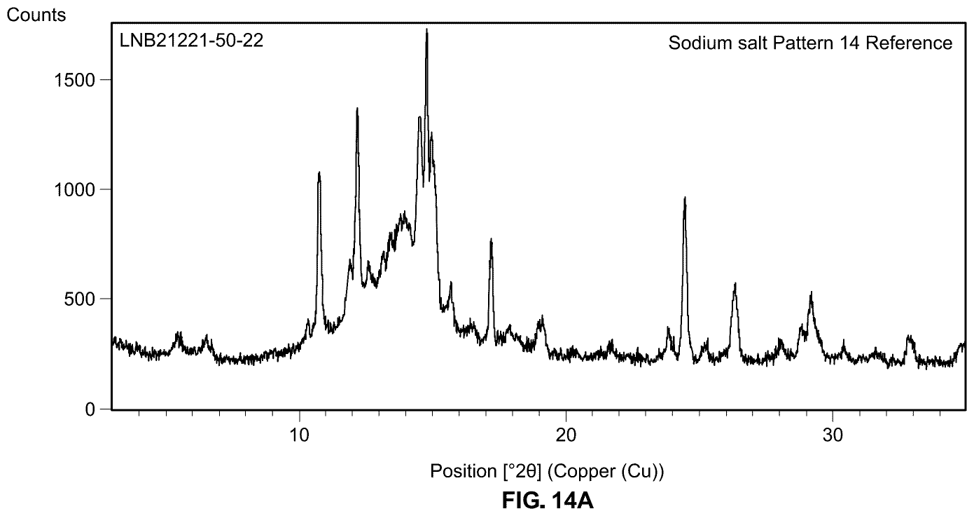

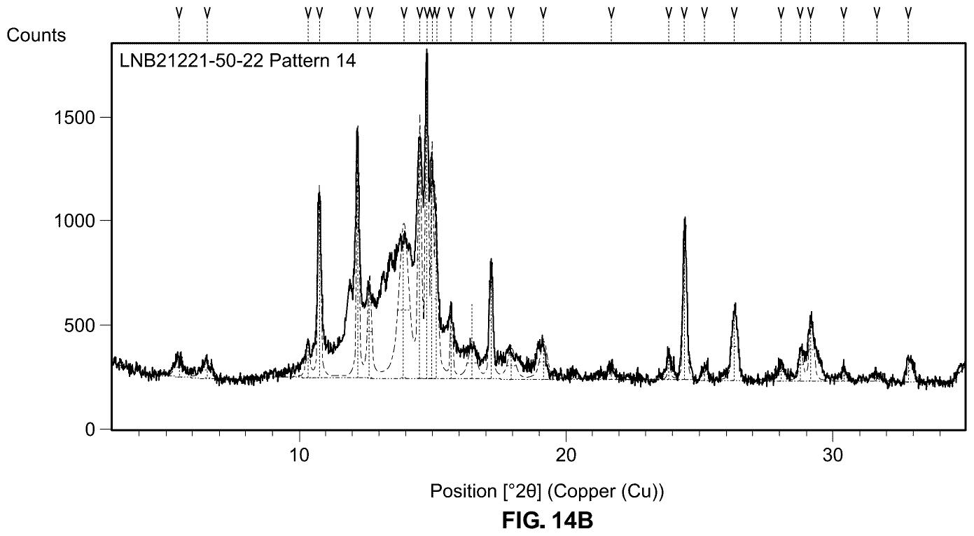

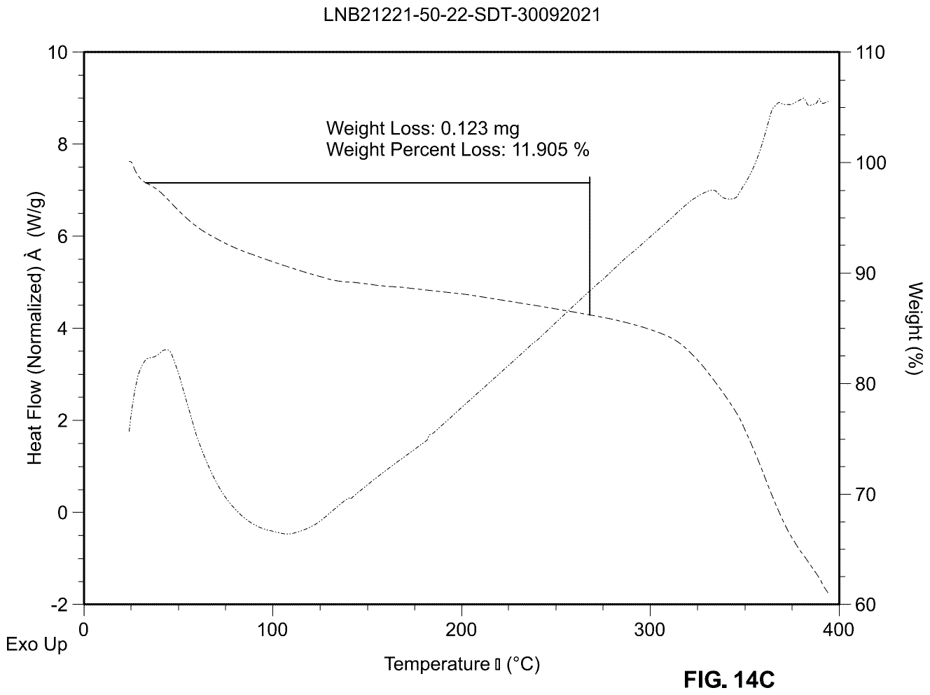

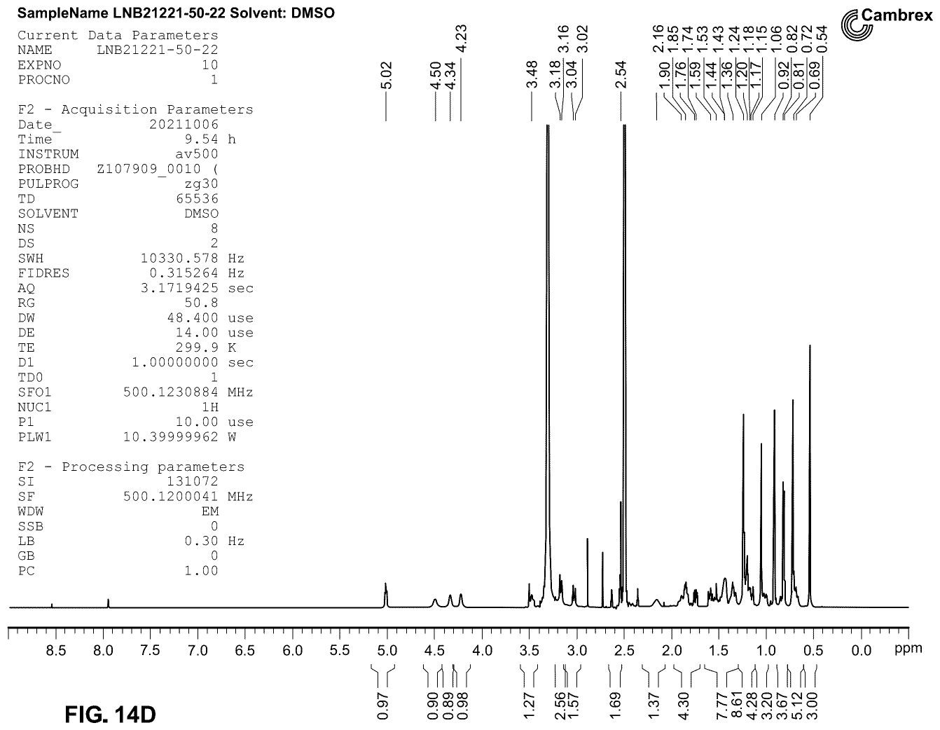

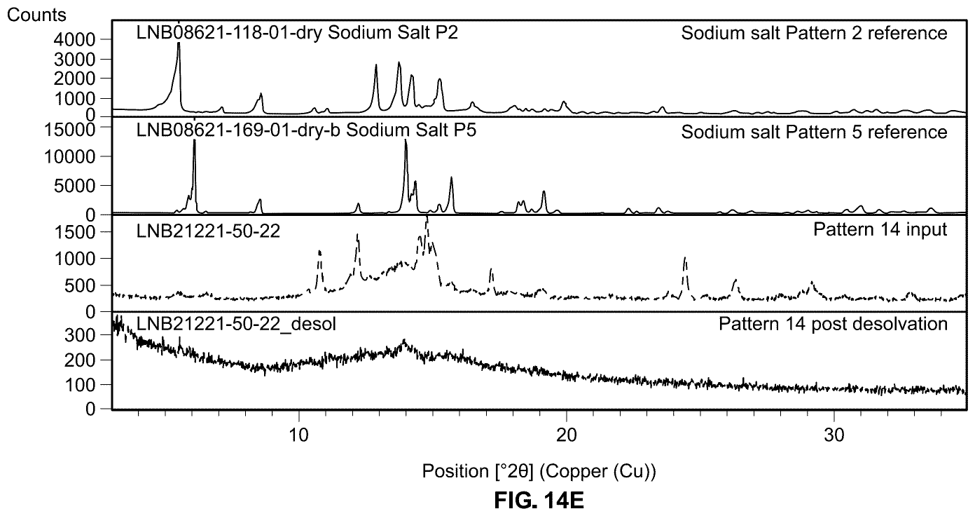

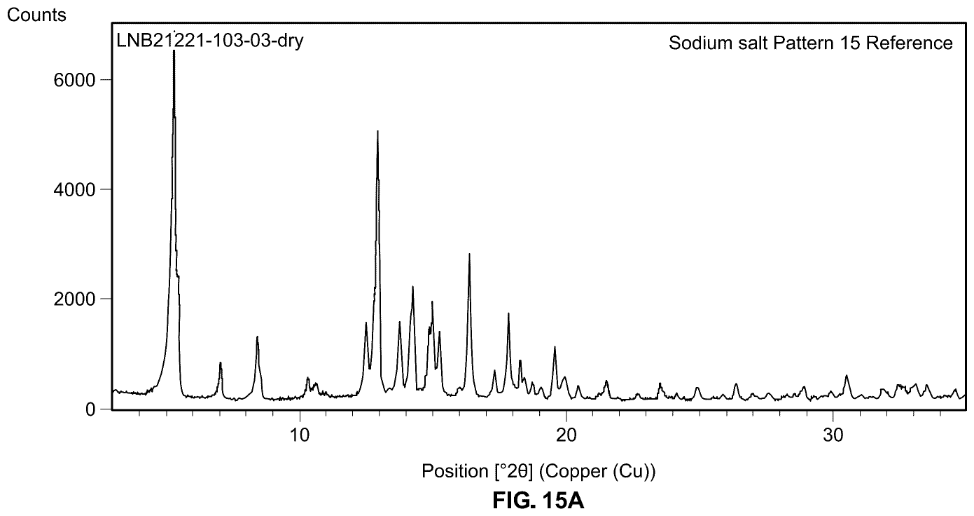

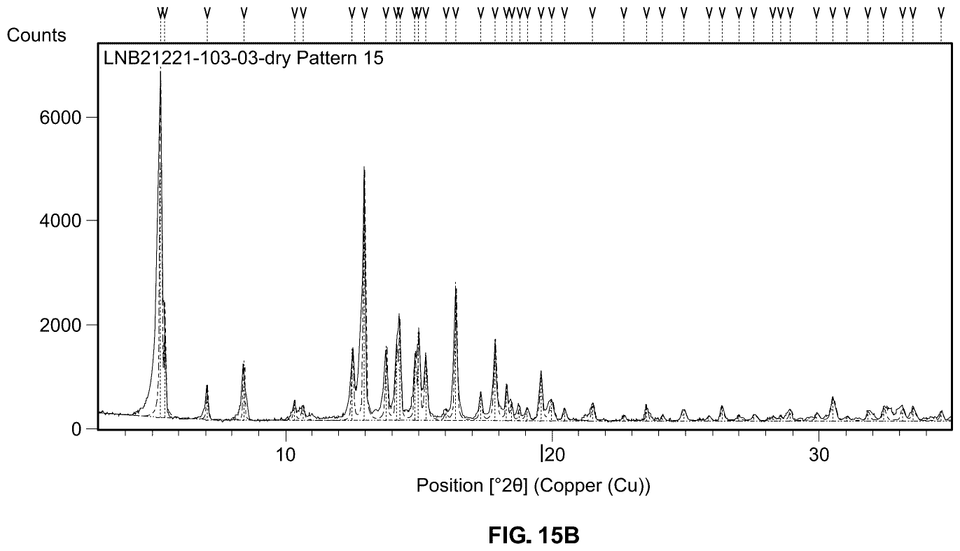

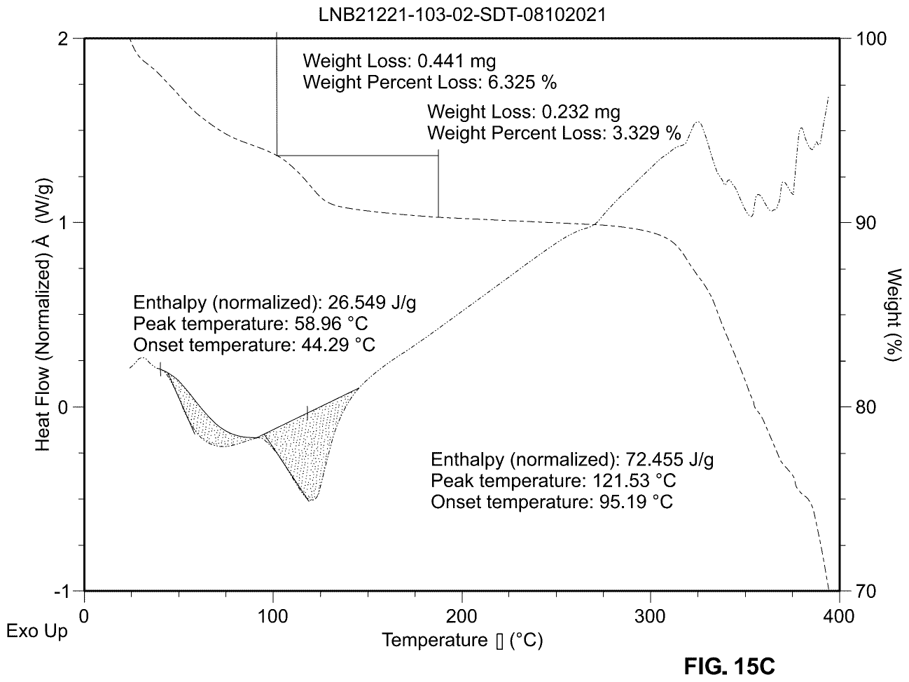

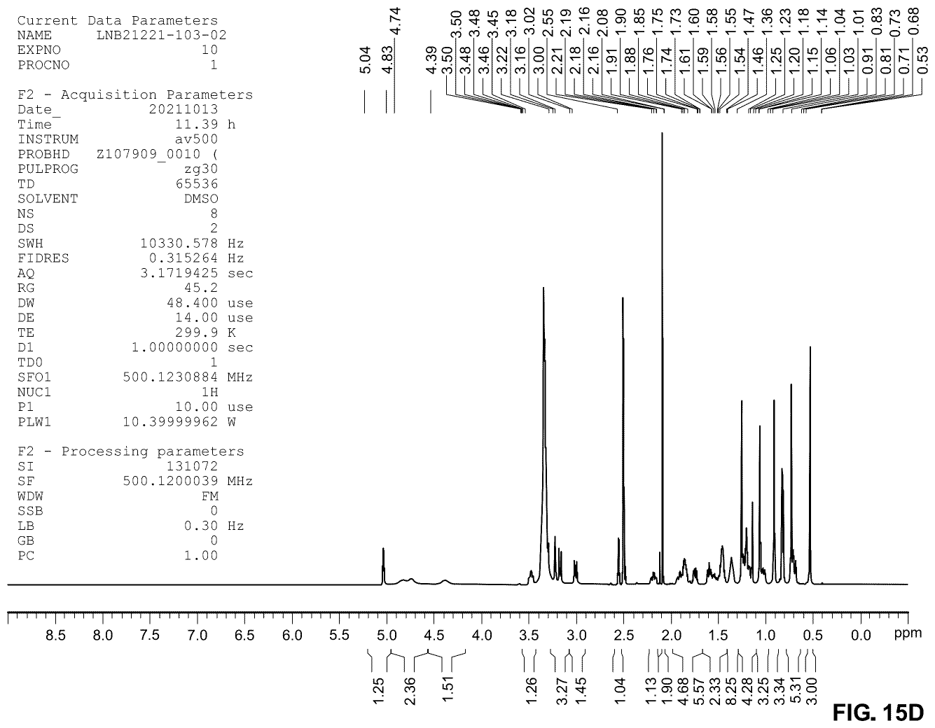

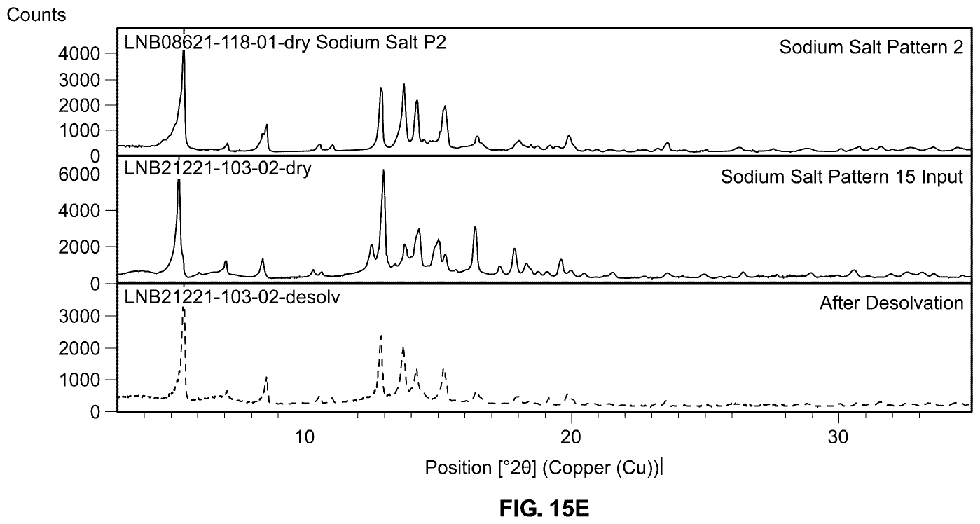

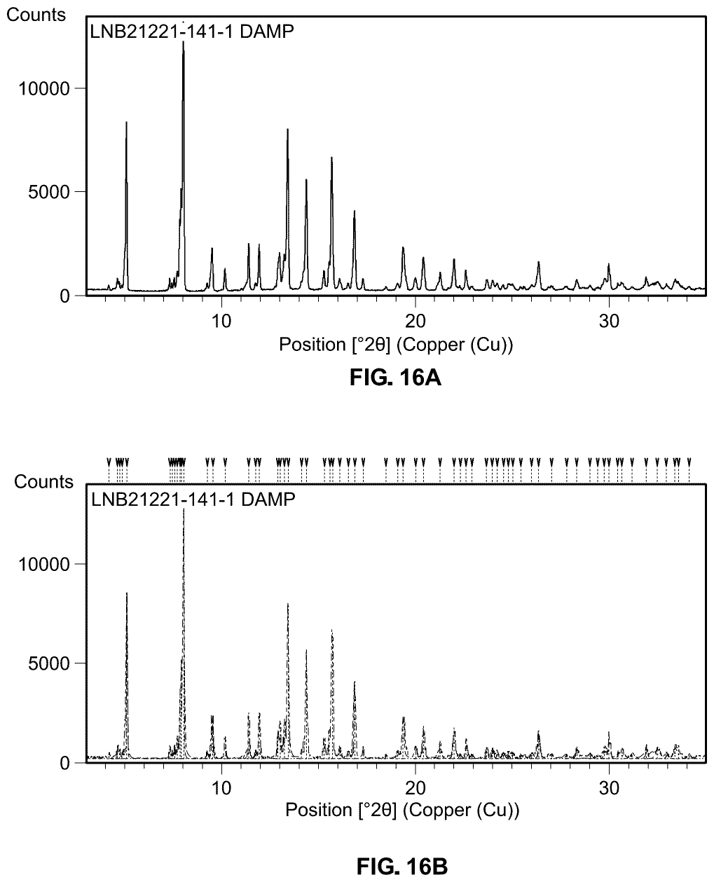

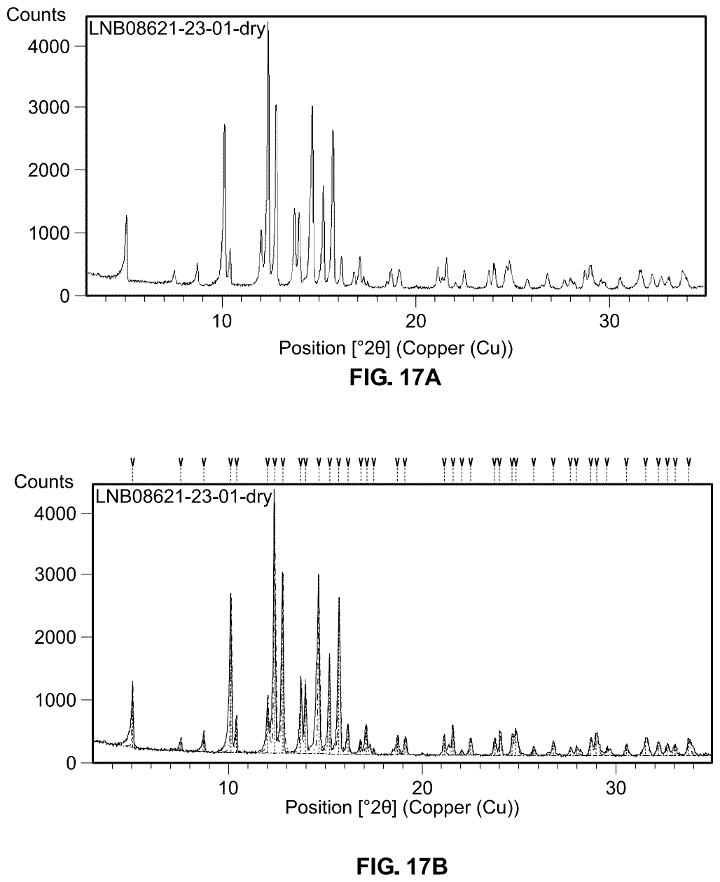

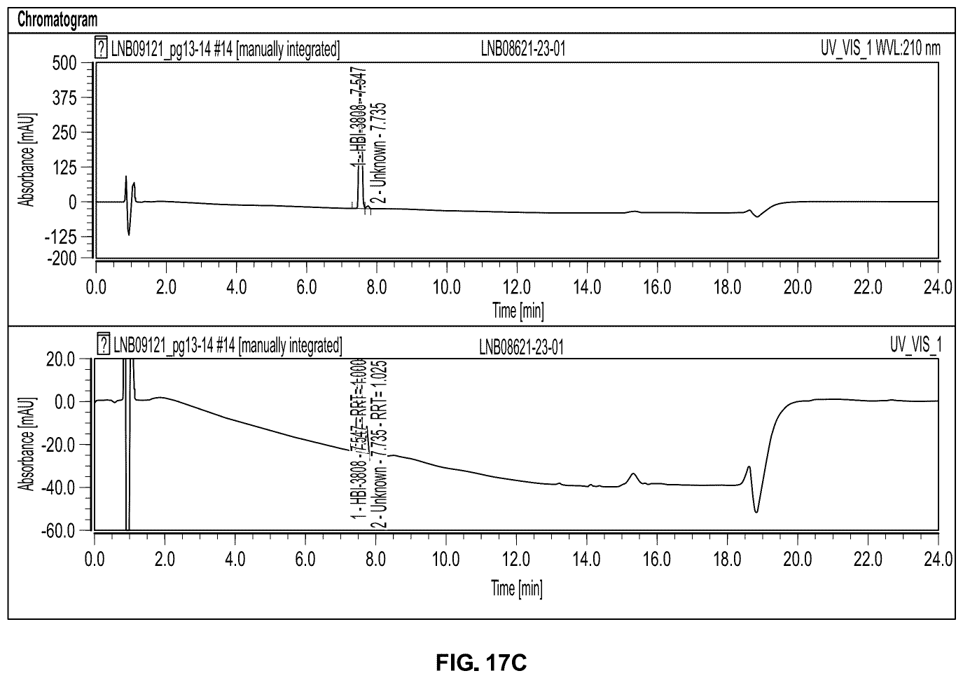

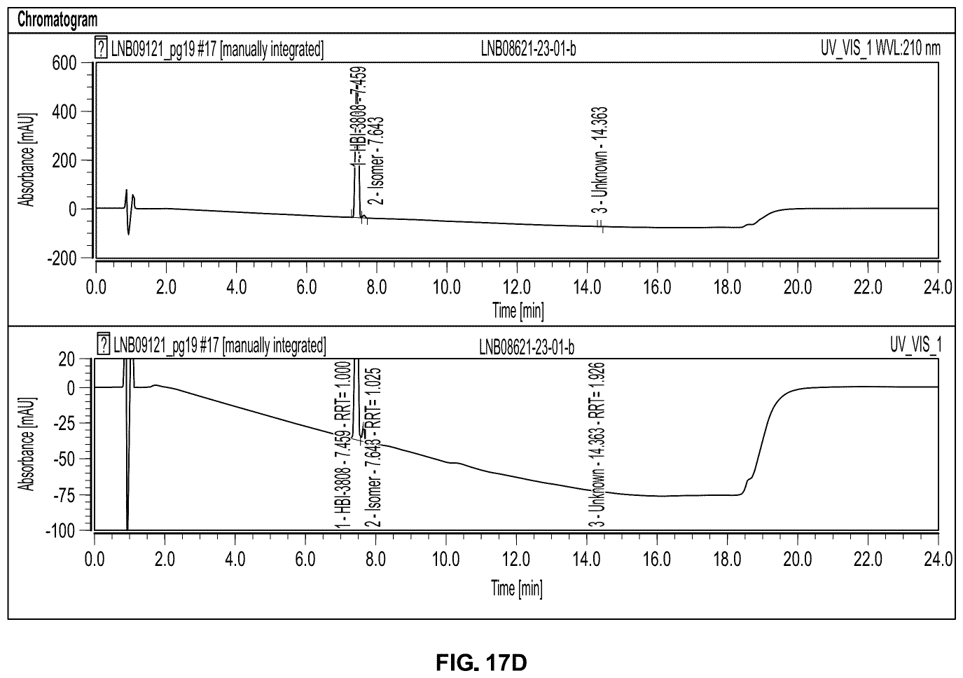

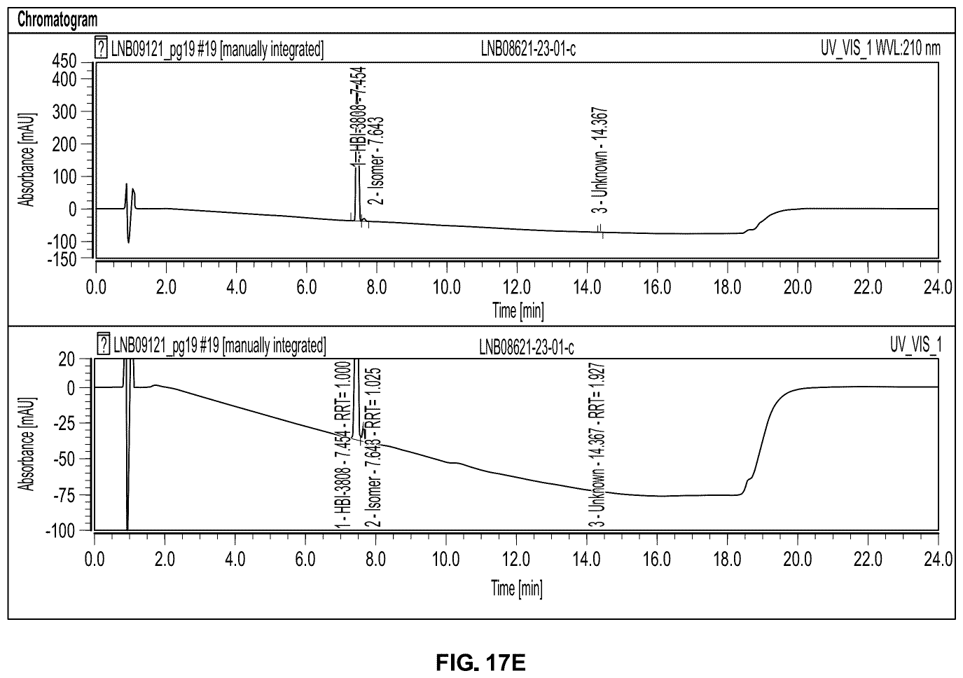

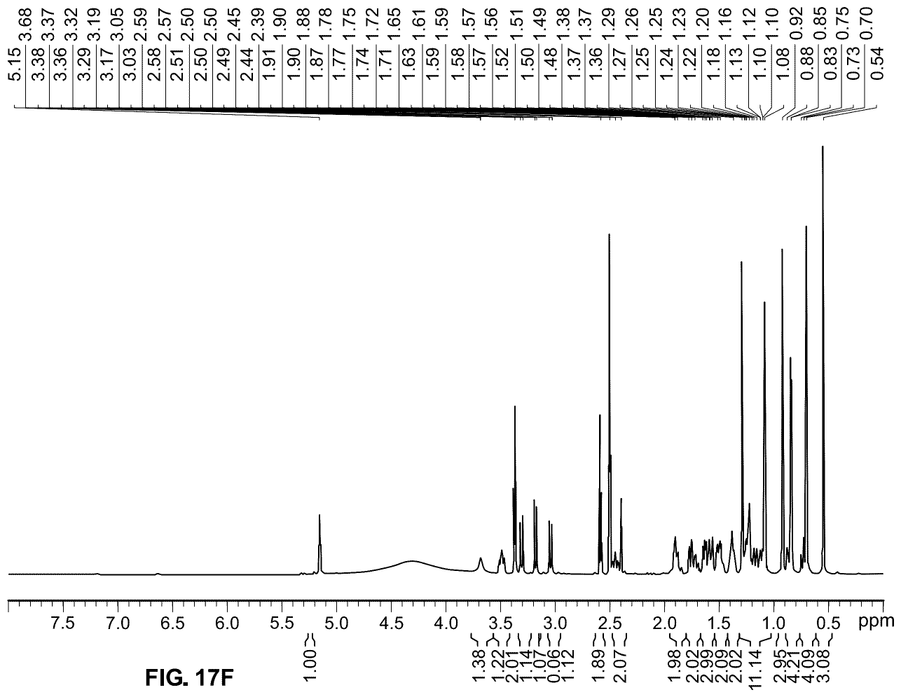

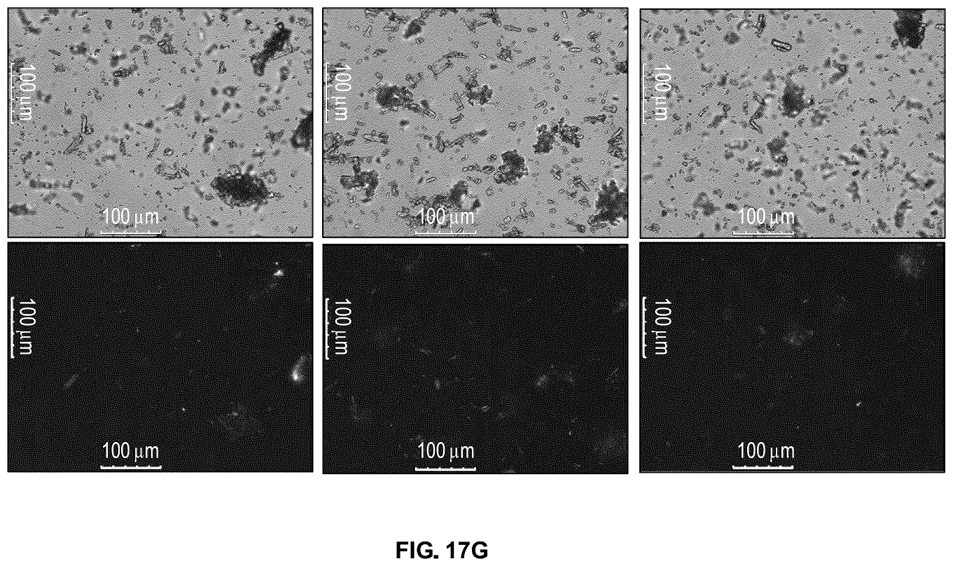

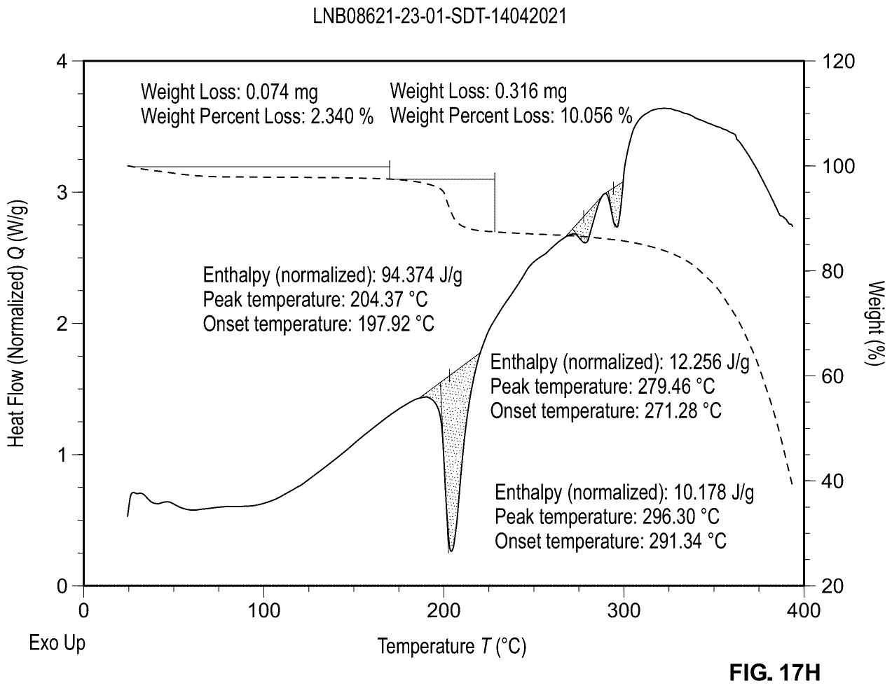

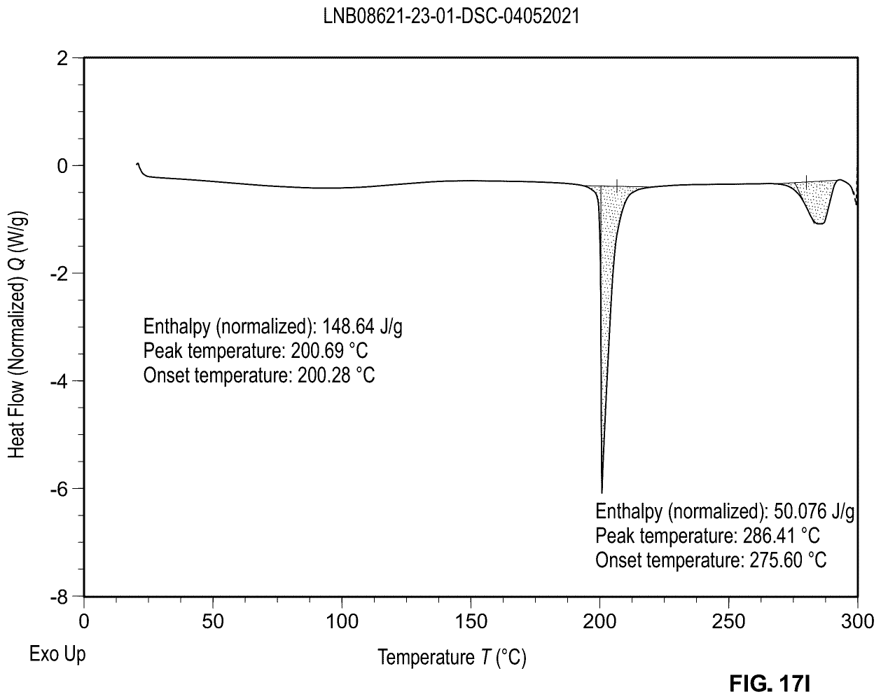

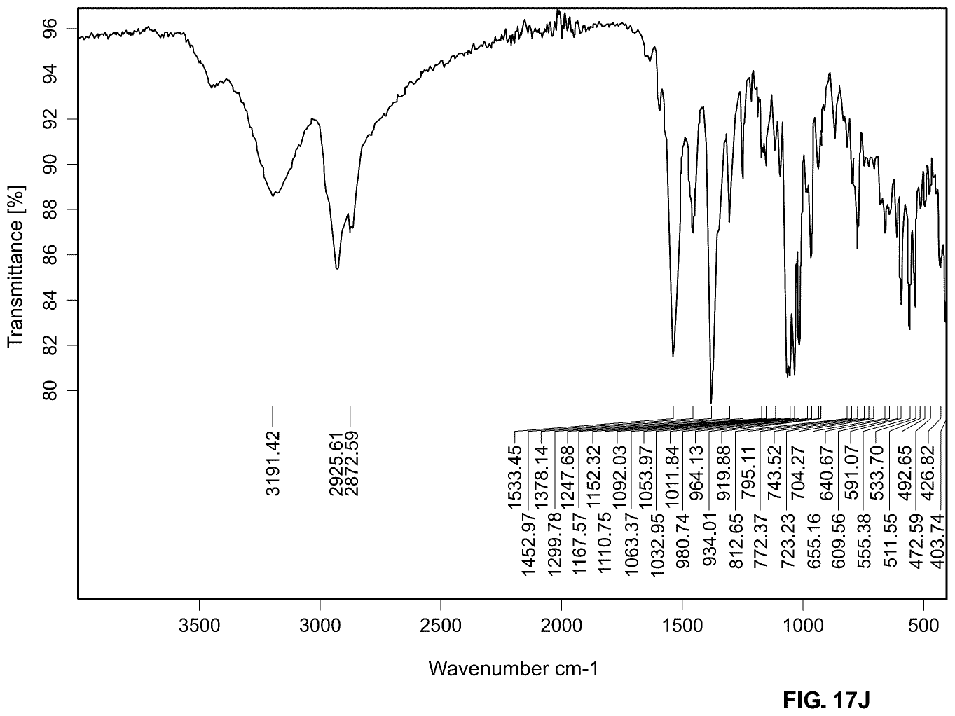

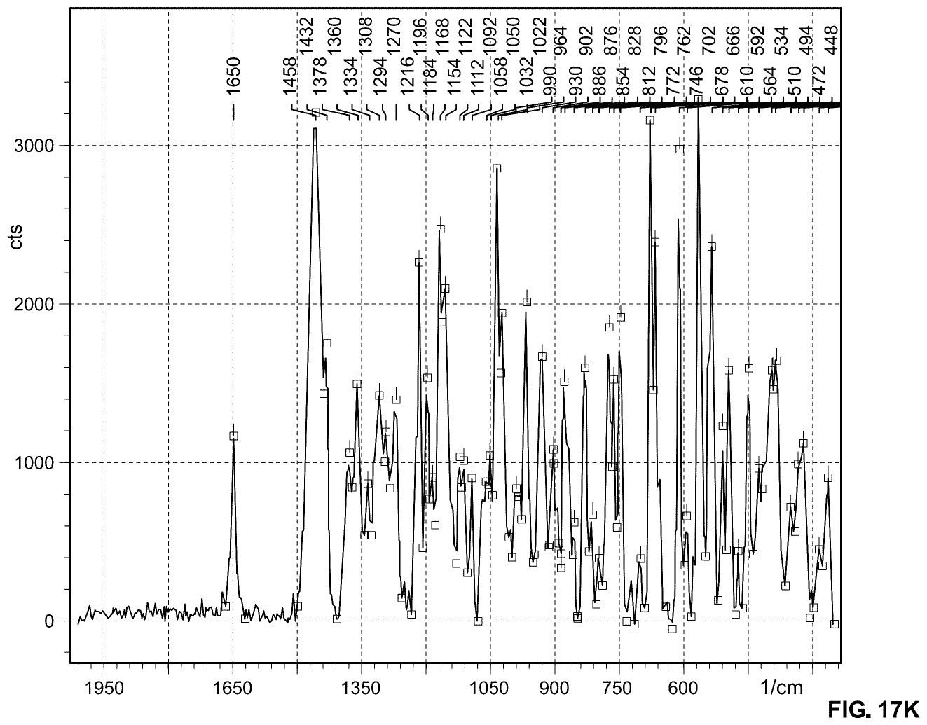

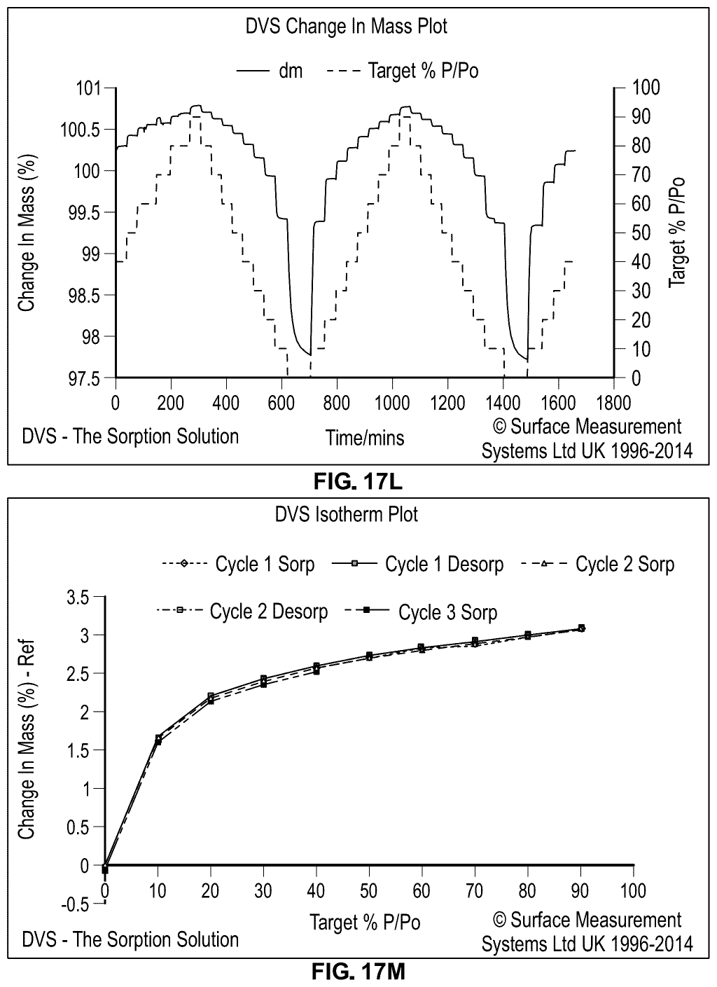

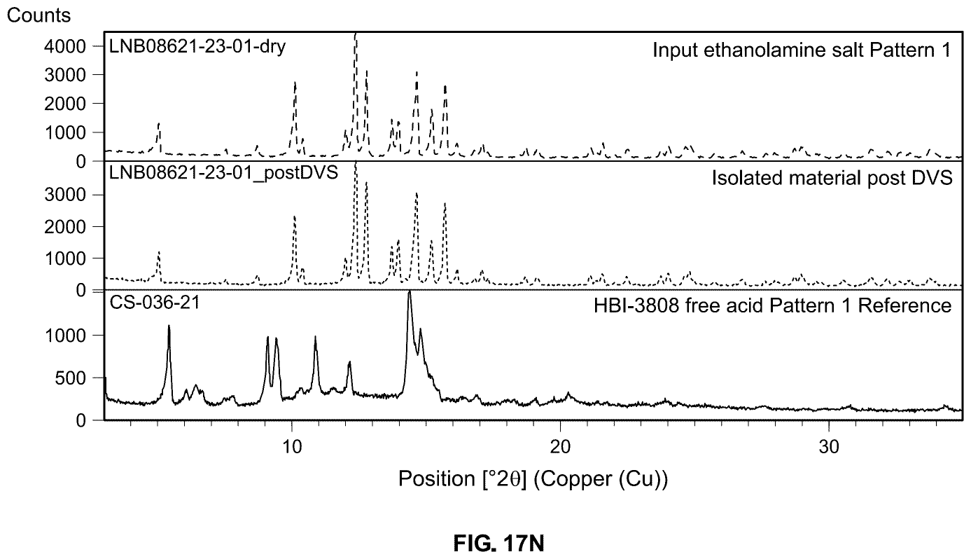

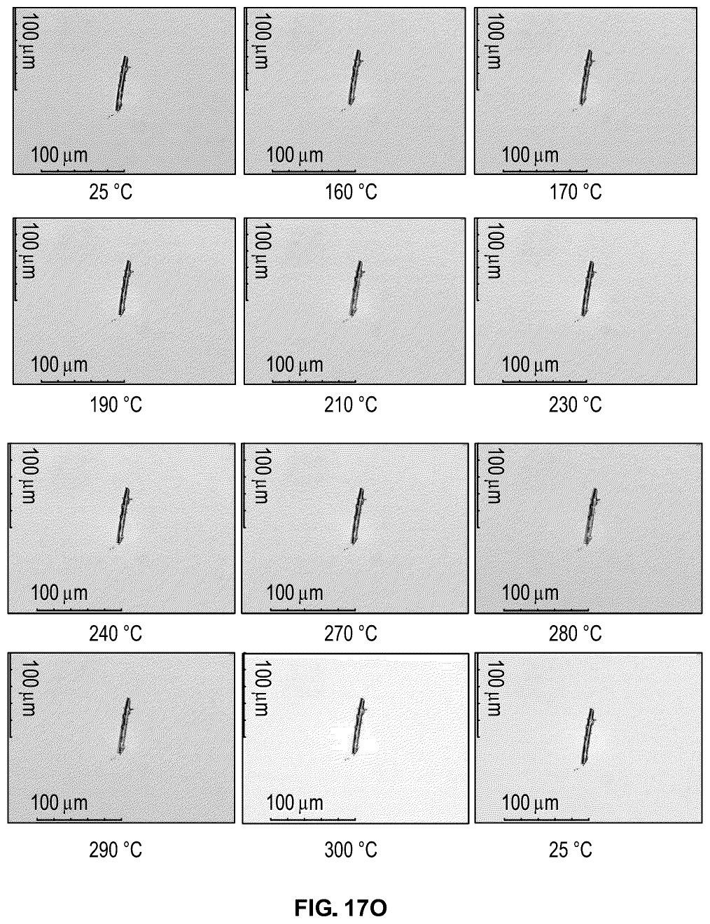

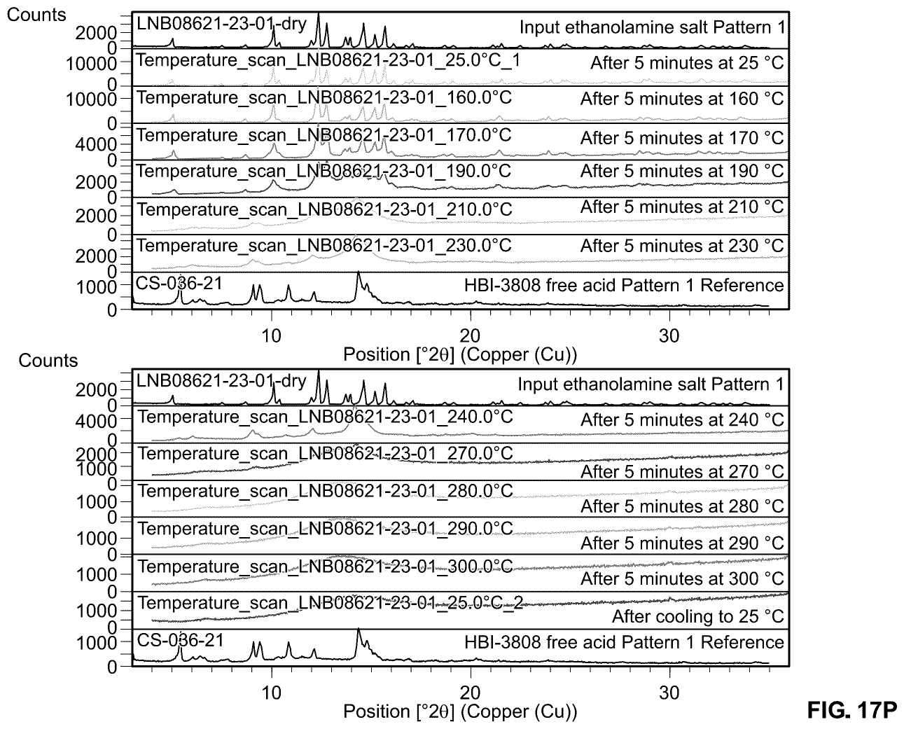

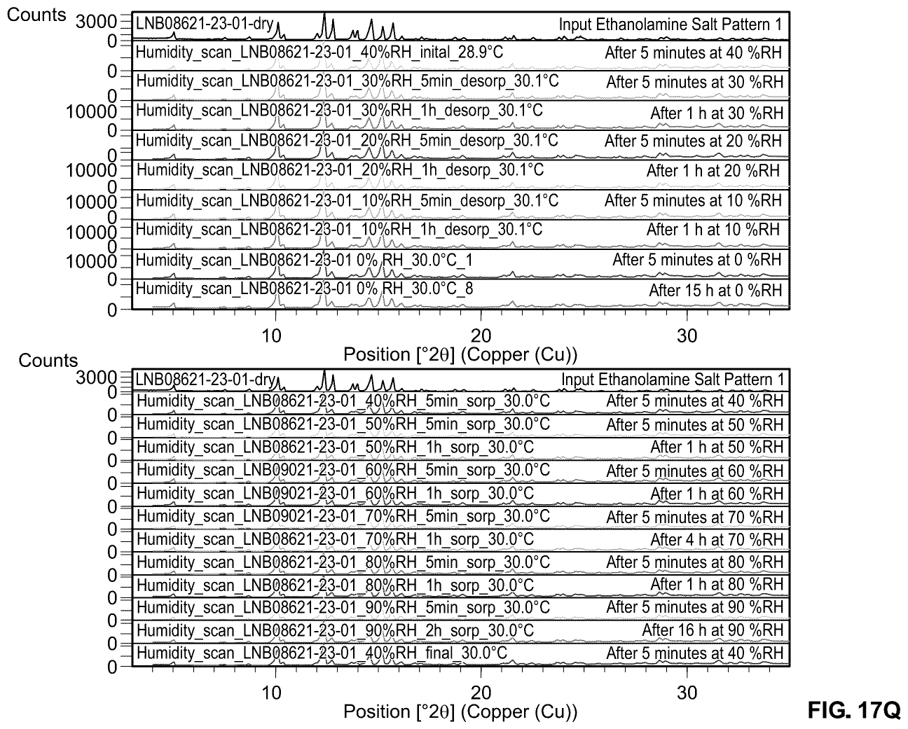

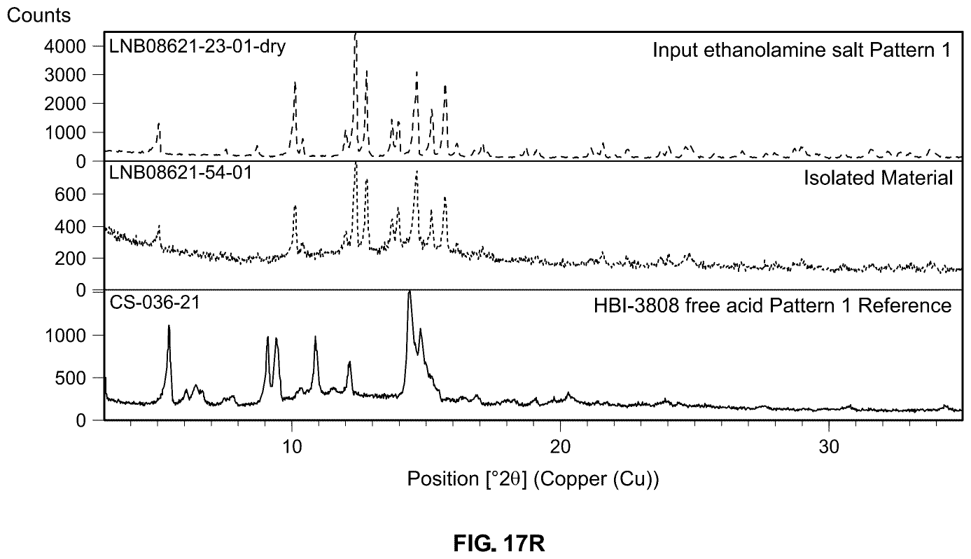

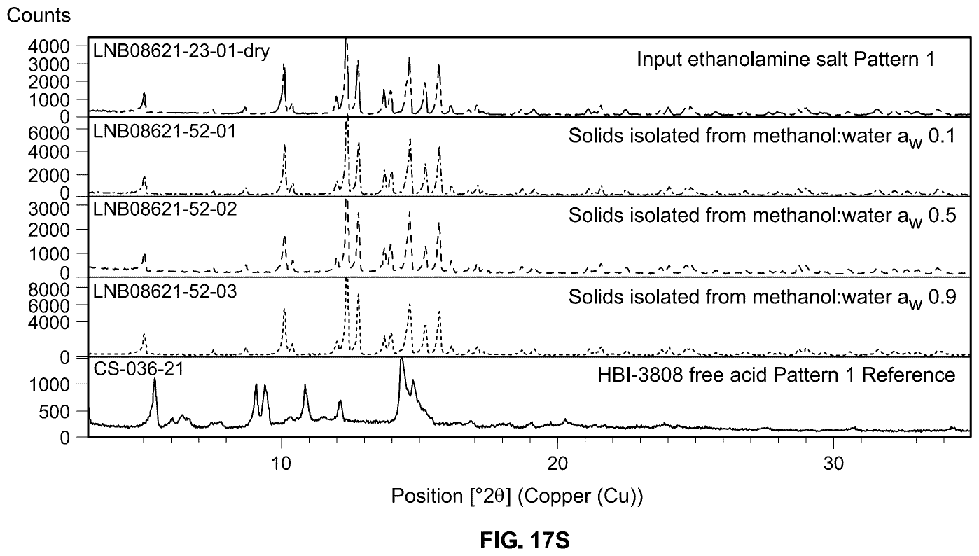

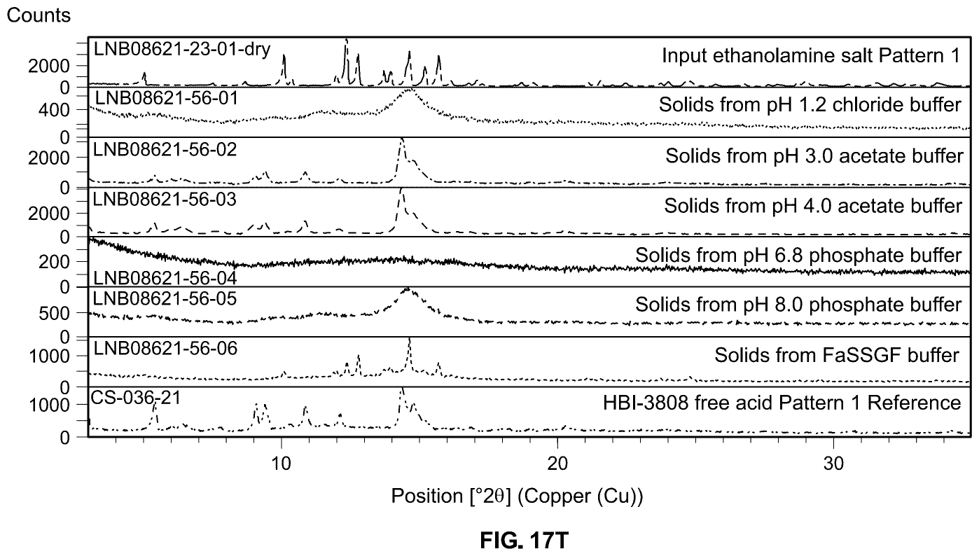

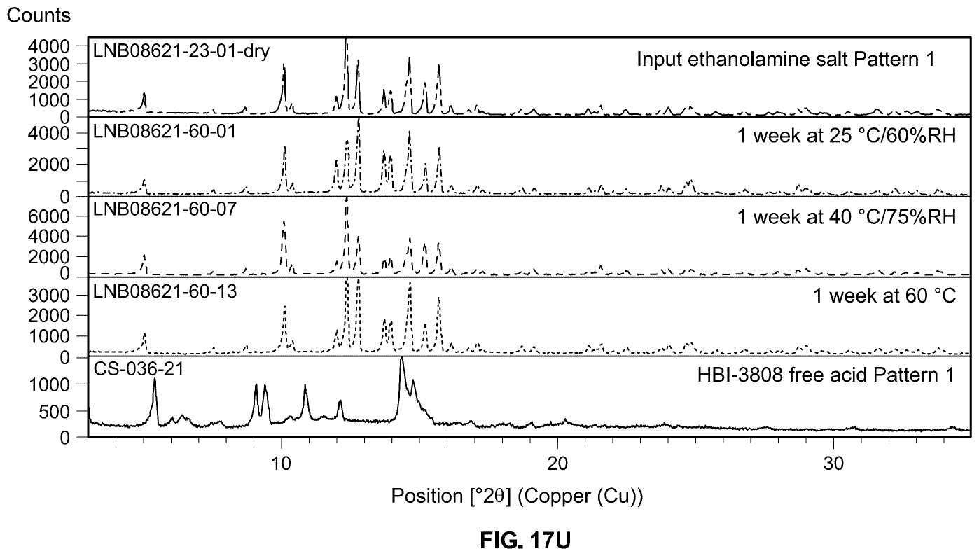

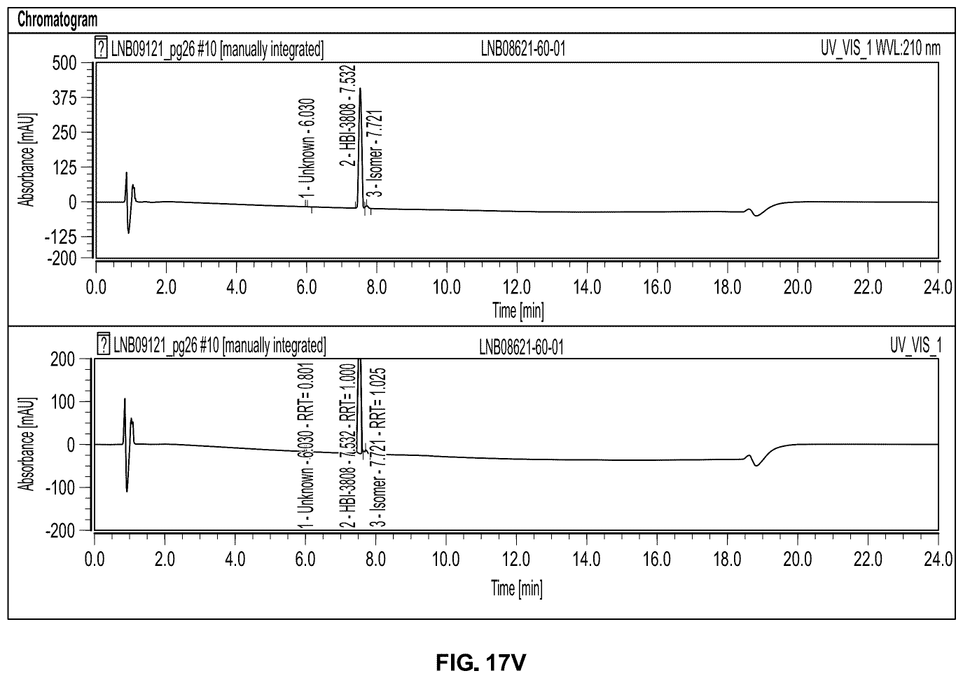

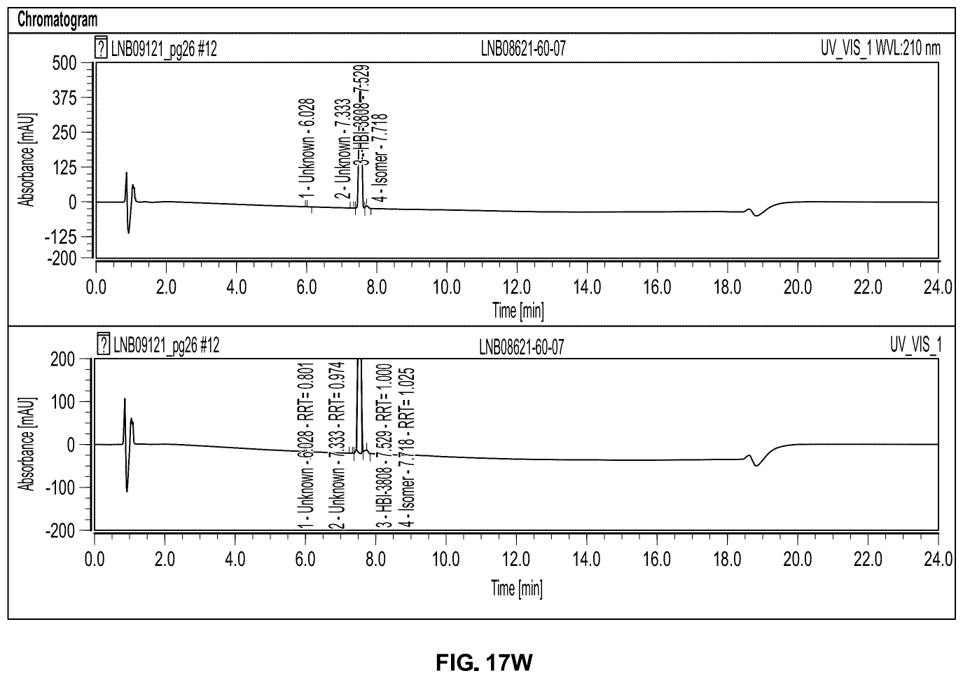

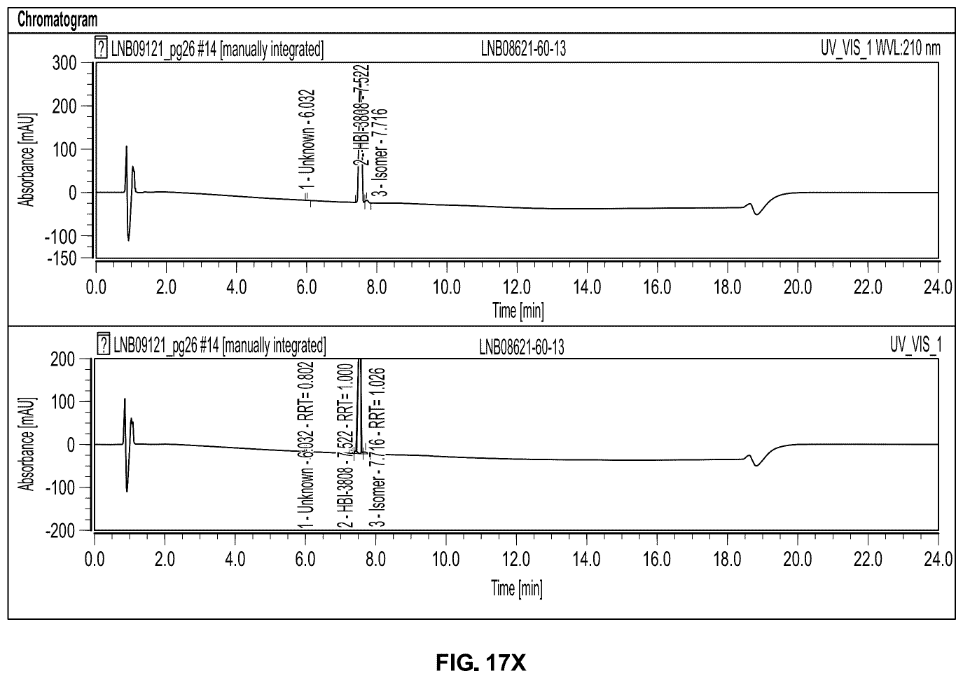

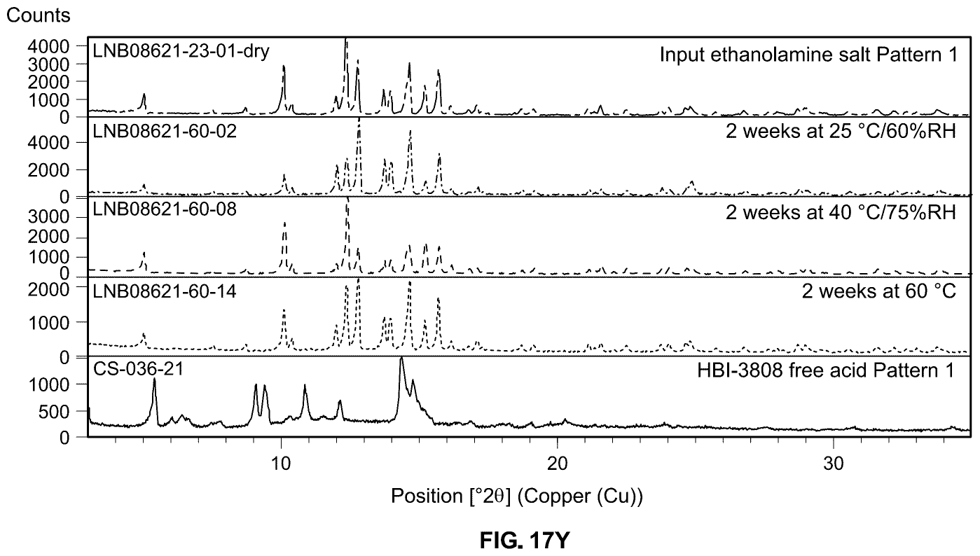

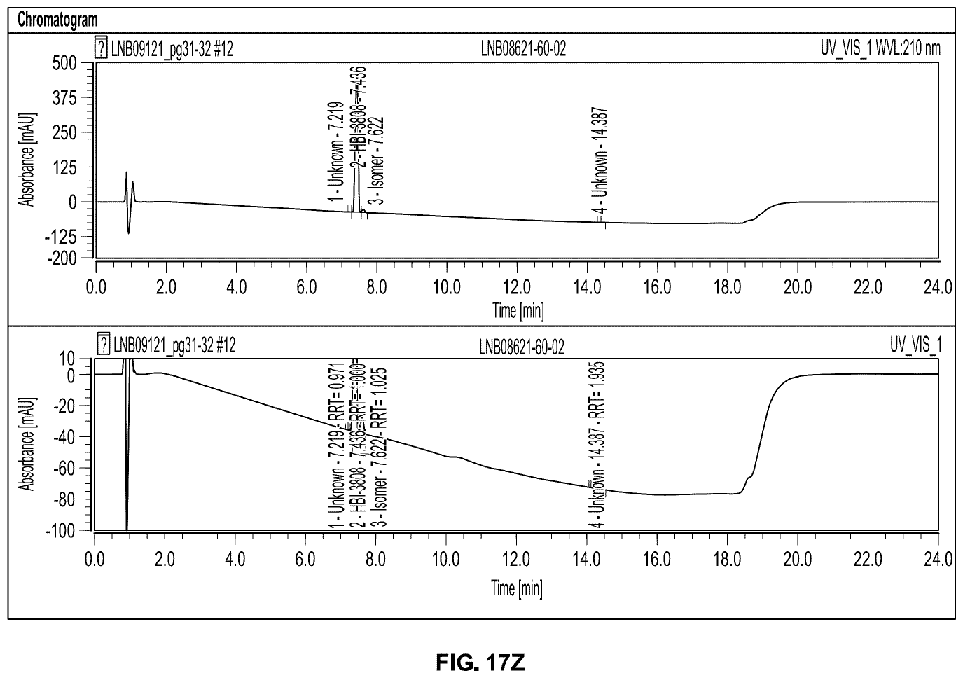

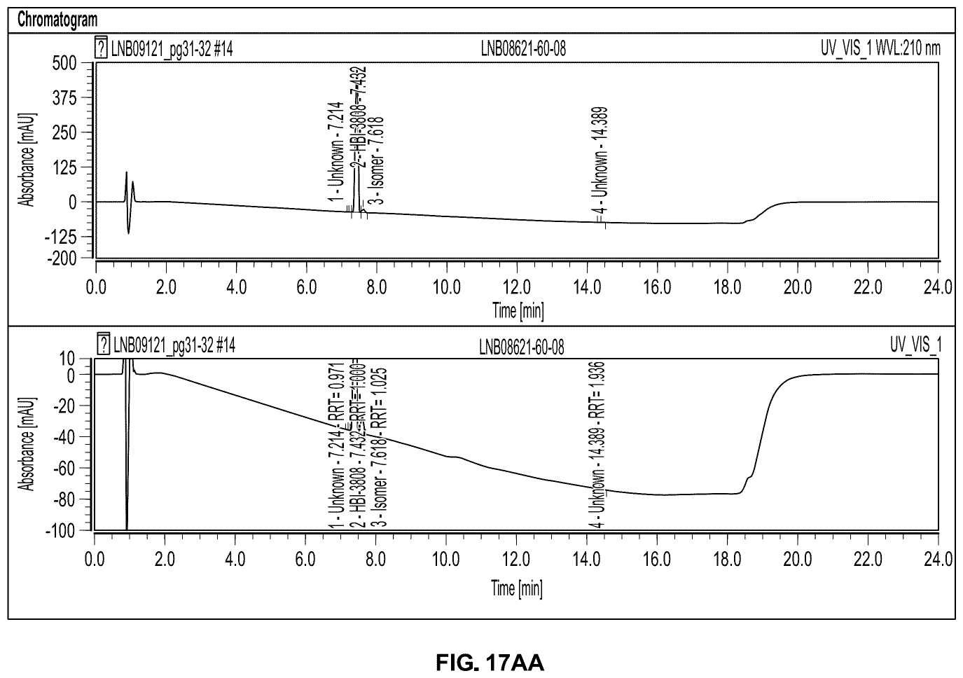

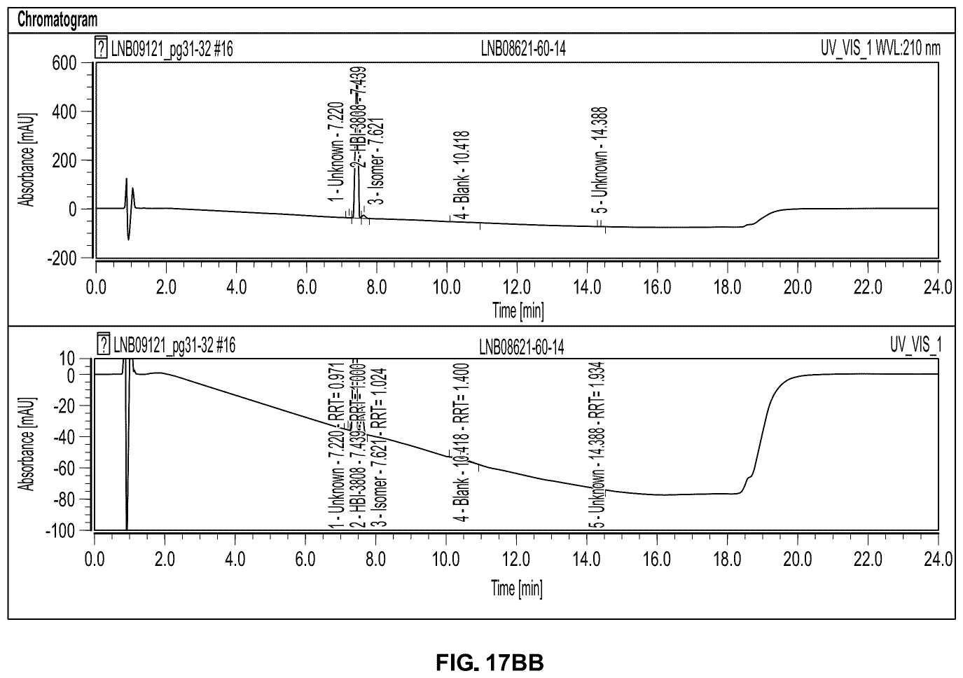

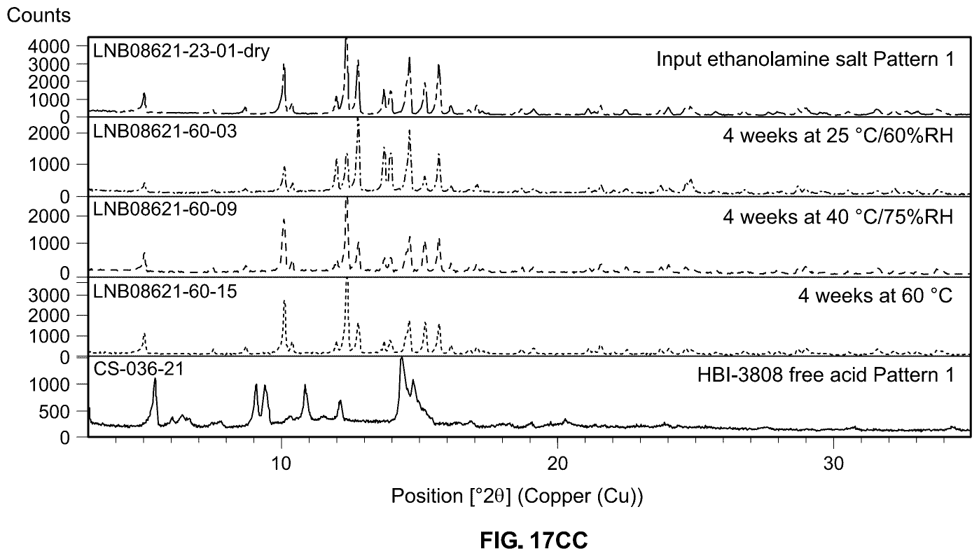

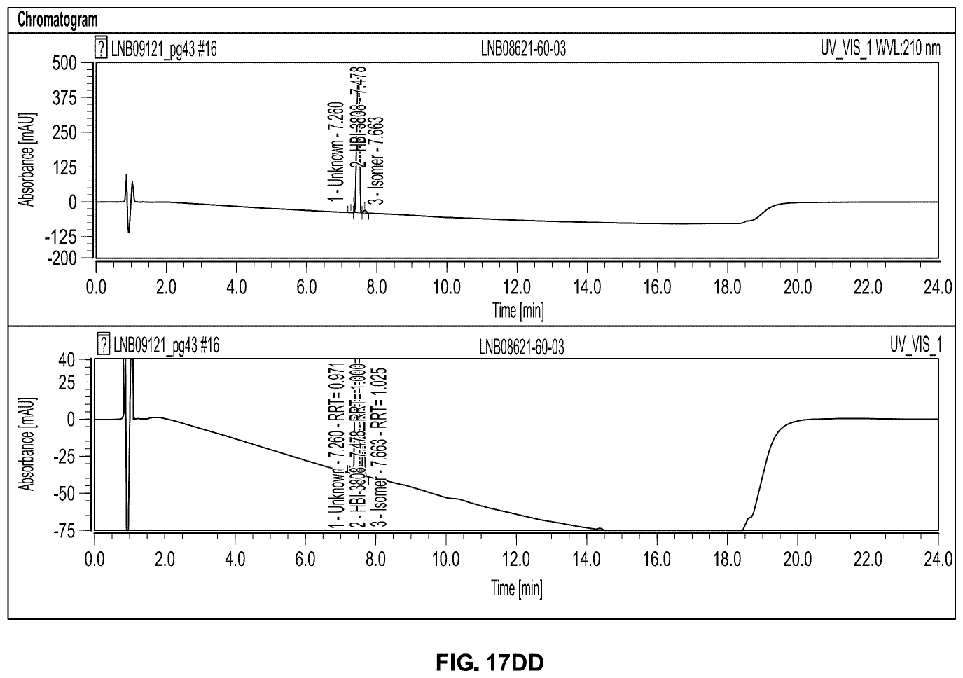

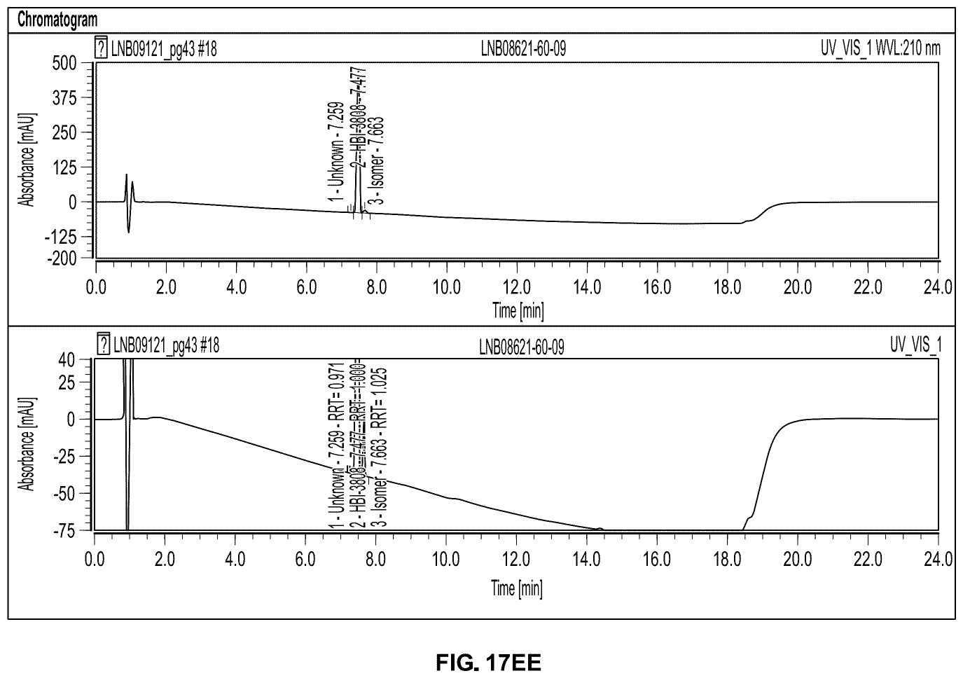

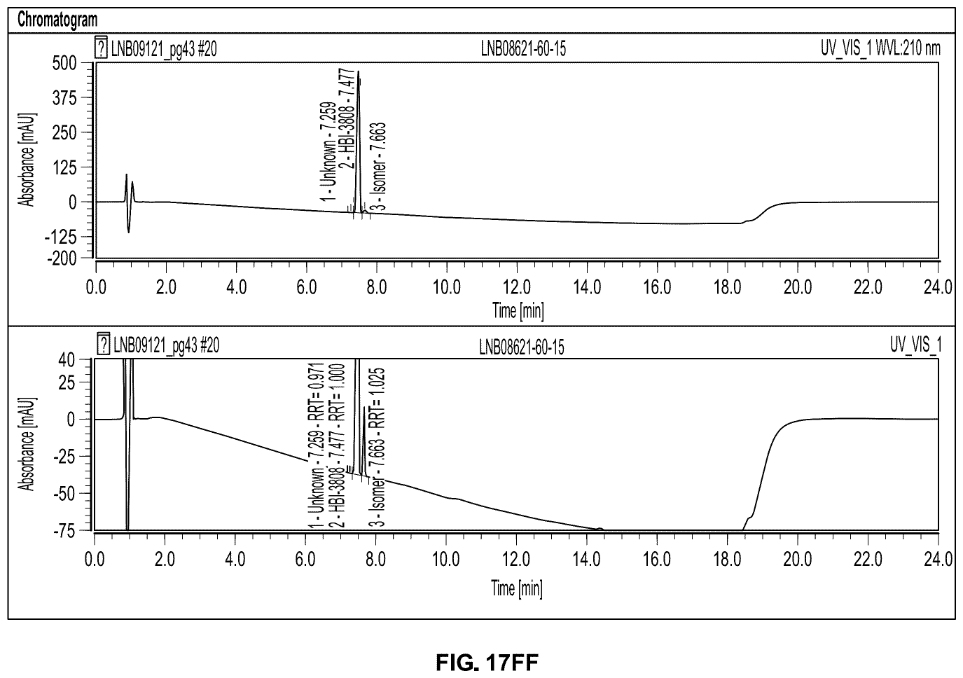

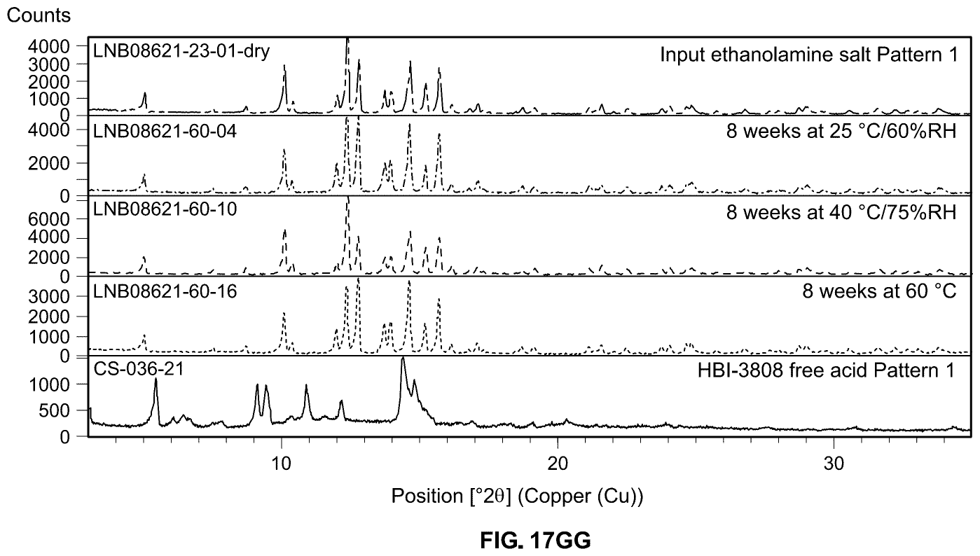

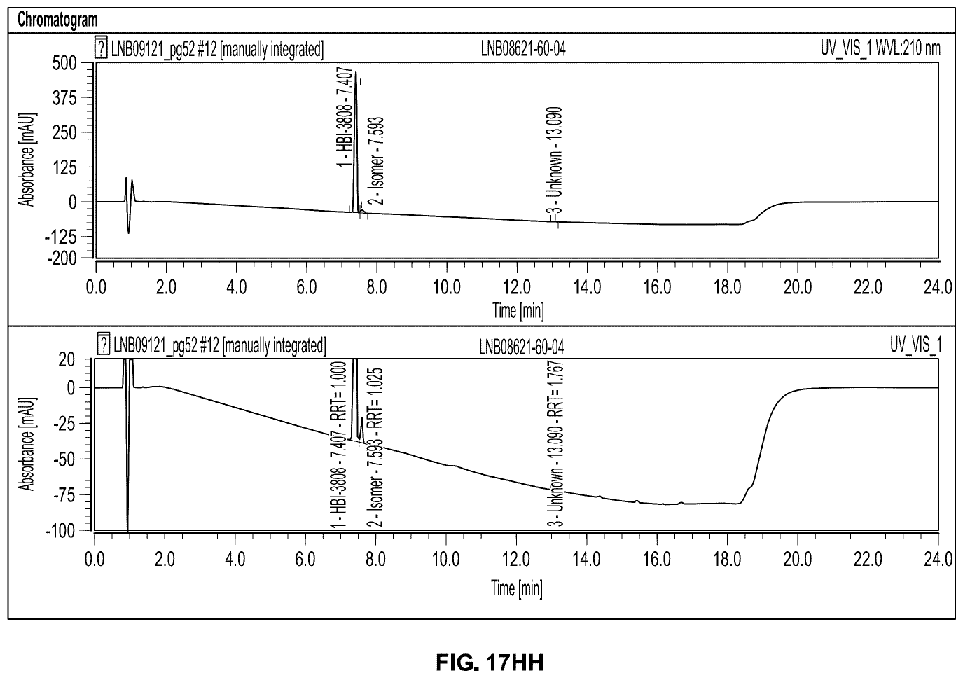

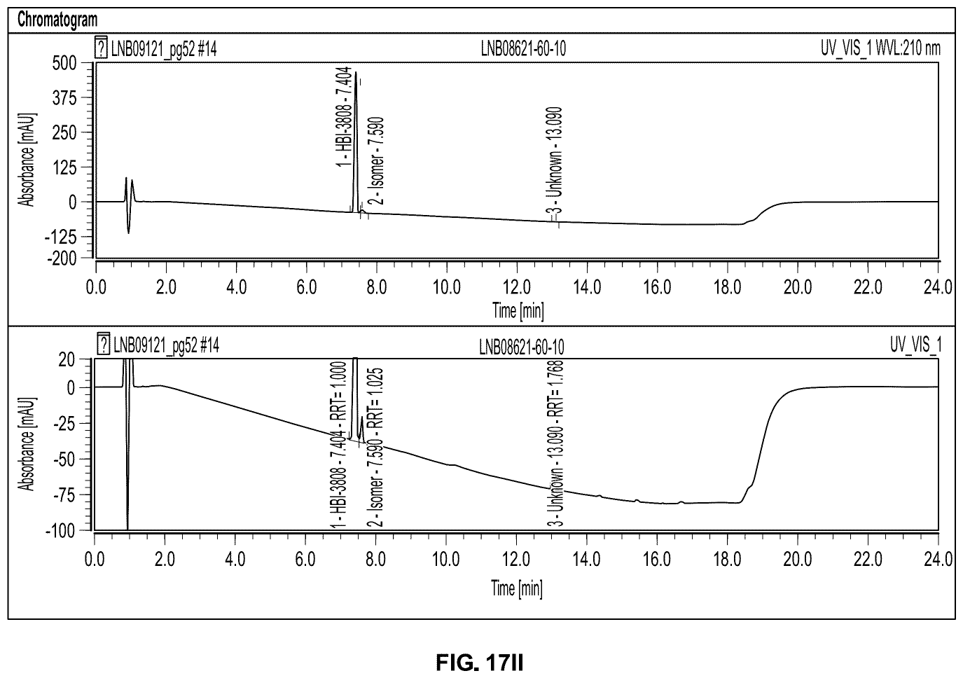

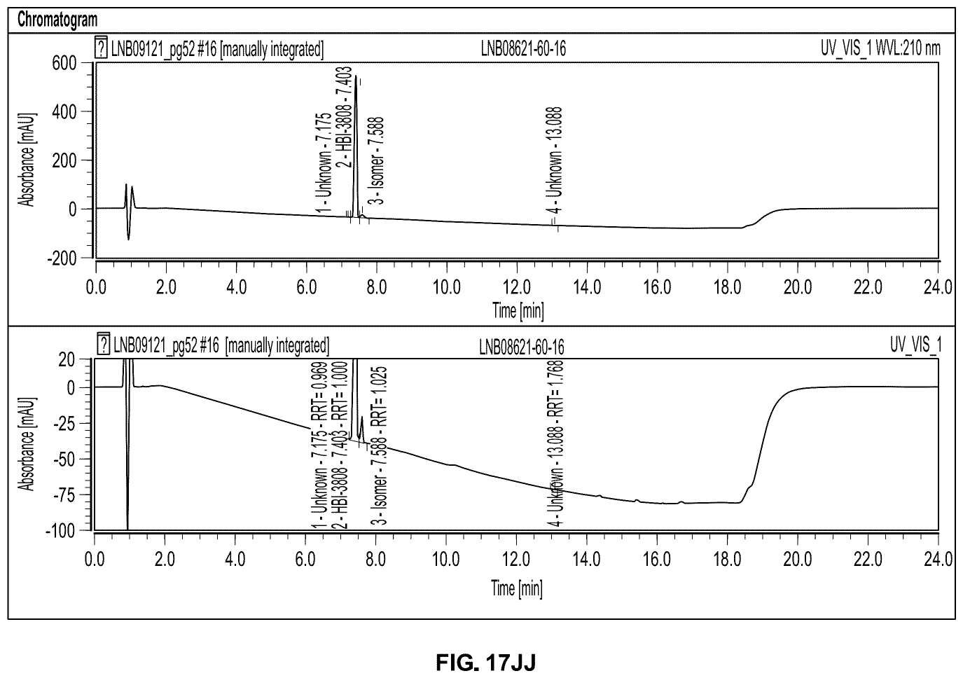

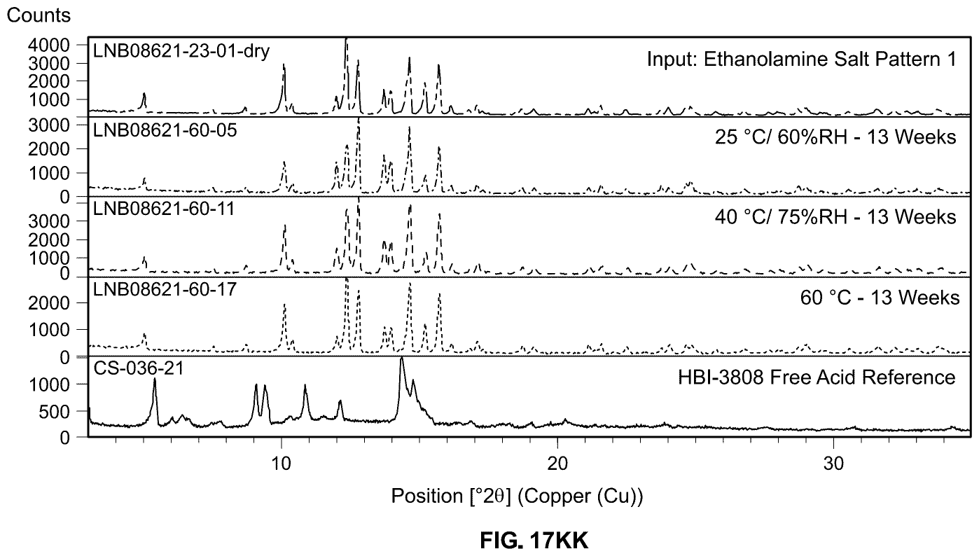

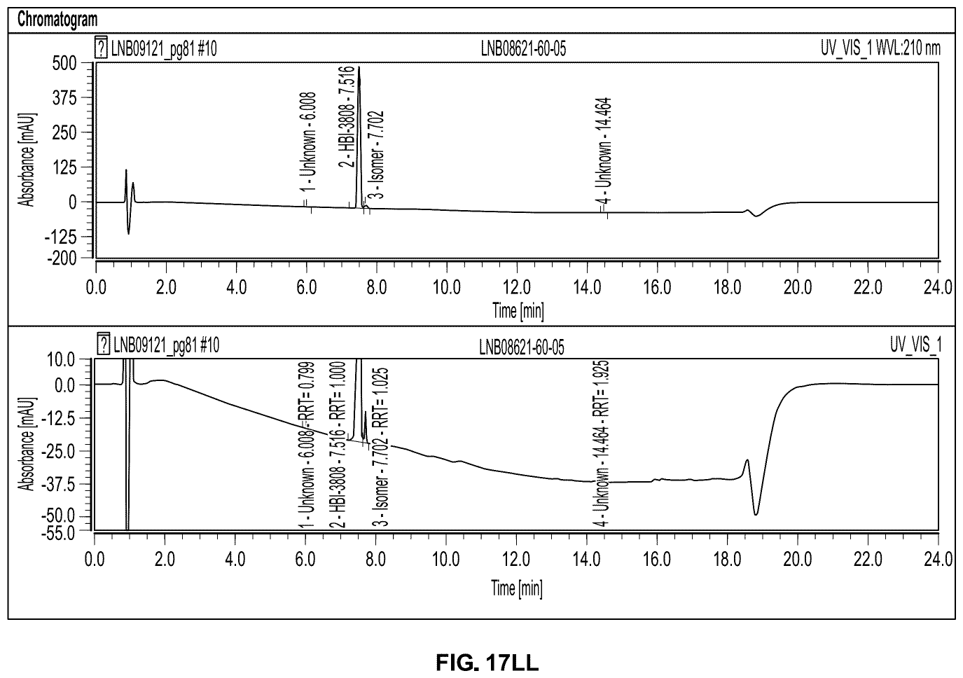

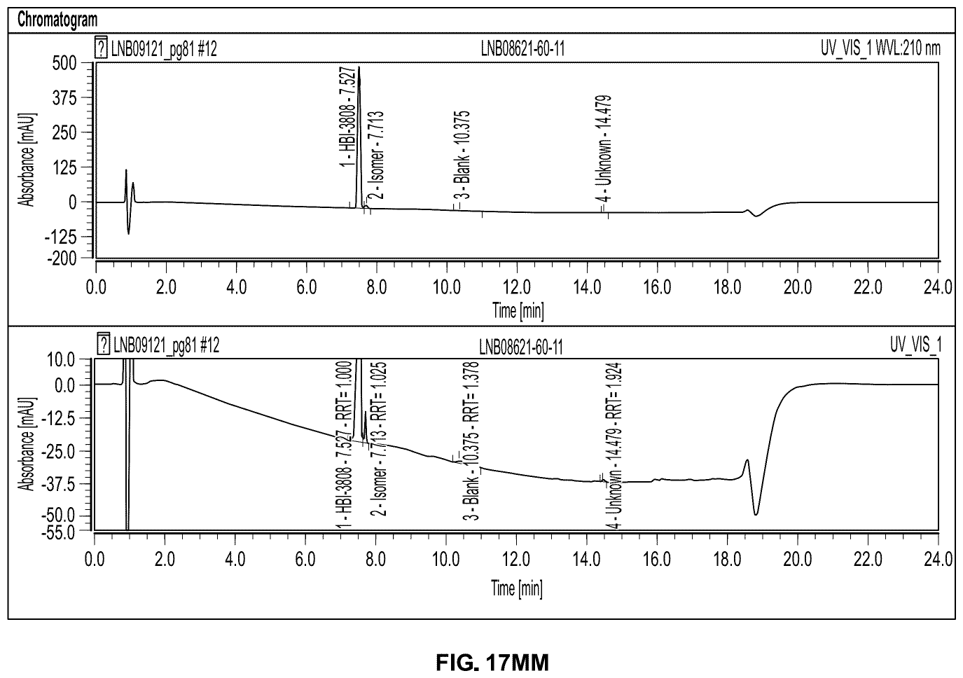

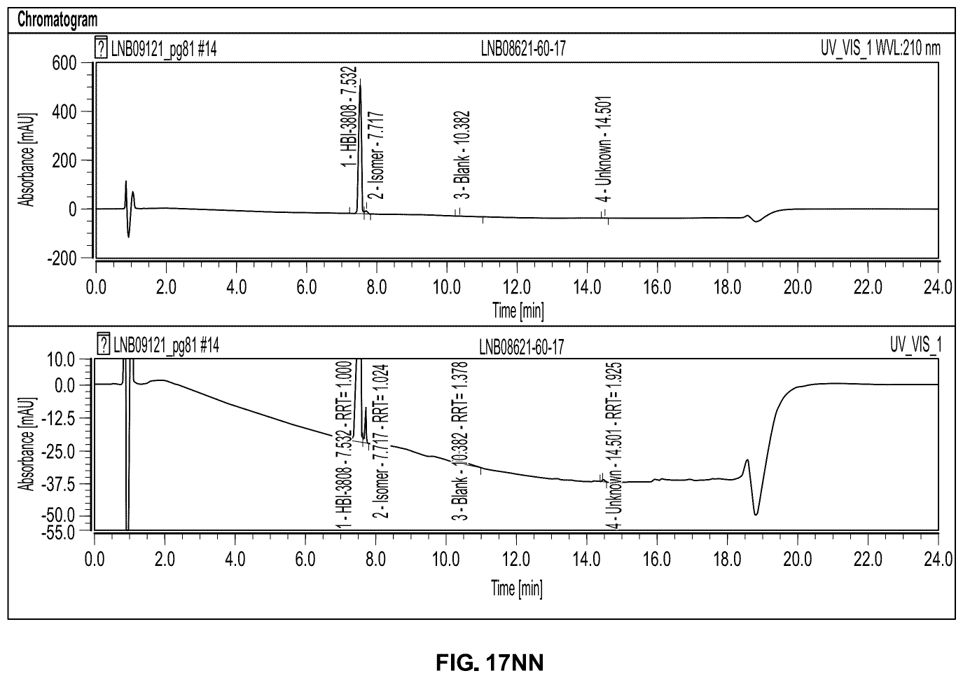

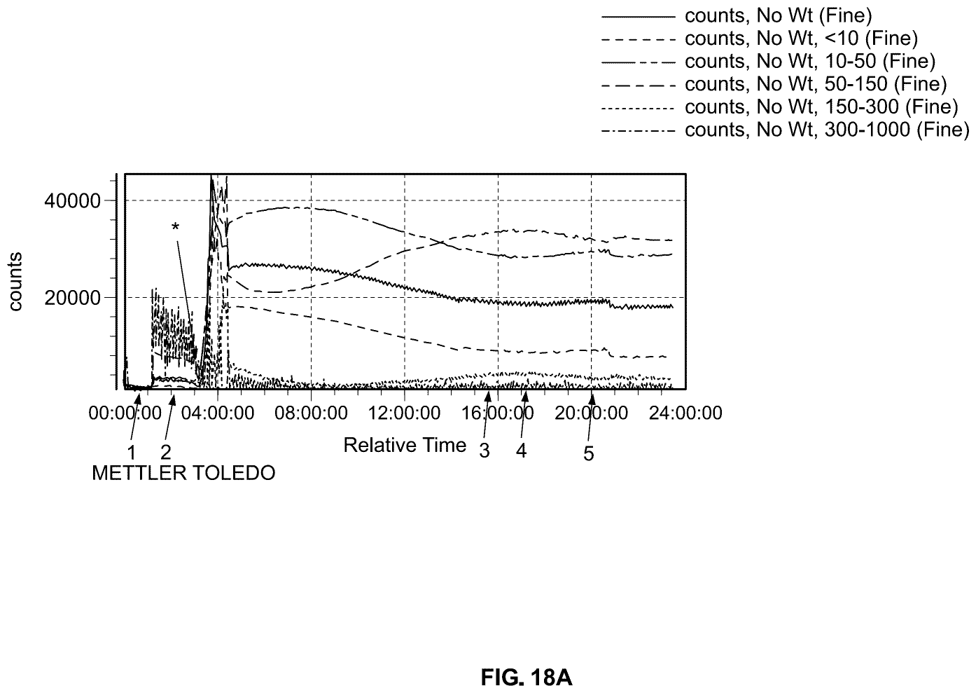

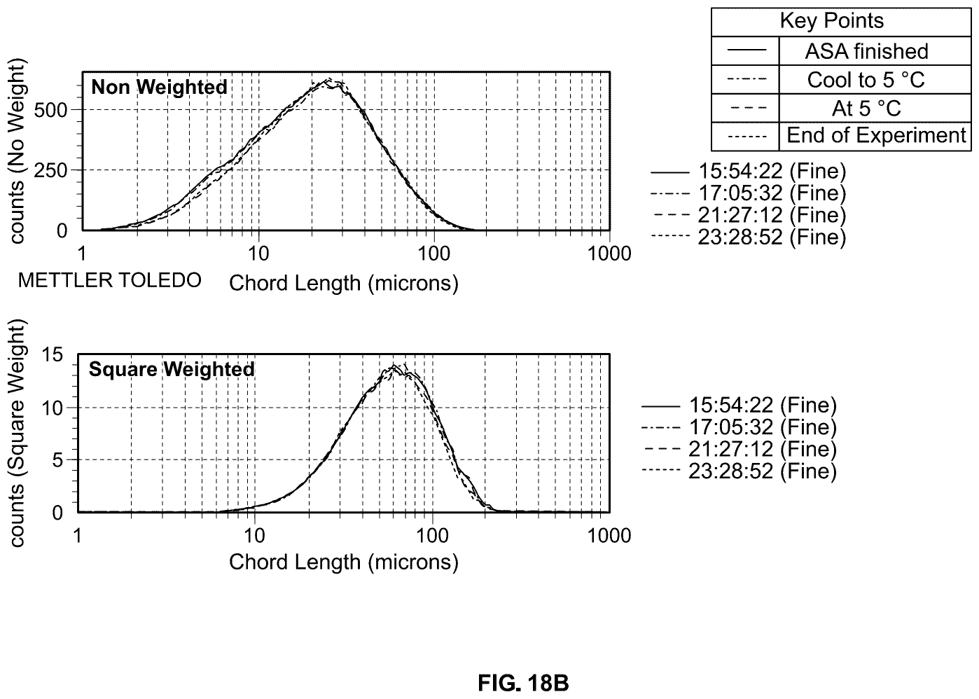

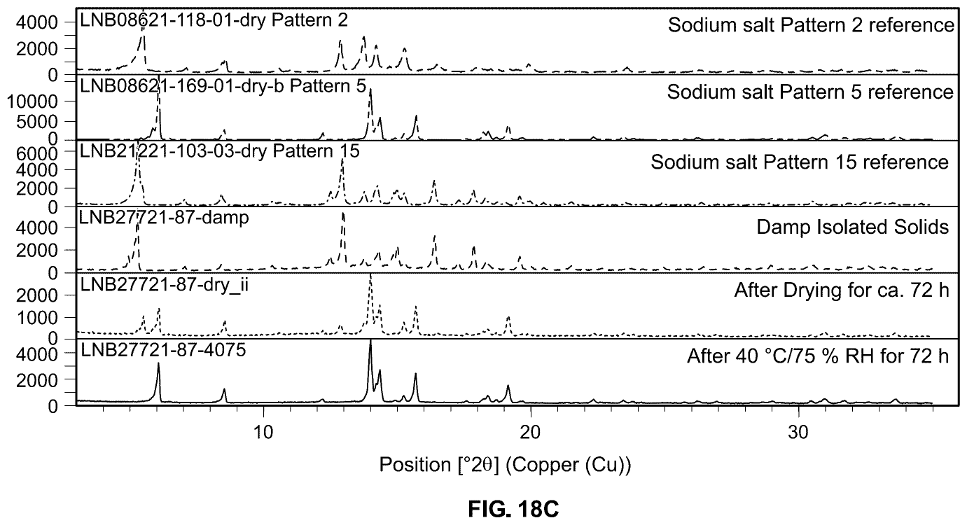

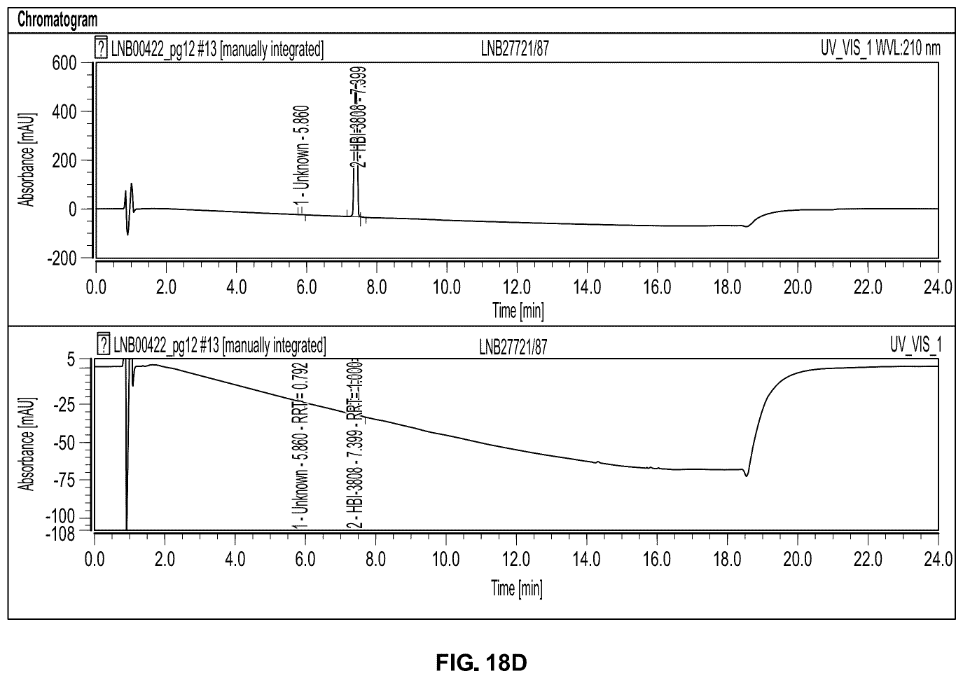

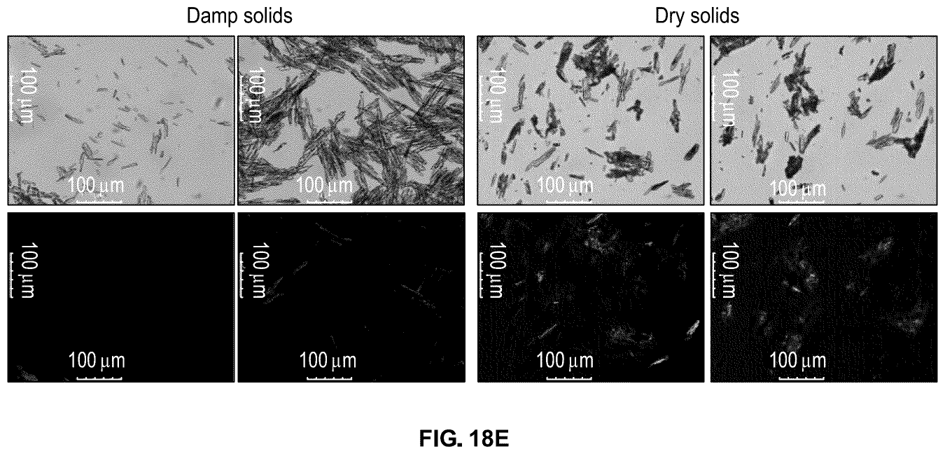

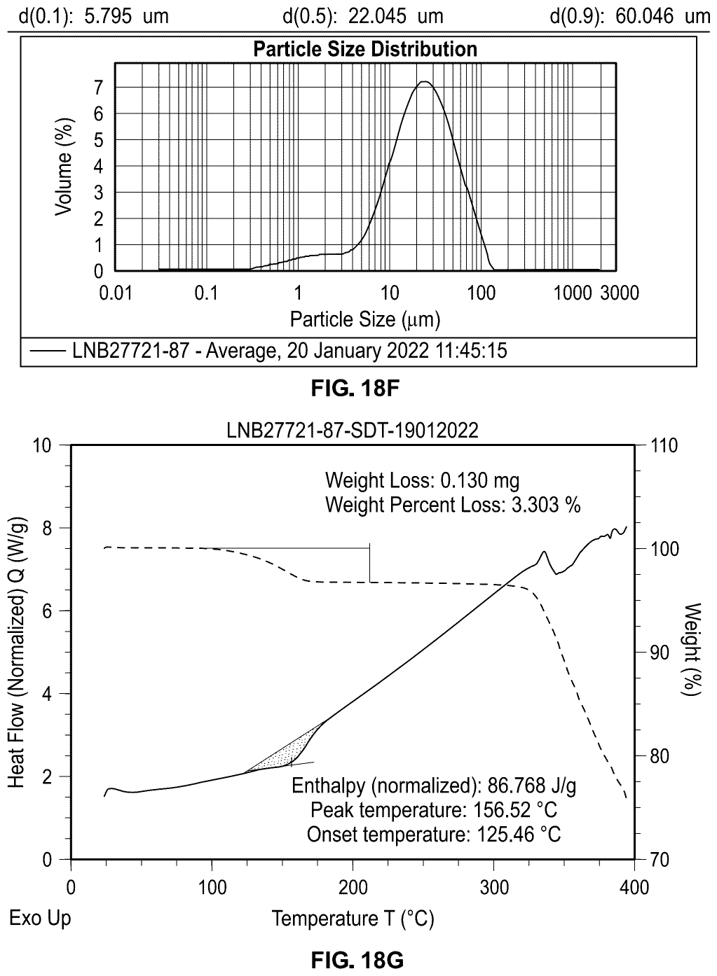

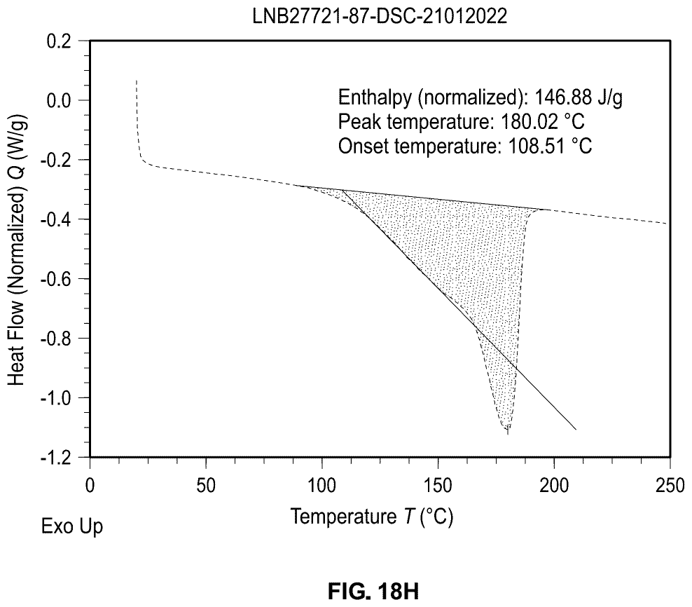

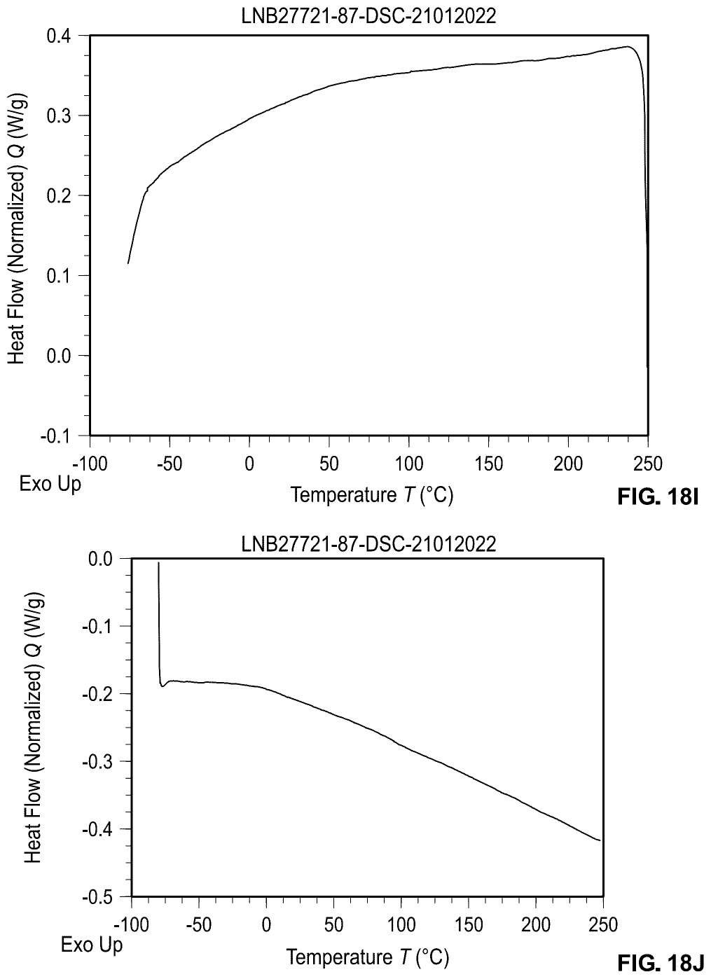

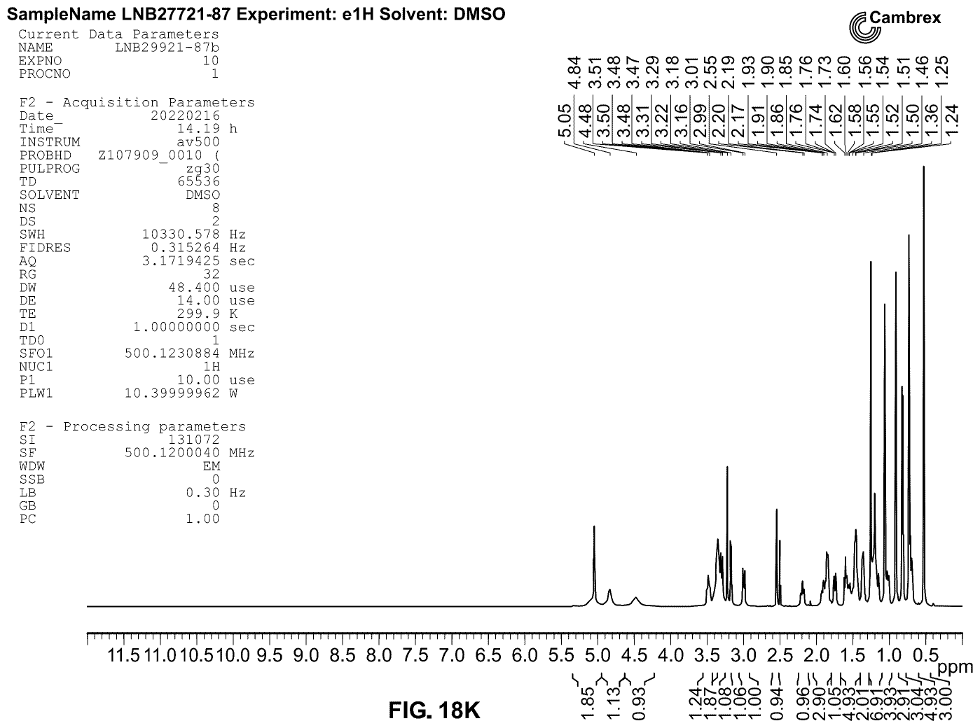

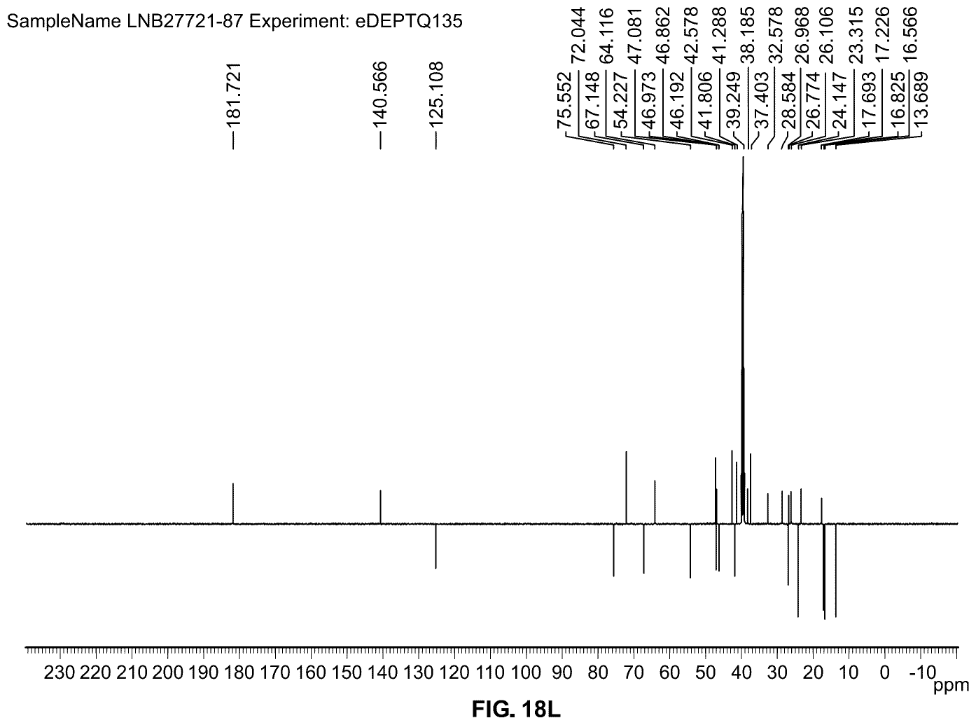

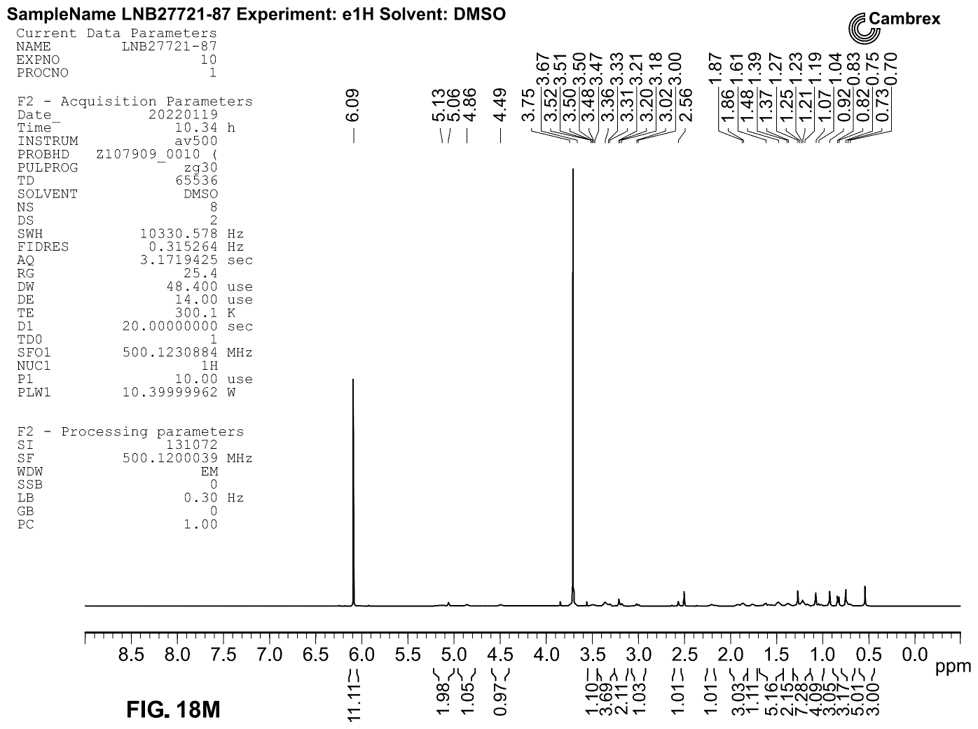

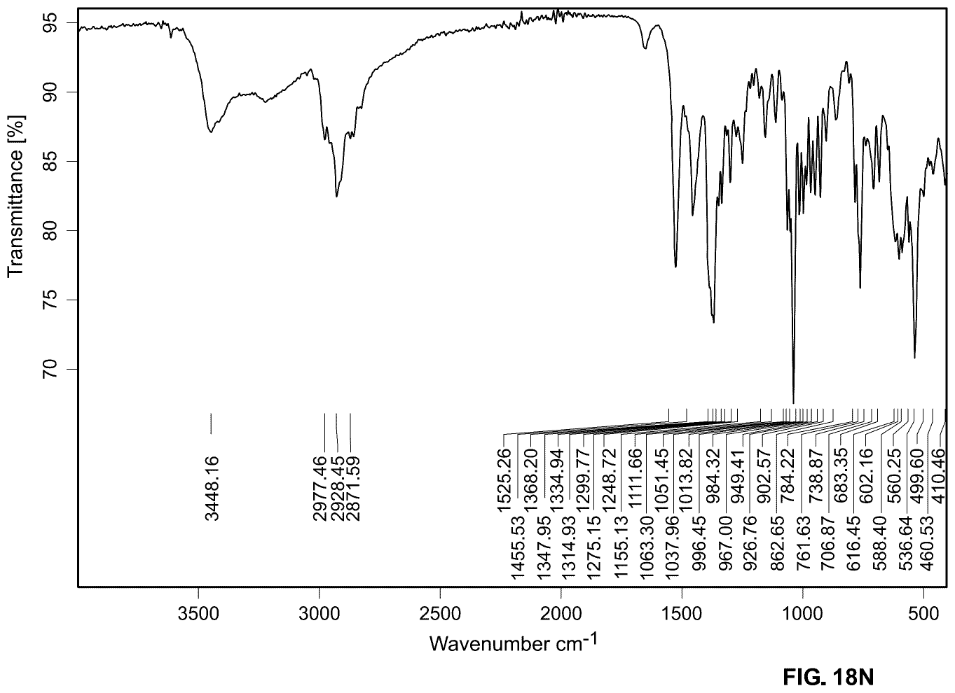

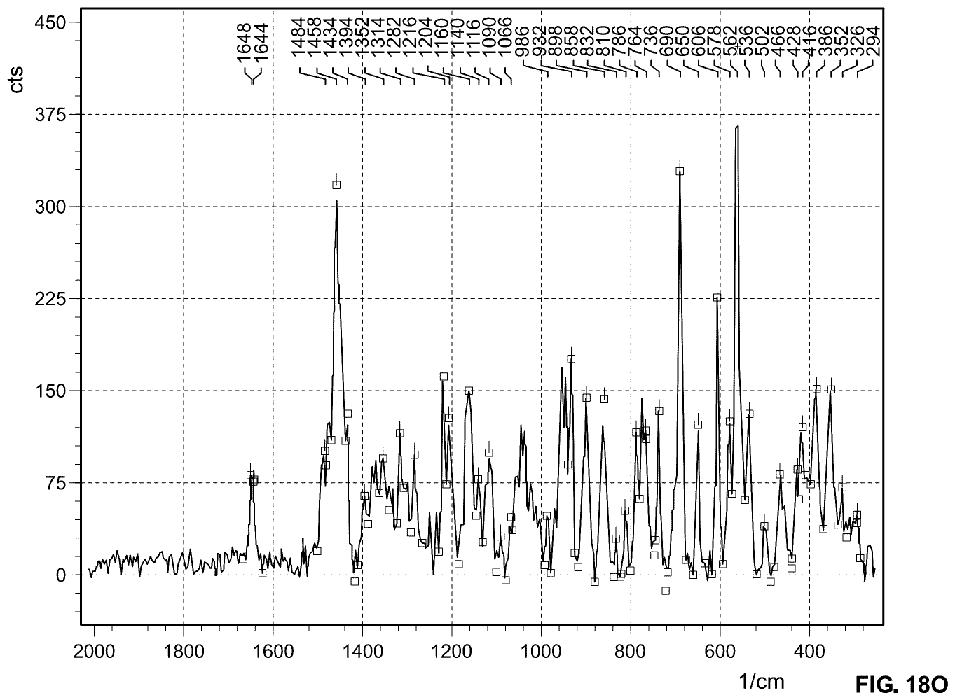

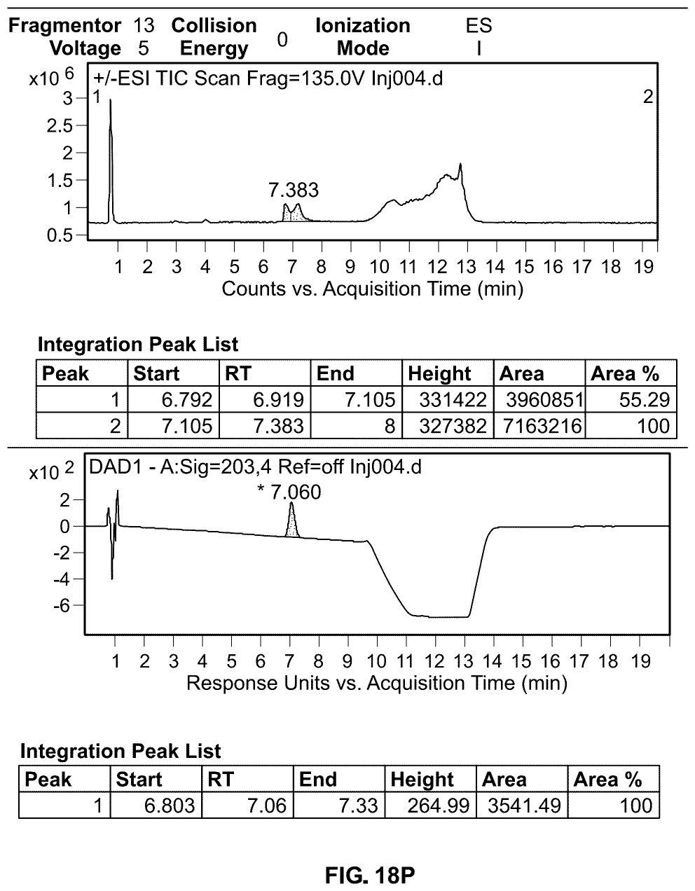

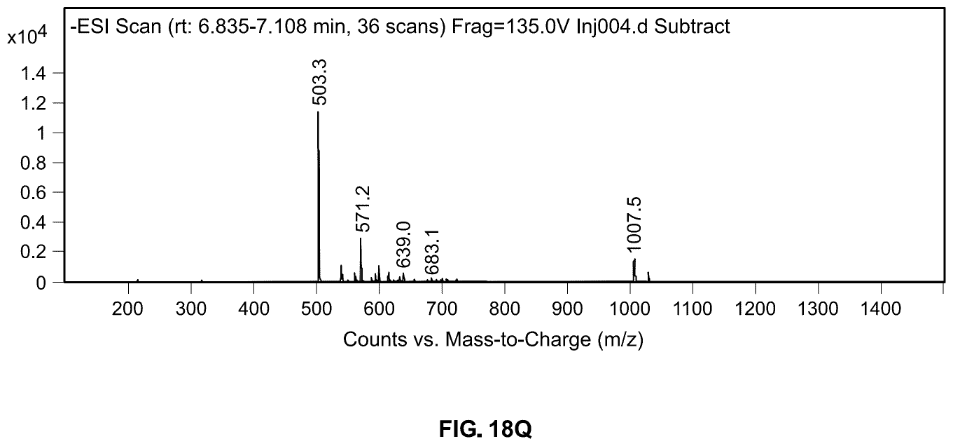

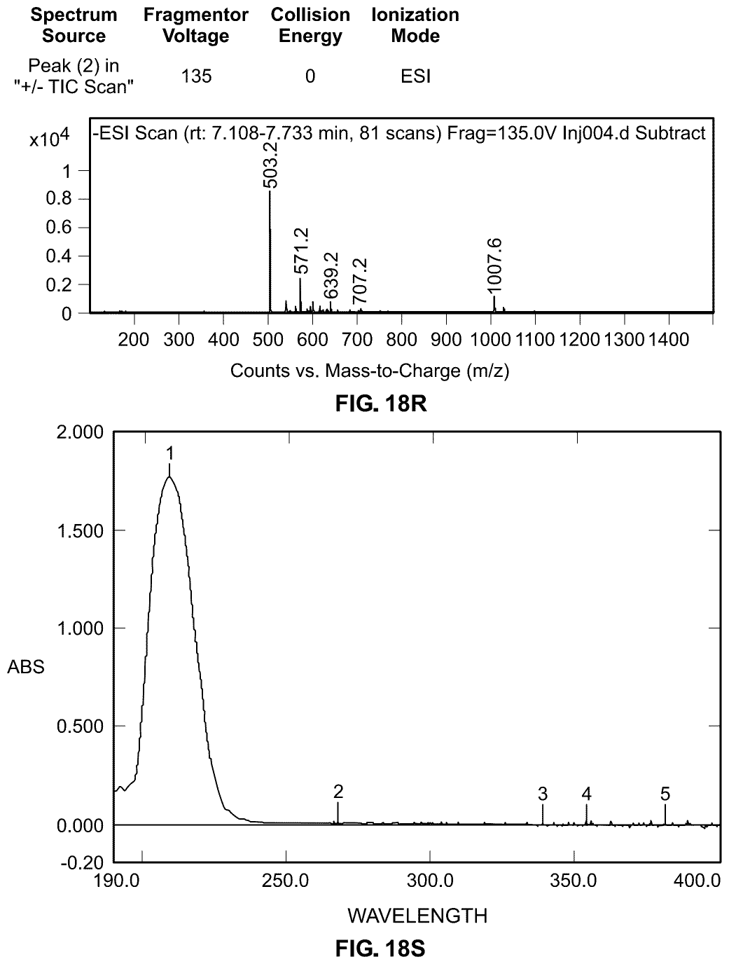

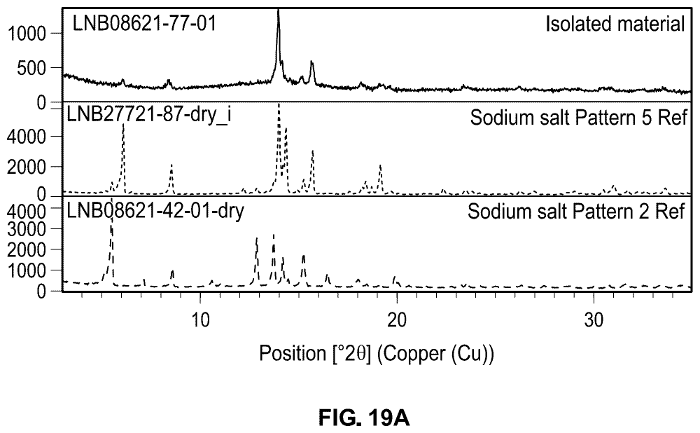

FIG. 2P: VT- XRPD diffractogram of the sodium salt of HBI-3808 of XRPD Pattern 2.

FIG. 2Q: VH- XRPD diffractogram of the sodium salt of HBI-3808 of XRPD Pattern 2, during desorption from 40% to 0%.

FIG. 2R: VH- XRPD diffractogram of the sodium salt of HBI-3808 of XRPD Pattern 2, during sorption from 40% to 90%.

FIG. 2S: VH- XRPD diffractogram of the sodium salt of HBI-3808 of XRPD Pattern 2, during desorption from 80% to 40%.

FIG. 2T: XRPD diffractogram of isolated solids from salt disproportionation study with sodium salt of HBI-3808 of XRPD Pattern 2. Input, sodium salt of HBI-3808 of XRPD Pattern 2 (top), isolated material from the salt disproportionation study (middle), and HBI-3808 acid of XRPD Pattern 1, for reference (bottom).

FIG. 2U: XRPD diffractogram of isolated solids from hydration study with sodium salt of HBI-3808 of XRPD Pattern 2. From the top—Input, sodium salt of HBI-3808 of XRPD Pattern 2; solids isolated from methanol:water a_{w} 0.1; solids isolated from methanol:water a_{w} 0.5; solids isolated from methanol:water a_{W} 0.9; and HBI-3808 acid of XRPD Pattern 1, for reference.

FIG. 2V: XRPD diffractogram of isolated solids from pH dependent solubility study with sodium salt of HBI-3808 of XRPD Pattern 2. From the top—Input, sodium salt of HBI-3808 of XRPD Pattern 2; solids from pH 1.2 chloride buffer; solids from pH 3.0 acetate buffer; solids from pH 4.0 acetate buffer; solids from pH 6.8 phosphate buffer; solids from pH 8.0 phosphate buffer; and HBI-3808 acid of XRPD Pattern 1, for reference.

FIG. 2W: XRPD diffractogram of isolated solids from a 1-week stability study with sodium salt of HBI-3808 of XRPD Pattern 2. From the top—Input, sodium salt of HBI-3808 of XRPD Pattern 2; 1 week at 25° C./60% RH; 1 week at 40° C./75% RH; 1 week at 60° C.; and HBI-3808 acid of XRPD Pattern 1, for reference.

FIG. 2X: HPLC chromatogram of solids obtained from sodium salt of HBI-3808 of XRPD Pattern 2 after 1 week at 25° C./60% RH.

FIG. 2Y: HPLC chromatogram of solids obtained from sodium salt of HBI-3808 of XRPD Pattern 2 after 1 week at 40° C./75% RH.

FIG. 2Z: HPLC chromatogram of solids obtained from sodium salt of HBI-3808 of XRPD Pattern 2 after 1 week at 60° C.

FIG. 2AA: XRPD diffractogram of isolated solids from a 2-week stability study with sodium salt of HBI-3808 of XRPD Pattern 2. From the top—Input, sodium salt of HBI-3808 of XRPD Pattern 2; 2 weeks at 25° C./60% RH; 2 weeks at 40° C./75% RH; 2 weeks at 60° C.; and HBI-3808 acid of XRPD Pattern 1, for reference.

FIG. 2BB: HPLC chromatogram of solids obtained from sodium salt of HBI-3808 of XRPD Pattern 2 after 2 weeks at 25° C./60% RH.

FIG. 2CC: HPLC chromatogram of solids obtained from HBI-3808 sodium salt of Pattern 2 sodium salt of HBI-3808 of XRPD Pattern 2 after 2 weeks at 40° C./75% RH.

FIG. 2DD: HPLC chromatogram of solids obtained from sodium salt of HBI-3808 of XRPD Pattern 2 after 2 weeks at 60° C.

FIG. 2EE: XRPD diffractogram of isolated solids from a 4-week stability study with sodium salt of HBI-3808 of XRPD Pattern 2. From the top—Input, sodium salt of HBI-3808 of XRPD Pattern 2; 4 weeks at 25° C./60% RH; 4 weeks at 40° C./75% RH; 4 weeks at 60° C.; sodium salt of HBI-3808 of XRPD Pattern 5, for reference; and HBI-3808 acid of XRPD Pattern 1, for reference.

FIG. 2FF: HPLC chromatogram of solids obtained from sodium salt of HBI-3808 of XRPD Pattern 2 after 4 weeks at 25° C./60% RH.

FIG. 2GG: HPLC chromatogram of solids obtained from sodium salt of HBI-3808 of XRPD Pattern 2 after 4 weeks at 40° C./75% RH.

FIG. 2HH: HPLC chromatogram of solids obtained from sodium salt of HBI-3808 of XRPD Pattern 2 after 4 weeks at 60° C.

FIG. 2II: XRPD diffractogram of isolated solids from an 8-week stability study with sodium salt of HBI-3808 of XRPD Pattern 2. From the top—Input, sodium salt of HBI-3808 of XRPD Pattern 2; 8 weeks at 25° C./60% RH; 8 weeks at 40° C./75% RH; 8 weeks at 60° C.; sodium salt of HBI-3808 of XRPD Pattern 5, for reference; and HBI-3808 acid of XRPD Pattern 1, for reference.

FIG. 2JJ: HPLC chromatogram of solids obtained from sodium salt of HBI-3808 of XRPD Pattern 2 after 8 weeks at 25° C./60% RH.

FIG. 2KK: HPLC chromatogram of solids obtained from sodium salt of HBI-3808 of XRPD Pattern 2 after 8 weeks at 40° C./75% RH.

FIG. 2LL: HPLC chromatogram of solids obtained from sodium salt of HBI-3808 of XRPD Pattern 2 after 8 weeks at 60° C.

FIG. 2MM: XRPD diffractogram of isolated solids from a 13-week stability study with sodium salt of HBI-3808 of XRPD Pattern 2. From the top—Input, sodium salt of HBI-3808 of XRPD Pattern 2; 13 weeks at 25° C./60% RH; 13 weeks at 40° C./75% RH; 13 weeks at 60° C.; sodium salt of HBI-3808 of XRPD Pattern 5, for reference; and HBI-3808 acid of XRPD Pattern 1, for reference.

FIG. 2NN: HPLC chromatogram of solids obtained from sodium salt of HBI-3808 of XRPD Pattern 2 after 13 weeks at 25° C./60% RH.

FIG. 2OO: HPLC chromatogram of solids obtained from sodium salt of HBI-3808 of XRPD Pattern 2 after 13 weeks at 40° C./70% RH.

FIG. 2PP: HPLC chromatogram of solids obtained from sodium salt of HBI-3808 of XRPD Pattern 2 after 13 weeks at 60° C.

FIG. 3A: XRPD diffractogram of the sodium salt of HBI-3808 of XRPD Pattern 3.

FIG. 3B: Peak fitting of the XRPD diffractogram of the sodium salt of HBI-3808 of XRPD Pattern 3 (FIG. 3A).

FIG. 4A: XRPD diffractogram of the sodium salt of HBI-3808 of XRPD Pattern 4.

FIG. 4B: Peak fitting of the XRPD diffractogram of the sodium salt of HBI-3808 of XRPD Pattern 4 (FIG. 4A).

FIG. 5A: XRPD diffractogram of the sodium salt of HBI-3808 of XRPD Pattern 5.

FIG. 5B: Peak fitting of the XRPD diffractogram of the sodium salt of HBI-3808 of XRPD Pattern 5 (FIG. 5A).

FIG. 5C: XRPD diffractogram of the solids obtained after exposing the sodium salt of HBI-3808 of XRPD Pattern 2, to 25° C./60% RH within a stability chamber for 96 h (middle). Sodium salt of HBI-3808 of XRPD Pattern 2 (top) and, sodium salt of HBI-3808 of XRPD Pattern 5 (bottom) are shown for reference.

FIG. 5D: PLM image of the isolated solids, as described in Example 5. Objective: ×20.

Scale bar 100 μm.

FIG. 5E: HPLC chromatogram of the isolated solids, as described in Example 5. Objective: ×20. Scale bar 100 μm.

FIG. 5F: ¹H NMR spectrum of the isolated solids as described in Example 5, in d⁶-DMSO.

FIG. 5G: TG-DSC thermogram of the isolated solids, as described in Example 5.

FIG. 5H: DSC thermogram of the isolated solids, as described in Example 5.

FIG. 51: DVS mass plot of the isolated solids, as described in Example 5.

FIG. 5J: DVS isotherm plot of the isolated solids, as described in Example 5.

FIG. 5K: XRPD diffractogram of recovered material from DVS analysis of the isolated solids, as described in Example 5.

FIG. 5L: VH- XRPD diffractogram of the sodium salt of HBI-3808 of XRPD Pattern 5.

FIG. 5M: VT- XRPD diffractogram of the sodium salt of HBI-3808 of XRPD Pattern 5.

FIG. 5N: XRPD diffractogram of isolated solids from hydration study with sodium salt of HBI-3808 of XRPD Pattern 5. From the top—Input, sodium salt of HBI-3808 of XRPD Pattern 5; solids isolated from methanol:water a_{w} 0.1; solids isolated from methanol:water a_{w} 0.5; solids isolated from methanol:water a_{W} 0.9; and sodium salt of HBI-3808 of XRPD Pattern 2, for reference.

FIG. 5O: XRPD diffractogram of isolated solids from pH dependent solubility study with sodium salt of HBI-3808 of XRPD Pattern 5. From the top—HBI-3808 acid; sodium salt of HBI-3808 of XRPD Pattern 5, for reference; solids isolated from pH 1.2; solids isolated from pH 3.0; solids isolated from pH 4.0; solids isolated from pH 6.8; and solids isolated from pH 8.

FIG. 5P: XRPD diffractogram of isolated solids from a 1-week stability study with sodium salt of HBI-3808 of XRPD Pattern 5. From the top—Input, sodium salt of HBI-3808 of XRPD Pattern 5; 1 week at 25° C./60% RH; 1 week at 40° C./75% RH; 1 week at 60° C.; and HBI-3808 acid of XRPD Pattern 1, for reference.

FIG. 5Q: HPLC chromatogram of solids obtained from sodium salt of HBI-3808 of XRPD Pattern 5 after 1 week at 25° C./60% RH.

FIG. 5R: HPLC chromatogram of solids obtained from sodium salt of HBI-3808 of XRPD Pattern 5 after 1 week at 40° C./75% RH.

FIG. 5S: HPLC chromatogram of solids obtained from sodium salt of HBI-3808 of XRPD Pattern 5 after 1 week at 60° C.

FIG. 5T: XRPD diffractogram of isolated solids from a 2-week stability study with sodium salt of HBI-3808 of XRPD Pattern 5. From the top—Input, sodium salt of HBI-3808 of XRPD Pattern 5; 2 weeks at 25° C./60% RH; 2 weeks at 40° C./75% RH; 2 weeks at 60° C.; and HBI-3808 acid of XRPD Pattern 1, for reference.

FIG. 5U: HPLC chromatogram of solids obtained from sodium salt of HBI-3808 of XRPD Pattern 5 after 2 weeks at 25° C./60% RH.

FIG. 5V: HPLC chromatogram of solids obtained from HBI-3808 of XRPD Pattern 5 after 2 weeks at 40° C./75% RH.

FIG. 5W: HPLC chromatogram of solids obtained from sodium salt of HBI-3808 of XRPD Pattern 5 after 2 weeks at 60° C.

FIG. 5X: XRPD diffractogram of isolated solids from a 4-week stability study with sodium salt of HBI-3808 of XRPD Pattern 5. From the top—Input, sodium salt of HBI-3808 of XRPD Pattern 5; 4 weeks at 25° C./60% RH; 4 weeks at 40° C./75% RH; 4 weeks at 60° C.; and HBI-3808 acid of XRPD Pattern 1, for reference.

FIG. 5Y: HPLC chromatogram of solids obtained from sodium salt of HBI-3808 of XRPD Pattern 5 after 4 weeks at 25° C./60% RH.

FIG. 5Z: HPLC chromatogram of solids obtained from sodium salt of HBI-3808 of XRPD Pattern 5 after 4 weeks at 40° C./75% RH.

FIG. 5AA: HPLC chromatogram of solids obtained from sodium salt of HBI-3808 of XRPD Pattern 5 after 4 weeks at 60° C.

FIG. 6A: XRPD diffractogram of the sodium salt of HBI-3808 of XRPD Pattern 6.

FIG. 6B: Peak fitting of the XRPD diffractogram of the sodium salt of HBI-3808 of XRPD Pattern 6 (FIG. 6A).

FIG. 7A: XRPD diffractogram of the sodium salt of HBI-3808 of XRPD Pattern 7.

FIG. 7B: Peak fitting of the XRPD diffractogram of the sodium salt of HBI-3808 of XRPD Pattern 7 (FIG. 7A).

FIG. 8A: XRPD diffractogram of the sodium salt of HBI-3808 of XRPD Pattern 8.

FIG. 8B: Peak fitting of the XRPD diffractogram of the sodium salt of HBI-3808 of XRPD Pattern 8 (FIG. 8A).

FIG. 8C: PLM image of the damp and dried solids, as described in Example 8.

Objective: ×20. Scale bar 100 μm.

FIG. 8D: TG-DSC thermogram of the isolated solids, as described in Example 8.

FIG. 8E: ¹H NMR spectrum of the isolated solids as described in Example 8, in d⁶-DMSO.

FIG. 8F: HPLC chromatogram of the isolated solids, as described in Example 8.

FIG. 8G: PSD image of the isolated solids, as described in Example 8.

FIG. 9A: XRPD diffractogram of the sodium salt of HBI-3808 of XRPD Pattern 9.

FIG. 9B: Peak fitting of the XRPD diffractogram of the sodium salt of HBI-3808 of XRPD Pattern 9 (FIG. 9A).

FIG. 10A: XRPD diffractogram of the sodium salt of HBI-3808 of XRPD Pattern 10.

FIG. 10B: Peak fitting of the XRPD diffractogram of the sodium salt of HBI-3808 of XRPD Pattern 10 (FIG. 10A).

FIG. 11A: XRPD diffractogram of the sodium salt of HBI-3808 of XRPD Pattern 11.

FIG. 11B: Peak fitting of the XRPD diffractogram of the sodium salt of HBI-3808 of XRPD Pattern 11 (FIG. 11A).

FIG. 11C: TG-DSC thermogram of the isolated solids, as described in Example 11.

FIG. 12A: XRPD diffractogram of the sodium salt of HBI-3808 of XRPD Pattern 12.

FIG. 12B: Peak fitting of the XRPD diffractogram of the sodium salt of HBI-3808 of XRPD Pattern 12 (FIG. 12A).

FIG. 12C: TG-DSC thermogram of the isolated solids, as described in Example 12.

FIG. 12D: ¹H NMR spectrum of the isolated solids as described in Example 12, in d⁶-DMSO.

FIG. 12E: XRPD diffractogram after desolvation of the sodium salt of HBI-3808 of XRPD Pattern 12. From the top—Sodium salt of HBI-3808 of XRPD Pattern 2, for reference; Sodium salt of HBI-3808 of XRPD Pattern 5, for reference; Input, Sodium salt of HBI-3808 of XRPD Pattern 12; and solids post desolvation of the sodium salt of HBI-3808 of XRPD Pattern 12.

FIG. 13A: XRPD diffractogram of the sodium salt of HBI-3808 of XRPD Pattern 13.

FIG. 13B: Peak fitting of the XRPD diffractogram of the sodium salt of HBI-3808 of XRPD Pattern 13 (FIG. 13A).

FIG. 13C: TG-DSC thermogram of the isolated solids, as described in Example 13.

FIG. 13D: ¹H NMR spectrum of the isolated solids as described in Example 13, in d⁶-DMSO.

FIG. 13E: XRPD diffractogram after desolvation of the sodium salt of HBI-3808 of XRPD Pattern 13. From the top—Sodium salt of HBI-3808 of XRPD Pattern 2, for reference; Sodium salt of HBI-3808 of XRPD Pattern 5, for reference; Input, Sodium salt of HBI-3808 of XRPD Pattern 13; and solids post desolvation of the sodium salt of HBI-3808 of XRPD Pattern 13.

FIG. 14A: XRPD diffractogram of the sodium salt of HBI-3808 of XRPD Pattern 14.

FIG. 14B: Peak fitting of the XRPD diffractogram of the sodium salt of HBI-3808 of XRPD Pattern 14 (FIG. 14A).

FIG. 14C: TG-DSC thermogram of the isolated solids, as described in Example 14.

FIG. 14D: ¹H NMR spectrum of the isolated solids as described in Example 14, in d⁶-DMSO.

FIG. 14E: XRPD diffractogram after desolvation of the sodium salt of HBI-3808 of XRPD Pattern 14. From the top—Sodium salt of HBI-3808 of XRPD Pattern 2, for reference; Sodium salt of HBI-3808 of XRPD Pattern 5, for reference; Input, Sodium salt of HBI-3808 of XRPD Pattern 14; and solids post desolvation of the sodium salt HBI-3808 of XPRD Pattern 14.

FIG. 15A: XRPD diffractogram of the sodium salt of HBI-3808 of XRPD Pattern 15.

FIG. 15B: Peak fitting of the XRPD diffractogram of the sodium salt of HBI-3808 of XRPD Pattern 15 (FIG. 15A).

FIG. 15C: TG-DSC thermogram of the isolated solids, as described in Example 15.

FIG. 15D: ¹H NMR spectrum of the isolated solids as described in Example 15, in d⁶-DMSO.

FIG. 15E: XRPD diffractogram after desolvation of the sodium salt of HBI-3808 of XRPD Pattern 15. From the top—Sodium salt of HBI-3808 of XRPD Pattern 2, for reference; Input, Sodium salt of HBI-3808 of XRPD Pattern 15; and solids post desolvation of the sodium salt of HBI-3808 of XRPD Pattern 15.

FIG. 16A: XRPD diffractogram of the sodium salt of HBI-3808 of XRPD Pattern 16.

FIG. 16B: Peak fitting of the XRPD diffractogram of the sodium salt of HBI-3808 of XRPD Pattern 16 (FIG. 16A).

FIG. 17A: XRPD diffractogram of the ethanolamine salt of HBI-3808.

FIG. 17B: Peak fitting of the XRPD diffractogram of the ethanolamine salt of HBI-3808 (FIG. 17A).

FIG. 17C: HPLC chromatogram of the isolated solids of batch 1, as described in Example 17.

FIG. 17D: HPLC chromatogram of the isolated solids of batch 2, as described in Example 17.

FIG. 17E: HPLC chromatogram of the isolated solids of batch 3, as described in Example 17.

FIG. 17F: ¹H NMR spectrum of the isolated solids as described in Example 17, in d⁶-DMSO.

FIG. 17G: PLM image of the isolated solids, as described in Example 17. Objective: ×20. Scale bar 100 μm.

FIG. 17H: TG-DSC thermogram of the isolated solids, as described in Example 17.

FIG. 17I: DSC thermogram of the isolated solids, as described in Example 17.

FIG. 17J: FT-IR spectrum of the isolated solids, as described in Example 17.

FIG. 17K: Raman spectrum of the isolated solids, as described in Example 17.

FIG. 17L: DVS mass plot of the isolated solids, as described in Example 17.

FIG. 17M: DVS isotherm plot of the isolated solids, as described in Example 17.

FIG. 17N: XRPD diffractogram of recovered material from DVS analysis of the isolated solids, as described in Example 17. From the top—Input ethanolamine salt of HBI-3808 of XRPD Pattern 1, for reference; isolated material post DVS, and HBI-3808 acid of XRPD Pattern 1, for reference.

FIG. 17O: Hot stage microscopy of the isolated solids, as described in Example 17. Objective: ×10.

FIG. 17P: VT-XRPD diffractogram of the ethanolamine salt of HBI-3808.

FIG. 17Q: VH-XRPD diffractogram of the ethanolamine salt of HBI-3808.

FIG. 17R: XRPD diffractogram of isolated solids from salt disproportionation study with ethanolamine salt of HBI-3808. From the top—Input ethanolamine salt of HBI-3808 of XRPD Pattern 1, for reference; isolated material from salt disproportionation study, and HBI-3808 acid of XRPD Pattern 1, for reference.

FIG. 17S: XRPD diffractogram of isolated solids from hydration study with ethanolamine salt of HBI-3808. From the top—Input, ethanolamine salt of HBI-3808 of XRPD Pattern 1, for reference; solids isolated from methanol:water a_{w} 0.1; solids isolated from methanol:water a_{w} 0.5; solids isolated from methanol:water a_{w} 0.9; and HBI-3808 acid of XRPD Pattern 1, for reference.

FIG. 17T: XRPD diffractogram of isolated solids from pH dependent solubility study with ethanolamine salt of HBI-3808. From the top—Input, ethanolamine salt of HBI-3808 of XRPD Pattern 1, for reference; solids from pH 1.2 chloride buffer; solids from pH 3.0 acetate buffer; solids from pH 4.0 acetate buffer; solids from pH 6.8 phosphate buffer; solids from pH 8.0 phosphate buffer; solids from FaSSGF buffer; and HBI-3808 acid of XRPD Pattern 1, for reference.

FIG. 17U: XRPD diffractogram of isolated solids from a 1-week stability study with ethanolamine salt of HBI-3808. From the top—Input, ethanolamine salt of HBI-3808 of XRPD Pattern 1, for reference; 1 week at 25° C./60% RH; 1 week at 40° C./75% RH; 1 week at 60° C.; and HBI-3808 acid of XRPD Pattern 1, for reference.

FIG. 17V: HPLC chromatogram of solids obtained from ethanolamine salt of HBI-3808 after 1 week at 25° C./60% RH.

FIG. 17W: HPLC chromatogram of solids obtained from ethanolamine salt of HBI-3808 after 1 week at 40° C./75% RH.

FIG. 17X: HPLC chromatogram of solids obtained from ethanolamine salt of HBI-3808 after 1 week at 60° C.

FIG. 17Y: XRPD diffractogram of isolated solids from a 2-week stability study with ethanolamine salt of HBI-3808. From the top—Input, ethanolamine salt of HBI-3808 of XRPD Pattern 1, for reference; 2 weeks at 25° C./60% RH; 2 weeks at 40° C./75% RH; 2 weeks at 60° C.; and HBI-3808 acid of XRPD Pattern 1, for reference.

FIG. 17Z: HPLC chromatogram of solids obtained from ethanolamine salt of HBI-3808 after 2 weeks at 25° C./60% RH.

FIG. 17AA: HPLC chromatogram of solids obtained from ethanolamine salt of HBI-3808 after 2 weeks at 40° C./75% RH.

FIG. 17BB: HPLC chromatogram of solids obtained from ethanolamine salt of HBI-3808 after 2 weeks at 60° C.

FIG. 17CC: XRPD diffractogram of isolated solids from a 4-week stability study with ethanolamine salt of HBI-3808. From the top—Input, ethanolamine salt of HBI-3808 of XRPD Pattern 1, for reference; 4 weeks at 25° C./60% RH; 4 weeks at 40° C./75% RH; 4 weeks at 60° C.; and HBI-3808 acid of XRPD Pattern 1, for reference.

FIG. 17DD: HPLC chromatogram of solids obtained from ethanolamine salt of HBI-3808 after 4 weeks at 25° C./60% RH.

FIG. 17EE: HPLC chromatogram of solids obtained from ethanolamine salt of HBI-3808 after 4 weeks at 40° C./75% RH.

FIG. 17FF: HPLC chromatogram of solids obtained from ethanolamine salt of HBI-3808 after 4 weeks at 60° C.

FIG. 17GG: XRPD diffractogram of isolated solids from an 8-week stability study with ethanolamine salt of HBI-3808. From the top—Input, ethanolamine salt of HBI-3808 of XRPD Pattern 1, for reference; 8 weeks at 25° C./60% RH; 8 weeks at 40° C./75% RH; 8 weeks at 60° C.; and HBI-3808 acid of XRPD Pattern 1, for reference.

FIG. 17HH: HPLC chromatogram of solids obtained from ethanolamine salt of HBI-3808 after 8 weeks at 25° C./60% RH.

FIG. 17II: HPLC chromatogram of solids obtained from ethanolamine salt of HBI-3808 after 8 weeks at 40° C./75% RH.

FIG. 17JJ: HPLC chromatogram of solids obtained from ethanolamine salt of HBI-3808 after 8 weeks at 60° C.

FIG. 17KK: XRPD diffractogram of isolated solids from a 13-week stability study with ethanolamine salt of HBI-3808. From the top—Input, ethanolamine salt of HBI-3808 of XRPD Pattern 1, for reference; 13 weeks at 25° C./60% RH; 13 weeks at 40° C./75% RH; 13 weeks at 60° C.; and HBI-3808 acid of XRPD Pattern 1, for reference.

FIG. 17LL: HPLC chromatogram of solids obtained from ethanolamine salt of HBI-3808 after 13 weeks at 25° C./60% RH.

FIG. 17MM: HPLC chromatogram of solids obtained from ethanolamine salt of HBI-3808 after 13 weeks at 40° C./75% RH.

FIG. 17NN: HPLC chromatogram of solids obtained from ethanolamine salt of HBI-3808 after 13 weeks at 60° C.

FIG. 18A: Focused Beam Reflectance Measurement (FBRM) trend obtained during the process of preparing sodium salt of HBI-3808 of XRPD Pattern 5, as described in Example 18.

FIG. 18B: Non-weighted and square-weighted size distributions from FBRM, as described in Example 18.

FIG. 18C: XRPD diffractogram of the damp isolated solids (4^{th} from top), obtained drying for ca. 72 h (5^{th} from top), and after exposing to 40° C./75% RH for 72 h (bottom), as described in Example 18. XRPD of sodium salt of HBI-3808 pattern 2 (top), 5 (2ⁿ d from top) and 15 (3^{rd} from top) are shown for reference.

FIG. 18D: HPLC chromatogram of the isolated solids, as described in Example 18.

FIG. 18E: PLM image of the damp (left) and dry (right) isolated solids, as described in Example 18.

FIG. 18F: PSD of the isolated solids, as described in Example 18.

FIG. 18G: TG-DSC thermogram of the isolated solids, as described in Example 18.

FIGS. 18H-18J: DSC thermogram of the isolated solids, as described in Example 18.

First during heating from 20° C. and 250° C. (FIG. 18H); then cooling from 250° C. to −80° C.

(FIG. 18I); and then heating from −80° C. to 250° C. (FIG. 18J).

FIG. 18K ¹H NMR spectrum of the isolated solids, as described in Example 18.

FIG. 18L: ¹³C DEPTQ spectrum of the isolated solids, as described in Example 18.

FIG. 18M: Quantitative ¹H NMR spectrum of the isolated solids, as described in Example 18.

FIG. 18N: IR spectrum of the isolated solids, as described in Example 18.

FIG. 18O: Raman spectrum of the isolated solids, as described in Example 18.

FIG. 18P: TIC (top) and DAD (bottom) scan from LC-MS of the isolated solids, as described in Example 18.

FIG. 18Q: LC-MS Spectrum for TIC Signal (FIG. 18P) with retention time 6.92 min.

FIG. 18R: LC-MS Spectrum for TIC Signal (FIG. 18P) with retention time 7.38 min.

FIG. 18S: UV spectrum of the isolated solids, as described in Example 18.

FIG. 19: XRPD diffractogram of the isolated solids (top), as described in Example 19. XRPD of sodium salt of HBI-3808 pattern 5 (middle), and 2 (bottom) are shown for reference.

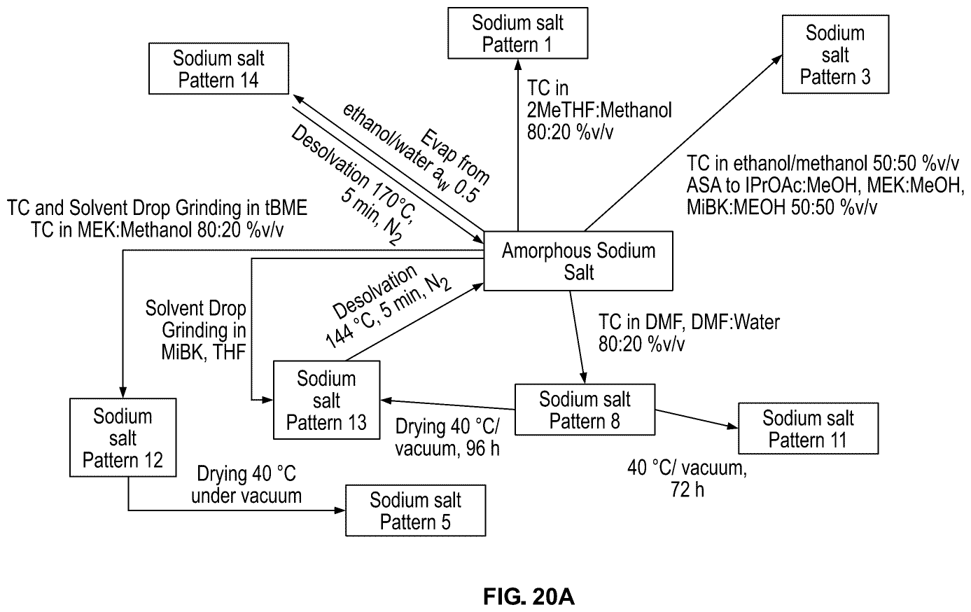

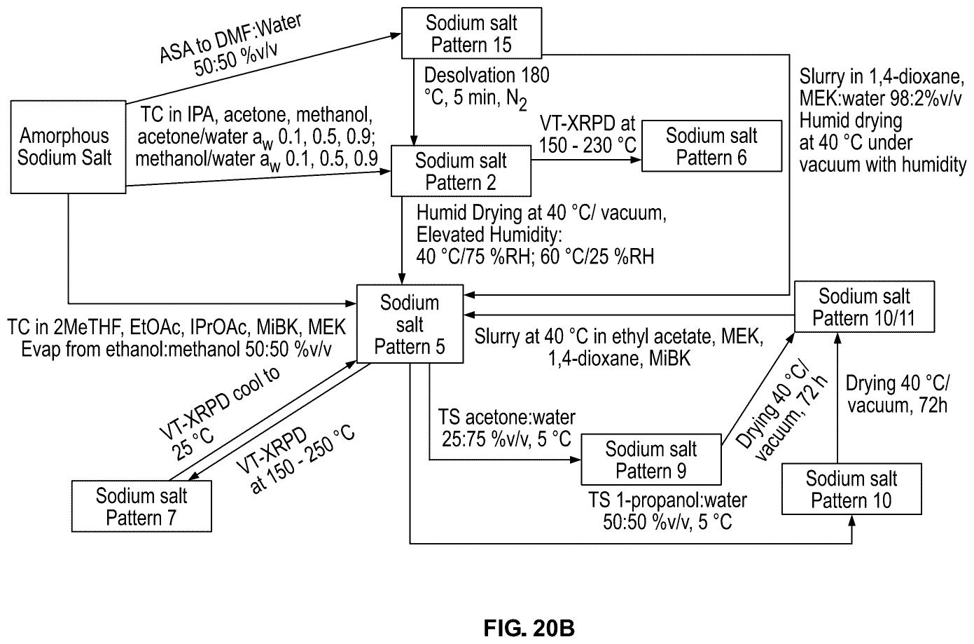

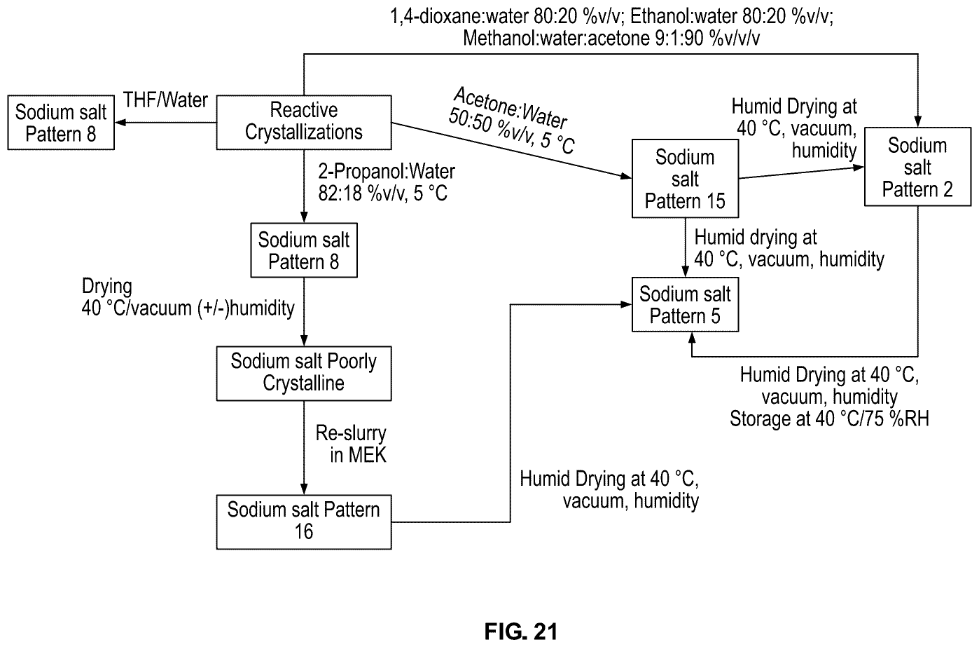

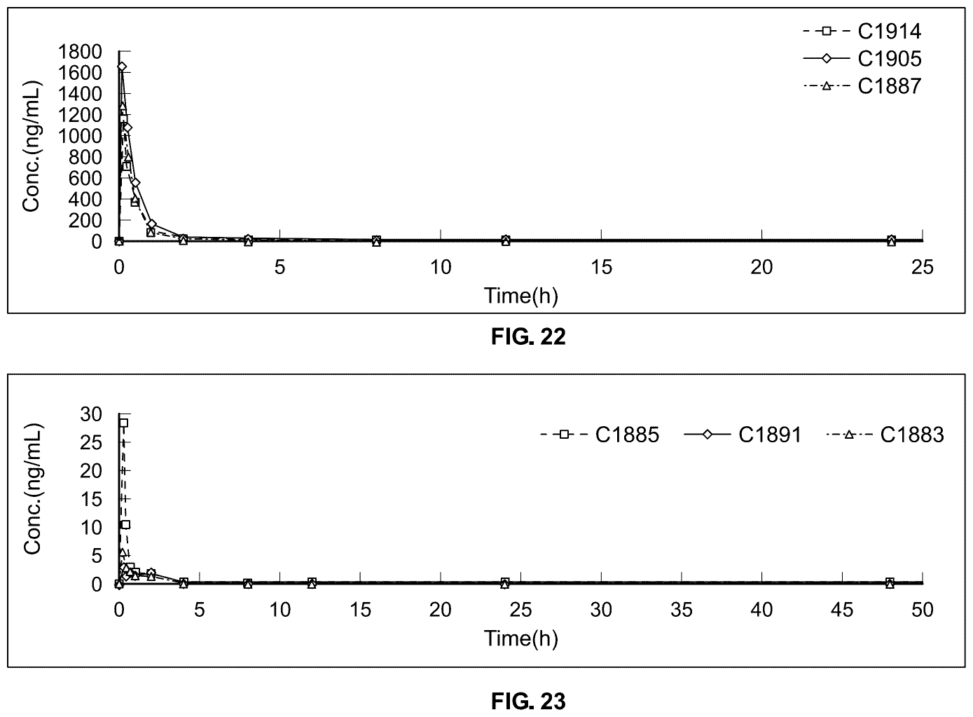

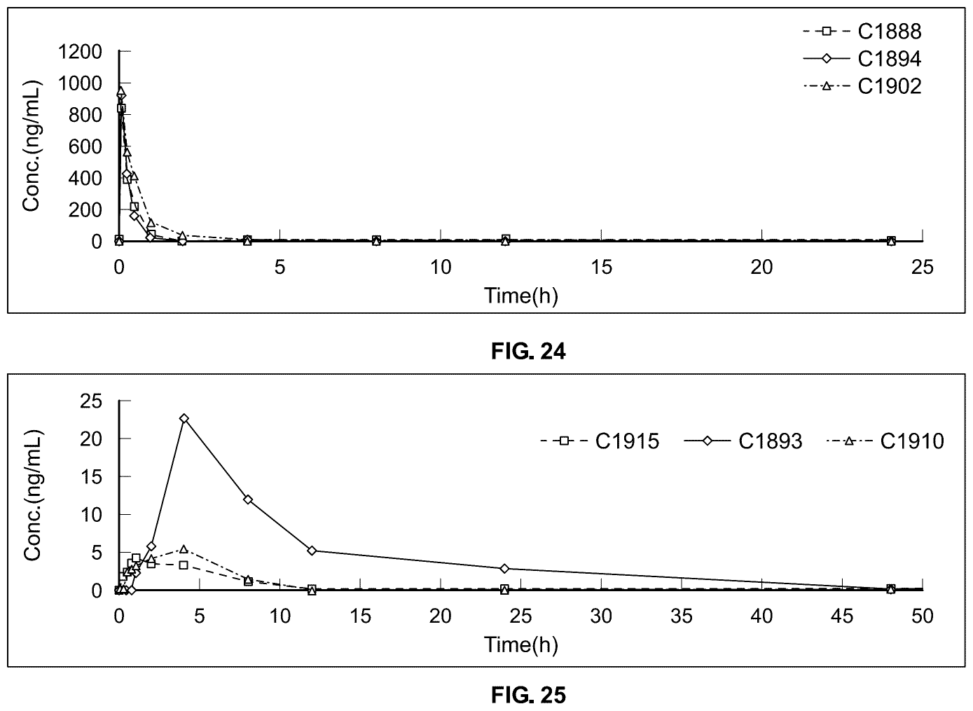

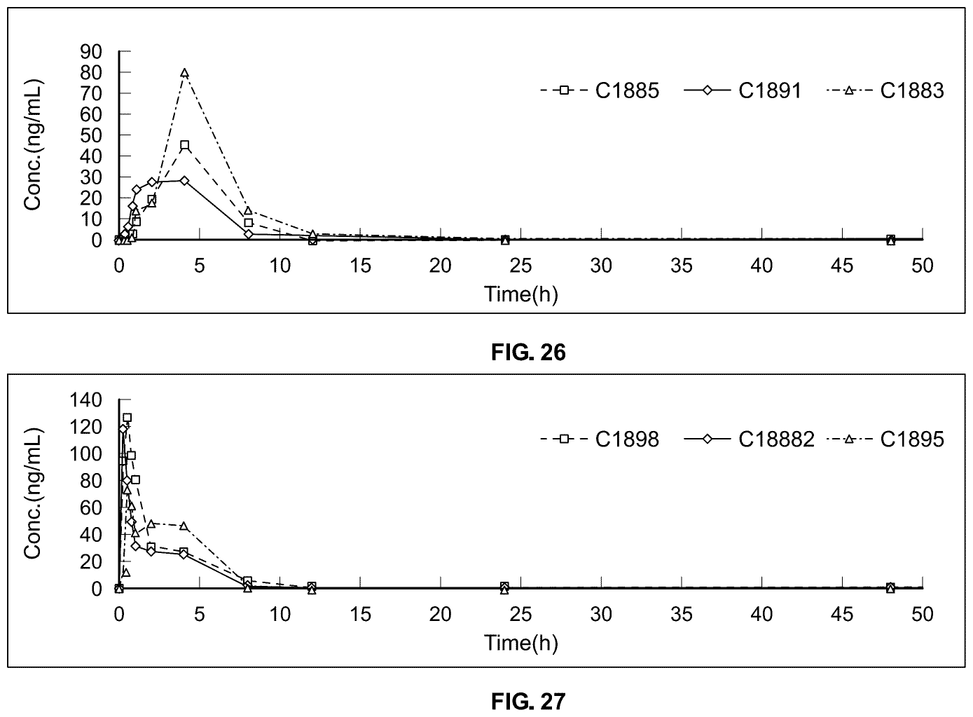

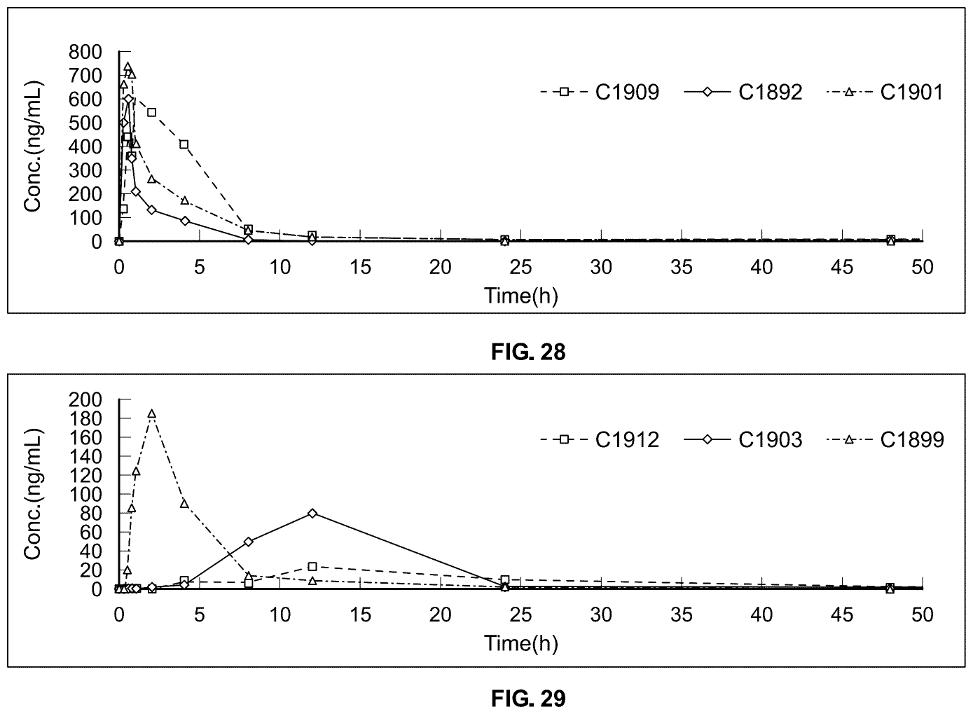

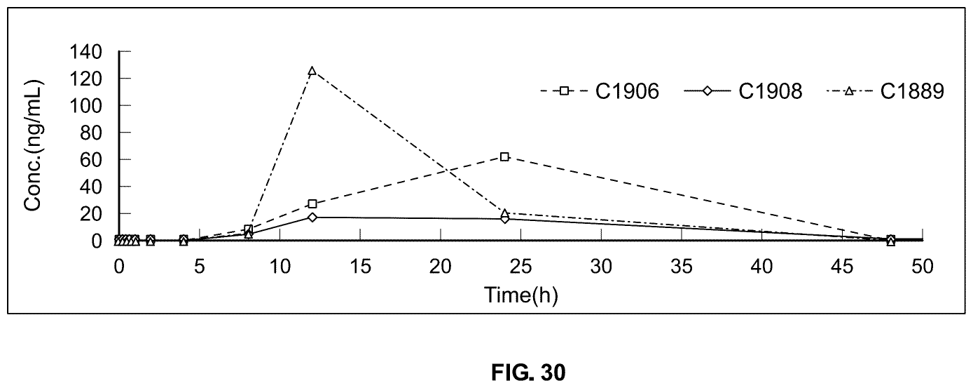

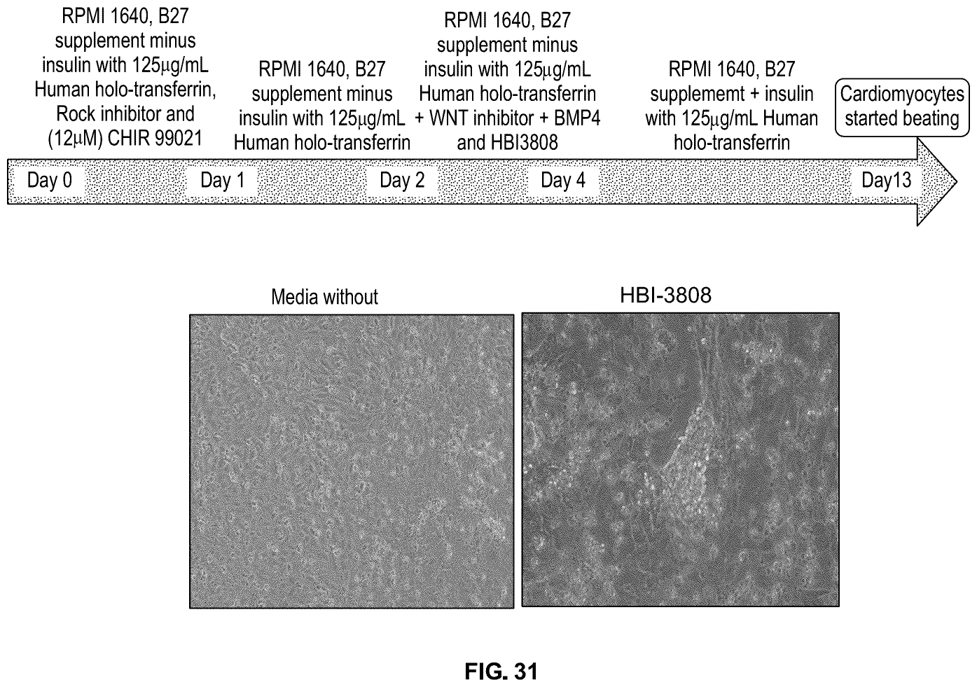

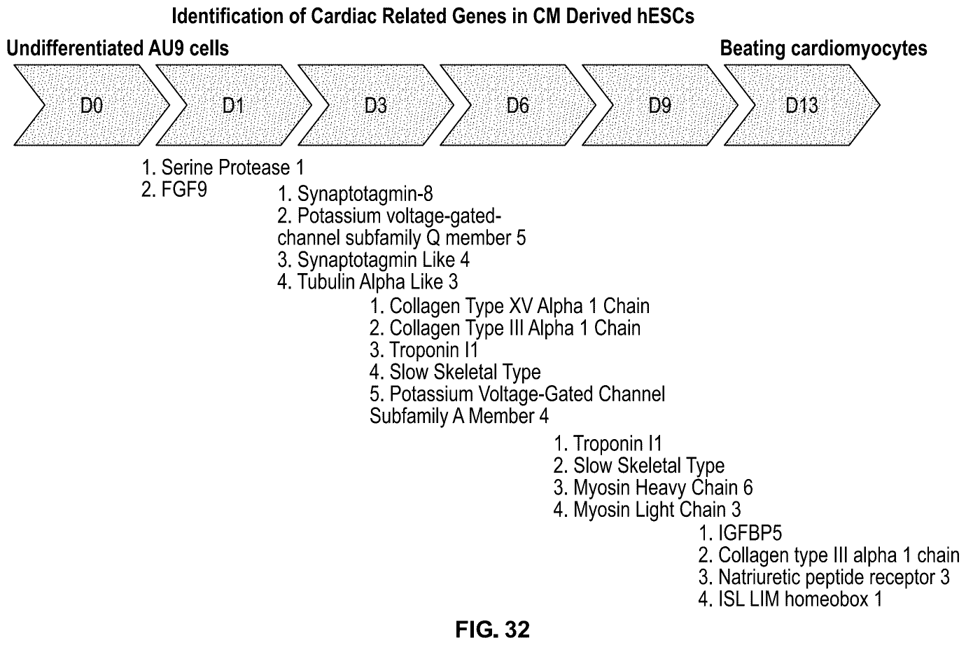

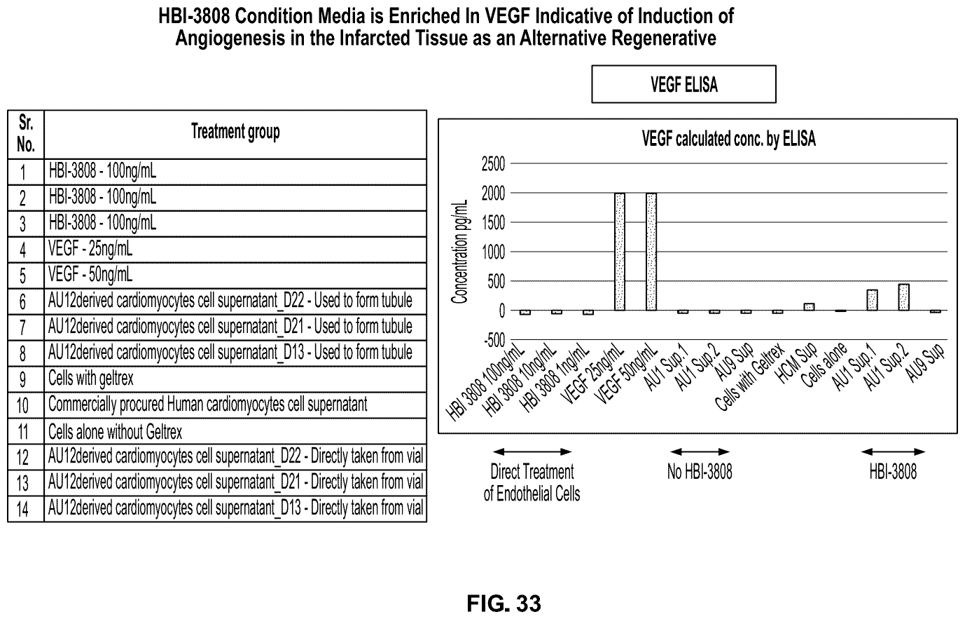

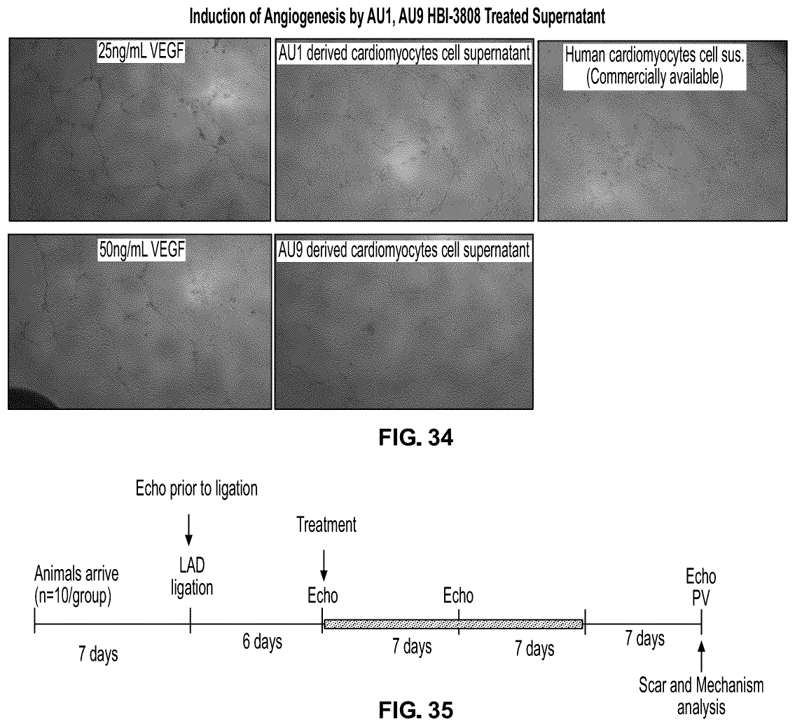

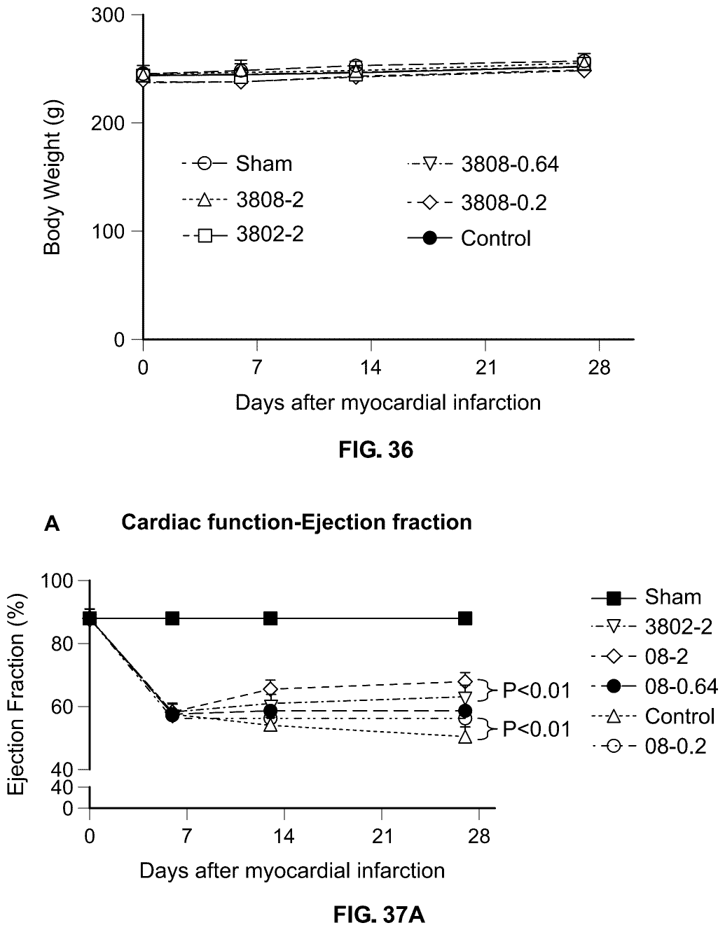

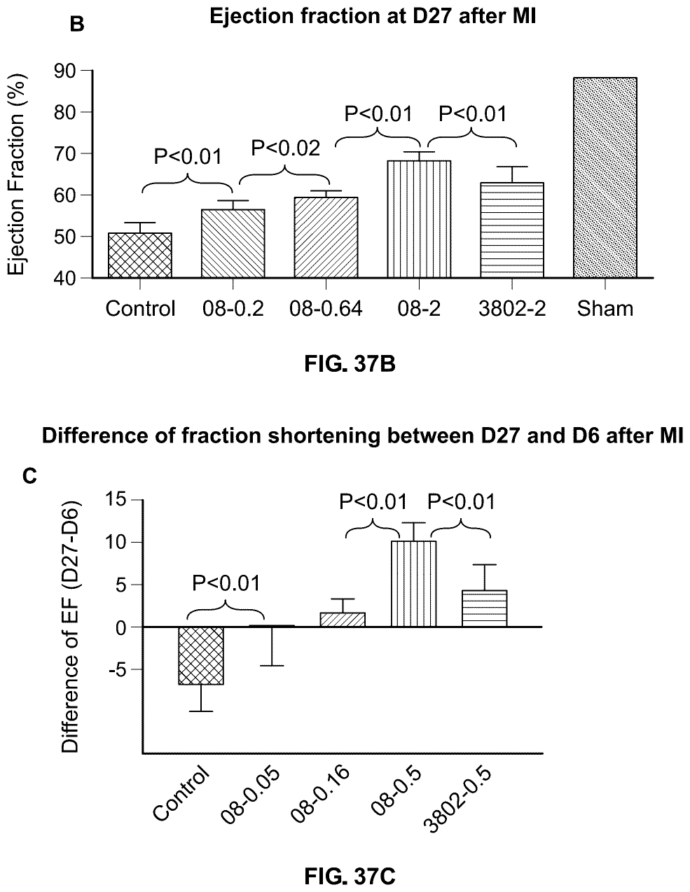

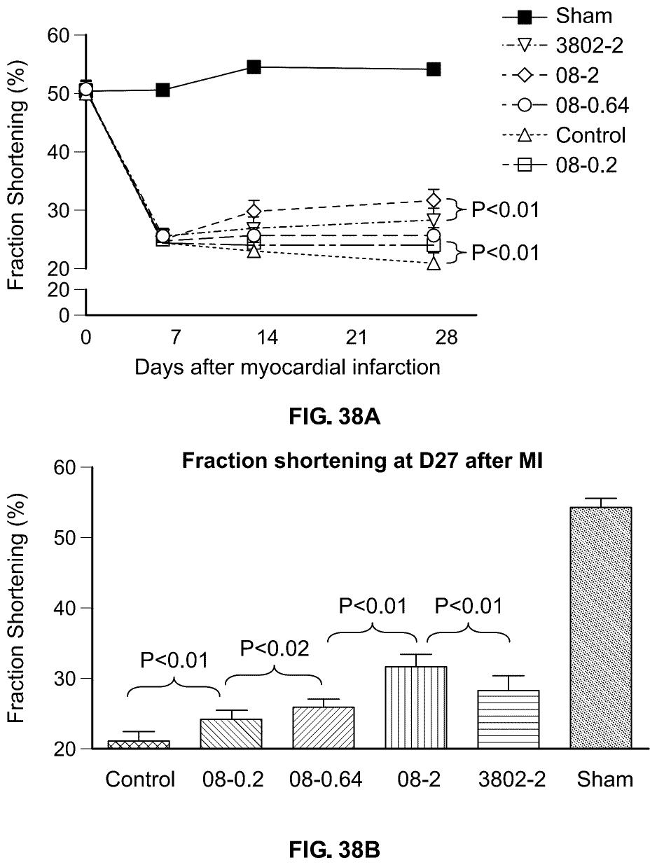

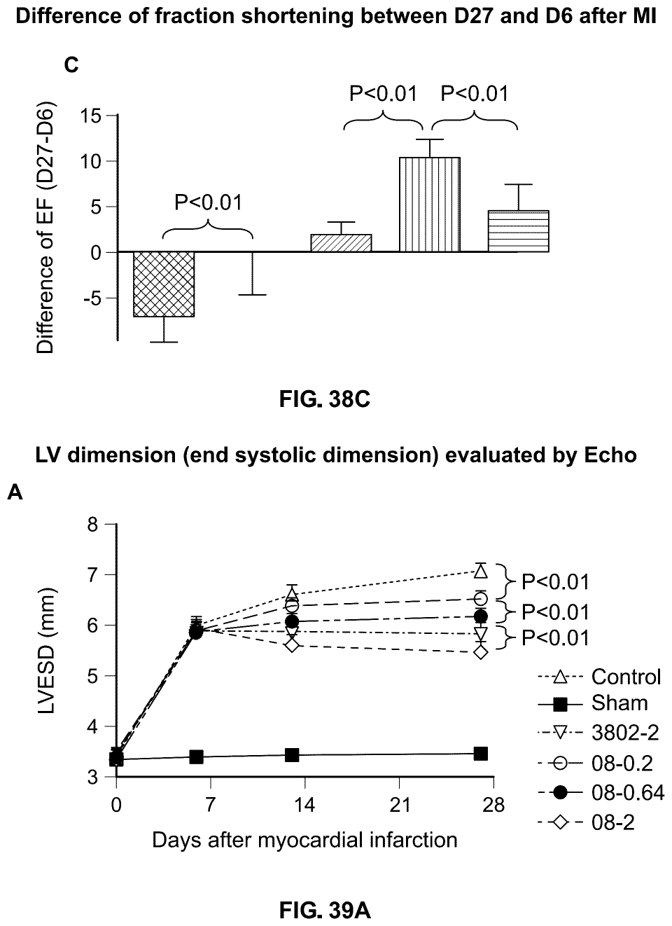

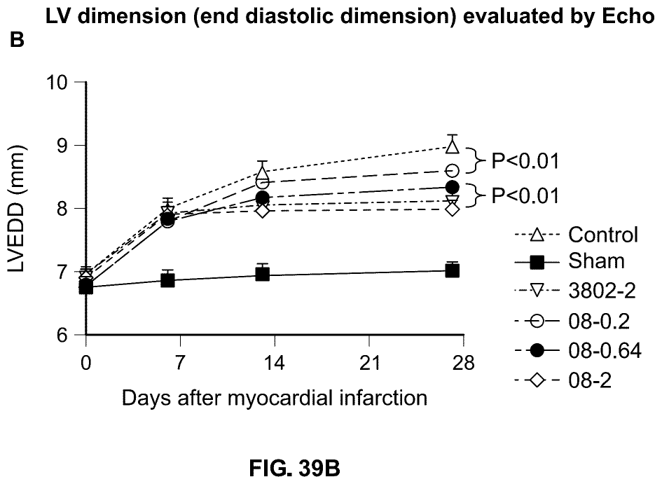

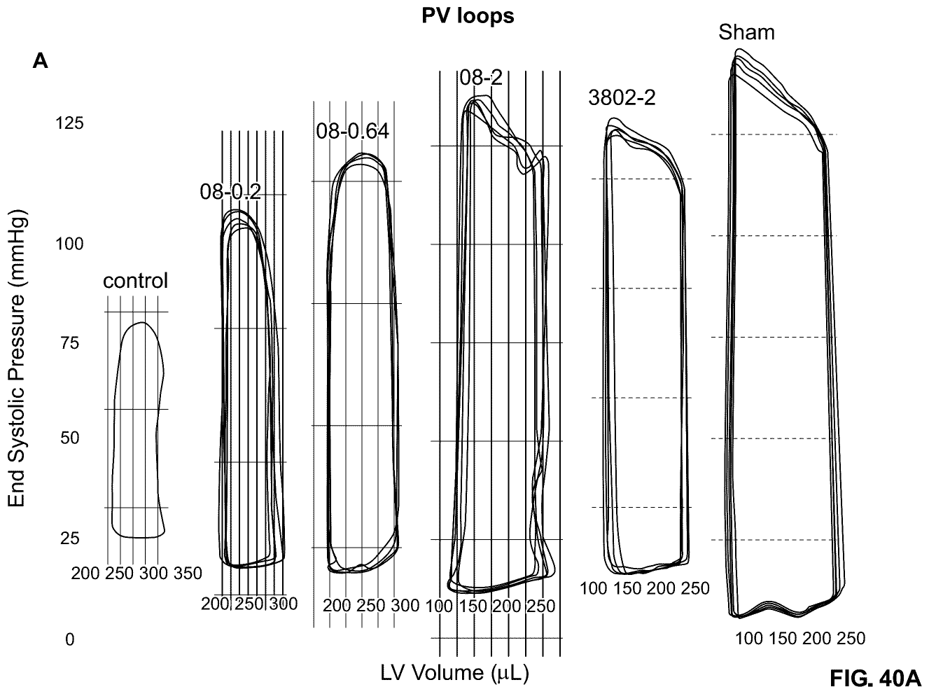

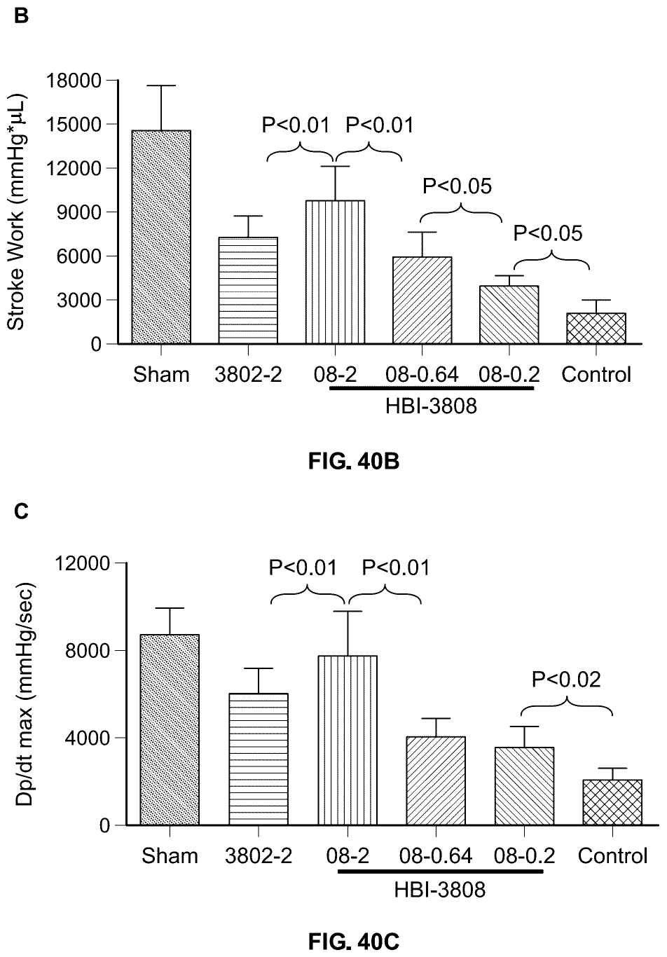

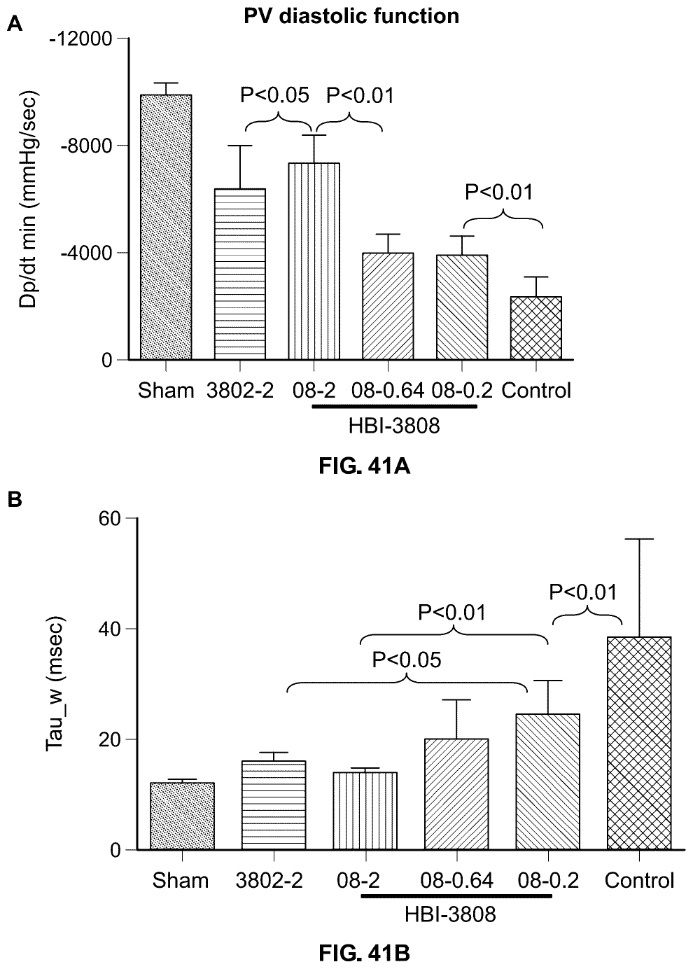

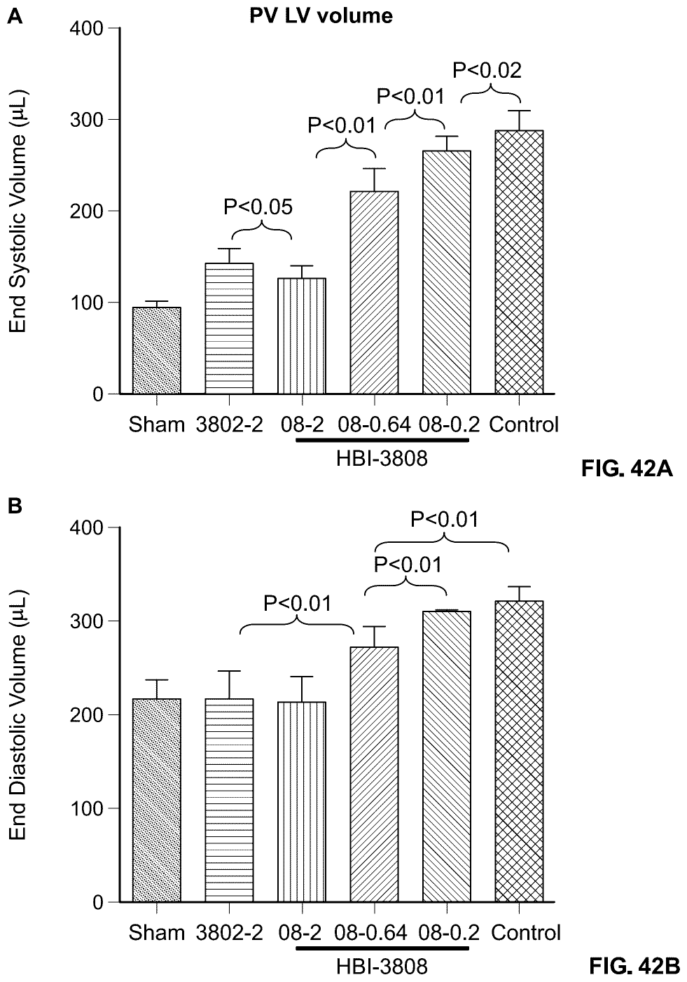

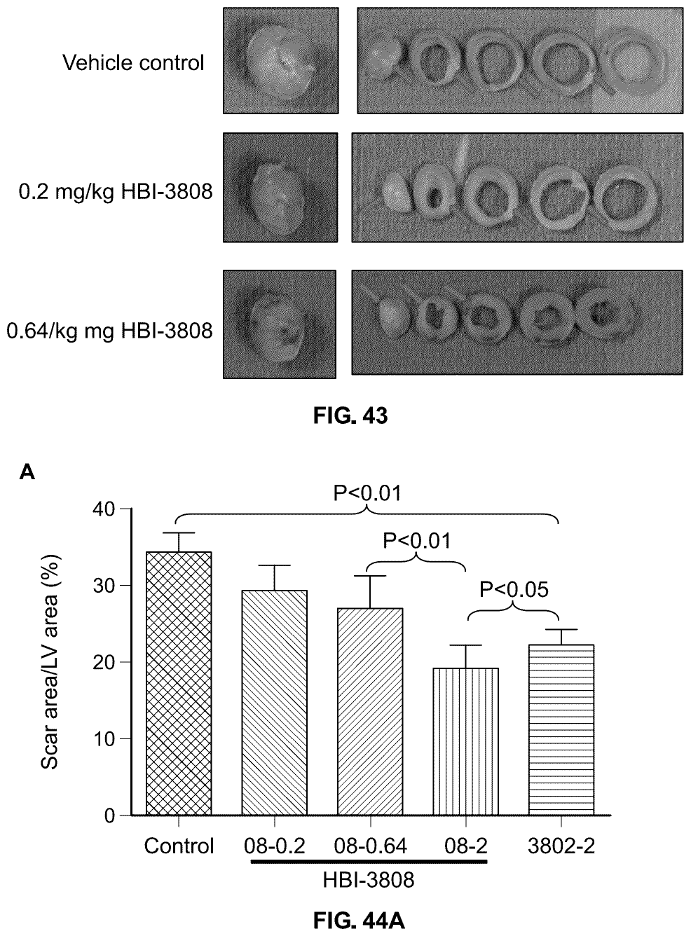

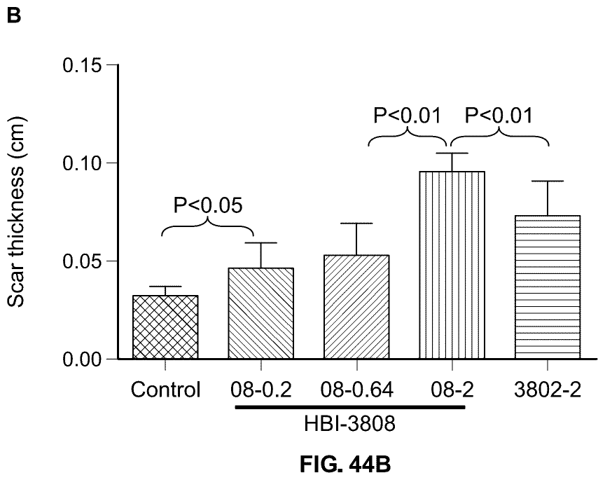

FIGS. 20A-B: A schematic showing steps for preparing sodium salts of HBI-3808 from amorphous sodium salt of HBI-3808, according to one set of examples of the current disclosure.

FIG. 21: A schematic showing steps for preparing sodium salts of HBI-3808 through reactive crystallization, according to another set of examples of the current disclosure.

FIG. 22: Individual Plasma Concentration-Time Profile of HBI-3802 after an IV dose of 0.3 mg/kg in Rabbit.

FIG. 23: Individual Plasma Concentration-Time Profile of HBI-3802 after a PO dose of 5 mg/kg in Rabbit.

FIG. 24: Individual Plasma Concentration-Time Profile of HBI-3808 after an IV dose of 0.3 mg/kg in Rabbit.

FIG. 25: Individual Plasma Concentration-Time Profile of HBI-3808 after an IV dose of 1 mg/kg in Rabbit.

FIG. 26: Individual Plasma Concentration-Time Profile of HBI-3808 after an IV dose of 5 mg/kg in Rabbit.

FIG. 27: Individual Plasma Concentration-Time Profile of HBI-3808 after a PO Dose of 1 mg/kg in Rabbit.

FIG. 28: Individual Plasma Concentration-Time Profile of HBI-3808 after a PO Dose of 5 mg/kg in Rabbit.

FIG. 29: Individual Plasma Concentration-Time Profile of HBI-3808 after a PO Dose of 5 mg/kg (normal capsule) in Rabbit.

FIG. 30: Individual Plasma Concentration-Time Profile of HBI-3808 after a PO Dose of 5 mg/kg (enteric coated capsule) in Rabbit.

FIG. 31: Induction of Differentiation of hESCs to CMs by HBI-3808.

FIG. 32: Identification of Cardiac Related Genes in CM Derived hESCs.

FIG. 33: HBI-3808 Condition Media is Enriched in VEGF Indicative of Induction of Angiogenesis in the Infarcted Tissue as an Alternative Regenerative Mechanism.

FIG. 34: Induction of Angiogenesis by AU1, AU9 HBI-3808-Treated Supernatant.

FIG. 35: Schematic plan of treatment of rats by gastric gavage.

FIG. 36: Body weights of rats in a pre-clinical adult rat model study of HBI-3808.

FIG. 37A: Cardiac function-ejection fraction for test subjects. EF % is average for each cohort.

FIG. 37B: Ejection fraction at day 27 after myocardial infarction. Bars show average for each cohort of test subjects.

FIG. 37C: Difference in ejection fraction between days 27 and 6 after myocardial infarction. Bars show average for each cohort of test subjects.

FIG. 38A: Fractional shortening (%) for all test subjects from days 0 to 27 after myocardial infarction. Each point shows the average for the corresponding cohort.

FIG. 38B: Fractional shortening (%) for all test subjects from on day 27 after myocardial infarction. Bars show average for each cohort of test subjects.

FIG. 38C: Difference in fractional shortening (%) between days 27 and 6 after myocardial infarction. Bars show average for each cohort of test subjects.

FIG. 39A: LV dimension (end systolic dimension) by echocardiogram of rat hearts at days 0 (infarct induced), 6 (immediately before treatment), and days 13 and 27 (7 and 21 days after treatment).

FIG. 39B: LV dimension (end diastolic dimension) by echocardiogram of rat hearts at days 0 (infarct induced), 6 (immediately before treatment), and days 13 and 27 (7 and 21 days after treatment).

FIG. 40A: Representative cardiac performance loops recorded for each of the 6 cohorts of test subjects.

FIG. 40B: Stroke work (in mm Hg×μL) for each of the 6 cohorts of test subjects.

FIG. 40C: Maximal rise of left ventricular pressure (dP/dt max (mm Hg/sec) for each of the 6 cohorts of test subjects.

FIG. 41A: PV diastolic function at 21 days after treatment.

FIG. 41B: Isovolumic relaxation constant (Tau_w) at 21 days after treatment.

FIG. 42A: Left ventricular end systolic volume for each of the 6 cohorts at day 21 post treatment.

FIG. 42B: Left ventricular end diastolic volume for each of the 6 cohorts at day 21 post treatment.

FIG. 43: Representative sections of hearts from the 6 groups after formalin fixation (arrows indicate infarct areas).

FIG. 44A: Scar areas from computed planimetric analysis of heart sections in the 6 cohorts.

FIG. 44B: Scar thickness from computed planimetric analysis of heart sections in the 6 cohorts.

### DETAILED DESCRIPTION

Disclosed herein are sodium salts of HBI-3808, and methods of making and using the sodium salts, as well as pharmaceutical compositions comprising the salts and methods of making and using the pharmaceutical compositions comprising the sodium salts of HBI-3808. As shown in a non-limiting manner in the examples, multiple polymorphic forms of the sodium salt of HBI-3808 exist and have been isolated and characterized. Further, as shown in Examples 2 and 5, a sodium salt polymorph having XRPD of pattern 2, and a sodium salt polymorph having XRPD of pattern 5, can have relatively high stability over a range of temperature and humidity. Such, properties may benefit use of the salt in various chemical and biological process. Also disclosed herein are ethanolamine salts of HBI-3808, and methods of making and using the ethanolamine salts.

These and other non-limiting aspects of the present disclosure are discussed in further detail in the following sections.

### HBI-3808

HBI-3808 has the chemical formula of depicted below and the CAS no. 89786-84-5. HBI-3808, is also chemically known as 23-hydroxytormentic acid, 19-alpha-hydroxyasiatic acid, and 19α-hydroxyasiatic acid.

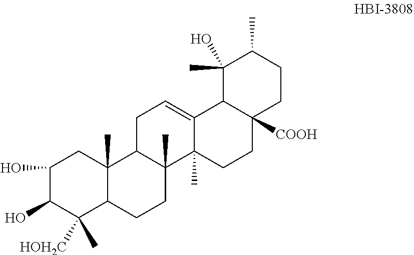

### Sodium Salt of HBI-3808

In certain embodiments, the sodium salt of HBI-3808 is a monosodium salt. In certain embodiments, the sodium salt of HBI-3808 is an isolated sodium salt of HBI-3808. In certain aspects, the sodium salt of HBI-3808 is in a solid form. In certain aspects, the sodium salt of HBI-3808 is in a solid amorphous form. In certain aspects, the sodium salt of HBI-3808 is in a solid crystalline form. The crystalline sodium salt of HBI-3808 may or may not contain one or more molecules of solvates in the crystal assembly. In certain embodiments, the crystalline sodium salt of HBI-3808 contains one or more molecules of water, acetone, or any combination thereof, in the crystal assembly. In certain embodiments, the solid crystalline sodium salt of HBI-3808 contains about 0, 1, 2, 3, 4, 5, 6, 7, 8, 9 or 10, equivalents of water of crystallization. In certain embodiments, the solid crystalline sodium salt of HBI-3808 comprises an anhydrous salt. In certain embodiments, the solid crystalline sodium salt of HBI-3808 comprises a mono-hydrate salt. In certain embodiments, the solid crystalline sodium salt of HBI-3808 comprises a di-hydrate salt. In certain embodiments, the solid crystalline sodium salt of HBI-3808 comprises a mono-hydrate, mono acetone solvate salt. In certain embodiments, the solid crystalline sodium salt of HBI-3808 comprises a monosodium, anhydrous salt. In certain embodiments, the solid crystalline sodium salt of HBI-3808 comprises a monosodium, mono-hydrate salt. In certain embodiments, the solid crystalline sodium salt of HBI-3808 comprises a monosodium, di-hydrate salt. In certain embodiments, the solid crystalline sodium salt of HBI-3808 comprises a monosodium, mono-hydrate, mono acetone solvate salt. The sodium salt of HBI-3808 can have a purity of at least about 95%, at least about 96%, at least about 97%, at least about 98%, at least about 99%, at least about 99.5%, at least about 99.7%, or about 98% to 100%. In certain embodiments, the sodium salt of HBI-3808 can have a purity of about 90% to about 100%. In certain embodiments, the sodium salt of HBI-3808 can have a purity of about 90% to about 95%, about 90% to about 96%, about 90% to about 97%, about 90% to about 98%, about 90% to about 99%, about 90% to about 99.2%, about 90% to about 99.5%, about 90% to about 99.7%, about 90% to about 99.8%, about 90% to about 99.9%, about 90% to about 100%, about 95% to about 96%, about 95% to about 97%, about 95% to about 98%, about 95% to about 99%, about 95% to about 99.2%, about 95% to about 99.5%, about 95% to about 99.7%, about 95% to about 99.8%, about 95% to about 99.9%, about 95% to about 100%, about 96% to about 97%, about 96% to about 98%, about 96% to about 99%, about 96% to about 99.2%, about 96% to about 99.5 % about 96% to about 99.7%, about 96% to about 99.8%, about 96% to about 99.9%, about 96% to about 100%, about 97% to about 98%, about 97% to about 99%, about 97% to about 99.2%, about 97% to about 99.5%, about 97% to about 99.7%, about 97% to about 99.8%, about 97% to about 99.9%, about 97% to about 100%, about 98% to about 99%, about 98% to about 99.2%, about 98% to about 99.5%, about 98% to about 99.7%, about 98% to about 99.8%, about 98% to about 99.9%, about 98% to about 100%, about 99% to about 99.2%, about 99% to about 99.5%, about 99% to about 99.7%, about 99% to about 99.8%, about 99 % to about 99.9%, about 99% to about 100%, about 99.2% to about 99.5%, about 99.2% to about 99.7%, about 99.2% to about 99.8%, about 99.2% to about 99.9%, about 99.2% to about 100%, about 99.5% to about 99.7%, about 99.5% to about 99.8%, about 99.5% to about 99.9 %, about 99.5% to about 100%, about 99.7% to about 99.8%, about 99.7% to about 99.9%, about 99.7% to about 100 % about 99.8% to about 99.9%, about 99.8% to about 100%, or about 99.9% to about 100%. In certain embodiments, the sodium salt of HBI-3808 can have a purity of about 90%, about 95%, about 96%, about 97%, about 98%, about 99%, about 99.2 %, about 99.5%, about 99.7%, about 99.8%, about 99.9%, or about 100%. In certain embodiments, the sodium salt of HBI-3808 can have a purity of at least about 90%, about 95%, about 96%, about 97%, about 98%, about 99%, about 99.2%, about 99.5%, about 99.7%, about 99.8%, or about 99.9%. In certain embodiments, the sodium salt of HBI-3808 can have a purity of at most about 95%, about 96%, about 97%, about 98%, about 99%, about 99.2%, about 99.5%, about 99.7%, about 99.80% about 99.9%, or about 100%.

The sodium salt of HBI-3808 can have an x-ray power diffraction (XRPD) diffractogram of Pattern 1, 2, 3, 4, 5, 6, 7, 8, 9, 10, 11, 12, 13, 14, 15, or 16. In certain embodiments, the sodium salt of HBI-3808 has a thermogravimetry (TG)—differential scanning calorimetry (DSC) thermogram substantially matching that of FIG. 2G, 2N, 5G, 8D, 11C, 12C, 13C, 14C or 15C. In certain embodiments, the sodium salt of HBI-3808 has a differential scanning calorimetry (DSC) thermogram substantially matching that of FIG. 2H, or 5H. In certain embodiments, the sodium salt of HBI-3808 has a dynamic vapor sorption (DVS) mass plot substantially matching to that of FIG. 2K or 5I. In certain embodiments, the sodium salt of HBI-3808 has a DVS isotherm plot substantially matching to that of FIG. 2L or 5J. In certain embodiments, the sodium salt of HBI-3808 has a Fourier-transform infrared spectroscopy (FT-IR) spectrum substantially matching the FT-IR spectrum of FIG. 2I. In certain embodiments, the sodium salt of HBI-3808 has a Raman spectrum substantially matching the Raman spectrum of FIG. 2J.

Sodium salt of HBI-3808 having XRPD diffractogram of Pattern 1

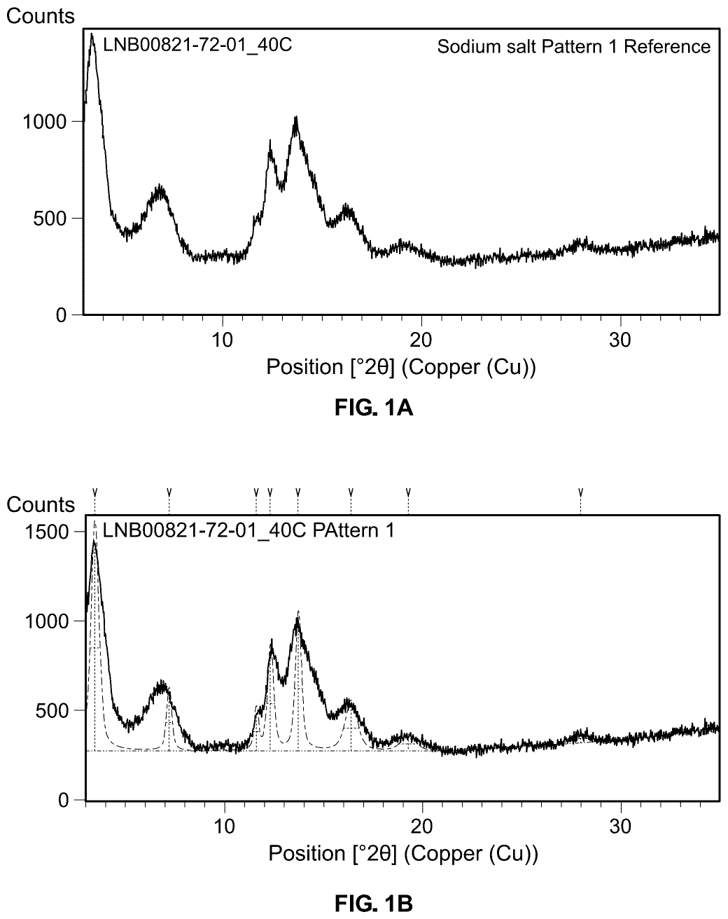

XRPD diffractogram of the sodium salt of HBI-3808 having XRPD diffractogram of Pattern 1, comprises one or more peaks of the XRPD diffractogram of FIG. 1A, and/or Table 1. In certain embodiments, the XRPD diffractogram comprises i) 1, 2, 3, 4, 5, 6, 7, 8, or any range there between, peaks of, or substantially matches, FIG. 1A; ii) 1, 2, 3, 4, 5, 6, 7, 8, or any range there between, or all peaks (±0.2 °2θ) listed in Table 1, or both. In certain embodiments, the XRPD diffractogram substantially matches, FIG. 1A.

Sodium salt of HBI-3808 having X RPD diffractogram of Pattern 2

XRPD diffractogram of the sodium salt of HBI-3808 having XRPD diffractogram of Pattern 2, comprises one or more peaks of the XRPD diffractogram of FIG. 2A, and/or Table 2A. In certain embodiments, the XRPD diffractogram comprises i) 1, 2, 3, 4, 5, 6, 7, 8, 9, or any range there between, peaks of, or substantially matches, FIG. 2A; ii) 1, 2, 3, 4, 5, 6, 7, 8, 9, 10, 11, 12, 13, 14,15,16,17,18, 19,20,21,22,23,24,25,26,27,28,29,30,31,32,33,34,35, or any range there between, or all peaks (±0.2 °2θ) listed in Table 2A, or both. In certain embodiments, the XRPD diffractogram substantially matches, FIG. 2A.

In certain embodiments, the sodium salt of HBI-3808 having XRPD diffractogram of Pattern 2, has any one of, any combination of, or all of i) a XRPD diffractogram comprising a) 1, 2, 3, 4, 5, 6, 7, 8, 9, or any range there between, peaks of, or substantially matches, FIG. 2A, b) 1, 2,3,4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, or any range there between, or all peaks (±0.2°2θ) listed in Table 2A, or both; ii) a TG-DSC thermogram substantially matching to that of FIG. 2G; iii) a DSC thermogram substantially matching to that of FIG. 2H; iv) a DVS mass plot substantially matching to that of FIG. 2K; v) a DVS isotherm plot substantially matching to that of FIG. 2L; vi) a FT-IR spectrum substantially matching to that of FIG. 2I; and vii) a Raman spectrum substantially matching to that of FIG. 2J.

Sodium salt of HBI-3808 having XRPD diffractogram of Pattern 3

XRPD diffractogram of the sodium salt of HBI-3808 having XRPD diffractogram of Pattern 3, comprises one or more peaks of the XRPD diffractogram of FIG. 3A, and/or Table 3. In certain embodiments, the XRPD diffractogram comprises i) 1, 2, 3, 4, 5, 6, 7, 8, or any range there between, peaks of, or substantially matches, FIG. 3A; ii) 1, 2, 3, 4, 5, 6, 7, 8, 9, 10, 11 or any range there between, or all peaks (±0.2 °2θ) listed in Table 3, or both. In certain embodiments, the XRPD diffractogram substantially matches, FIG. 3A.

Sodium salt of HBI-3808 having XRPD diffractogram of Pattern 4

XRPD diffractogram of the sodium salt of HBI-3808 having XRPD diffractogram of Pattern 4, comprises one or more peaks of the XRPD diffractogram of FIG. 4A, and/or Table 4. In certain embodiments, the XRPD diffractogram comprises i) 1, 2, 3, 4, 5, 6, 7, 8, or any range there between, peaks of, or substantially matches, FIG. 4A; ii) 1, 2, 3, 4, 5, 6, 7, 8, 9, 10, 11, 12, 13, 14, 15, 16, 17, 18, 19, 20, 21, 22, 23, 24, 25, 26, 27, 28, or any range there between, or all peaks (±0.2 °2θ) listed in Table 4, or both. In certain embodiments, the XRPD diffractogram substantially matches, FIG. 4.

Sodium salt of HBI-3808 having XRPD diffractogram of Pattern 5

XRPD diffractogram of the sodium salt of HBI-3808 having XRPD diffractogram of Pattern 5, comprises one or more peaks of the XRPD diffractogram of FIG. 5A, and/or Table 5A.

In certain embodiments, the XRPD diffractogram comprises i) 1, 2, 3, 4, 5, 6, 7, 8, 9, 10, or any range there between peaks of, or substantially matches, FIG. 5A; ii) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or any range there between, or all peaks (±0.2°2θ) listed in Table 5A, or both. In certain embodiments, the XRPD diffractogram substantially matches, FIG. 5A.

In certain embodiments, the sodium salt of HBI-3808 having XRPD diffractogram of Pattern 5, has any one of, any combination of, or all of i) a XRPD diffractogram comprising a) 1, 2, 3, 4, 5, 6, 7, 8, 9, 10, or any range there between peaks of, or substantially matches, FIG. 5A, b) 1,2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or any range there between, or all peaks (±0.2 °2θ) listed in Table 5A, or both; ii) a TG-DSC thermogram substantially matching to that of FIG. 5G; iii) a DSC thermogram substantially matching to that of FIG. 5H; iv) a DVS mass plot substantially matching the mass plot of FIG. 5I; and v) a DVS isotherm plot substantially matching the isotherm plot of FIG. 5J.

In certain embodiments, the sodium salt of HBI-3808 having XRPD diffractogram of Pattern 5, can be a mono sodium, mono-hydrate salt.

Sodium salt of HBI-3808 having XRPD diffractogram of Pattern 6

XRPD diffractogram of the sodium salt of HBI-3808 having XRPD diffractogram of Pattern 6, comprises one or more peaks of the XRPD diffractogram of FIG. 6A, and/or Table 6. In certain embodiments, the XRPD diffractogram comprises i) 1, 2, 3, 4, 5, 6, 7, 8, 9, 10, or any range there between, peaks of, or substantially matches, FIG. 6A; ii) 1, 2, 3, 4, 5, 6, 7, 8, 9, 10, 11, 12, 13, 14,15,16,17,18, 19,20,21,22,23,24,25,26,27,28,29,30,31,32,33,34,35, or any range there between, or all peaks (±0.2 °2θ) listed in Table 6, or both. In certain embodiments, the XRPD diffractogram substantially matches, FIG. 6A.

In certain embodiments, the sodium salt of HBI-3808 having XRPD diffractogram of Pattern 6, can be a mono sodium, anhydrous salt.

Sodium salt of HBI-3808 having X RPD diffractogram of Pattern 7

XRPD diffractogram of the sodium salt of HBI-3808 having XRPD diffractogram of Pattern 7, comprises one or more peaks of the XRPD diffractogram of FIG. 7A, and/or Table 7. In certain embodiments, the XRPD diffractogram comprises i) 1, 2, 3, 4, 5, 6, 7, 8, 9, 10, or any range there between, peaks of, or substantially matches, FIG. 7A; ii) 1, 2, 3, 4, 5, 6, 7, 8, 9, 10, 11, 12, 13, 14, 15, 16, 17, 18, 19, 20, 21, 22, 23, 24, 25, 26, 27, 28, or any range there between, or all peaks (±0.2 °2θ) listed in Table 7, or both. In certain embodiments, the XRPD diffractogram substantially matches, FIG. 7A.

Sodium salt of HBI-3808 having XRPD diffractogram of Pattern 8

XRPD diffractogram of the sodium salt of HBI-3808 having XRPD diffractogram of Pattern 8, comprises one or more peaks of the XRPD diffractogram of FIG. 8A, and/or Table 8. In certain embodiments, the XRPD diffractogram comprises i) 1, 2, 3, 4, 5, 6, 7, 8, 9, 10, 11, 12, 13, 14, 15, or any range there between, peaks of, or substantially matches, FIG. 8A; ii) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or any range there between, or all peaks (±0.2°2θ) listed in Table 8, or both. In certain embodiments, the XRPD diffractogram substantially matches, FIG. 8A.

In certain embodiments, the sodium salt of HBI-3808 of XRPD diffractogram of Pattern 8, has a i) XRPD diffractogram comprising a) 1, 2, 3, 4, 5, 6, 7, 8, 9, 10, 11, 12, 13, 14, 15, or any range there between, peaks of, or substantially matches, FIG. 8A, b) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or any range there between, or all peaks (±0.2°2θ) listed in Table 8, or both; ii) TG-DSC thermogram substantially matching to that of FIG. 8D; or both.

Sodium salt of HBI-3808 having X RPD diffractogram of Pattern 9

XRPD diffractogram of the sodium salt of HBI-3808 having XRPD diffractogram of Pattern 9, comprises one or more peaks of the XRPD diffractogram of FIG. 9A, and/or Table 9. In certain embodiments, the XRPD diffractogram comprises i) 1, 2, 3, 4, 5, 6, 7, 8, 9, 10, 11, 12, 13, 14, 15, or any range there between, peaks of, or substantially matches, FIG. 9A; ii) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or any range there between, or all peaks (±0.2 °2θ) listed in Table 9, or both. In certain embodiments, the XRPD diffractogram substantially matches, FIG. 9A.

Sodium salt of HBI-3808 having X RPD diffractogram of Pattern 10

XRPD diffractogram of the sodium salt of HBI-3808 having XRPD diffractogram of Pattern 10, comprises one or more peaks of the XRPD diffractogram of FIG. 10A, and/or Table 10. In certain embodiments, the XRPD diffractogram comprises i) 1, 2, 3, 4, 5, 6, 7, 8, 9, 10, 11, 12, 13, 14, 15, 16, 17, 18, 19, 20, or any range there between, peaks of, or substantially matches, FIG. 10A; ii) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or any range there between, or all peaks (±0.2 °2θ) listed in Table 10, or both. In certain embodiments, the XRPD diffractogram substantially matches, FIG. 10A.

Sodium salt of HBI-3808 having X RPD diffractogram of Pattern 11

XRPD diffractogram of the sodium salt of HBI-3808 having XRPD diffractogram of Pattern 11, comprises one or more peaks of the XRPD diffractogram of FIG. 11A, and/or Table 11. In certain embodiments, the XRPD diffractogram comprises i) 1, 2, 3, 4, 5, 6, 7, 8, 9, 10, 11, 12, 13, 14, 15, or any range there, between peaks of, or substantially matches, FIG. 11A; ii) 1, 2, 3,4, 5,6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, or any range there between, or all peaks (±0.2°2θ) listed in Table 11, or both. In certain embodiments, the XRPD diffractogram substantially matches, FIG. 11A.

In certain embodiments, the sodium salt of HBI-3808 of XRPD diffractogram of Pattern 11, has a i) XRPD diffractogram comprising a) 1, 2, 3, 4, 5, 6, 7, 8, 9, 10, 11, 12, 13, 14, 15, or any range there, between peaks of, or substantially matches, FIG. 11A, b) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, or any range there between, or all peaks (±0.2 °2θ) listed in Table 11, or both; ii) TG-DSC thermogram substantially matching that of FIG. 11C; or both.

Sodium salt of HBI-3808 having XRPD diffractogram of Pattern 12

XRPD diffractogram of the sodium salt of HBI-3808 having XRPD diffractogram of Pattern 12, comprises one or more peaks of the XRPD diffractogram of FIG. 12A, and/or Table 12. In certain embodiments, the XRPD diffractogram comprises i) 1, 2, 3, 4, 5, 6, 7, 8, 9, 10, 11, 12, 13, 14, 15, or any range there between, peaks of, or substantially matches, FIG. 12A; ii) 1, 2, 3,4, 5,6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, or any range there between, or all peaks (±0.2 °2θ) listed in Table 12, or both. In certain embodiments, the XRPD diffractogram substantially matches, FIG. 12A.

In certain embodiments, the sodium salt of HBI-3808 of XRPD diffractogram of Pattern 12, has i) XRPD diffractogram comprising a) 1, 2, 3, 4, 5, 6, 7, 8, 9, 10, 11, 12, 13, 14, 15, or any range there, between peaks of, or substantially matches, FIG. 12A, b) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, or any range there between, or all peaks (±0.2 °2θ) listed in Table 12, or both; ii) a TG-DSC thermogram substantially matching that of FIG. 12C; or both.

Sodium salt of HBI-3808 having XRPD diffractogram of Pattern 13

XRPD diffractogram of the sodium salt of HBI-3808 having XRPD diffractogram of Pattern 13, comprises one or more peaks of the XRPD diffractogram of FIG. 13A, and/or Table 13. In certain embodiments, the XRPD diffractogram comprises i) 1, 2, 3, 4, 5, 6, 7, 8, 9, 10, 11, 12, 13, 14, 15, 16, 17, 18, 19, 20, or any range there between, peaks of, or substantially matches, FIG. 13A; ii) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or any range there between, or all peaks (±0.2°2θ) listed in Table 13, or both. In certain embodiments, the XRPD diffractogram substantially matches, FIG. 13A.

In certain embodiments, the sodium salt of HBI-3808 of XRPD diffractogram of Pattern 13, has i) XRPD diffractogram comprising a) 1, 2, 3, 4, 5, 6, 7, 8, 9, 10, 11, 12, 13, 14, 15, 16, 17, 18, 19, 20, or any range there, between peaks of, or substantially matches, FIG. 13A, b) 1, 2, 3, 4, 5,6,7, 8, 9, 10, 11, 12, 13, 14, 15, 16, 17, 18, 19, 20, 21, 22, 23, 24, 25, 26, 27, 28, 29, 30, or any range there between, or all peaks (±0.2 °2θ) listed in Table 13, or both; ii) a TG-DSC thermogram substantially matching that of FIG. 13C; or both.

Sodium salt of HBI-3808 having X RPD diffractogram of Pattern 14

XRPD diffractogram of the sodium salt of HBI-3808 having XRPD diffractogram of Pattern 14, comprises one or more peaks of the XRPD diffractogram of FIG. 14A, and/or Table 14. In certain embodiments, the XRPD diffractogram comprises i) 1, 2, 3, 4, 5, 6, 7, 8, 9, 10, 11, 12, 13, 14, 15, or any range there between, peaks of, or substantially matches, FIG. 14A; ii) 1, 2, 3,4, 5, 6, 7, 8, 9, 10, 11, 12, 13, 14, 15, 16, 17, 18, 19, 20, 21, 22, 23, 24,25, 26, 27, 28, 29, 30, or any range there between, or all peaks (±0.2 °2θ) listed in Table 14, or both. In certain embodiments, the XRPD diffractogram substantially matches, FIG. 14A.

In certain embodiments, the sodium salt of HBI-3808 of XRPD diffractogram of Pattern 14, has i) XRPD diffractogram comprising a) 1, 2, 3, 4, 5, 6, 7, 8, 9, 10, 11, 12, 13, 14, 15, or any range there between, peaks of, or substantially matches, FIG. 14A, b) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or any range there between, or all peaks (0.2 °2θ) listed in Table 14, or both; ii) a TG-DSC thermogram substantially matching that of FIG. 14C; or both.

Sodium salt of HBI-3808 having XRPD diffractogram of Pattern 15

XRPD diffractogram of the sodium salt of HBI-3808 having XRPD diffractogram of Pattern 15, comprises one or more peaks of the XRPD diffractogram of FIG. 15A, and/or Table 15. In certain embodiments, the XRPD diffractogram comprises i) 1, 2, 3, 4, 5, 6, 7, 8, 9, 10, 11, 12, 13, 14, 15, or any range there between, peaks of, or substantially matches, FIG. 15A; ii) 1, 2, 3,4, 5,6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, or any range there between, or all peaks (±0.2°2θ) listed in Table 15, or both. In certain embodiments, the XRPD diffractogram substantially matches, FIG. 15A.

In certain embodiments, the sodium salt of HBI-3808 of XRPD diffractogram of Pattern 15, has i) XRPD diffractogram comprising a) 1, 2, 3, 4, 5, 6, 7, 8, 9, 10, 11, 12, 13, 14, 15, or any range there between, peaks of, or substantially matches, FIG. 15A, b) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, or any range there between, or all peaks (±0.2 °2θ) listed in Table 15, or both; ii) a TG-DSC thermogram substantially matching that of FIG. 15C; or both.

In certain embodiments, the sodium salt of HBI-3808 having XRPD diffractogram of Pattern 15, is a mono sodium, mono-hydrate, mono acetone solvate salt.

Sodium salt of HBI-3808 having XRPD diffractogram of Pattern 16 XRPD diffractogram of the sodium salt of HBI-3808 having XRPD diffractogram of Pattern 16, comprises one or more peaks of the XRPD diffractogram of FIG. 16A, and/or Table 16. In certain embodiments, the XRPD diffractogram comprises i) 1, 2, 3, 4, 5, 6, 7, 8, 9, 10, 11, 12, 13, 14, 15, or any range there between, peaks of, or substantially matches, FIG. 16A; ii) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 45, 50, 55, 60, 65, 70 or any range, or value there between, or all peaks (±0.2 °2θ) listed in Table 16, or both. In certain embodiments, the XRPD diffractogram substantially matches, FIG. 16A.
Polymorphic mixtures of sodium salt of HBI-3808

In certain embodiments, the sodium salt of HBI-3808 is a polymorphic mixture and contains a mixture of at least 2 polymorphs, wherein the at least 2 polymorphs are independently a sodium salt of HBI-3808 having XRPD diffractogram of Pattern 1, 2, 3, 4, 5, 6, 7, 8, 9, 10, 11, 12, 13, 14, 15, or 16, and the at least 2 polymorphs have different XRPD diffractogram. In certain embodiments, the sodium salt of HBI-3808 can be a polymorphic mixture containing the sodium salt of HBI-3808 of XRPD Pattern 2, and at least one of the sodium salt of HBI-3808 of XRPD Pattern 1, 3, 4, 5, 6, 7, 8, 9, 10, 11, 12, 13, 14, 15, or 16. In certain embodiments, the sodium salt of HBI-3808 can be a polymorphic mixture containing the sodium salt of HBI-3808 of XRPD Pattern 5, and at least one of the sodium salt of HBI-3808 of XRPD Pattern 1, 2, 3, 4, 6, 7, 8, 9, 10, 11, 12, 13, 14, 15, or 16. In certain embodiments, the sodium salt of HBI-3808 can be a polymorphic mixture containing the sodium salt of HBI-3808 of XRPD Pattern 2, and the sodium salt of HBI-3808 of XRPD Pattern 5.

Method of making Sodium Salts of HBI-3808

In certain embodiments, the method of making the sodium salt of HBI-3808, can be method A, wherein the method A includes any one of, any combination of, or all of steps (a) to (f). Step (a) can include contacting HBI-3808 with first solvent to form a first slurry. Step (b) can include dissolving sodium hydroxide in a second solvent to form a first solution. Step (c) can include contacting the first slurry of step (a) with the first solution to form a mixture at a first temperature. Step (d) can include contacting the mixture of step (c) with an anti-solvent to form a second slurry. Step (e) can include cooling the second slurry to a second temperature, wherein the second temperature is lower than the first temperature. Step (f) can include isolating solids from the second slurry. The first solvent of method A can contain methanol. In certain embodiments, the first solvent of method A is methanol. The second solvent of method A can contain water. In certain embodiments, the second solvent of method A is water. The anti-solvent of method A can contain acetone and/or tert-butyl methyl ether (TBME). In certain embodiments, the anti-solvent of method A is acetone. In certain embodiments, the anti-solvent of method A is TBME. The first temperature of method A can be about 20° C. to 60° C., about 25° C. to 55° C., about 30° C. to 55° C., about 35° C. to 45° C., about 40° C., or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., or any range, or value there between. In certain embodiments, the first temperature of method A is about 40° C. The second temperature of method A can be about 0° C. to 20° C., about 2° C. to 15° C., about 3° C. to 10° C., or about 5° C., or 0, 1, 2, 3, 4, 5, 6, 7, 8, 9, 10, 11, 12, 13, 14, 15, 16, 17, 18, 19, or 20° C., or any range, or value there between. In certain embodiments, the second temperature of method A is about 5° C. In certain embodiments, the method A, further includes washing the solids (e.g., isolated in step f) with a rinse solution. In certain embodiments, the rinse solution of method A contains methanol, water, and/or acetone. In certain embodiments, the rinse solution comprises methanol, water, and acetone. In certain embodiments, the rinse solution of method A contains about 3 vol. % to 15 vol. %, or 3, 4, 5, 6, 7, 8, 9, 10, 11, 12, 13, 14, or 15 vol. %, or any range, or value there between of methanol, about 0.1 vol. % to 5 vol. %, or 0.1, 0.5, 1, 2, 3, 4, or 5, vol. %, or any range, or value there between of water, and about 84.9 vol. % to 96 vol. %, or, or 84.9, 85, 86, 87, 88, 89, 90, 91, 92, 93, 94, 95, or 96 vol. %, any range, or value there between of acetone. In certain embodiments, the rinse solution of method A contains methanol, water, and acetone in a ratio of about 9:1:90% v:% v:% v. In certain embodiments, the method A, further comprises drying the separated solids. In certain embodiments, the separated solids are dried under vacuum. In certain embodiments, the isolated solids and/or separated solids, of the method A, contains the sodium salt of HBI-3808 of XRPD Pattern 2.

In certain embodiments, the method of making the sodium salt of HBI-3808 can be method B, wherein the method B includes any one of, any combination of, or all of steps (a′) to (d′). Step (a′) can include contacting HBI-3808 with a first solvent at a first temperature to form a slurry.

Step (b′) can include contacting the slurry of step (a′) with sodium hydroxide and optionally an additional solvent. Step (c′) can include temperature cycling the slurry (e.g., formed in step (b′)) between the first temperature and a second temperature, wherein the second temperature is lower than the first temperature. The step (d′) can include isolating solids from the slurry (e.g., formed during and/or after the temperature cycling). The first solvent of method B can be a lower alcohol, e.g., a C₁₋₄ alcohol. The C₁₋₄ alcohol can be methanol, ethanol, 1-propanol, 2-propanol, 1-butanol, 2-butanol, iso-butanol, tert-butanol, or any combination thereof. In certain embodiments, the lower alcohol is 2-propanol. In certain embodiments, the method B can further include drying the solids isolated in step (d′). In certain embodiments, the solids are be dried under vacuum. The first temperature of method B can be about 20° C. to 60° C., about 25° C. to 55° C., about 30° C. to 55° C., about 35° C. to 45° C., or about 40° C., or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., or any range, or value there between. In certain embodiments, the first temperature of method B is about 40° C. The second temperature of method B can be about 0° C. to 20° C., about 2° C. to 15° C., about 3° C. to 10° C., or about 5° C., or 0, 1, 2, 3, 4, 5, 6, 7, 8, 9, 10, 11, 12, 13, 14, 15, 16, 17, 18, 19, or 20° C., or any range, or value there between. In certain embodiments, the second temperature of method B is about 5° C. In certain embodiments, the isolated solids, of the method B, contains the sodium salt of HBI-3808 of XRPD Pattern 3.

Certain aspects are directed to method of making a HBI-3808 sodium salt polymorph. In certain embodiments, the method of making HBI-3808 sodium salt polymorph, can be method C, wherein the method C can include any one of, or any combination of, or all of steps (a″) to (d″). Step (a″) can include dissolving a sodium salt of HBI-3808 in a solvent to form a solution at a first temperature. Step (b″) can include contacting the solution of step (a″) with a counter solvent. Step (c″) can include optionally, cycling the solution (e.g., formed in step b″) between the first temperature and a second temperature, where the second temperature is lower than the first temperature. Step d″ can include isolating solids from the solution (e.g., formed in step (b″) or (c″)). In certain embodiments, the solvent of method C is water. In certain embodiments, the counter solvent is tetrahydrofuran (THF). In certain embodiments, the method C further includes drying the isolated solids (e.g., isolated in step d″). In certain embodiments, the isolated solids can be dried under vacuum. The first temperature of method C can be about 20° C. to 60° C., about 25° C. to 55° C., about 30° C. to 55° C., about 35° C. to 45° C., or about 40° C. or about 40° C., or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., or any range, or value there between. In certain embodiments, the first temperature of method C is about 40° C. The second temperature of method C can be about 0° C. to 20° C., about 2° C. to 15° C., about 3° C. to 10° C., or about 5° C., or about 5° C., or 0, 1, 2, 3, 4, 5, 6, 7, 8, 9, 10, 11, 12, 13, 14, 15, 16, 17, 18, 19, or 20° C., or any range, or value there between. In certain embodiments, the second temperature of method C is about 5° C. In certain embodiments, the isolated solids, of the method C, contains the sodium salt of HBI-3808 of XRPD Pattern 4.

In certain embodiments, the method of making a HBI-3808 sodium salt polymorph, can be method D, wherein method D includes temperature cycling a solution containing a sodium salt of HBI-3808 between a first temperature and a second temperature, wherein the second temperature is lower than the first temperature. In certain embodiments, the method D further contains, isolating solid from the solution. In certain embodiments, in method D, the solution is formed by dissolving an amorphous sodium salt of HBI-3808. The first temperature of method D can be about 20° C. to 60° C., about 25° C. to 55° C., about 30° C. to 55° C., about 35° C. to 45° C., or about 40° C., or about 40° C., or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., or any range, or value there between. In certain embodiments, the first temperature of method D is about 40° C.

The second temperature of method D can be about 0° C. to 20° C., about 2° C. to 15° C., about 3° C. to 10° C., or about 5° C., or about 5° C., or 0, 1, 2, 3, 4, 5, 6, 7, 8, 9, 10, 11, 12, 13, 14, 15, 16, 17, 18, 19, or 20° C., or any range, or value there between. In certain embodiments, the second temperature of method D is about 5° C. In certain embodiments, in method D, the solution contains the sodium salt of HBI-3808 and dimethylformamide (DMF). In certain embodiments, in method D, the solution contains the sodium salt of HBI-3808, DMF and water. In certain embodiments, in method D, the solution contains the sodium salt of HBI-3808, DMF and water, wherein the % v: % v ratio of DMF:water is about 70:30 to 90:10, or about 75:25 to 85:15, or about 72:28, 74:26, 75:25, 76:24, 78:22, 80:20, 82:18, 84:16, 85:15, 86:14, 88:12, or 90:10, or any range, or value there between. In certain embodiments, in method D, the solution contains the sodium salt of HBI-3808, DMF and water, wherein the % v:% v ratio of DMF: water is about 80:20. In certain embodiments, in method D, the solution contains the sodium salt of HBI-3808, DMF and water, and the solids isolated contains sodium salt of HBI-3808 of Pattern 8. In certain embodiments, in method D, the solution contains a sodium salt of HBI-3808 and tert-butyl methyl ether (tBME). In certain embodiments, in method D, the solution contains a sodium salt of HBI-3808 and tert-butyl methyl ether (tBME), and the solids isolated contains sodium salt of HBI-3808 of Pattern 12.

In certain embodiments, the method of making a HBI-3808 sodium salt polymorph, can be method E, wherein the method E include exposing a solution of a sodium salt of HBI-3808 to 0° C. to 20° C., or about 5° C., or 0, 1,2,3,4,5,6,7,8,9,10,11,12,13,14,15,16,17,18,19, or 20° C., or any range, or value there between, for 10 h to 40 h, or 10, 12, 14, 16, 18, 20, 22, 24, 26, 28, 30, 32, 34, 36, 38, or 40 h. In certain embodiments, the method E further includes isolating the solids from the solution. In certain embodiments, in the method E, the solution is formed by dissolving the sodium salt of HBI-3808 of Pattern 5. In certain embodiments, the solution of method E contains the sodium salt of HBI-3808, acetone and water. In certain embodiments, the solution of method E contains the sodium salt of HBI-3808, acetone and water, wherein the % v:% v ratio of acetone: water is about 10:90 to 40:60:10, or, or 10:90, 15:85, 20:80, 25:75, 30:70, 35:65, 40:60, or any range, or value there between. In certain embodiments, the solution of method E contains the sodium salt of HBI-3808, 1-propanol and water. In certain embodiments, the solution of method E contains the sodium salt of HBI-3808, 1-propanol and water, wherein the % v:% v ratio of 1-propanol: water is about 25:75 to 75:25, about 35:65 to 65:35, about 40:60 to 60:40, about 45:55 to 55:45, or about 50:50. In certain embodiments, in the method E, the isolated solids contain the sodium salt of HBI-3808 of Pattern 9. In certain embodiments, in the method E, the isolated solids contain the sodium salt of HBI-3808 of Pattern 10. In certain embodiments, in the method E, the isolated solids contain the sodium salt of HBI-3808 of Pattern 10, and the solution contains the sodium salt of HBI-3808, 1-propanol and water, at a 1-propanol: water % v: % v ratio of about 25:75 to 75:25, about 35:65 to 65:35, about 40:60 to 60:40, about 45:55 to 55:45, or about 50:50. In certain embodiments, in the method E, the isolated solids contain the sodium salt of HBI-3808 of Pattern 9, and the solution contains the sodium salt of HBI-3808, DMF and water, at a % v: % v ratio of about. 10:90 to 40:60.

In certain embodiments, the method of making a HBI-3808 sodium salt polymorph, can be method F, wherein the method F include evaporating a solution containing a sodium salt of HBI-3808. In certain embodiments, the solution of method F contains, the sodium salt of HBI-3808, ethanol and water. In certain embodiments, the water activity (a_{w}) of the solution of the method F is about 0.2, to 0.8, about 0.3 to 0.7, about 0.4 to 0.6, or about 0.5. In certain embodiments, ethanol: water % v: % v ratio of the solution of method F is about 99:1 to 80:20, about 95:5 to 85:15, or about 90:10. In certain embodiments, sodium salt of HBI-3808 of XRPD Pattern 14 is obtained by method F.

In certain embodiments, the method of making a HBI-3808 sodium salt polymorph, can be method G, wherein method G includes steps (a″′), (b″′) and/or (c″′). Step (a″′) can include forming a slurry containing a sodium salt of HBI-3808. Step (b″′) can include isolating solids from the slurry formed in step (a″′). Step (c″′) can include drying the isolated solids. In certain aspects, the slurry is formed by contacting a poorly crystalline sodium salt of HBI-3808 with solvent. In certain embodiment, the solvent of the method G is methyl ethyl ketone (MEK). In certain embodiment, step (a″′) includes keeping the slurry at a first temperature for 6 h to 40 h, or 10 h to 30 h, or 20 h to 28 h, or about 24 h. In certain embodiment, step (a″′) includes stirring the slurry.

In certain embodiments, the first temperature of method G is about 20° C. to 60° C., about 25° C. to 55° C., about 30° C. to 55° C., about 35° C. to 45° C., or about 40° C., or about 40° C., or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., or any range, or value there between. In certain embodiments, the first temperature of the method G is about 40° C. In certain embodiments, in step (c″′) the isolated solids are dried at i) a temperature of about 20° C. to 60° C., about 25° C. to 55° C., about 30° C. to 55° C., about 35° C. to 45° C., or about 40° C.; ii) at a humidity of about 0 % RH to 20% RH, or about 0, 2, 4, 6, 8, 10, 12, 14, 16, 18, or 20% RH, or any value, or range there between; iii) for 5 h to 30 h, or 10 h to 25 h, or 15 h to 20 h, or about 18 h; or any combination thereof. In certain embodiments, the isolated solids, of the method G, contains the sodium salt of HBI-3808 of XRPD Pattern 16.

In certain embodiments, the method of making the sodium salt of HBI-3808, can be method H, wherein the method H includes any one of, any combination of, or all of steps (aa), (bb), (cc), (dd), (ee), (ff), and (gg). Step (aa) can include contacting HBI-3808 with sodium hydroxide to form a first solution, at a first temperature. Step (bb) can include heating the first solution from the first temperature to a second temperature, where the second temperature is higher than the first temperature. Step (cc) can include seeding the solution obtained in (bb) with a crystalline sodium salt of HBI-3808 to obtain a seeded solution. Step (dd) can include adding an anti-solvent to the seeded solution to obtain a slurry. Step (ee) can include isolating solids from the slurry. Step (ff) can include drying the isolated solids. Step (gg) can include exposing the dried solids to conditions including a combination of temperature and/or humidity, for a time sufficient to form sodium salt of HBI-3808 of XRPD pattern 5. In certain embodiments, in step (aa) HBI-3808 is contacted with an aqueous solution of sodium hydroxide to form the first solution. In certain embodiments, in step (aa) the HBI-3808 is contacted with sodium hydroxide at a HBI-3808:NaOH molar ratio of about 1:1 to 1:1.4, or about 1:1.1 to 1:1.3, or about 1:1.15 to 1:1.25, or about 1.2, or 1:1, 1:1.1, 1:1.2, 1:1.3, or 1:1.4 or any value or range there between. In certain embodiments, the first temperature of method H is about 3° C. to 20° C., or about 5° C. to 15° C., or, about 10° C., or 3, 4, 5, 6, 7, 8, 9, 10, 11, 12, 13, 14, 15, 16, 17, 18, 19, or 20° C. or any value or range there between. In certain embodiments, the second temperature of method H is about 15° C. to 35° C., or about 20° C. to 30° C., or about 25° C., or, 15, 16, 17, 18, 19, 20, 21, 22, 23, 24, 25, 26, 27, 28, 29, 30, 31, 32, 33, 34, or 35° C., or any value or range there between. In certain embodiments, in step (cc) the solution is seeded with the sodium salt of HBI-3808 of XRPD Pattern 5. In certain embodiments, in step (cc) the solution is seeded with about 0.1 wt. % to 5 wt. %, or about 0.3 wt. % to 3 wt. %, or about 0.5 wt. % to 2 wt. %, or about 1 wt. %, or 0.1, 0.2, 0.3, 0.4, 0.5, 0.6, 0.7, 0.8, 0.9, 1, 2, 3, 4, or 5 wt. % of the sodium salt of HBI-3808 of XRPD Pattern 5. In certain embodiments, the acetone, water ratio in the slurry obtained in step (dd) is about 70:30 to 90:10% v:% v, or about 75:25 to 85:15 % v:% v, or about 80:20% v:% v, or 70:30, 75:25, 80:20, 85:15 or 90:10 vol./vol., or any value or range there between. In certain embodiments, in step (ff), the isolated solids are dried at about 30° C. to 80° C., or about 30° C. to 70° C., or about 30° C. to 60° C., or about 30° C. to 50° C., or about 35° C. to 45° C., or about 45° C., or 30, 35, 40, 45, 50, 55, 60, 65, 70, 75, or 80° C., or any range or value there between. In certain embodiments, in step (gg), the dried solids are exposed to i) a temperature of about 30° C. to 80° C., or about 30° C. to 70° C., or about 30° C. to 60° C., or about 30° C. to 50° C., or about 35° C. to 45° C., or about 45° C., or 30, 35, 40, 45, 50, 55, 60, 65, 70, 75, or 80° C., or any range or value there between; and/or ii) to a humidity of about 40% RH to 100 % RH, or about 45% RH to 90% RH, or about 50% RH to 85% RH, or about 55% RH to 85% RH, about 60% RH to 80% RH, about 70% RH to 80% RH, or about 75% RH, or 40, 45, 50, 55, 60, 65, 70, 75, 80, 85, 90, 95, or 100% RH, or any range or value there between, for about 24 hrs. to 240 hrs., or about 72 hrs. or 20, 24, 30, 40, 50, 60, 70, 80, 90, 100, 110, 120, 130, 140, 150, 160, 170, 180, 190, 200, 210, 220, 230, or 240 hrs. or any range or value there between.

Certain aspects are directed to a method of making a sodium salt of HBI-3808 having XRPD of Pattern 5. In certain embodiments, the method includes providing a sample of a sodium salt of HBI-3808 having XRPD of Pattern 2, and/or exposing the Pattern 2 sodium salt (e.g., sodium salt of HBI-3808 having XRPD of Pattern 2) to conditions including a combination of temperature and humidity for a time sufficient to convert the Pattern 2 sodium salt to a Pattern 5 sodium salt. In certain embodiments, the Pattern 2 sodium salt is exposed to the temperature of at least about 40° C. In certain embodiments, the Pattern 2 sodium salt is exposed to the temperature of about 10, 15, 20, 25, 30, 35, 40, 45, 50, 55, 60, 65, 70, 75, 80, 85, 90, 95, 100, 105, 110, 115, 120, 125, 130, 135, 140, 145, or 150° C., or any range or value there between. In certain embodiments, the Pattern 2 sodium salt is exposed to the humidity of at least about 40% RH. In certain embodiments, the Pattern 2 sodium salt is exposed to the humidity of 40% RH to 90% RH, or 40% RH to 80% RH, or 50% RH to 90% RH, or 50% RH to 80% RH, 50% RH to 70% RH, 55% RH to 65% RH, or 40, 45, 50, 55, 60, 65, 70, 75, 80, 85, 90, 95, or 100% RH, or any range or value there between. In certain embodiments, the Pattern 2 sodium salt is exposed the suitable temperature and/or humidity, of about 24 hours to about eight (8) weeks. In certain embodiments, the Pattern 2 sodium salt is exposed to i) the temperature of about 15° C. to 45° C., or about 20° C. to 40° C., or 15, 16, 17, 18, 19, 10, 21, 22, 23, 24, 25, 26, 27, 28, 29, 30, 31, 32, 33, 34, 35, 36, 37, 38, 39, 40, 41, 42, 43, 44 or 45° C., or any range or value there between; and ii) to the humidity of 40% RH to 90% RH, or 40% RH to 80% RH, or 50% RH to 90% RH, or 50% RH to 80% RH, 50 % RH to 70% RH, 55% RH to 65% RH, or 40, 45, 50, 55, 60, 65, 70, 75, 80, 85, 90, 95, or 100 % RH, or any range or value there between, for 24 hrs. to 240 hrs. or 20, 24, 30, 40, 50, 60, 70, 80, 90, 100, 110, 120, 130, 140, 150, 160, 170, 180, 190, 200, 210, 220, 230, or 240 hrs. or any range or value there between, to form the sodium salt of HBI-3808 having XRPD of Pattern 5. In certain embodiments, the Pattern 2 sodium salt is exposed to i) the temperature of about 20° C. to 40° C., and ii) to the humidity of 50% RH to 70% RH, for 60 hrs. to 120 hrs. to form the sodium salt of HBI-3808 having XRPD of Pattern 5. In certain embodiments, sodium salt of HBI-3808 having XRPD of Pattern 2 can be prepared by the method A. In certain embodiments, the method of making the sodium salt of HBI-3808, can include making the sodium salt of HBI-3808 having XRPD of Pattern 2 according to the method A, and making the sodium salt of HBI-3808 having XRPD of Pattern 5 from the sodium salt of HBI-3808 having XRPD of Pattern 2.

In certain embodiments, the method of forming the sodium salt of HBI-3808 having XRPD of Pattern 5 includes, contacting the sodium salt of HBI-3808 having XRPD of Pattern 2 with water to form a slurry; exposing the slurry to conditions suitable to form the sodium salt of Pattern 5; and isolating the sodium salt of Pattern 5 from the slurry. In certain embodiments, the slurry is exposed to a temperature of about 15° C. to 45° C., or about 20° C. to 40° C., or 20° C. to 30° C., or 25^{°} ° C. or 15, 16, 17, 18, 19, 10, 21, 22, 23, 24, 25, 26, 27, 28, 29, 30, 31, 32, 33, 34, 35, 36, 37, 38, 39, 40, 41, 42, 43, 44 or 45° C., or any range or value there between; for 2 hrs. to 40 hrs. or 20 hrs. to 30 hrs. or 24 hrs. or 2, 4, 6, 8, 10, 12, 14, 16, 18, 20, 22, 24, 26, 28, 30, 32, 34, 36, 38, or 40 hrs. or any range or value there between, to form the sodium salt of Pattern 5. In certain embodiments, the slurry is exposed to a temperature of about 20° C. to 30° C., for 20 hrs. to 30 hrs. to form the sodium salt of Pattern 5.

Certain aspects are directed to a method of making a sodium salt of HBI-3808 having XRPD of Pattern 6. The method includes providing a sample of a sodium salt of HBI-3808 having XRPD of Pattern 2, and/or exposing the Pattern 2 sodium salt (e.g., sodium salt of HBI-3808 having XRPD of Pattern 2) to a temperature suitable to convert the Pattern 2 sodium salt to a Pattern 6 sodium salt. In certain embodiments, the Pattern 2 sodium salt is exposed to the temperature of at least about 100° C. In certain embodiments, the Pattern 2 sodium salt is exposed to the temperature of about 100° C. to 250° C., or about 110, 120, 130, 140, 150, 160, 170, 180, 190, 200, 210, 220, 230, 240, or 250° C., or any range or value there between. In certain embodiments, sodium salt of HBI-3808 having XRPD of Pattern 2 can be prepared by the method A.

Certain aspects are directed to a method of making a sodium salt of HBI-3808 having XRPD of Pattern 7. The method includes providing a sample of a sodium salt of HBI-3808 having XRPD of Pattern 5, and/or exposing the Pattern 5 sodium salt (e.g., sodium salt of HBI-3808 having XRPD of Pattern 5) to a temperature suitable to convert the Pattern 5 sodium salt to a Pattern 7 sodium salt. In certain embodiments, the Pattern 7 sodium salt is exposed to the temperature of at least about 100° C. In certain embodiments, the Pattern 2 sodium salt is exposed to the temperature of about 100° C. to 250° C., or about 110, 120, 130, 140, 150, 160, 170, 180, 190, 200, 210, 220, 230, 240, or 250° C., or any range or value there between.

Certain aspects are directed to a method of making a sodium salt of HBI-3808 having XRPD of Pattern 11. The method includes providing a sample of a sodium salt of HBI-3808 having XRPD of Pattern 8, and/or exposing the Pattern 8 sodium salt (e.g., sodium salt of HBI-3808 having XRPD of Pattern 8) to a condition suitable to convert the Pattern 8 sodium salt to a Pattern 11 sodium salt. In certain embodiments, the Pattern 8 sodium salt is dried to form the Pattern 11 sodium salt. In certain embodiments, the Pattern 8 sodium salt is dried in 2 propanol and water to form the Pattern 11 sodium salt. In certain embodiments, the Pattern 8 sodium salt is dried in 2-propanol and water having 2-propanol: water ratio of about 99:1% v:% v. to 80:20% v:% v, or about 95:5% v:% v to 85:15% v:% v, or about 92:8% v:% v to 88:12% v:% v, or about 90:10% % v:% v, or 99:1, 97:3, 95:5, 93:7, 91:9, 90:10, 89:11, 87:13, 85:5, 83:17, 81:19, or 80:20% v:% v, or any range or value there between, to form the Pattern 11 sodium salt. In certain embodiments, the Pattern 8 sodium salt is dried under i) vacuum, and/or ii) at about 20° C. to 60° C., about 25° C. to 55° C., about 30° C. to 55° C., about 35° C. to 45° C., or about 40° C., or about 40° C., or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., or any range, or value there between, to form the Pattern 11 sodium salt. In certain embodiments, the Pattern 8 sodium salt is dried in 2-propanol: water under i) vacuum, and/or ii) at about 20° C. to 60° C., about 25° C. to 55° C., about 30° C. to 55° C., about 35° C. to 45° C., or about 40° C., or about 40° C., or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., or any range, or value there between, to form the Pattern 11 sodium salt.

In certain embodiments, the method of making the sodium salt of HBI-3808 having XRPD of Pattern 5, includes slurring the sodium salt of HBI-3808 having XRPD of Pattern 10 or 11, or a mixture thereof, in a solvent. In certain embodiments, the sodium salt of HBI-3808 having XRPD of Pattern 10, or 11, or a mixture thereof is contacted with MEK, MiBK, ethyl acetate, dioxane, or any combination thereof, at 20° C. to 60° C., about 25° C. to 55° C., about 30° C. to 55° C., about 35° C. to 45° C., or about 40° C., or about 40° C., or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., or any range, or value there between, to form the Pattern 5 sodium salt.

Certain aspects are directed to a method of making a sodium salt of HBI-3808 having XRPD of Pattern 13. The method includes providing a sample of a sodium salt of HBI-3808 having XRPD of Pattern 8, and/or exposing the Pattern 8 sodium salt (e.g. sodium salt of HBI-3808 having XRPD of Pattern 8) to a condition suitable to convert the Pattern 8 sodium salt to a Pattern 13 sodium salt. In certain embodiments, the Pattern 8 sodium salt is dried in DMF to form the Pattern 13 sodium salt. In certain embodiments, the Pattern 8 sodium salt is dried in DMF under i) vacuum, and/or ii) at about 20° C. to 60° C., about 25° C. to 55° C., about 30° C. to 55° C., about 35° C. to 45° C., or about 40° C., or about 40° C., or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., or any range, or value there between, to form the Pattern 13 sodium salt.

In certain embodiments, the method of making a sodium salt of HBI-3808 having XRPD of Pattern 13 includes solvent drop grinding of an amorphous sodium salt of HBI-3808. In certain embodiments, the solvent drop grinding is performed in THK, MiBK, or a mixture thereof.

In certain embodiments, a sodium salt of HBI-3808 is prepared according to one or more steps described in FIG. 20A. In certain embodiments, amorphous sodium salt of HBI-3808 of XRPD is prepared according to one or more steps described in FIG. 20A. In certain embodiments, the sodium salt of HBI-3808 of XRPD pattern 1 is prepared according to one or more steps described in FIG. 20A. In certain embodiments, the sodium salt of HBI-3808 of XRPD pattern 3 is prepared according to one or more steps described in FIG. 20A. In certain embodiments, the sodium salt of HBI-3808 of XRPD pattern 5 is prepared according to one or more steps described in FIG. 20A. In certain embodiments, the sodium salt of HBI-3808 of XRPD pattern 8 is prepared according to one or more steps described in FIG. 20A. In certain embodiments, the sodium salt of HBI-3808 of XRPD pattern 11 is prepared according to one or more steps described in FIG. 20A.

In certain embodiments, the sodium salt of HBI-3808 of XRPD pattern 12 is prepared according to one or more steps described in FIG. 20A. In certain embodiments, the sodium salt of HBI-3808 of XRPD pattern 13 is prepared according to one or more steps described in FIG. 20A. In certain embodiments, the sodium salt of HBI-3808 of XRPD pattern 14 is prepared according to one or more steps described in FIG. 20A.

In certain embodiments, a sodium salt of HBI-3808 is prepared according to one or more steps described in FIG. 20B. In certain embodiments, amorphous sodium salt of HBI-3808 of XRPD is prepared according to one or more steps described in FIG. 20B. In certain embodiments, the sodium salt of HBI-3808 of XRPD pattern 2 is prepared according to one or more steps described in FIG. 20B. In certain embodiments, the sodium salt of HBI-3808 of XRPD pattern 5 is prepared according to one or more steps described in FIG. 20B. In certain embodiments, the sodium salt of HBI-3808 of XRPD pattern 6 is prepared according to one or more steps described in FIG. 20B. In certain embodiments, the sodium salt of HBI-3808 of XRPD pattern 7 is prepared according to one or more steps described in FIG. 20B. In certain embodiments, the sodium salt of HBI-3808 of XRPD pattern 9 is prepared according to one or more steps described in FIG. 20B.

In certain embodiments, the sodium salt of HBI-3808 of XRPD pattern 10 is prepared according to one or more steps described in FIG. 20B. In certain embodiments, the sodium salt of HBI-3808 of XRPD pattern 11 is prepared according to one or more steps described in FIG. 20B. In certain embodiments, the sodium salt of HBI-3808 of XRPD pattern 15 is prepared according to one or more steps described in FIG. 20B.

In certain embodiments, a sodium salt of HBI-3808 is prepared according to one or more steps described in FIG. 2I. In certain embodiments, the sodium salt of HBI-3808 of XRPD pattern 2 is prepared according to one or more steps described in FIG. 2I. In certain embodiments, the sodium salt of HBI-3808 of XRPD pattern 5 is prepared according to one or more steps described in FIG. 2I.

In certain embodiments, the sodium salt of HBI-3808 of XRPD pattern 5 is prepared by temperature cycling of a slurry containing i) an amorphous sodium salt of HBI-3808 and ii) 2-methyl THF, ethyl acetate, iso-propyl acetate, MiBK, MEK, or any combination thereof, between a first temperature and a second temperature, where the first temperature is 0° C. to 10° C., and the second temperature is 30° C. to 50° C.

In certain embodiments, the sodium salt of HBI-3808 of XRPD pattern 5 is prepared by evaporating a slurry containing an amorphous sodium salt of HBI-3808. The certain embodiments, the slurry containing amorphous sodium salt of HBI-3808 further contains ethanol, methanol, MEK, or a combination thereof. In certain embodiments, the slurry contains ethanol and methanol at 45:55% v:% v to 55:45% v:% v, or 45:55, 47:53, 49:51, 50:50, 51:49, 53:47 or 55:45% v:% v, or any range or value there between. In certain embodiments, the slurry contains MEK and methanol at 45:55% v:% v to 55:45% v:% v, or 45:55, 47:53, 49:51, 50:50, 51:49, 53:47 or 55:45% v:% v, or any range or value there between. The slurry can be evaporated at 15° C. to 25° C., or 10, 12, 14, 15, 16, 18, 20, 22, 24, 25, 26, 28, or 30° C., or any range or values there between. In certain embodiments, the sodium salt of HBI-3808 of XRPD pattern 5 is prepared by evaporating a slurry containing an amorphous sodium salt of HBI-3808, and ethanol and methanol at 45:55% v:% v to 55:45% v:% v, at 15° C. to 25° C. In certain embodiments, the sodium salt of HBI-3808 of XRPD pattern 5 is prepared by evaporating a slurry containing an amorphous sodium salt of HBI-3808, and MEK and methanol at 45:55% v:% v to 55:45% v:% v, at 15° C. to 25° C.

In certain embodiments, the sodium salt of HBI-3808 of XRPD pattern 5 is prepared by drying a slurry containing i) the sodium salt of HBI-3808 of XRPD pattern 15, and ii) 1,4 -dioxane, or MEK:water at 95:1% v:% v to 99.5:0.5% v:% v, at 30° C. to 50° C. In certain embodiments, the sodium salt of HBI-3808 of XRPD pattern 5 is prepared by humid drying a slurry containing i) a sodium salt of HBI-3808 of XRPD pattern 15, and ii) 1,4 - dioxane, or MEK:water at 95:1% v: % v to 99.5:0.5% v: % v, at 30° C. to 50° C., and under vacuum.

In certain embodiments, the sodium salt of HBI-3808 of XRPD pattern 5 is prepared by drying sodium salt of HBI-3808 of XRPD pattern 12, at 30° C. to 50° C. and/or under vacuum.

In certain embodiments, the sodium salt of HBI-3808 of XRPD pattern 5 is prepared by exposing a slurry containing i) the sodium salt of HBI-3808 of XRPD pattern 10, the sodium salt of HBI-3808 of XRPD pattern 11, or both, and ii) ethyl acetate, MEK, 1, 4- dioxane, MiBK or any combination thereof, to a temperature of 30° C. to 50° C.

In certain embodiments, the sodium salt of HBI-3808 of XRPD pattern 5 is prepared by cooling the sodium salt of HBI-3808 of XRPD pattern 7 to a temperature of 5° C. to 30° C.

In certain embodiments, the sodium salt of HBI-3808 of XRPD pattern 5 is prepared by drying a slurry containing i) the sodium salt of HBI-3808 of XRPD pattern 16, and ii) 1,4 -dioxane, or MEK:water at 95:1% v:% v to 99.5:0.5% v:% v, at 30° C. to 50° C. In certain embodiments, the sodium salt of HBI-3808 of XRPD pattern 5 is prepared by humid drying a slurry containing i) a sodium salt of HBI-3808 of XRPD pattern 16, and ii) 1,4 - dioxane, or MEK:water at 95:1% v: % v to 99.5:0.5% v: % v, at 30° C. to 50° C., and under vacuum.

Compositions containing salt sodium salt of HBI-3808

Certain aspects are directed to a composition containing one or more sodium salt(s) of HBI-3808. The sodium salt of HBI-3808 can be a sodium salt described herein, and/or can be prepared by a method described herein. Sodium salt of HBI-3808, content in the composition can be 1, 2, 3,4, 5,6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 81, 82, 83, 84, 85, 86, 87, 88, 89, 90, 91, 92, 93, 94, 95, 96, 97, 98, 99 or 100 wt. %, or any value or range there between.

In some embodiments, the sodium salt HBI-3808 may be formulated as pharmaceutical compositions. Such pharmaceutical compositions may be administered to a subject, such as a human patient suffering from myocardial infarction or the sequelae thereof, at a dosage sufficient to treat myocardial infarction. The sodium salts of HBI-3808 may be formulated as oral compositions or may be used to manufacture parenteral formulations, such as intravenous (IV), intraperitoneal, or intramuscular formulations. For parenteral administration, the pharmaceutical composition may be formulated as a sterile injectable liquid stored in a suitable container such as a sterile syringe, a sterile bag, or an ampule, or in the form of an aqueous or non-aqueous liquid suspension. The nature and composition of the pharmaceutical carrier, diluent or excipient will, of course, depend on the intended route of administration, for example whether by intravenous or intramuscular injection.

Ethanolamine salt of HBI-3808

The ethanolamine salt of HBI-3808, can be a monoethanolamine salt. In certain embodiments, the ethanolamine salt of HBI-3808 is an isolated ethanolamine salt of HBI-3808. In certain embodiments, the ethanolamine salt of HBI-3808 is in a solid form. In some embodiments, the ethanolamine salt of HBI-3808 is in a solid amorphous form. In certain embodiments, the ethanolamine salt of HBI-3808 is in a solid crystalline form. In certain embodiments, the solid crystalline ethanolamine salt of HBI-3808 contains about 0, 1, 2, 3, 4, 5, 6, 7, 8, 9 or 10, equivalents of water of crystallization. The ethanolamine salt of HBI-3808 can have a purity of at least about 95%, at least about 96%, at least about 97%, at least about 98%, at least about 99%, at least about 99.5%, at least about 99.7%, or about 98% to 100%. In certain embodiments, the ethanolamine salt of HBI-3808 can have a purity of about 90% to about 100%. In certain embodiments, the ethanolamine salt of HBI-3808 can have a purity of about 90% to about 95%, about 90% to about 96%, about 90% to about 97%, about 90% to about 98%, about 90% to about 99%, about 90% to about 99.2%, about 90% to about 99.5%, about 90% to about 99.7 %, about 90% to about 99.8%, about 90% to about 99.9%, about 90% to about 100%, about 95% to about 96%, about 95% to about 97%, about 95% to about 98%, about 95% to about 99%, about 95% to about 99.2%, about 95% to about 99.5%, about 95% to about 99.7%, about 95% to about 99.8%, about 95% to about 99.9%, about 95% to about 100%, about 96% to about 97%, about 96% to about 98%, about 96% to about 99%, about 96% to about 99.2%, about 96% to about 99.5%, about 96% to about 99.7%, about 96% to about 99.8%, about 96 % to about 99.9%, about 96% to about 100%, about 97% to about 98%, about 97% to about 99%, about 97% to about 99.2%, about 97% to about 99.5%, about 97% to about 99.7%, about 97% to about 99.8%, about 97% to about 99.9%, about 97% to about 100%, about 98% to about 99%, about 98% to about 99.2%, about 98% to about 99.5%, about 98% to about 99.7 %, about 98% to about 99.8%, about 98% to about 99.9%, about 98% to about 100%, about 99% to about 99.2%, about 99% to about 99.5%, about 99% to about 99.7%, about 99% to about 99.8%, about 99% to about 99.9%, about 99% to about 100%, about 99.2% to about 99.5 % about 99.2% to about 99.7%, about 99.2% to about 99.8%, about 99.2% to about 99.9%, about 99.2% to about 100%, about 99.5% to about 99.7%, about 99.5% to about 99.8%, about 99.5% to about 99.9%, about 99.5% to about 100%, about 99.7% to about 99.8%, about 99.7 % to about 99.9%, about 99.7% to about 100%, about 99.8% to about 99.9%, about 99.8% to about 100%, or about 99.9% to about 100%. In certain embodiments, the ethanolamine salt of HBI-3808 can have a purity of about 90%, about 95%, about 96%, about 97%, about 98%, about 99%, about 99.2%, about 99.5%, about 99.7%, about 99.8%, about 99.9%, or about 100 %. In certain embodiments, the ethanolamine salt of HBI-3808 can have a purity of at least about 90%, about 95%, about 96%, about 97%, about 98%, about 99%, about 99.2%, about 99.5%, about 99.7%, about 99.8%, or about 99.9%. In certain embodiments, the ethanolamine salt of HBI-3808 can have a purity of at most about 95%, about 96%, about 97% about 98%, about 99 %, about 99.2%, about 99.5%, about 99.7%, about 99.8%, about 99.9%, or about 100%.

The ethanolamine salt of HBI-3808, has an XRPD diffractogram comprises one or more peaks of the XRPD diffractogram of FIG. 17A, and/or Table 17A. In certain embodiments, the XRPD diffractogram of the ethanolamine salt of HBI-3808 comprises i) 1, 2, 3, 4, 5, 6, 7, 8, 9, 10, 11, 12, 13, 14, 15, 16, 17, 18, 19 or any range there between, peaks of, or substantially matches, FIG. 17A; ii) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or any range there between, or all peaks (±0.2 °2θ) listed in Table 17A, or both.

In certain embodiments, the XRPD diffractogram of the ethanolamine salt of HBI-3808 substantially matches, FIG. 17A.

In certain embodiments, the ethanolamine salt of HBI-3808, has any one of, any combination of, or all of i) an XRPD diffractogram comprising 1, 2, 3, 4, 5, 6, 7, 8, 9, 10, 11, 12, 13, 14, 15, 16, 17, 18, 19 or any range there between, peaks of, or substantially matches, FIG. 17A, or 1,2, 3,4, 5,6,7, 8, 9, 10, 11, 12, 13, 14, 15, 16, 17, 18, 19, 20, 21, 22, 23, 24, 25, 26, 27, 28, 29, 30, or any range there between, or all peaks (±0.2 °2θ) listed in Table 17A, or both; ii) a TG-DSC thermogram substantially matching to that of FIG. 17H; iii) a DSC thermogram substantially matching to that of FIG. 17I; iv) an FT-IR spectrum substantially matching to that of FIG. 17J; v) a Raman spectrum substantially matching to that of FIG. 17K; vi) a DVS mass plot substantially matching to that of FIG. 17L; and vii) a DVS isotherm plot substantially matching to that of FIG. 17M.

Method of making Ethanolamine Salts of HBI-3808

Certain aspects are directed to a method of making an ethanolamine salt of HBI-3808. The method of making an ethanolamine salt of HBI-3808 can include any one of, any combination of, or all of steps (v), (w), (x), (y) and (z). Step (v) can include contacting HBI-3808 with a first solvent to form a slurry. Step (w) can include contacting the slurry of step (v) with an ethanolamine solution at a first temperature to form a solution. In certain embodiments, step (w) can include adding a volume of the ethanolamine solution, the volume being sufficient to form the solution of step (w). The ethanolamine solution contains ethanolamine and a second solvent. Step (x) can include adding a counter solvent to the solution formed in step (w). The step (y) can include cooling the solution (e.g., formed in step (y)) to a second temperature for a time sufficient to precipitate solids from the solution, wherein the second temperature is lower than the first temperature. Step (z) can include isolating the solids from the solution. In certain embodiments, the first solvent (e.g., of step (v)) contains water and methanol. In certain embodiments, the second solvent (e.g., of step (w) contains water and methanol. In certain embodiments, the first solvent (e.g., of step (v)) and the second solvent (e.g., of step (w) contains water and methanol. In certain embodiments, the first solvent (e.g., of step (v)) and the second solvent (e.g., of step (w)) independently contains water and methanol in a ratio of about 80:20 (% v:% v) to 99:1 (% v:% v), about 85:15 (% v:% v) to 95:5 % v:% v, or about 90:10% v:% v, or 80:20, 82:18, 84:16, 86:14, 88:12, 90:10, 92:8, 94:6, 96:4, 98:2, or 99:1 or any range, or value there between. In certain embodiments, the first solvent (e.g., of step (v)) and the second solvent (e.g., of step (w)) contains water and methanol in a ratio of about 90:10 % v:% v. In certain embodiments, the first temperature (e.g., of step (w)) is about 20° C. to 60° C., about 25° C. to 55° C., about 30° C. to 55° C., about 35° C. to 45° C., or about 40° C., or about 40° C., or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., or any range, or value there between. In certain embodiments, the first temperature (e.g., of step (w)) is about 40° C. In certain embodiments, the counter solvent contains TBME. In certain embodiments, the second temperature (e.g., of step (y)) is about 0° C. to 20° C., about 2° C. to 15° C., about 3° C. to 10° C., or about 5° C., or 0, 1, 2, 3, 4, 5, 6, 7, 8, 9, 10, 11, 12, 13, 14, 15, 16, 17, 18, 19, or 20° C., or any range, or value there between. In certain embodiments, the second temperature (e.g., of step (y)) is about 5° C. In certain aspects, the cooling (e.g., of step (y)) is carried out at a rate of about 0.01 to 3° C./min, about 0.01 to 2° C./min, about 0.01 to 1° C./min, about 0.05 to 0.5° C./min, or about 0.1° C./min. In certain embodiments, the cooling (e.g., of step (y)) is carried out at a rate of about 0.1° C./min. In certain embodiments, the time (e.g., of step (y)) sufficient to precipitate solids from the solution is about 1 to about 100 hr. In certain embodiments, in step (z), the solids can be isolated by pressure filtration, centrifuge filtration, and/or Buchner funnel filtration. In certain embodiments, isolating the solids from the solution can include drying the solids. The isolated solids can have a purity of at least about 95%, about 95% to 99.5%, about 95%, about 95% to 99.9%, about 95% to 99%. In some embodiments, the isolated solids can contain a monoethanolamine salt of HBI-3808.

Compositions containing an ethanolamine salt of HBI-3808

Certain aspects are directed to a composition containing an ethanolamine salt of HBI-3808. The ethanolamine salt of HBI-3808 can be an ethanolamine salt described herein, and/or can be prepared by a method described herein. Ethanolamine salt of HBI-3808, content in the composition can be 1,2,3,4,5, 6,7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 81, 82, 83, 84, 85, 86, 87, 88, 89, 90, 91, 92, 93, 94, 95, 96, 97, 98, 99 or 100 wt. %, or any value or range there between. The ethanolamine salts of HBI-3808 may be formulated as pharmaceutical compositions. Such pharmaceutical compositions may be administered to a subject, such as a human patient suffering from myocardial infarction or the sequelae thereof, at a dosage sufficient to treat myocardial infarction. The ethanolamine salts of HBI-3808 may be formulated as oral compositions or may be used to manufacture parenteral formulations, such as intravenous (IV), intraperitoneal, intramuscular, or inhalation formulations. For parenteral administration, the pharmaceutical composition may be formulated as a sterile injectable liquid stored in a suitable container such as a sterile syringe, a sterile bag, an ampule, in the form of an aqueous or non-aqueous liquid suspension, micelle, liposome or nanoparticle formulation. The nature and composition of the pharmaceutical carrier, diluent or excipient will, of course, depend on the intended route of administration, for example whether by intravenous or intramuscular injection.

Pharmaceutical compositions and methods

The salts of HBI-3808 described herein, e.g. the sodium and ethanolamine salts of HBI-3808, may be formulated as pharmaceutical compositions. Such pharmaceutical compositions may be administered to a subject, such as a human patient suffering from myocardial infarction or the sequelae thereof, at a dosage sufficient to treat myocardial infarction. The salts of HBI-3808 may be formulated as oral compositions or may be used to manufacture parenteral formulations, such as intravenous (IV), intraperitoneal, or intramuscular formulations. For parenteral administration, the pharmaceutical composition may be formulated as a sterile injectable liquid stored in a suitable container such as a sterile syringe, a sterile bag, or an ampule, or in the form of an aqueous or non-aqueous liquid suspension. The nature and composition of the pharmaceutical carrier, diluent or excipient will, of course, depend on the intended route of administration, for example whether by intravenous or intramuscular injection.

Treatment of myocardial infarction may include improving injection fraction, improving stroke work, increasing the area within pressure-volume loops, enhancing cardiogenic differentiation efficiency of endogenous mesenchymal stem cells (MSCs), increasing expression of MSC-specific biomarkers, facilitating transplantation and differentiation of MSCs into infarcted cardiac tissue, stimulating myocardial regeneration in infarcted cardiac tissue, stimulating stem cell differentiation into functional cardiomyocytes, replacing and remodeling the myocardium with new functional tissue, limiting infarct size, preventing or treating cardiomyocyte death, revascularizing infarcted myocardial tissue and border regions by promoting capillary replacement, enhancing elastin deposition in infarcted myocardial tissue and border regions and preventing sequelae of heart failure.

Pharmaceutical compositions of the compounds of salts of HBI-3808, e.g., the sodium and ethanolamine salts of HBI-3808, for use in methods according to the present invention may be formulated as solutions or lyophilized powders for parenteral administration. Lyophilized powders for reconstitution may contain excipients to facilitate lyophilization and rapid reconstitution such as carbohydrates (e.g. lactose, mannitol, sucrose, glucose, and trehalose) and excipients to stabilize pH such as acetate, citrate, phosphate, lysine and other amino acid buffer systems). Such powders may be reconstituted by addition of a suitable diluent, e.g. sterile normal (0.9%) saline, water for injection (WFI), or other pharmaceutically acceptable carrier prior to use. Other suitable diluents include standard 5% dextrose in water, or lactated ringers solution, half-normal (0.45%) saline.

Such formulations may be buffered if necessary to maintain the pH of the formulation during storage. Such formulation is especially suitable for parenteral administration, but may also be used for oral or inhalation administration. In the case of oral administration, it may be advantageous or necessary to add one or more sweeteners, flavor masking agents, or stabilizers. Alternatively solid oral dosage forms can be prepared from standard excipients such as tablets, orally dissolving tablets (ODT), lozenges, hard or soft gelatin or HPMC capsules, and powders for constitution or reconstitution.

Pharmaceutical compositions of the salts of HBI-3808 described herein, e.g., the sodium and ethanolamine slats of HBI-3808 for use in methods according to the present invention may be formulated as oral compositions, which may be tablets, orally dissolving tablets (ODT), lozenges, hard or soft gelatin or HPMC capsules, buccal films, and powders for constitution or reconstitution, syrups, or oral emulsions. Tablets and capsules may be enteric coated in order to ensure that the formulation delivers its dose of HBI-3808 to the small intestine. Solid oral compositions may contain one or more inactive ingredients, including diluents, stabilizers, glidants, disintegrants, enteric coatings, time release coatings, etc.

It will be appreciated that the actual preferred dosages of the salts of HBI-3808 used in the pharmaceutical compositions will vary according to the particular composition formulated, the mode of administration, the particular site of administration and the host being treated. Exemplary dosages of salts of HBI-3808 are in the range of 0.001 to 100 mg/kg, 0.001 to 50 mg/kg, 0.001 to 20 mg/kg, 0.001 to 10 mg/kg, 0.001 to 5 mg/kg, 0.01 to 100 mg/kg, 0.01 to 50 mg/kg, 0.01 to 20 mg/kg, 0.01 to 10 mg/kg, 0.01 to 5 mg/kg, 0.05 to 100 mg/kg, 0.05 to 50 mg/kg, 0.05 to 20 mg/kg, 0.05 to 10 mg/kg, 0.05 to 5 mg/kg, 0.1 to 100 mg/kg, 0.1 to 50 mg/kg, 0.1 to 20 mg/kg, 0.1 to 10 mg/kg, 0.1 to 5 mg/kg, 1 to 100 mg/kg, 1 to 50 mg/kg, 1 to 20 mg/kg. The salts of HBI-3808 described herein be administered as 1 to 4 (divided) doses per day for a period sufficient to treat a subject who has had a myocardial infarction.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

Sodium salt of HBI-3808 having a XRPD of Pattern 1

Sodium salt of HBI-3808 having an X-ray powder diffraction (XRPD) of Pattern 1 (FIG. 1A) was prepared from HBI-3808 and sodium hydroxide. 25 mg of HBI-3808 was weighed into a 1.5 mL screw cap vial. 200 μL of methanol:water 90:10% v: % v was added to the HBI-3808 in the vial. 1.1 equivalents of sodium hydroxide were added to the vial. The base was added as a solid and was rinsed in with a 50 L of methanol:water 90:10% v:O % v. The contents of the vial were stirred for 1 hour (h) at 40° C., and additional solvent was added as required to obtain a mobile slurry. The experiment temperature was then cycled between 40° C. and 5° C. at 0.1° C./min with 1 h holds at 40° C. and 5° C. for about 24 h. After 24 hours the reaction mixture was checked and 100 μL aliquots of tert-butyl methyl ether (TBME) anti-solvent were added as required. The final solvent system has methanol:water:TBME ratio of 41:2:57% v:% v:% v. The experiment temperature was then cycled between 40° C. and 5° C. at 0.1° C./min with 1 h holds at 40° C. and 5° C. for a 24 h. Solids were then isolated by centrifuge filtration (nylon, 0.22 m) and damp solids analyzed by XRPD. Solids were dried at 40° C. at ambient pressure, or 40° C. under vacuum, for ca. 24 h and were re-analyzed by XRPD. Solids were stored at 40° C./75% RH for ca. 24 h, and were re-analyzed by XRPD. FIG. 1A shows the XRPD diffractogram of the solids after drying at 40° C. at ambient pressure. FIG. 1B shows the peak fitting of the XRPD diffractogram (FIG. 1A) using pseudo-Voigt function with HighScore 4.9.

TABLE 1
XRPD peak lists of HBI-3808 sodium salt of Pattern 1.

| | Height | FWHM Left | | |
|---|---|---|---|---|
| Pos. [°2θ] | [cts] | [°2θ] | d-spacing [Å] | Rel. Int. [%] |
| 3.4738 | 1173.25 | 0.4605 | 25.43498 | 100.00 |
| 7.2061 | 322.68 | 0.3070 | 12.26747 | 27.50 |
| 11.6066 | 214.87 | 0.2558 | 7.62445 | 18.31 |
| 12.3166 | 538.06 | 0.3582 | 7.18650 | 45.86 |
| 13.7199 | 711.21 | 0.3582 | 6.45444 | 60.62 |
| 16.3836 | 256.24 | 0.7164 | 5.41055 | 21.84 |
| 19.2619 | 82.14 | 0.8187 | 4.60807 | 7.00 |
| 27.9606 | 44.86 | 1.0234 | 3.19112 | 3.82 |

### Example 2

Sodium salt of HBI-3808 having a XRPD of Pattern 2

Sodium salt of HBI-3808 having a XRPD of Pattern 2 (FIG. 2A) was prepared from HBI-3808 and sodium hydroxide. 1.5 g of HBI-3808, was weighed and transferred into a 100 mL vessel. 6.5 mL of methanol was added to the vessel, at around 40° C., and the mixture was stirred at 100 rpm with an overhead stirrer to form a slurry. Separately, 1.1 equivalents of sodium hydroxide was dissolved in 0.75 mL of water. The sodium hydroxide solution was added to the slurry of HBI-3808, which resulted in dissolution. After the addition of sodium hydroxide dissolution was initially observed, followed by precipitation within about 30 minutes. After 1 h at 40° C., 67.5 mL of acetone, an anti-solvent, was added over 4 h (0.3 mL/min) to the vessel. After anti-solvent addition was complete, experiment was stirred for 1 h at 40° C. then cooledto 5° C. at 0.1° C./min. The experiment was held at 5° C. for 7 h to from solids. The slurry was sub-sampled and solids isolated whereby centrifuge filtration (nylon, 0.22 μm). Bulk solids were isolated by Buchner filtration (ø42.5 mm, grade 1 paper) and washed with 3 mL of methanol:water:acetone 9:1:90% v:% v:% v. Isolated solids were dried at 40° C. under vacuum with elevated humidity for 48 h. Isolated yield was 86%. The isolated solids were analyzed by XRPD. FIG. 2A shows the XRPD diffractogram of the isolated solids of Sodium salt of HBI-3808 of Pattern 2. FIG. 2B shows the peak fitting of the XRPD (FIG. 2A) using pseudo-Voigt function with HighScore 4.9. The fitted parameters are listed in Table 2A.

FIG. 2C shows XRPD spectra of Sodium salt of HBI-3808 of Pattern 2, damp isolated solid (as prepared in Example 2), dried solids (as prepared in Example 2), and HBI-3808 acid of Pattern 1.

TABLE 2A
XRPD peak lists of HBI-3808 sodium salt of Pattern 2.

| | | FWHM Left | | |
|---|---|---|---|---|
| Pos. [°2θ] | Height [cts] | [°2θ] | d-spacing [Å] | Rel. Int. [%] |
| 5.1053 | 477.31 | 0.0640 | 17.30979 | 11.60 |
| 5.5278 | 4116.23 | 0.0768 | 15.98775 | 100.00 |
| 7.1400 | 334.84 | 0.0512 | 12.38088 | 8.13 |
| 8.6036 | 884.74 | 0.0768 | 10.27783 | 21.49 |
| 10.5996 | 308.75 | 0.0768 | 8.34643 | 7.50 |
| 11.0743 | 185.27 | 0.1023 | 7.98971 | 4.50 |
| 12.8914 | 2354.26 | 0.1023 | 6.86731 | 57.19 |
| 13.7560 | 2470.36 | 0.1023 | 6.43760 | 60.02 |
| 14.1989 | 1435.16 | 0.0768 | 6.23776 | 34.87 |
| 14.4830 | 404.48 | 0.0640 | 6.11602 | 9.83 |
| 15.2501 | 1669.41 | 0.1023 | 5.81006 | 40.56 |
| 16.4612 | 691.10 | 0.0895 | 5.38523 | 16.79 |
| 18.0628 | 378.22 | 0.0768 | 4.91117 | 9.19 |
| 18.4802 | 154.59 | 0.1023 | 4.80118 | 3.76 |
| 19.1721 | 180.52 | 0.1279 | 4.62945 | 4.39 |
| 19.8927 | 523.40 | 0.0895 | 4.46335 | 12.72 |
| 20.6091 | 98.38 | 0.1023 | 4.30980 | 2.39 |
| 20.8927 | 60.39 | 0.1279 | 4.25192 | 1.47 |
| 21.3955 | 112.39 | 0.1791 | 4.15313 | 2.73 |
| 21.9426 | 51.61 | 0.1535 | 4.05080 | 1.25 |
| 23.2596 | 116.24 | 0.1535 | 3.82432 | 2.82 |
| 23.5575 | 262.48 | 0.0895 | 3.77664 | 6.38 |
| 24.2200 | 83.61 | 0.2047 | 3.67482 | 2.03 |
| 25.0404 | 57.60 | 0.1535 | 3.55624 | 1.40 |
| 26.3318 | 120.82 | 0.1023 | 3.38469 | 2.94 |
| 27.1547 | 46.64 | 0.1535 | 3.28397 | 1.13 |
| 27.4755 | 91.05 | 0.1791 | 3.24634 | 2.21 |
| 28.8688 | 96.37 | 0.2047 | 3.09276 | 2.34 |
| 30.0649 | 109.94 | 0.1023 | 2.97239 | 2.67 |
| 30.7422 | 146.26 | 0.1791 | 2.90843 | 3.55 |
| 31.2153 | 90.42 | 0.1279 | 2.86542 | 2.20 |
| 31.5923 | 172.65 | 0.1791 | 2.83208 | 4.19 |
| 32.5004 | 92.41 | 0.1791 | 2.75500 | 2.25 |
| 33.5635 | 134.61 | 0.1023 | 2.67012 | 3.27 |
| 34.3528 | 103.88 | 0.2558 | 2.61057 | 2.52 |

The isolated solids had a chemical purity of 97.2%, as determined by high performance liquid chromatography (HPLC). The concentration of HBI-3808 in the filtered mother liquor was 0.1 mg/mL, and in the wash solution of 0.1 mg/mL (of HBI-3808), as determined by HPLC. Gas chromatography (GC) analysis indicated residual solvent contents of 39 ppm methanol; and 269 ppm acetone. Charged aerosol detector (CAD) indicated a sodium content of 5.2% w/w, equal to ca. 1.1 equivalents of sodium, indicating that the sodium salt Pattern 2 is a mono-sodium salt. KF (Karl Fischer titration) analysis of the solids indicated a water content of 6.4 wt. %, equal to about 2 equivalents of water. FIG. 2D shows the HPLC chromatogram of isolated solids.

The ¹H NMR spectrum was recorded in d6-DMSO and indicated a residual acetone content of 0.07 wt. % (FIG. 2E). Polarized light microscopy (PLM) analysis on the dried solids indicated the material was birefringent with plate-like morphology. Some aggregation was observed (FIG. 2F).

Thermogravimetry (TG)—differential scanning calorimetry (DSC) analysis indicated two weight losses (FIG. 2G). The first weight loss of 3.0 wt. % was observed between 20-95° C., and theoretically equal to about 1 equivalent of water. The second weight loss of 3.3 wt. %ccurred between 95-149° C., and is also theoretically equal to about 1 equivalent of water. In the DSC thermogram, one endothermic event was observed with an onset temperature of 93° C. and a peak temperature of 113° C. DSC analysis was also recorded which indicated two endothermic events between 20-300° C. (FIG. 2H). The first event had an onset temperature of 22° C., and a peak temperature of 68° C. The second endothermic event had an onset temperature of 116° C., and a peak temperature of 144° C.

Quantitative ¹H NMR spectroscopic analysis was carried out in d6-DMSO and indicated a purity of 99% w/w. FT-IR (FIG. 2I) and Raman (FIG. 2J) spectra were recorded for reference.

Dynamic vapor sorption (DVS) analysis was carried out and showed an irreversible mass loss of about 2.4 wt. % during the first sorption cycle between 60-80% RH. During the second DVS cycle the mass uptake was observed to be reversible, with a water uptake of 0.8 wt. % at 80 % RH/25° C. FIG. 2K shows DVS mass plot of isolated solids. FIG. 2L shows DVS isotherm plot of the isolated solids. Post-DVS, XRPD analysis on the recovered material showed it was crystalline with a diffractogram consistent with sodium salt of Pattern 5 (see Example 5) (FIG. 2M).

TG-DSC analysis was carried out on the material recovered from the DVS. A weight loss of 0.5 wt. % was observed between 20-111° C. A second weight loss of 3.3 wt. % was observed between 111-172° C., theoretically equal to about 1 equivalent of water. One endothermic event was observed with an onset temperature of 107° C., and a peak temperature of 146° C. This data suggests that sodium salt Pattern 5 may be a mono-hydrate sodium salt. FIG. 2N shows TG-DSC thermogram of the post-DVS material.

Hot stage microscopy was carried out between 25-300° C. The crystals were observed to move on the microscope slide, likely as a result of dehydration steps, however no visual change in the crystals was observed (FIG. 20).

Varying temperature (VT)-XRPD was carried out between 25-250° C. Diffractograms are consistent with sodium salt Pattern 2 were observed from 25° C. to 90° C. At 115° C., a change in XRPD Pattern was observed, and appears to be a mixture of sodium salt Pattern 2 and sodium salt Pattern 6 (see Example 6). At temperatures of 150° C. to 230° C., Pattern 6 is observed, consistent with loss of water observed in the TG-DSC and indicating that Pattern 6 is an anhydrous form. At 250° C. the material is observed to be largely amorphous, and does not recrystallize after cooling to 25° C. FIG. 2P shows VT-XRDP spectra of the isolated solids. Temperature profile for the VT-XRPD analysis is presented in Table 2B.

TABLE. 2B
Temperature Profile for the VT-XRPD
of HBI-3808 sodium salt of Pattern 2.

| Temperature / | Heating Rate / |
|---|---|
| ° C. | ° C./min |
| 25 | n/a |
| 80 | 10 |
| 90 | 2 |
| 115 | 2 |
| 150 | 2 |
| 170 | 2 |
| 210 | 2 |
| 230 | 2 |
| 250 | 2 |
| 25 | n/a |

Varying humidity (VH)-XRPD indicated that no change in XRPD Pattern occurred during desorption from 40-0% relative humidity (RH), even after 16 h at 0 RH Between 40-0 RH sodium salt Pattern 2 was observed. During the sorption phase, sodium salt Pattern 2 was observed between 40-60% RH. At 70% RH, a composite XRPD diffractogram of sodium salt Pattern 2 and 5 was observed. A mixture of sodium salt Pattern 2 and 5 was also observed at 80% RH. At 90% RH, only sodium salt Pattern 5 was observed by XRPD. During the second desorption, from 90% RH to 40% RH, sodium salt Pattern 5 was maintained. Humidity profile for the VH-XRPD analysis is presented in Table 2C.

TABLE 2C
Humidity profile for the VH-XRPD analysis.

| Relative Humidity / | | Temperature / | |
|---|---|---|---|
| % RH | Time / min | ° C. | Step |
| 40 | 5 | 30 | Desorption 1 |
| 30 | 5, 60 | | |
| 20 | 5, 60 | | |
| 10 | 5, 60 | | |
| 0 | 5, 60, 120, 960 | | |
| 40 | 5 | | Sorption 1 |
| 50 | 5, 60 | | |
| 60 | 5, 60 | | |
| 70 | 5, 60, 120, | | |
| | 180, 240 | | |
| 80 | 5, 60 | | |
| 90 | 5, 60 | | |
| 80 | 5, 60 | | Desorption 2 |
| 70 | 5, 60, 660 | | |
| 60 | 5, 60 | | |
| 50 | 5, 60 | | |
| 40 | 5 | | |

FIG. 2Q shows VH-XRPD spectra of the isolated solids during desorption from 40% to 0% (Table 2C, desorption 1). FIG. 2R shows VH-XRPD spectra of the isolated solids during sorption from 40 00 to 90 00 (Table 2C, sorption 1). FIG. 2S shows VH-XRPD spectra of the isolated solids during desorption from 80 00 to 40 00 (Table 2C, desorption 2).

Disproportion of the HBI- 3808 sodium salt of Pattern 2 was studied. The recovered solids had a XRPD diffractogram consistent with HBI-3808 sodium salt of Pattern 5 (FIG. 2T).

The pH of the filtered mother liquor was 9.77. The ¹H NMR spectrum of the isolated Pattern 5 material from the salt disproportionation study did not display a carboxylic acid signal, indicating the material likely remained as a sodium salt of HBI-3808.

Hydration of the HBI- 3808 sodium salt of Pattern 2 was studied. Hydration studies showed all solids recovered at the end of the experiment in solvent systems of methanol/water a_{w}0.1, 0.5 and 0.9 had XRPD Patterns consistent with sodium salt Pattern 2 (FIG. 2U).

pH-dependent solubility of the HBI- 3808 sodium salt of Pattern 2 was studied. In the range pH 1.2-4.0, the concentration was <0.1 mg/mL. At pH 6.8, the concentration was 0.4 mg/mL. At pH 8.0, the concentration was 15.3 mg/mL. In FaSSGF, a clear solution was observed despite additional solids added. The final pH was 9.35, and the concentration was >221.2 mg/mL. XRPD indicated that amorphous solids were recovered from experiments at pH 1.2, and pH 8.0 (FIG. 2V). Solids with a XRPD diffractogram consistent with HBI-3808 free acid Pattern 1 were recovered from experiments at pH 3.0, pH 4.0, and pH 6.8 indicating disproportionation is occurring at these pH levels. Starting concentration of HBI- 3808 sodium salt of Pattern 2, for the solubility studies were 200 mg/mL. Table 2D shows the results of pH solubility study of the HBI-3808 sodium salt of Pattern 2.

TABLE 2D
Summary of Results from pH Solubility Study with HBI-3808 sodium salt of Pattern 2.

| | | | | | | | | HPLC | |
|---|---|---|---|---|---|---|---|---|---|
| Target | Measured | Adjusted | Measured | Adjusted | Measured | Adjusted | Measured | Conc./ | |
| pH | pH | pH | pH | pH | pH | pH | pH | mg/mL * | XRPD |
| 1.2 | 9.01 | 1.29 | 1.55 | 1.21 | 1.17 | n/a | 1.20 | <0.005 | amorphous |
| 3.0 | 4.23 | 3.09 | 3.14 | 3.09 | 3.06 | n/a | 3.09 | 0.006 | Free acid, 1 |
| 4.0 | 4.88 | 4.06 | 3.60 | 4.09 | 4.04 | n/a | 4.04 | <0.005 | Free acid, 1 |
| 6.8 | 8.34 | 6.82 | 7.05 | 6.83 | 6.73 | n/a | 6.80 | 0.4 | Free acid 1, |
| | | | | | | | | | PC |
| 8.0 | 9.38 | 8.02 | 8.42 | 8.00 | 8.45 | 8.03 | 8.27 | 15.3 | amorphous |
| FaSSGF | n/a | n/a | n/a | n/a | n/a | n/a | 9.35 | >221.2 | n/a |

* Based on free acid

Stability of HBI-3808 Sodium Salt Pattern 2 was studied over 13 weeks. The input salt (of Pattern 2) purity was 97.2% area by HPLC. After 1 week at both 25° C./60% RH and 40° C./75% RH, conversion from sodium salt Pattern2 to sodium salt Pattern 5 was observed. Sodium salt Pattern 5 was then observed from these stability conditions for the duration of the 13-week study. After 13 weeks at 60° C., sodium salt Pattern 2 was maintained. There was no significant decrease in chemical purity across the stability conditions assessed over the 13 weeks. At 25° C./60% RH, the purity was 97.1% area at 13 weeks. After 13 weeks at 40° C./60% RH, the chemical purity was 97.0% area. Solids recovered from 13 weeks at 60° C. had a chemical purity of 97.3% area. FIG. 2W shows XRPD diffractogram of isolated solids from a 1-week stability study with HBI-3808 sodium salt of Pattern 2. FIG. 2X shows HPLC chromatogram of solids obtained from HBI-3808 sodium salt of Pattern 2 after 1 week at 25° C./60% RH. FIG. 2Y shows HPLC chromatogram of solids obtained from HBI-3808 sodium salt of Pattern 2 after 1 week at 40° C./75% RH. FIG. 2Z shows HPLC chromatogram of solids obtained from HBI-3808 sodium salt of Pattern 2 after 1 week at 60° C. FIG. 2AA shows XRPD diffractogram of isolated solids from a 2-week stability study with HBI-3808 sodium salt of Pattern 2. FIG. 2BB shows HPLC chromatogram of solids obtained from HBI-3808 sodium salt of Pattern 2 after 2 weeks at 25° C./60% RH. FIG. 2CC shows HPLC chromatogram of solids obtained from HBI-3808 sodium salt of Pattern 2 after 2 weeks at 40° C./75% RH. FIG. 2DD shows HPLC chromatogram of solids obtained from HBI-3808 sodium salt of Pattern 2 after 2 weeks at 60° C. FIG. 2EE shows XRPD diffractogram of isolated solids from a 4-week stability study with HBI-3808 sodium salt of Pattern 2. FIG. 2FF shows HPLC chromatogram of solids obtained from HBI-3808 sodium salt of Pattern 2 after 4 weeks at 25° C./60% RH. FIG. 2GG shows HPLC chromatogram of solids obtained from HBI-3808 sodium salt of Pattern 2 after 4 weeks at 40° C./75% RH. FIG. 2HH shows HPLC chromatogram of solids obtained from HBI-3808 sodium salt of Pattern 2 after 4 weeks at 60° C. FIG. 2H shows XRPD diffractogram of isolated solids from an 8-week stability study with HBI-3808 sodium salt of Pattern 2. FIG. 2JJ shows HPLC chromatogram of solids obtained from HBI-3808 sodium salt of Pattern 2 after 8 weeks at 25° C./60% RH. FIG. 2KK shows HPLC chromatogram of solids obtained from HBI-3808 sodium salt of Pattern 2 after 8 weeks at 40° C./75% RH. FIG. 2LL shows HPLC chromatogram of solids obtained from HBI-3808 sodium salt of Pattern 2 after 8 weeks at 60° C. FIG. 2MM shows XRPD diffractogram of isolated solids from a 13-week stability study with HBI-3808 sodium salt of Pattern 2. FIG. 2NN shows HPLC chromatogram of solids obtained from HBI-3808 sodium salt of Pattern 2 after 13 weeks at 25° C./60% RH.

FIG. 2OO shows HPLC chromatogram of solids obtained from HBI-3808 sodium salt of Pattern 2 after 13 weeks at 40° C./75% RH. FIG. 2PP shows HPLC chromatogram of solids obtained from HBI-3808 sodium salt of Pattern 2 after 13 weeks at 60° C. Table 2E presents the results of a 13-week stability study of HBI-3808 sodium salt of Pattern 2. Table 2F presents impurity tracking results from a 13-week stability study of HBI-3808 sodium salt of Pattern 2.

TABLE 2E
Results of a 13-week stability study of HBI-3808 sodium salt of Pattern 2.

| | 1 Week | | 2 Weeks | | 4 Weeks | | 8 Weeks | | 13 Weeks | |
|---|---|---|---|---|---|---|---|---|---|---|
| Stability | | HPLC/ | | HPLC/ | | HPLC/ | | HPLC/ | | HPLC/ |
| Condition | XRPD | % area | XRPD | % area | XRPD | % area | XRPD | % area | XRPD | % area |
| 25 | 5 * | 97.3 | 5 * | 97.2 | 5 * | 97.3 | 5 * | 96.7 | 5 * | 97.1 |
| ° C./60% RH | | | | | | | | | | |
| (open vial) | | | | | | | | | | |
| 40 | 5 | 97.1 | 5 | 97.0 | 5 | 97.3 | 5 | 96.3 | 5 | 97.0 |
| ° C./75% RH | | | | | | | | | | |
| (open vial) | | | | | | | | | | |
| 60° C. | 2 | 97.2 | 2 | 97.3 | 2 | 97.3 | 2 | 96.4 | 2 | 97.3 |
| (closed vial) | | | | | | | | | | |

c.f.: input purity 97.2% area HPLC results based on free acid
* Traces of sodium salt Pattern 2

TABLE F
Impurity tracking results from a 13-week stability study of HBI-3808 sodium salt of Pattern 2.

| | | RRT | | | | | | Total |
|---|---|---|---|---|---|---|---|---|
| | | 0.801 | 0.970 | 1.000 # | 1.025 * | 1.767 | 1.935 | Impurities |
| 25° C./ | T = 0 | n/a | n/a | 97.82 | 2.18 | n/a | n/a | 2.18 |
| | 1 week | 0.10 | 0.10 | 97.31 | 2.48 | n/a | n/a | 2.69 |
| | 2 weeks | n/a | 0.12 | 97.29 | 2.39 | n/a | 0.20 | 2.71 |
| | 4 weeks | n/a | 0.11 | 97.47 | 2.41 | n/a | n/a | 2.53 |
| | 8 weeks | n/a | 0.10 | 96.99 | 2.51 | 0.29 | n/a | 3.01 |
| | 13 | 0.12 | n/a | 97.33 | 2.37 | n/a | 0.19 | 2.67 |
| | weeks | | | | | | | |
| 40° C./ | T = 0 | n/a | n/a | 97.82 | 2.18 | n/a | n/a | 2.18 |
| 75% RH | 1 week | 0.10 | 0.14 | 97.28 | 2.48 | n/a | n/a | 2.72 |
| | 2 weeks | n/a | 0.12 | 97.26 | 2.40 | n/a | 0.22 | 2.74 |
| | 4 weeks | n/a | 0.11 | 27.48 | 2.41 | n/a | n/a | 2.52 |
| | 8 weeks | n/a | 0.21 | 96.88 | 2.51 | 0.40 | n/a | 3.12 |
| | 13 | n/a | n/a | 97.41 | 2.40 | n/a | 0.20 | 2.59 |
| | weeks | | | | | | | |
| 60° C. | T = 0 | n/a | n/a | 97.82 | 2.18 | n/a | n/a | 2.18 |
| (% area) | 1 week | 0.12 | n/a | 97.46 | 2.43 | n/a | n/a | 2.54 |
| | 2 weeks | n/a | 0.12 | 97.27 | 2.41 | n/a | 0.20 | 2.73 |
| | 4 weeks | n/a | 0.11 | 97.49 | 2.40 | n/a | n/a | 2.51 |
| | 8 weeks | n/a | 0.12 | 97.04 | 2.49 | 0.35 | n/a | 2.96 |
| | 13 | n/a | n/a | 97.41 | 2.41 | n/a | 0.18 | 2.59 |
| | weeks | | | | | | | |

# HBI-3808;
* Isomer of HBI-3808

### Example 3

Sodium salt of HBI-3808 having a XRPD of Pattern 3

Sodium salt of HBI-3808 having a XRPD of Pattern 3 (FIG. 3A) was prepared from HBI-3808 and sodium hydroxide. 25 mg of HBI-3808, was weighed into 1.5 mL screw cap vial. 200 μL of 2-propanol was added to the HBI-3808. 1.1 equivalents of sodium hydroxide were added, at 40° C. The base was added as a solid and was rinsed in with a further 50 μL of 2-propanol. Experiments were stirred for 1 h at 40° C., and then additional solvent was added as required to obtain a mobile slurry. Experiments were temperature cycled between 40° C. and 5° C. at 0.1° C./min with 1 h holds at 40° C. and 5° C. for about 48 h. Solids were isolated by centrifuge filtration (nylon, 0.22 μm) and damp solids analyzed by XRPD. Solids were dried at 40° C. at ambient pressure, or 40° C. under vacuum, for ca. 24 h and were re-analyzed by XRPD. Solids were stored at 40° C./75% RH for ca. 24 h, and were re-analyzed by XRPD.

FIG. 3A shows the XRPD diffractogram of the solids after drying at 40° C. at ambient pressure. FIG. 3B shows the peak fitting of the XRPD diffractogram (FIG. 3A) using pseudo-Voigt function with HighScore 4.9. The fitted parameters are listed in Table 3.

TABLE 3
XRPD peak lists of HBI- 3808 Sodium Salt of Pattern 3.

| | | FWHM Left | | |
|---|---|---|---|---|
| Pos. [°2θ] | Height [cts] | [°2θ] | d-spacing [Å] | Rel. Int. [%] |
| 5.0986 | 1452.03 | 0.0895 | 17.33249 | 100.00 |
| 5.4280 | 480.85 | 0.1279 | 16.28155 | 33.12 |
| 6.4440 | 32.78 | 0.6140 | 13.71645 | 2.26 |
| 7.9003 | 288.48 | 0.1279 | 11.19109 | 19.87 |
| 10.9365 | 137.91 | 0.2047 | 8.09009 | 9.50 |
| 11.8958 | 348.41 | 0.1535 | 7.43976 | 23.99 |
| 13.2123 | 898.22 | 0.1791 | 6.70123 | 61.86 |
| 14.5029 | 458.82 | 0.2558 | 6.10768 | 31.60 |
| 15.2426 | 449.25 | 0.1535 | 5.81292 | 30.94 |
| 18.4356 | 88.13 | 0.3582 | 4.81269 | 6.07 |
| 20.4113 | 27.70 | 0.3070 | 4.35111 | 1.91 |

### Example 4

Sodium salt of HBI-3808 having a XRPD of Pattern 4

Sodium salt of HBI-3808 having a XRPD of Pattern 4 (FIG. 4A) was prepared from sodium salt Pattern 2 of HBI-3808. Approximately 15 mg of HBI-3808 sodium salt of Pattern 2 was weighed into 2×2 mL pushcap vials. Solids were dissolved in 80 μL of water, at 25° C., to obtain a concentration of 188 mg/mL (with respect to sodium salt Pattern 2). Solutions were cooled to 5° C. over 20 minutes, then tetrahydrofuran (THF) was added in 100 μL aliquots to the vials until precipitation was observed. The final solvent system was water:THF 4:96 % v: % v. Experiments were temperature cycled between 40-5° C. at 0.1° C./min, with 1 h holds at 40° C. and 5° C. for about 15 h. Solids were isolated from slurries by centrifuge filtration (nylon, 0.22 μm) and analyzed byXRPD. Solids were dried at 40° C. under vacuum for ca. 7 h, then re-analyzed by XRPD.

FIG. 4A shows the XRPD diffractogram of the solids after drying. FIG. 4B shows the peak fitting of the XRPD diffractogram (FIG. 4A) using pseudo-Voigt function with HighScore 4.9. The fitted parameters are listed in Table 4.

TABLE 4
XRPD peak lists of HBI- 3808 Sodium Salt of Pattern 4.

| | | FWHM Left | | |
|---|---|---|---|---|
| Pos. [°2θ] | Height [cts] | [°2θ] | d-spacing [Å] | Rel. Int. [%] |
| 5.7267 | 1336.27 | 0.0768 | 15.43300 | 93.84 |
| 5.9898 | 678.89 | 0.0768 | 14.75558 | 47.67 |
| 7.5025 | 65.36 | 0.2047 | 11.78355 | 4.59 |
| 8.0945 | 136.98 | 0.1023 | 10.92308 | 9.62 |
| 8.5571 | 123.25 | 0.2047 | 10.33354 | 8.65 |
| 9.2342 | 109.78 | 0.3070 | 9.57723 | 7.71 |
| 10.3025 | 101.21 | 0.4093 | 8.58644 | 7.11 |
| 11.4509 | 185.89 | 0.0768 | 7.72779 | 13.05 |
| 12.6975 | 498.37 | 0.0768 | 6.97177 | 35.00 |
| 13.1579 | 799.02 | 0.1151 | 6.72882 | 56.11 |
| 13.5877 | 1065.22 | 0.1023 | 6.51696 | 74.80 |
| 13.7628 | 1424.02 | 0.1151 | 6.43442 | 100.00 |
| 14.7165 | 600.47 | 0.1023 | 6.01951 | 42.17 |
| 15.0210 | 1107.92 | 0.1151 | 5.89815 | 77.80 |
| 15.6378 | 288.47 | 0.3070 | 5.66687 | 20.26 |
| 16.2493 | 188.22 | 0.3070 | 5.45498 | 13.22 |
| 16.9740 | 210.89 | 0.1535 | 5.22368 | 14.81 |
| 18.0414 | 134.98 | 0.2047 | 4.91696 | 9.48 |
| 19.1605 | 156.31 | 0.1535 | 4.63222 | 10.98 |
| 19.7067 | 99.63 | 0.3070 | 4.50506 | 7.00 |
| 21.0462 | 118.72 | 0.2047 | 4.22126 | 8.34 |
| 22.9481 | 83.66 | 0.3070 | 3.87553 | 5.87 |
| 25.0215 | 97.92 | 0.2047 | 3.55889 | 6.88 |
| 26.4710 | 81.00 | 0.2047 | 3.36721 | 5.69 |
| 28.5144 | 37.60 | 0.6140 | 3.13039 | 2.64 |
| 30.6921 | 44.70 | 0.3070 | 2.91306 | 3.14 |
| 32.9447 | 36.80 | 0.3070 | 2.71885 | 2.58 |

### Example 5

Sodium salt of HBI-3808 having a XRPD of Pattern 5

Sodium salt of HB-3808 having a XRPD of Pattern 5 (FIG. 5A) was prepared from sodium salt Pattern 2 of HBI-3808. About 320 mg of sodium salt Pattern 2 was weighed out into a 20 mL screw cap vial. The vial was uncapped and the solids were exposed to 25° C./60% RH within a stability chamber for 96 h. Approximately every 24 h, the solids were agitated, and the solids were monitored by XRPD for complete conversion to Pattern 5. The recovered solids were analyzed using selected techniques from the secondary salt screen characterizations. FIG. 5A shows the XRPD diffractogram of HBI-3808 sodium salt of Pattern 5. FIG. 5B shows the peak fitting of the XRPD diffractogram (FIG. 5A) using pseudo-Voigt function with HighScore 4.9.

TABLE 5A
XRPD peak lists of HBI- 3808 Sodium Salt of Pattern 5.

| | Height | | | |
|---|---|---|---|---|
| Pos. [°2θ] | [cts] | FWHM Left [°2θ] | d-spacing [Å] | Rel. Int. [%] |
| 3.3964 | 158.28 | 0.8187 | 26.01470 | 2.25 |
| 5.5165 | 377.90 | 0.0768 | 16.02058 | 5.37 |
| 6.0842 | 2084.32 | 0.0895 | 14.52695 | 29.60 |
| 6.5288 | 166.84 | 0.0895 | 13.53854 | 2.37 |
| 8.5404 | 1392.00 | 0.0768 | 10.35375 | 19.77 |
| 12.2073 | 157.25 | 0.1535 | 7.25057 | 2.23 |
| 12.8818 | 200.56 | 0.1023 | 6.87244 | 2.85 |
| 14.0153 | 7040.91 | 0.0895 | 6.31905 | 100.00 |
| 14.2086 | 2600.21 | 0.0640 | 6.23350 | 36.93 |
| 14.3665 | 3555.97 | 0.1023 | 6.16535 | 50.50 |
| 14.9229 | 299.04 | 0.0768 | 5.93673 | 4.25 |
| 15.2551 | 973.95 | 0.1151 | 5.80817 | 13.83 |
| 15.7043 | 3311.42 | 0.1023 | 5.64303 | 47.03 |
| 17.5444 | 143.07 | 0.1791 | 5.05511 | 2.03 |
| 18.1938 | 681.22 | 0.0895 | 4.87611 | 9.68 |
| 18.4094 | 645.09 | 0.1023 | 4.81949 | 9.16 |
| 18.7092 | 305.52 | 0.1023 | 4.74294 | 4.34 |
| 19.1297 | 2058.98 | 0.1151 | 4.63963 | 29.24 |
| 19.6625 | 254.54 | 0.1535 | 4.51509 | 3.62 |
| 21.2345 | 36.41 | 0.4093 | 4.18426 | 0.52 |
| 22.3391 | 300.86 | 0.1151 | 3.97978 | 4.27 |
| 23.4818 | 441.29 | 0.1407 | 3.78865 | 6.27 |
| 23.8180 | 195.33 | 0.0895 | 3.73591 | 2.77 |
| 25.7263 | 138.72 | 0.1279 | 3.46297 | 1.97 |
| 26.2450 | 363.88 | 0.1151 | 3.39569 | 5.17 |
| 26.8457 | 119.49 | 0.2047 | 3.32106 | 1.70 |
| 28.2070 | 63.13 | 0.1535 | 3.16380 | 0.90 |
| 28.6360 | 184.80 | 0.1023 | 3.11738 | 2.62 |
| 29.0314 | 185.57 | 0.2047 | 3.07581 | 2.64 |
| 30.4358 | 216.09 | 0.1535 | 2.93701 | 3.07 |
| 30.9534 | 612.83 | 0.1791 | 2.88906 | 8.70 |
| 31.7204 | 245.64 | 0.1535 | 2.82094 | 3.49 |
| 32.2844 | 147.98 | 0.1535 | 2.77294 | 2.10 |
| 32.6026 | 217.43 | 0.1279 | 2.74659 | 3.09 |
| 33.1623 | 108.67 | 0.1535 | 2.70151 | 1.54 |
| 33.5630 | 383.18 | 0.2047 | 2.67016 | 5.44 |
| 34.0818 | 112.66 | 0.1279 | 2.63070 | 1.60 |

FIG. 5C shows XRPD diffractogram obtained after exposing the HBI-3808 sodium salt of Pattern 2 to 25° C./60% RH within a stability chamber for 96 h. XRPD diffractogram of HBI-3808 sodium salt of Pattern 2 and 5 are shown for reference. As can be seen from FIG. 5C the XRPD of the solids after about 96 h at 25° C./60% RH was consistent with HBI-3808 sodium salt of Pattern 5. HBI-3808 sodium salt of Pattern 5 was also observed in the post-DVS XRPD analysis of HBI-3808 sodium salt of Pattern 2 (see Example 2, FIG. 2M). PLM imaging indicated that the solids were birefringent with a lathe-like morphology (FIG. 5D). The chemical purity of the solids was 96.8% area by HPLC, FIG. 5E. FIG. 5F shows ¹H NMR Spectrum of HBI-3808 sodium salt of Pattern 5, in d₆-DMSO. ¹H NMVR analysis of HBI-3808 sodium salt of Pattern 5 was consistent with a sodium salt of HBI-3808. No residual solvents were detected by NMR. CAD indicates a sodium content of 4.5%w/w, indicating that HBI-3808 sodium salt Pattern 5 is a monosodium salt.

TG-DSC analysis of HBI-3808 sodium salt of Pattern 5 (FIG. 5G) showed two weight losses. The first weight loss of 0.5 wt. % was observed between 20-87° C. The second weight loss of 3.2 wt. % was observed between 87-168° C. and is consistent with the loss of 1 equivalent of water. In the DSC thermogram HBI-3808 sodium salt of Pattern 5 (FIG. 5H), one endothermic event was observed with an onset temperature of 104° C. and a peak temperature of 147° C. DSC analysis was carried out and showed two endothermic events. The first endothermic event had an onset temperature of 114° C., and a peak temperature of 166° C. The second endothermic event had an onset temperature of 183° C., and a peak temperature of 197° C.

DVS analysis showed a mass uptake of 0.8 wt. % at 80% RH/25° C. The mass uptake/loss was observed to be reversible. FIG. 5I shows DVS mass plot for HBI-3808 sodium salt of Pattern 5. FIG. 5J shows DVS isotherm plots for HBI-3808 sodium salt of Pattern 5. At the end of the DVS analysis, the recovered were observed to have a diffractogram consistent with HBI-3808 sodium salt of Pattern 5 (FIG. 5K).

VH-XRPD analysis was carried out between 40-0% RH (FIG. 5L). In the VH-XRPD of HBI-3808 sodium salt of Pattern 5, no change in diffractogram was observed between 40-0% RH, indicating that once HBI-3808 sodium salt of Pattern 5 is formed it is stable to relative humidity between 90-0% RH. Table 5B shows temperature program for the VH-XRPD analysis.

TABLE 5B
Temperature program for the VH-XRPD analysis

| Relative Humidity / | | Temperature / | |
|---|---|---|---|
| % RH | Time / min | ° C. | Step |
| 40 | 5 | 30 | Desorption 1 |
| 30 | 5, 60 | | |
| 20 | 5, 60 | | |
| 10 | 5, 60 | | |
| 0 | 5, 60, 900 | | |
| 40 | 5 | | Sorption 1 |

VT-XRPD of HBI-3808 sodium salt of Pattern 5 was carried out between 25-250° C. (FIG. 5M). VT-XRPD diffractograms consistent with of HBI-3808 sodium salt of Pattern 5 was observed between 25-115° C. At 150° C., the diffractogram changed and was labelled as Pattern 7, indicating formation of HBI-3808 sodium salt of Pattern 7 (see Example 7). Diffractogram of Pattern 7, was observed between 150-250° C. Upon cooling to 25° C., Diffractogram of Pattern 5 was observed again, indicating reformation of HBI-3808 sodium salt of Pattern 5. Pattern 7 has some similarities with Pattern 6 (See Example 6). Both Patterns 6 and 7 may represent anhydrous forms of the HBI-3808 sodium salt. Starting concentration of the of HBI-3808 sodium salt of Pattern 5. Table 5C shows temperature program for the VT-XRPD analysis.

TABLE 5C
Temperature program for the VT-XRPD analysis

| Temperature / ° C. | Heating Rate / ° C./min |
|---|---|
| 25 | 10 |
| 90 | 2 |
| 115 | 2 |
| 150 | 2 |
| 170 | 2 |
| 210 | 2 |
| 230 | 2 |
| 250 | 2 |
| 25 | n/a |

Hydration of the HBI-3808 sodium salt of Pattern 5 was studied. Hydration studies showed all solids recovered at the end of the experiment in solvent systems of methanol/water a_{w}0.1, 0.5 and 0.9 had XRPD Patterns consistent with sodium salt Pattern 2. FIG. 5N shows XRPD diffractogram of isolated solids from hydration study HBI-3808 sodium salt of Pattern 5.

pH dependent solubility of the HBI- 3808 sodium salt of Pattern 5 was studied. In the range pH 1.2 to pH 4.0 the concentration was <0.001 mg/mL. At pH 6.8, the concentration was 0.25 mg/mL. At pH 8.0, the concentration was 12.84 mg/mL. In FaSSGF, a clear solution was observed, the final pH was 9.34, and the concentration was >150.33 mg/mL. XRPD (FIG. 50) indicated that solids with a diffractogram of HBI-3808 free acid Pattern 1 were isolated from pH 1.2, 3.0, 4.0, 6.8 and 8.0. Solids isolated from pH 1.2, 6.8 and 8.0 were poorly crystalline. Starting concentration of HBI- 3808 sodium salt of Pattern 5, for the solubility studies were 200 mg/mL. Table 5D shows the results of pH solubility study of the HBI-3808 sodium salt of Pattern 5.

TABLE 5D

| | pH solubility study results of the HBI-3808 sodium salt of Pattern 5. | | | | | | | | |
|---|---|---|---|---|---|---|---|---|---|
| | 1 h | | 3 h | | 22 h | | 24 h | HPLC | |
| Target | Measured | Adjusted | Measured | Adjusted | Measured | Adjusted | Measured | Conc./ | |
| pH | pH | pH | pH | pH | pH | pH | pH | mg/mL * | XRPD |
| 1.2 | 8.36 | 1.23 | 1.32 | 1.26 | 1.19 | n/a | 1.23 | <0.001 | 1** |
| 3.0 | 4.07 | 3.17 | 3.36 | 3.09 | 3.02 | n/a | 3.08 | <0.001 | 1** |
| 4.0 | 5.40 | 4.07 | 4.41 | 4.07 | 4.13 | 4.06 | 4.13 | <0.001 | 1** |
| 6.8 | 8.75 | 6.85 | 6.76 | 6.81 | 6.76 | n/a | 6.81 | 0.25 | 1** |
| 8.0 | 9.97 | 8.08 | 8.45 | 8.02 | 8.15 | 7.97 | 8.16 | 12.84 | 1** |
| FaSS | n/a | n/a | n/a | n/a | n/a | n/a | 9.34 | >150.33 | n/a |
| GF | | | | | | | | | |

* Based on free acid
**Free acid

Stability of HBI-3808 sodium salt of Pattern 5 over 4 weeks was studied. The input material for this set of studies had a chemical purity of 96.8% area, by HPLC. Sodium salt Pattern 5 was retained after 4 weeks at 25° C./60% RH; 40° C./75% RH; and 60° C. After 4 weeks, there was no significant decrease in chemical purity across all stability conditions (25° C./60% RH; 40° C./75 % RH; and 60° C.). Table 5E summarizes the impurities observed at each time point by HPLC which integrated >0.05% area. Table 5F present impurity tracking from a 4-week stability study of HBI-3808 sodium salt of Pattern 5. FIG. 5P shows XRPD diffractogram of isolated solids from a 1-week stability study with HBI salt sodium salt of Pattern 5. FIG. 5Q shows HPLC chromatogram of solids after 1 week at 25° C./60% RH (HBI salt sodium salt of Pattern 5 as input). FIG. 5R shows HPLC chromatogram of solids after 1 week at 40° C./75% RH (HBI salt sodium salt of Pattern 5 as input). FIG. 5S shows HPLC chromatogram of solids after 1 week at 60° C. (HBI salt sodium salt of Pattern 5 as input). FIG. 5T shows XRPD diffractogram of isolated solids from a 2-week stability study with HBI salt sodium salt of Pattern 5. FIG. 5U shows HPLC chromatogram of solids after 2 weeks at 25° C./60% RH (HBI salt sodium salt of Pattern 5 as input). FIG. 5V shows HPLC chromatogram of solids after 2 weeks at 40° C./75 % RH (HBI salt sodium salt of Pattern 5 as input). FIG. 5W shows HPLC chromatogram of solids after 2 weeks at 60° C. (HBI salt sodium salt of Pattern 5 as input). FIG. 5X shows XRPD diffractogram of isolated solids from a 4-week stability study with HBI salt sodium salt of Pattern 5. FIG. 5Y shows HPLC chromatogram of solids after 4 weeks at 25° C./60% RH (HBI salt sodium salt of Pattern 5 as input). FIG. 5Z shows HPLC chromatogram of solids after 4 weeks at 40° C./75% RH (HBI salt sodium salt of Pattern 5 as input). FIG. 5AA shows HPLC

TABLE 5E
Impurities observed at each time point by HPLC which
integrated ≥0.05% area.

| | 1 Week | | 2 Weeks | | 4 Weeks | |
|---|---|---|---|---|---|---|
| Stability | | HPLC/ | | HPLC/ | | HPLC/ |
| Condition | XRPD | % area | XRPD | % area | XRPD | % area |
| 25° C./60% RH | 5 | 96.4 | 5 | 96.7 | 5 | 96.3 |
| (open vial) | | | | | | |
| 40° C./75% RH | 5 | 96.7 | 5 | 97.5 | 5 | 96.6 |
| (open vial) | | | | | | |
| 60° C. | 5 | 96.1 | 5 | 97.3 | 5 | 96.1 |
| (closed vial) | | | | | | |

c.f.: input purity was 96.8% area

TABLE 5F
Impurity tracking from a 4-week stability study of HBI-3808
sodium salt of Pattern 5.

| | | | | | | | | Total |
|---|---|---|---|---|---|---|---|---|
| | | RRT | | | | | | Impu- |
| | | 0.801 | 0.959 | 1.000 ^{#} | 1.025 * | 1.767 | 1.932 | rities |
| 25° C./ | T = 0 | 0.35 | n/a | 96.83 | 2.66 | 0.17 | n/a | 3.17 |
| 60% | 1 week | 0.37 | 0.14 | 96.39 | 2.67 | 0.43 | n/a | 3.61 |
| RH | 2 weeks | 0.30 | n/a | 96.96 | 2.74 | n/a | n/a | 3.04 |
| | 4 weeks | 0.26 | 0.13 | 96.33 | 2.76 | 0.30 | 0.21 | 3.67 |
| 40° C./ | T = 0 | 0.35 | n/a | 96.83 | 2.66 | 0.17 | n/a | 3.17 |
| 75% | 1 week | 0.34 | 0.13 | 96.67 | 2.52 | 0.34 | n/a | 3.33 |
| RH | 2 weeks | 0.37 | n/a | 97.47 | 2.16 | n/a | n/a | 2.53 |
| | 4 weeks | 0.42 | 0.16 | 96.60 | 2.34 | 0.25 | 0.22 | 3.40 |
| 60° C. | T = 0 | 0.35 | n/a | 96.83 | 2.66 | 0.17 | n/a | 3.17 |
| (% | 1 week | 0.28 | 0.15 | 96.23 | 2.94 | 0.40 | n/a | 3.77 |
| area) | 2 weeks | 0.38 | n/a | 97.33 | 2.29 | n/a | n/a | 2.67 |
| | 4 weeks | 0.37 | 0.19 | 96.13 | 2.71 | 0.36 | 0.23 | 3.87 |

^{#} HBI-3808;
* Isomer of HBI-3808

### Example 6

Sodium salt of HBI-3808 having a XRPD of Pattern 6

Sodium salt of HBI-3808 having a XRPD of Pattern 6 (FIG. 6A) was prepared from sodium salt of Pattern 2, by heating a sample of sodium salt of Pattern 2, to 150° C. From XRPD analysis it can be seen that, Pattern 6 persists until 250° C. at which point solids become amorphous.

FIG. 6A shows the XRPD diffractogram of sodium salt of Pattern 6. FIG. 6B shows the peak fitting of the XRPD diffractogram (FIG. 6A) using pseudo-Voigt function with HighScore 4.9. The fitted parameters are listed in Table 6.

TABLE 6
XRPD peak lists of Sodium Salt of Pattern 6.

| | | FWHM Left | | |
|---|---|---|---|---|
| Pos. [°2θ] | Height [cts] | [°2θ] | d-spacing [Å] | Rel. Int. [%] |
| 5.7322 | 7209.60 | 0.0315 | 15.41808 | 53.75 |
| 5.8122 | 8093.22 | 0.0630 | 15.20610 | 60.33 |
| 5.9264 | 4159.12 | 0.0866 | 14.91324 | 31.01 |
| 6.8256 | 265.56 | 0.2519 | 12.95050 | 1.98 |
| 7.6733 | 207.66 | 0.1574 | 11.52156 | 1.55 |
| 9.1638 | 2187.77 | 0.1023 | 9.65070 | 16.31 |
| 9.2469 | 1462.37 | 0.0472 | 9.56418 | 10.90 |
| 11.1908 | 1048.93 | 0.0787 | 7.90684 | 7.82 |
| 11.5361 | 1226.03 | 0.1260 | 7.67089 | 9.14 |
| 11.6837 | 980.34 | 0.0630 | 7.57435 | 7.31 |
| 12.0985 | 363.32 | 0.1260 | 7.31554 | 2.71 |
| 12.4141 | 435.09 | 0.1260 | 7.13029 | 3.24 |
| 12.9174 | 4090.77 | 0.0394 | 6.85357 | 30.50 |
| 13.5442 | 13109.88 | 0.0960 | 6.53236 | 97.73 |
| 13.5910 | 13414.30 | 0.0480 | 6.52616 | 100.00 |
| 13.6662 | 9598.10 | 0.0768 | 6.47433 | 71.55 |
| 14.7071 | 3006.14 | 0.2112 | 6.01834 | 22.41 |
| 15.3190 | 518.18 | 0.1920 | 5.77929 | 3.86 |
| 16.1558 | 2373.01 | 0.2688 | 5.48179 | 17.69 |
| 16.7376 | 565.18 | 0.2688 | 5.29255 | 4.21 |
| 17.3462 | 2279.67 | 0.2112 | 5.10819 | 16.99 |
| 17.4840 | 1894.25 | 0.0768 | 5.06824 | 14.12 |
| 17.8822 | 534.49 | 0.1344 | 4.95628 | 3.98 |
| 18.8193 | 1440.25 | 0.2304 | 4.71154 | 10.74 |
| 20.2959 | 125.30 | 0.2304 | 4.37195 | 0.93 |
| 20.9226 | 362.79 | 0.1536 | 4.24240 | 2.70 |
| 21.5710 | 359.87 | 0.2304 | 4.11633 | 2.68 |
| 22.0668 | 230.24 | 0.2304 | 4.02495 | 1.72 |
| 22.4398 | 100.54 | 0.2304 | 3.95887 | 0.75 |
| 23.3685 | 334.51 | 0.2304 | 3.80360 | 2.49 |
| 24.2010 | 230.15 | 0.1152 | 3.67461 | 1.72 |
| 24.9112 | 86.11 | 0.3072 | 3.57145 | 0.64 |
| 25.8535 | 121.05 | 0.3840 | 3.44336 | 0.90 |
| 26.9597 | 454.98 | 0.2304 | 3.30454 | 3.39 |
| 27.4828 | 335.02 | 0.3840 | 3.24282 | 2.50 |
| 28.1483 | 141.60 | 0.1920 | 3.16764 | 1.06 |
| 29.1000 | 137.24 | 0.4608 | 3.06618 | 1.02 |
| 29.9553 | 336.06 | 0.2304 | 2.98054 | 2.51 |
| 30.9522 | 306.45 | 0.2304 | 2.88678 | 2.28 |
| 31.7430 | 168.23 | 0.2304 | 2.81665 | 1.25 |
| 33.0609 | 134.61 | 0.3072 | 2.70732 | 1.00 |
| 34.1328 | 57.46 | 0.4608 | 2.62471 | 0.43 |

### Example 7

Sodium salt of HBI-3808 having a XRPD of Pattern 7

Sodium salt of HB-3808 having a XRPD of Pattern 7 (FIG. 7A) was prepared from sodium salt of Pattern 5, by heating a sample of sodium salt of Pattern 5, to 150° C. From XRPD analysis it can be seen that, Pattern 7 persists until 250° C., and Pattern 7 did not persist after returning to 25° C.

FIG. 7A shows the XRPD diffractogram of sodium salt of Pattern 7. FIG. 7B shows the peak fitting of the XRPD diffractogram (FIG. 7A) using pseudo-Voigt function with HighScore 4.9. The fitted parameters are listed in Table 7.

TABLE 7
XRPD peak lists of Sodium Salt of Pattern 7.

| | | FWHM Left | | |
|---|---|---|---|---|
| Pos. [°2θ] | Height [cts] | [°2θ] | d-spacing [Å] | Rel. Int. [%] |
| 5.7897 | 4908.13 | 0.1181 | 15.26522 | 59.80 |
| 6.7400 | 87.00 | 0.6298 | 13.11479 | 1.06 |
| 8.7284 | 1024.04 | 0.1417 | 10.13105 | 12.48 |
| 11.5092 | 1160.65 | 0.1732 | 7.68879 | 14.14 |
| 13.1547 | 1143.93 | 0.0708 | 6.73048 | 13.94 |
| 13.8258 | 8207.90 | 0.0630 | 6.40524 | 100.00 |
| 14.5080 | 768.36 | 0.0945 | 6.10554 | 9.36 |
| 15.2244 | 1758.58 | 0.1732 | 5.81981 | 21.43 |
| 15.8399 | 1307.46 | 0.1102 | 5.59504 | 15.93 |
| 17.3514 | 1246.83 | 0.1260 | 5.11091 | 15.19 |
| 18.3211 | 1314.67 | 0.1732 | 4.84252 | 16.02 |
| 18.9414 | 601.34 | 0.0787 | 4.68532 | 7.33 |
| 20.2477 | 66.82 | 0.1889 | 4.38590 | 0.81 |
| 20.7899 | 75.91 | 0.1889 | 4.27271 | 0.92 |
| 21.5249 | 90.03 | 0.3779 | 4.12845 | 1.10 |
| 24.4386 | 133.98 | 0.1260 | 3.64244 | 1.63 |
| 26.4714 | 245.71 | 0.2204 | 3.36716 | 2.99 |
| 27.9789 | 85.60 | 0.3149 | 3.18907 | 1.04 |
| 29.1438 | 144.34 | 0.2519 | 3.06420 | 1.76 |
| 30.1268 | 108.15 | 0.2519 | 2.96642 | 1.32 |
| 32.0750 | 163.09 | 0.1889 | 2.79056 | 1.99 |
| 35.0953 | 252.01 | 0.1889 | 2.55702 | 3.07 |

### Example 8

Sodium salt of HBI-3808 having a XRPD of Pattern 8

Sodium salt of HBI-3808 having a XRPD of Pattern 8 (FIG. 8A) was obtained by temperature cycling (40-5° C. at 0.1° C./min with 1 h holds at 40° C. and 5° C., for 72 h) of amorphous sodium salts of HBI-3808 in DMVF (at 50 mg/mL), and in DMVF:water 80:20% v:% v (100 mg/mL). Pattern 8 is unstable and was converted to Pattern 11 (see Example 11) and Pattern 13 (see Example 13) after drying (at 40° C., under vacuum).

Pattern 8 was also prepared by HBI-3808 and sodium hydroxide. Approximately 7 g of HBI-3808 was weighed out and transferred to a 100 mL EasyMax vessel and was suspended in 21 mL of 2-propanol, at 50° C. The slurry was stirred at 200 rpm initially. A solution of 1.2 equivalents of sodium hydroxide (665 mg) in water (14 mL) was prepared. The solution of sodium hydroxide was added over 5 minutes into the EasyMax vessel. During the addition of the sodium hydroxide the stirring speed was increased to 350 rpm. The initial concentration of HBI-3808 was 200 mg/mL. After the sodium hydroxide was added a clear solution was observed and FBRM measurement was started. After dissolution was achieved, the experiment was cooled to 30° C. at 0.5° C./min and held at 30° C. The experiment was seeded with 1 wt. % (70 mg) of sodium salt Pattern 5, and then stirred for 1 h. After this, 2-propanol (42 mL) was added at 1 vol./h (7 mL/h).

The final solvent system was 2-propanol:water 82:18% v:% v. After the anti-solvent (2-propanol) addition was complete, the experiment was held for 1 h at 30° C. and then cooled to 5° C. at 0.1° C./min. The experiment was held at 5° C. for ca. 11 h. Solids were isolated by Buchner filtration (ø42.5 mm, grade 1) and washed with 2 volumes (14 mL) of 2-propanol:water 75:25% v:% v. A sub-sample was taken for XRPD and PLM. The solids were dried at 40° C. under vacuum for ca. 21 h. The damp isolated solids had a diffractogram consistent with sodium salt Pattern 8 (FIG. 8A). FIG. 8B shows the peak fitting of the XRPD diffractogram (FIG. 8A) using pseudo-Voigt function with HighScore 4.9. The fitted parameters are listed in Table 8. After drying at 40° C. under vacuum the solid were observed to be poorly crystalline by XRPD. The isolated yield of the solids was 94%, and the theoretical yield was 95%, based on the isolation of a mono-sodium mono-hydrate salt. The isolated solids had a chemical purity of 99.7%. The concentration in the mother liquor was 4.88 mg/mL, and the concentration in the wash liquor was 7.98 mg/mL. CAD indicated a sodium content of 4.3% w/w, approximately equal 1 equivalent of sodium. GC indicated a residual 2-propanol content of 69199 ppm. PLM analysis showed the damp and dried solids had a lathe-like morphology and were birefringent (FIG. 8C). TG-DSC analysis on the dried solids showed a weight loss of 9.4 wt. % between 20-133° C., followed by a second weight loss of 5.4 wt. % between 133 −255° C. (FIG. 8D). ¹H NMR spectroscopy was consistent with a sodium salt of HBI-3808 (FIG. 8E). The solids had a residual 2-propanol content of 9.98 wt. %, equal to ca. 0.7 equivalents. FIG. 8F shows the HPLC chromatogram of the isolated solids of sodium salt of Pattern 8. Particle size distribution (PSD) analysis indicated a d10 of 30.4 μm, a d50 of 65.2 μm, and a d90 of 118.4 μm, and was monomodal (FIG. 8G).

TABLE 8
XRPD peak lists of Sodium Salt of Pattern 8.

| | | FWHM Left | | |
|---|---|---|---|---|
| Pos. [°2θ] | Height [cts] | [°2θ] | d-spacing [Å] | Rel. Int. [%] |
| 5.1605 | 1364.98 | 0.0512 | 17.12498 | 63.47 |
| 5.4333 | 126.83 | 0.0512 | 16.26554 | 5.90 |
| 8.1626 | 2150.61 | 0.0768 | 10.83202 | 100.00 |
| 9.6720 | 1165.94 | 0.0768 | 9.14472 | 54.21 |
| 10.3383 | 112.52 | 0.0768 | 8.55684 | 5.23 |
| 11.5759 | 753.90 | 0.1151 | 7.64459 | 35.06 |
| 12.1345 | 596.55 | 0.0768 | 7.29396 | 27.74 |
| 13.1966 | 953.60 | 0.0768 | 6.70920 | 44.34 |
| 13.6733 | 1119.23 | 0.0640 | 6.47631 | 52.04 |
| 14.6374 | 986.90 | 0.0895 | 6.05187 | 45.89 |
| 15.5481 | 194.47 | 0.0768 | 5.69939 | 9.04 |
| 15.9720 | 1700.01 | 0.1279 | 5.54906 | 79.05 |
| 16.4088 | 203.64 | 0.0768 | 5.40232 | 9.47 |
| 16.8014 | 113.99 | 0.1023 | 5.27694 | 5.30 |
| 17.1684 | 409.13 | 0.0768 | 5.16497 | 19.02 |
| 17.6062 | 110.93 | 0.0768 | 5.03750 | 5.16 |
| 19.3848 | 242.00 | 0.0512 | 4.57913 | 11.25 |
| 19.7759 | 595.74 | 0.0640 | 4.48944 | 27.70 |
| 20.8052 | 337.93 | 0.1151 | 4.26962 | 15.71 |
| 21.7267 | 246.99 | 0.1023 | 4.09056 | 11.48 |
| 22.3700 | 439.08 | 0.0895 | 3.97436 | 20.42 |
| 22.9798 | 210.27 | 0.1023 | 3.87026 | 9.78 |
| 24.1383 | 371.69 | 0.1023 | 3.68707 | 17.28 |
| 24.6718 | 220.92 | 0.1023 | 3.60854 | 10.27 |
| 24.9703 | 292.16 | 0.1023 | 3.56608 | 13.58 |
| 25.4609 | 216.81 | 0.1535 | 3.49846 | 10.08 |
| 26.5656 | 315.14 | 0.0768 | 3.35544 | 14.65 |
| 26.8252 | 356.50 | 0.1023 | 3.32355 | 16.58 |
| 28.8524 | 195.47 | 0.1535 | 3.09448 | 9.09 |
| 30.4840 | 234.35 | 0.0768 | 2.93247 | 10.90 |
| 31.0648 | 100.69 | 0.3070 | 2.87896 | 4.68 |
| 32.4711 | 151.42 | 0.0768 | 2.75742 | 7.04 |
| 33.0625 | 60.58 | 0.2047 | 2.70943 | 2.82 |
| 33.5660 | 83.12 | 0.1535 | 2.66993 | 3.86 |

### Example 9

Sodium salt of HBI-3808 having a XRPD of Pattern 9

Sodium salt of HBI-3808 having a XRPD of Pattern 9 (FIG. 9A) was prepared from sodium salt of Pattern 5. Sodium salt of Pattern 5 in acetone:water 25:75% v:% v, at 5° C. for ca. 24 h produces sodium salt of Pattern 9. Pattern 9 was unstable and converted to a mixture of sodium salt of Pattern 10 (see Example 10) and sodium salt of Pattern 11 (see Example 11) after drying (40° C. under vacuum).

FIG. 9A shows the XRPD diffractogram of sodium salt of Pattern 9. FIG. 9B shows the peak fitting of the XRPD diffractogram (FIG. 9A) using pseudo-Voigt function with HighScore 4.9. The fitted parameters are listed in Table 9.

TABLE 9
XRPD peak lists of Sodium Salt of Pattern 9.

| | | FWHM Left | | |
|---|---|---|---|---|
| Pos. [°2θ] | Height [cts] | [°2θ] | d-spacing [Å] | Rel. Int. [%] |
| 3.7644 | 154.35 | 0.1023 | 23.47218 | 5.91 |
| 5.3332 | 2170.49 | 0.0895 | 16.57053 | 83.06 |
| 5.9336 | 844.56 | 0.1023 | 14.89535 | 32.32 |
| 6.4256 | 376.01 | 0.1535 | 13.75571 | 14.39 |
| 7.4003 | 73.07 | 0.1535 | 11.94609 | 2.80 |
| 8.3486 | 2613.19 | 0.1535 | 10.59111 | 100.00 |
| 9.9502 | 2218.60 | 0.1023 | 8.88970 | 84.90 |
| 10.4684 | 294.95 | 0.2047 | 8.45079 | 11.29 |
| 10.7903 | 285.96 | 0.2047 | 8.19940 | 10.94 |
| 11.9892 | 721.86 | 0.2303 | 7.38203 | 27.62 |
| 12.4476 | 812.02 | 0.1023 | 7.11117 | 31.07 |
| 13.1986 | 1353.43 | 0.0895 | 6.70819 | 51.79 |
| 13.5783 | 2442.08 | 0.0768 | 6.52141 | 93.45 |
| 13.9256 | 2326.43 | 0.1023 | 6.35956 | 89.03 |
| 14.4934 | 1110.21 | 0.0895 | 6.11169 | 42.48 |
| 14.9426 | 1984.19 | 0.1279 | 5.92893 | 75.93 |
| 16.5638 | 1361.31 | 0.1407 | 5.35211 | 52.09 |
| 17.1691 | 372.65 | 0.1535 | 5.16475 | 14.26 |
| 17.6470 | 611.91 | 0.1535 | 5.02595 | 23.42 |
| 18.1332 | 514.09 | 0.1279 | 4.89228 | 19.67 |
| 19.4346 | 173.84 | 0.2558 | 4.56751 | 6.65 |
| 19.9852 | 228.52 | 0.1535 | 4.44291 | 8.74 |
| 20.5461 | 403.72 | 0.1791 | 4.32286 | 15.45 |
| 21.4469 | 149.60 | 0.3070 | 4.14329 | 5.72 |
| 22.1270 | 184.19 | 0.1535 | 4.01745 | 7.05 |
| 23.1161 | 292.34 | 0.1023 | 3.84774 | 11.19 |
| 23.4143 | 189.06 | 0.1535 | 3.79942 | 7.23 |
| 25.2244 | 205.96 | 0.3582 | 3.53072 | 7.88 |
| 25.8441 | 195.56 | 0.1791 | 3.44745 | 7.48 |
| 27.5983 | 431.30 | 0.2047 | 3.23219 | 16.50 |
| 30.1024 | 103.02 | 0.6140 | 2.96877 | 3.94 |
| 32.5523 | 96.84 | 0.2558 | 2.75072 | 3.71 |
| 33.5957 | 52.40 | 0.4093 | 2.66764 | 2.01 |

### Example 10

Sodium salt of HBI-3808 having a XRPD of Pattern 10

Sodium salt of HB-3808 having a XRPD of Pattern 10 (FIG. 1A) was prepared from sodium salt of Pattern 5. Sodium salt of Pattern 5 in 1-propanol:water 50:50 3v:1v, at 5° C. for ca. 24 h produces sodium salt of Pattern 10.

FIG. 10A shows the XRPD diffractogram of sodium salt of Pattern 10. FIG. 10B shows the peak fitting of the XRPD diffractogram (FIG. 10A) using pseudo-Voigt function with HighScore 4.9. The fitted parameters are listed in Table 10.

TABLE 10
XRPD peak lists of Sodium Salt of Pattern 10.

| | | FWHM Left | | |
|---|---|---|---|---|
| Pos. [°2θ] | Height [cts] | [°2θ] | d-spacing [Å] | Rel. Int. [%] |
| 5.8172 | 5784.74 | 0.0640 | 15.19314 | 96.84 |
| 6.3687 | 5879.99 | 0.0768 | 13.87849 | 98.43 |
| 8.2796 | 553.85 | 0.0640 | 10.67926 | 9.27 |
| 8.5220 | 561.67 | 0.0512 | 10.37599 | 9.40 |
| 8.6788 | 948.79 | 0.0895 | 10.18894 | 15.88 |
| 10.3761 | 1136.69 | 0.0640 | 8.52570 | 19.03 |
| 10.5286 | 664.70 | 0.0640 | 8.40253 | 11.13 |
| 11.8067 | 111.27 | 0.1535 | 7.49568 | 1.86 |
| 13.1775 | 5371.82 | 0.0895 | 6.71886 | 89.93 |
| 13.4167 | 5973.61 | 0.0895 | 6.59961 | 100.00 |
| 13.9194 | 2842.90 | 0.0768 | 6.36239 | 47.59 |
| 14.0788 | 2526.27 | 0.1407 | 6.29068 | 42.29 |
| 14.5766 | 5956.39 | 0.0895 | 6.07699 | 99.71 |
| 14.6964 | 4676.39 | 0.0768 | 6.02771 | 78.28 |
| 15.0975 | 285.52 | 0.1023 | 5.86846 | 4.78 |
| 15.3375 | 188.31 | 0.0768 | 5.77718 | 3.15 |
| 17.1389 | 1567.45 | 0.1151 | 5.17380 | 26.24 |
| 17.4888 | 1464.15 | 0.1535 | 5.07107 | 24.51 |
| 17.8955 | 1081.66 | 0.0895 | 4.95672 | 18.11 |
| 18.1527 | 1587.69 | 0.1023 | 4.88707 | 26.58 |
| 18.5084 | 1200.05 | 0.1151 | 4.79393 | 20.09 |
| 19.1888 | 455.00 | 0.0895 | 4.62546 | 7.62 |
| 19.4535 | 480.04 | 0.1279 | 4.56311 | 8.04 |
| 19.7500 | 400.23 | 0.1023 | 4.49528 | 6.70 |
| 20.1035 | 528.85 | 0.2047 | 4.41701 | 8.85 |
| 20.8273 | 574.40 | 0.1023 | 4.26514 | 9.62 |
| 21.1388 | 189.07 | 0.1535 | 4.20297 | 3.17 |
| 22.0566 | 530.17 | 0.1151 | 4.03012 | 8.88 |
| 22.7520 | 151.06 | 0.1279 | 3.90848 | 2.53 |
| 23.4497 | 703.15 | 0.1151 | 3.79376 | 11.77 |
| 23.7165 | 180.18 | 0.1023 | 3.75169 | 3.02 |
| 24.1977 | 137.77 | 0.1023 | 3.67815 | 2.31 |
| 24.4273 | 300.63 | 0.1279 | 3.64411 | 5.03 |
| 25.1924 | 443.00 | 0.1279 | 3.53513 | 7.42 |
| 25.9438 | 334.10 | 0.1279 | 3.43443 | 5.59 |
| 26.2497 | 231.89 | 0.1535 | 3.39510 | 3.88 |
| 26.5160 | 294.53 | 0.1279 | 3.36160 | 4.93 |
| 27.2163 | 488.44 | 0.0895 | 3.27668 | 8.18 |
| 27.7427 | 107.70 | 0.1535 | 3.21568 | 1.80 |
| 28.0401 | 156.69 | 0.1023 | 3.18225 | 2.62 |
| 28.5094 | 261.51 | 0.2558 | 3.13093 | 4.38 |
| 29.1282 | 208.30 | 0.1791 | 3.06581 | 3.49 |
| 29.6235 | 182.57 | 0.2303 | 3.01566 | 3.06 |
| 30.3318 | 236.45 | 0.1023 | 2.94684 | 3.96 |
| 30.7664 | 654.30 | 0.1535 | 2.90620 | 10.95 |
| 31.3423 | 428.44 | 0.1791 | 2.85410 | 7.17 |
| 32.1881 | 381.91 | 0.1535 | 2.78101 | 6.39 |
| 32.4871 | 300.66 | 0.1535 | 2.75609 | 5.03 |
| 33.0550 | 357.93 | 0.1023 | 2.71003 | 5.99 |
| 33.8666 | 106.53 | 0.1791 | 2.64692 | 1.78 |
| 34.2070 | 101.01 | 0.1535 | 2.62136 | 1.69 |
| 34.7545 | 320.93 | 0.1279 | 2.58130 | 5.37 |

### Example 11

Sodium salt of HBI-3808 having a XRPD of Pattern 11

Sodium salt of HBI-3808 having a XRPD of Pattern 11 (FIG. 11A) was obtained from drying (40° C. under vacuum) solids sodium salt of Pattern 8. Sodium salt of Pattern 8 was isolated during solubility experiment of sodium salt of Pattern 5 in 2-propanol:water 90:10 % v: % v. FIG. 11A shows the XRPD diffractogram of sodium salt of Pattern 11. FIG. 11B shows the peak fitting of the XRPD diffractogram (FIG. 11A) using pseudo-Voigt function with HighScore 4.9. The fitted parameters are listed in Table 11. TG-DSC analysis of sodium salt of Pattern 11 showed a broad weight loss of 11.4 wt. % between 20-230° C. (FIG. 11C). There were no thermal events in the DSC thermogram.

TABLE 11
XRPD peak lists of Sodium Salt of Pattern 11.

| | | FWHM Left | | |
|---|---|---|---|---|
| Pos. [°2θ] | Height [cts] | [°2θ] | d-spacing [Å] | Rel. Int. [%] |
| 5.4018 | 1415.05 | 0.0768 | 16.36046 | 58.66 |
| 5.8063 | 324.66 | 0.0768 | 15.22148 | 13.46 |
| 6.4675 | 1266.38 | 0.0895 | 13.66669 | 52.50 |
| 6.7736 | 434.31 | 0.0640 | 13.04981 | 18.00 |
| 8.0596 | 121.97 | 0.0768 | 10.97031 | 5.06 |
| 9.0295 | 341.89 | 0.1023 | 9.79396 | 14.17 |
| 9.5576 | 226.85 | 0.2047 | 9.25398 | 9.40 |
| 10.7872 | 970.37 | 0.1407 | 8.20167 | 40.23 |
| 11.7081 | 425.86 | 0.1279 | 7.55860 | 17.65 |
| 11.9416 | 470.10 | 0.0768 | 7.41133 | 19.49 |
| 12.1993 | 642.52 | 0.1279 | 7.25536 | 26.64 |
| 12.7578 | 1065.72 | 0.0512 | 6.93894 | 44.18 |
| 13.1215 | 1592.13 | 0.0768 | 6.74743 | 66.00 |
| 13.4126 | 2412.23 | 0.1023 | 6.60162 | 100.00 |
| 14.0380 | 1517.10 | 0.1791 | 6.30888 | 62.89 |
| 14.5135 | 2010.03 | 0.1151 | 6.10327 | 83.33 |
| 14.7206 | 1453.54 | 0.0768 | 6.01786 | 60.26 |
| 14.9503 | 1211.20 | 0.1023 | 5.92591 | 50.21 |
| 15.6641 | 327.04 | 0.1535 | 5.65744 | 13.56 |
| 16.0711 | 324.90 | 0.1023 | 5.51505 | 13.47 |
| 16.4220 | 351.97 | 0.2558 | 5.39800 | 14.59 |
| 17.2324 | 470.48 | 0.1279 | 5.14593 | 19.50 |
| 18.1522 | 311.47 | 0.1023 | 4.88720 | 12.91 |
| 18.7645 | 283.69 | 0.1535 | 4.72907 | 11.76 |
| 19.2021 | 254.94 | 0.1535 | 4.62228 | 10.57 |
| 20.4098 | 155.31 | 0.1535 | 4.35142 | 6.44 |
| 21.0387 | 118.52 | 0.3070 | 4.22275 | 4.91 |
| 21.5241 | 119.87 | 0.2558 | 4.12860 | 4.97 |
| 22.5300 | 100.67 | 0.3070 | 3.94651 | 4.17 |
| 23.8454 | 116.27 | 0.2047 | 3.73169 | 4.82 |
| 24.6391 | 67.45 | 0.3070 | 3.61325 | 2.80 |
| 25.9419 | 39.30 | 0.3070 | 3.43467 | 1.63 |
| 28.0251 | 84.85 | 0.4093 | 3.18392 | 3.52 |
| 28.7346 | 93.25 | 0.4093 | 3.10690 | 3.87 |
| 29.7707 | 63.80 | 0.3070 | 3.00109 | 2.64 |
| 30.7226 | 66.73 | 0.4093 | 2.91024 | 2.77 |
| 31.6171 | 88.58 | 0.3070 | 2.82991 | 3.67 |

### Example 12

Sodium salt of HBI-3808 having a XRPD of Pattern 12

Sodium salt of HBI-3808 having a XRPD of Pattern 12 (FIG. 12A) was prepared from drying (40 2C under vacuum) from temperature cycling (0.1° C./min with 1 h holds at 40° C. and 5° C., for 72 h) of amorphous solids of the sodium salt of HBI-3808, in tert-butyl methyl ether (tBMVE) for 72 h between 40-5° C. and were maintained after drying (40° C. under vacuum).Solids of Pattern 12 were also observed in the damp solids after solvent drop grinding in tBME, and after slurrying at 40° C. in methyl ethyl ketone (.EK):methanol 80:20 % v: % v. FIG. 12A shows the XRPD diffractogram of sodium salt of Pattern 12. FIG. 12B shows the peak fitting of the XRPD diffractogram (FIG. 12A) using pseudo-Voigt function with HighScore 4.9. The fitted parameters are listed in Table 12.

TABLE 12
XRPD peak lists of Sodium Salt of Pattern 12.

| | | FWHM Left | | |
|---|---|---|---|---|
| Pos. [°2θ] | Height [cts] | [°2θ] | d-spacing [Å] | Rel. Int. [%] |
| 4.9423 | 5572.94 | 0.1023 | 17.88041 | 94.77 |
| 5.5312 | 423.54 | 0.3070 | 15.97788 | 7.20 |
| 6.5634 | 63.50 | 0.1535 | 13.46737 | 1.08 |
| 8.2332 | 1627.39 | 0.0768 | 10.73926 | 27.68 |
| 9.8847 | 123.57 | 0.1023 | 8.94844 | 2.10 |
| 11.8886 | 1012.50 | 0.1151 | 7.44423 | 17.22 |
| 13.1803 | 555.71 | 0.0768 | 6.71746 | 9.45 |
| 13.5470 | 5880.34 | 0.1023 | 6.53641 | 100.00 |
| 14.0818 | 2509.78 | 0.0768 | 6.28935 | 42.68 |
| 14.2507 | 3341.50 | 0.0895 | 6.21519 | 56.83 |
| 15.0811 | 2831.21 | 0.1023 | 5.87481 | 48.15 |
| 16.0431 | 1027.82 | 0.0512 | 5.52461 | 17.48 |
| 16.2631 | 1848.19 | 0.1279 | 5.45039 | 31.43 |
| 17.3580 | 2480.75 | 0.1407 | 5.10897 | 42.19 |
| 18.2743 | 686.31 | 0.1023 | 4.85482 | 11.67 |
| 18.9447 | 302.61 | 0.1023 | 4.68450 | 5.15 |
| 19.5029 | 408.59 | 0.1023 | 4.55167 | 6.95 |
| 19.9287 | 1112.26 | 0.1023 | 4.45538 | 18.91 |
| 20.6129 | 1397.57 | 0.0895 | 4.30900 | 23.77 |
| 20.8510 | 493.90 | 0.0768 | 4.26034 | 8.40 |
| 22.2245 | 180.22 | 0.1535 | 4.00005 | 3.06 |
| 23.5991 | 1092.51 | 0.1279 | 3.77008 | 18.58 |
| 23.9265 | 304.16 | 0.1023 | 3.71923 | 5.17 |
| 24.8983 | 115.52 | 0.1535 | 3.57622 | 1.96 |
| 25.6482 | 144.49 | 0.1023 | 3.47333 | 2.46 |
| 26.2207 | 322.70 | 0.0895 | 3.39879 | 5.49 |
| 27.0782 | 200.52 | 0.1279 | 3.29308 | 3.41 |
| 28.2231 | 224.71 | 0.4093 | 3.16203 | 3.82 |
| 28.8206 | 569.15 | 0.1023 | 3.09783 | 9.68 |
| 29.1355 | 159.64 | 0.1279 | 3.06505 | 2.71 |
| 29.9387 | 311.11 | 0.1279 | 2.98463 | 5.29 |
| 30.5746 | 420.73 | 0.3070 | 2.92399 | 7.15 |
| 31.1646 | 690.02 | 0.1279 | 2.86997 | 11.73 |
| 32.0092 | 139.81 | 0.1535 | 2.79614 | 2.38 |
| 32.4021 | 410.13 | 0.0895 | 2.76313 | 6.97 |
| 32.8096 | 301.18 | 0.1279 | 2.72974 | 5.12 |
| 33.1928 | 151.68 | 0.1535 | 2.69909 | 2.58 |
| 33.9892 | 200.94 | 0.1535 | 2.63765 | 3.42 |
| 34.5259 | 249.10 | 0.2047 | 2.59787 | 4.24 |

TG-DSC analysis indicated a weight loss of 5.1 wt. % between 91-144° C., followed by a second weight loss of 6.0 wt. % between 144-295° C. (FIG. 12C). In the DSC thermogram, one endothermic event with an onset temperature of 86° C. and a peak temperature of 119° C. was observed. ¹H NMR analysis was carried out in d₆-DMSO and the resulting spectrum was consistent with that for a sodium salt of HBI-3808 (FIG. 12D). A residual tBME content of 5.4 wt. % was observed, equal to ca. 0.3 equivalents.

A desolvation experiment was carried out by heating a sample of sodium salt of Pattern 12 to 144° C. and holding for 5 minutes. After returning to ambient conditions the sample was analyzed by XRPD which indicated it was poorly crystalline, with a diffractogram consistent with Pattern 12 (FIG. 12E).

### Example 13

Sodium salt of HBI-3808 having a XRPD of Pattern 13

Sodium salt of HBI-3808 having a XRPD of Pattern 13 (FIG. 13A) was obtained from drying solids of sodium salt of Pattern 8 (isolated from temperature cycling in DMF), at 40° C. under vacuum. Solids of sodium salt of Pattern 13 were also observed by XRPD for solvent drop grinding experiments in THF and methyl isobutyl ketone (MiBK). FIG. 13A shows the XRPD diffractogram of sodium salt of Pattern 13. FIG. 13B shows the peak fitting of the XRPD diffractogram (FIG. 13A) using pseudo-Voigt function with HighScore 4.9. The fitted parameters are listed in Table 13.

TABLE 13
XRPD peak lists of sodium salt of Pattern 13.

| | | FWHM Left | | |
|---|---|---|---|---|
| Pos. [°2θ] | Height [cts] | [°2θ] | d-spacing [Å] | Rel. Int. [%] |
| 4.8671 | 1055.88 | 0.0768 | 18.15657 | 89.15 |
| 5.4768 | 727.99 | 0.1791 | 16.13650 | 61.47 |
| 7.0217 | 251.83 | 0.1279 | 12.58932 | 21.26 |
| 8.1941 | 243.31 | 0.1279 | 10.79051 | 20.54 |
| 8.5674 | 242.59 | 0.2047 | 10.32112 | 20.48 |
| 10.5686 | 64.68 | 0.3070 | 8.37088 | 5.46 |
| 11.7569 | 120.49 | 0.2047 | 7.52731 | 10.17 |
| 12.8756 | 1184.39 | 0.1151 | 6.87574 | 100.00 |
| 13.7594 | 725.69 | 0.2303 | 6.43601 | 61.27 |
| 14.2748 | 955.51 | 0.0895 | 6.20479 | 80.68 |
| 14.5568 | 622.38 | 0.1535 | 6.08521 | 52.55 |
| 15.2822 | 502.18 | 0.1023 | 5.79795 | 42.40 |
| 16.0073 | 856.83 | 0.0768 | 5.53690 | 72.34 |
| 17.2555 | 257.32 | 0.1023 | 5.13910 | 21.73 |
| 18.2768 | 233.51 | 0.1023 | 4.85416 | 19.72 |
| 20.0040 | 174.53 | 0.2047 | 4.43877 | 14.74 |
| 20.4080 | 215.20 | 0.1279 | 4.35181 | 18.17 |
| 23.6377 | 224.02 | 0.1791 | 3.76401 | 18.91 |
| 26.3214 | 97.42 | 0.2047 | 3.38601 | 8.23 |
| 28.8324 | 90.96 | 0.2558 | 3.09658 | 7.68 |
| 30.4812 | 113.42 | 0.2047 | 2.93273 | 9.58 |
| 32.4421 | 82.32 | 0.4093 | 2.75982 | 6.95 |
| 33.4885 | 39.39 | 0.3070 | 2.67594 | 3.33 |

TG-DSC analysis of sodium salt of Pattern 13 showed a weight loss of 11.1 wt. % between 20-171° C., followed by a second weight loss of 3.5 wt. R between 171-297° C. (FIG. 13C). No thermal events were observed in the DSC thermogram. The ¹H NR spectrum of sodium salt of Pattern 13 was consistent with a sodium salt of HBI-3808 (FIG. 13D). The residual DMVF content was 9.6 wt. %, equal to ca. 0.6 equivalents.

A desolvation experiment was carried out by heating the sodium salt of Pattern 13 to 170° C. and holding for 5 minutes. After the solids had been returned to ambient temperature, they were analyzed by XRPD which indicated they were poorly crystalline (FIG. 13E).

### Example 14

Sodium salt of HBI-3808 having a XRPD of Pattern 14

Sodium salt of HBI-3808 having a XRPD of Pattern 14 (FIG. 14A) was isolated by evaporation of a solution of sodium salt of HBI-3808 in ethanol:water aw 0.5 (90:10% v:% v). FIG. 14A shows the XRPD diffractogram of sodium salt of Pattern 14. FIG. 14B shows the peak fitting of the XRPD diffractogram (FIG. 14A) using pseudo-Voigt function with HighScore 4.9. The fitted parameters are listed in Table 14.

TABLE 14
XRPD peak lists of sodium salt of Pattern 14.

| | | FWHM Left | | |
|---|---|---|---|---|
| Pos. [°2θ] | Height [cts] | [°2θ] | d-spacing [Å] | Rel. Int. [%] |
| 5.5105 | 87.19 | 0.3070 | 16.03790 | 5.76 |
| 6.5613 | 79.66 | 0.4093 | 13.47151 | 5.26 |
| 10.3264 | 164.21 | 0.1535 | 8.56667 | 10.85 |
| 10.7623 | 878.54 | 0.1023 | 8.22062 | 58.03 |
| 12.1952 | 1186.12 | 0.0895 | 7.25776 | 78.34 |
| 12.6335 | 442.95 | 0.1535 | 7.00691 | 29.26 |
| 13.9109 | 656.07 | 0.4093 | 6.36624 | 43.33 |
| 14.5147 | 1145.90 | 0.1279 | 6.10275 | 75.68 |
| 14.7873 | 1514.07 | 0.0895 | 5.99084 | 100.00 |
| 14.9779 | 1072.88 | 0.0768 | 5.91505 | 70.86 |
| 15.1360 | 768.10 | 0.1279 | 5.85362 | 50.73 |
| 15.7091 | 353.64 | 0.0768 | 5.64132 | 23.36 |
| 16.4819 | 149.11 | 0.3070 | 5.37851 | 9.85 |
| 17.1907 | 558.45 | 0.1151 | 5.15832 | 36.88 |
| 17.9453 | 130.09 | 0.3070 | 4.94308 | 8.59 |
| 19.1554 | 163.60 | 0.3070 | 4.63346 | 10.81 |
| 21.7107 | 58.18 | 0.3070 | 4.09355 | 3.84 |
| 23.8369 | 151.49 | 0.0768 | 3.73300 | 10.01 |
| 24.4442 | 765.90 | 0.1023 | 3.64162 | 50.59 |
| 25.2176 | 63.62 | 0.2047 | 3.53165 | 4.20 |
| 26.3111 | 328.91 | 0.2047 | 3.38731 | 21.72 |
| 28.0668 | 85.71 | 0.2047 | 3.17929 | 5.66 |
| 28.7907 | 154.70 | 0.1279 | 3.10098 | 10.22 |
| 29.1823 | 302.09 | 0.1535 | 3.06025 | 19.95 |
| 30.4343 | 57.09 | 0.3070 | 2.93714 | 3.77 |
| 31.6425 | 36.85 | 0.3070 | 2.82771 | 2.43 |
| 32.8409 | 106.90 | 0.2558 | 2.72721 | 7.06 |

TG-DSC analysis of sodium salt of Pattern 14 indicated a broad weight loss of 11.9 wt. % between 20-268° C. There were no thermal events observed in the DSC thermogram (FIG. 14C).

The ¹H NMVR spectrum of sodium salt of Pattern 14 was consistent with a sodium salt of HBI-3 808 (FIG. 14D). No residual ethanol was observed, indicating that the weight loss observed in the TG-DSC is likely due to loss of water.

A desolvation (dehydration) experiment was carried out by heating of sodium salt of Pattern 14, to 170° C. XRPD analysis on the solids after cooling to ambient temperature indicated they were amorphous (FIG. 14E).

### Example 15

Sodium salt of HBI-3808 having a XRPD of Pattern 15

Sodium salt of HBI-3808 having a XRPD of Pattern 15 (FIG. 15A) were prepared from HBI-3808 and sodium hydroxide by a set of reactive crystallization experiments in acetone:water (final solvent system 50:50, or 75:25% v:% v). Solids of sodium salts of Pattern 15 were seen from acetone:water crystallizations where sodium salts of Pattern 15 was observed with damp solids, however after drying the solids had converted to Pattern 2 or Pattern 5. Approximately 5.5 g of HBI-3808 free acid was weighed out and transferred to a 100 mL EasyMax vessel. The solids were slurried in 9.82 mL of acetone, with stirring at 150 rpm at 50° C. Separately, 1.2 equivalents of sodium hydroxide (523 mg) was dissolved in water (29.46 mL). The solution of sodium hydroxide was added over 5 minutes into the vessel, at 50° C. During the addition the stirring rate was increased to 350 rpm. Initial concentration of HBI-3808 was 140 mg/mL Dissolution was not achieved over 1 h at 50° C. The experiment was cooled to 45° C. at 0.5° C./min and 19.64 mL of acetone was added at 5.5 mL/h (1 volume/h). After the anti-solvent addition was complete the experiment was stirred for 1 h at 45° C. and then cooled to 5° C. at 0.1° C./min. The final solvent system was acetone:water 50:50% v:% v. The experiment was held at 5° C. for ca. 11 h, and the solids were then isolated by Buchner filtration (070 mm, grade 1 paper). The solids were washed with 2 volumes (11 mL) of acetone:water 50:50% v:% v. A sub-sample of solids were taken for XRPD and PLM. The solids were dried at 40° C. under vacuum with exposure to elevated humidity for ca. 22 h. After drying conversion to sodium salt of Pattern 5 was observed. FIG. 15A shows the XRPD diffractogram of sodium salt of Pattern 15. FIG. 15B shows the peak fitting of the XRPD diffractogram (FIG. 15A) using pseudo-Voigt function with HighScore 4.9. The fitted parameters are listed in Table 15.

TABLE 15
XRPD peak lists of sodium salt of Pattern 15.

| | | FWHM Left | | |
|---|---|---|---|---|
| Pos. [°2θ] | Height [cts] | [°2θ] | d-spacing [Å] | Rel. Int. [%] |
| 5.3226 | 6786.30 | 0.0768 | 16.60353 | 100.00 |
| 5.4917 | 1982.84 | 0.0768 | 16.09282 | 29.22 |
| 7.0743 | 660.56 | 0.0768 | 12.49578 | 9.73 |
| 8.4397 | 1164.41 | 0.0640 | 10.47703 | 17.16 |
| 10.3380 | 377.99 | 0.0895 | 8.55706 | 5.57 |
| 10.6560 | 279.83 | 0.1279 | 8.30242 | 4.12 |
| 12.5140 | 1416.04 | 0.0768 | 7.07358 | 20.87 |
| 12.9598 | 4898.77 | 0.0895 | 6.83124 | 72.19 |
| 13.7704 | 1464.39 | 0.0895 | 6.43087 | 21.58 |
| 14.1632 | 1486.69 | 0.0768 | 6.25340 | 21.91 |
| 14.2810 | 2003.83 | 0.0768 | 6.20207 | 29.53 |
| 14.8567 | 1279.94 | 0.0768 | 5.96301 | 18.86 |
| 14.9994 | 1736.13 | 0.0768 | 5.90660 | 25.58 |
| 15.2644 | 1282.86 | 0.1151 | 5.80465 | 18.90 |
| 16.0018 | 230.11 | 0.0768 | 5.53880 | 3.39 |
| 16.3841 | 2710.87 | 0.0895 | 5.41039 | 39.95 |
| 17.3094 | 564.99 | 0.1023 | 5.12321 | 8.33 |
| 17.8543 | 1606.85 | 0.1151 | 4.96807 | 23.68 |
| 18.2785 | 726.53 | 0.0768 | 4.85372 | 10.71 |
| 18.4669 | 387.56 | 0.0768 | 4.80461 | 5.71 |
| 18.7576 | 300.44 | 0.0512 | 4.73080 | 4.43 |
| 19.0579 | 257.36 | 0.1279 | 4.65695 | 3.79 |
| 19.5614 | 931.32 | 0.1023 | 4.53818 | 13.72 |
| 19.9698 | 400.68 | 0.1791 | 4.44630 | 5.90 |
| 20.4594 | 252.91 | 0.0895 | 4.34100 | 3.73 |
| 21.5062 | 348.86 | 0.0895 | 4.13201 | 5.14 |
| 22.6758 | 98.48 | 0.1023 | 3.92146 | 1.45 |
| 23.5315 | 320.65 | 0.0640 | 3.78075 | 4.72 |
| 24.1389 | 105.12 | 0.1023 | 3.68698 | 1.55 |
| 24.9119 | 219.82 | 0.1535 | 3.57430 | 3.24 |
| 25.8592 | 98.66 | 0.1023 | 3.44547 | 1.45 |
| 26.3586 | 291.83 | 0.0895 | 3.38131 | 4.30 |
| 26.9766 | 104.92 | 0.1023 | 3.30525 | 1.55 |
| 27.5651 | 124.30 | 0.1279 | 3.23600 | 1.83 |
| 28.2481 | 67.74 | 0.2558 | 3.15929 | 1.00 |
| 28.5528 | 104.29 | 0.0768 | 3.12627 | 1.54 |
| 28.9183 | 221.86 | 0.1279 | 3.08758 | 3.27 |
| 29.9093 | 144.69 | 0.1279 | 2.98750 | 2.13 |
| 30.5133 | 442.65 | 0.1791 | 2.92972 | 6.52 |
| 31.0211 | 80.64 | 0.1535 | 2.88292 | 1.19 |
| 31.8153 | 205.53 | 0.1023 | 2.81274 | 3.03 |
| 32.4146 | 266.04 | 0.1279 | 2.76209 | 3.92 |
| 33.1137 | 295.05 | 0.1279 | 2.70536 | 4.35 |
| 33.5119 | 271.20 | 0.1279 | 2.67412 | 4.00 |
| 34.5641 | 181.53 | 0.1279 | 2.59508 | 2.67 |

TG-DSC analysis of sodium salt of Pattern 15 showed a weight loss of 6.4 wt. % between 20-102° C., followed by a second weight loss of 3.3 wt. % between 102-187° C. (FIG. 15C). In the DSC thermogram there were two endothermic events with onset (and peak) temperatures of 44(59) ° C. and 95(122) ° C. The ¹H NMR spectrum of sodium salt of Pattern 15 was consistent with the structure of a sodium salt of HBI-3808 (FIG. 15D). The residual acetone content was 3.4 wt. %, equal to ca. 0.3 equivalents.

A desolvation experiment was carried out by heating the sodium salt of Pattern 15, to 185° C. XRPD analysis on the solids after cooling to ambient temperature indicated they had a diffractogram consistent with sodium salt of Pattern 2 (FIG. 15E).

### Example 16

Sodium salt of HBI-3808 having a XRPD of Pattern 16

Sodium salt of HBI-3808 having a XRPD of Pattern 16 (FIG. 16A) were isolated after poorly crystalline solids were re-slurried in MEK. Approximately 2 g of sodium salt of HBI-3808 were weighed and transferred into a 100 mL EasyMax vessel. The solids were slurried in 25 mL MEK at 40° C., for ca. 24 h. Stirring was done at 300 rpm. Solids were isolated by Buchner filtration (ø42.5 mm, grade 1) and washed with 2 volumes (14 mL) of MEK. The solids were dried at 40 C under vacuum with exposure to high humidity for ca. 18 h. XRPD of the damp solids yielded a novel diffractogram, was labeled as Pattern 16, indicating formation of sodium salt of Pattern 16. After drying with exposure to humidity the XRPD was consistent with sodium salt of Pattern 5. FIG. 16A shows the XRPD diffractogram of sodium salt of Pattern 16. FIG. 16B shows the peak fitting of the XRPD diffractogram (FIG. 16A) using pseudo-Voigt function with HighScore 4.9. The fitted parameters are listed in Table 16.

TABLE 16
XRPD peak lists of sodium salt of Pattern 16.

| | | FWHM Left | | |
|---|---|---|---|---|
| Pos. [°2θ] | Height [cts] | [°2θ] | d-spacing [Å] | Rel. Int. [%] |
| 4.1617 | 302.79 | 0.0512 | 21.23220 | 2.29 |
| 4.6064 | 666.75 | 0.0512 | 19.18355 | 5.05 |
| 4.6989 | 483.97 | 0.0384 | 18.80601 | 3.67 |
| 4.8359 | 303.62 | 0.0384 | 18.27368 | 2.30 |
| 5.0753 | 8339.43 | 0.0640 | 17.41224 | 63.16 |
| 7.2967 | 665.51 | 0.0512 | 12.11547 | 5.04 |
| 7.4177 | 424.51 | 0.0384 | 11.91813 | 3.22 |
| 7.5470 | 677.25 | 0.0512 | 11.71413 | 5.13 |
| 7.6823 | 965.85 | 0.0640 | 11.50812 | 7.32 |
| 7.8119 | 3395.92 | 0.0384 | 11.31751 | 25.72 |
| 7.8907 | 5032.99 | 0.0256 | 11.20460 | 38.12 |
| 8.0135 | 13203.62 | 0.0768 | 11.03323 | 100.00 |
| 9.2379 | 395.82 | 0.0512 | 9.57350 | 3.00 |
| 9.5050 | 2123.84 | 0.0895 | 9.30502 | 16.09 |
| 10.1583 | 1132.17 | 0.0768 | 8.70801 | 8.57 |
| 11.3805 | 2368.73 | 0.0640 | 7.77542 | 17.94 |
| 11.7299 | 394.01 | 0.0640 | 7.54457 | 2.98 |
| 11.9241 | 2335.96 | 0.0640 | 7.42216 | 17.69 |
| 12.8713 | 1090.73 | 0.0895 | 6.87802 | 8.26 |
| 12.9851 | 1835.37 | 0.0640 | 6.81800 | 13.90 |
| 13.2117 | 1834.76 | 0.0768 | 6.70158 | 13.90 |
| 13.4036 | 7880.25 | 0.0895 | 6.60601 | 59.68 |
| 14.0843 | 477.81 | 0.0384 | 6.28824 | 3.62 |
| 14.3483 | 5535.70 | 0.1023 | 6.17314 | 41.93 |
| 15.2706 | 1021.86 | 0.1023 | 5.80233 | 7.74 |
| 15.5192 | 1368.22 | 0.0512 | 5.70991 | 10.36 |
| 15.6761 | 6522.73 | 0.0895 | 5.65311 | 49.40 |
| 16.0639 | 599.57 | 0.0640 | 5.51753 | 4.54 |
| 16.5019 | 406.73 | 0.0768 | 5.37204 | 3.08 |
| 16.8513 | 3986.37 | 0.0895 | 5.26142 | 30.19 |
| 17.2711 | 595.68 | 0.0768 | 5.13450 | 4.51 |
| 18.4596 | 234.39 | 0.0640 | 4.80650 | 1.78 |
| 19.0480 | 373.26 | 0.0895 | 4.65935 | 2.83 |
| 19.3277 | 2059.11 | 0.1151 | 4.59254 | 15.60 |
| 19.9935 | 668.85 | 0.1407 | 4.44109 | 5.07 |
| 20.4070 | 1679.43 | 0.0895 | 4.35202 | 12.72 |
| 21.2627 | 928.31 | 0.0895 | 4.17877 | 7.03 |
| 21.9727 | 1600.18 | 0.1023 | 4.04533 | 12.12 |
| 22.2839 | 253.35 | 0.0768 | 3.98952 | 1.92 |
| 22.5758 | 1055.92 | 0.0512 | 3.93860 | 8.00 |
| 22.8919 | 215.13 | 0.1279 | 3.88493 | 1.63 |
| 23.6593 | 547.79 | 0.0640 | 3.76062 | 4.15 |
| 23.9475 | 540.18 | 0.0640 | 3.71602 | 4.09 |
| 24.1995 | 410.34 | 0.0640 | 3.67789 | 3.11 |
| 24.5185 | 309.85 | 0.1151 | 3.63075 | 2.35 |
| 24.7660 | 366.23 | 0.0384 | 3.59502 | 2.77 |
| 24.9917 | 341.23 | 0.1023 | 3.56306 | 2.58 |
| 25.4163 | 222.19 | 0.0768 | 3.50449 | 1.68 |
| 25.9871 | 307.16 | 0.1279 | 3.42881 | 2.33 |
| 26.3500 | 1485.71 | 0.0768 | 3.38240 | 11.25 |
| 27.0228 | 234.46 | 0.1023 | 3.29970 | 1.78 |
| 27.7839 | 209.99 | 0.1535 | 3.21101 | 1.59 |
| 28.3158 | 590.80 | 0.1151 | 3.15189 | 4.47 |
| 29.0014 | 287.23 | 0.1023 | 3.07892 | 2.18 |
| 29.3897 | 187.97 | 0.1279 | 3.03912 | 1.42 |
| 29.7375 | 616.10 | 0.0768 | 3.00436 | 4.67 |
| 29.9547 | 1374.70 | 0.0624 | 2.98061 | 10.41 |
| 30.0382 | 754.24 | 0.0468 | 2.97989 | 5.71 |
| 30.4324 | 358.04 | 0.0468 | 2.93490 | 2.71 |
| 30.6373 | 372.60 | 0.1872 | 2.91573 | 2.82 |
| 31.1641 | 212.59 | 0.1872 | 2.86764 | 1.61 |
| 31.8798 | 663.35 | 0.0624 | 2.80487 | 5.02 |
| 32.4736 | 433.48 | 0.2184 | 2.75493 | 3.28 |
| 32.9350 | 356.09 | 0.0468 | 2.71738 | 2.70 |
| 33.3785 | 544.09 | 0.1560 | 2.68228 | 4.12 |
| 33.5582 | 451.37 | 0.0936 | 2.66833 | 3.42 |
| 34.1033 | 231.12 | 0.1248 | 2.62692 | 1.75 |

The solids were isolated in an 81% yield, based on the isolation of a mono-hydrate sodium salt. The solids had a chemical purity of 99.7%. The theoretical yield was 95%, accounting for the losses to the mother and wash liquors. The concentration of HBI-3808 in the mother liquor was 0.12 mg/mL and the concentration of the wash liquor was 0.03 mg/mL.

### Example 17

Ethanolamine salt of HBI-3808

Ethanolamine salt of HBI-3808 was prepared form HBI-3808 and ethanolamine. 1 g of HBI-3808, was slurried in 8 mL of methanol: water 90:10% v:O % v. 1 equivalent of ethanolamine (132 μL) in 2 mL of methanol:water 90:10% v:% v was added over 5 minutes via syringe pump.

The clear solution was held for 1 h at 40′° C. After 1 h, 2 mL of TBMVE was added over 10 minutes via syringe pump at 40° C., and then held at 40° C. for 1 h. The experiment was then cooled to 5° C. at 0.1° C./min and held at 5° C. for ca. 11 h. A sub-sample of slurry was taken, and solids isolated by centrifuge filtration (nylon, 0.22 μm). Bulk solids were isolated by Buchner filtration (ø42.5 mm, grade 1 paper). Solids were dried at 40° C. under vacuum for 23 h (batch 1). Isolated yield was 44%. Additional TBMVE anti-solvent (6 mL) was added to the filtered mother liquor, at 40 PC. The experiment was cooled from 40° C. to 5° C. at 0.1° C./min, and held at 5° C. for ca. 18 h. Solids were isolated by Buchner filtration (ø42.5 mm, grade 1 paper). Solids were dried at 40° C. under vacuum for 23 h (batch 2). Isolated yield was 12 00. Additional TBMVE anti-solvent (9 mL) was added to the filtered mother liquor, at 40° C. The experiment was cooled from 40° C. to 5° C. at 0.1 7° C./min, and held at 5° C. for ca. 18 h. Solids were isolated by Buchner filtration (ø42.5 mm, grade 1 paper). Solids were dried at 40° C. under vacuum for 18 h (batch 3). Isolated yield was 6 00 FIG. 17A shows the XRPD diffractogram of the isolated solids of the ethanolamine salt of HBI-3808. FIG. 17B shows the peak fitting of the XRPD diffractogram (FIG. 17A) using pseudo-Voigt function with HighScore 4.9. The fitted parameters are listed in Table 17A.

TABLE 17A
XRPD peak lists of ethanolamine salt of HBI-3808.

| | | FWHM Left | | |
|---|---|---|---|---|
| Pos. [°2θ] | Height [cts] | [°2θ] | d-spacing [Å] | Rel. Int. [%] |
| 5.0598 | 1066.78 | 0.0640 | 17.46547 | 26.10 |
| 7.5456 | 218.95 | 0.0640 | 11.71625 | 5.36 |
| 8.7215 | 348.92 | 0.0768 | 10.13909 | 8.54 |
| 10.1237 | 2637.36 | 0.0640 | 8.73769 | 64.53 |
| 10.4074 | 602.63 | 0.0768 | 8.50019 | 14.74 |
| 12.0250 | 889.98 | 0.1023 | 7.36007 | 21.78 |
| 12.3884 | 4087.10 | 0.0895 | 7.14501 | 100.00 |
| 12.8019 | 2899.25 | 0.0895 | 6.91512 | 70.94 |
| 13.7386 | 1253.41 | 0.0768 | 6.44568 | 30.67 |
| 13.9804 | 1204.70 | 0.0895 | 6.33475 | 29.48 |
| 14.6587 | 2925.02 | 0.0895 | 6.04310 | 71.57 |
| 15.2168 | 1647.11 | 0.0768 | 5.82270 | 40.30 |
| 15.7098 | 2581.12 | 0.1023 | 5.64106 | 63.15 |
| 16.1552 | 486.91 | 0.1023 | 5.48654 | 11.91 |
| 16.8204 | 250.54 | 0.0768 | 5.27102 | 6.13 |
| 17.1110 | 482.46 | 0.0895 | 5.18215 | 11.80 |
| 17.5085 | 86.49 | 0.0768 | 5.06540 | 2.12 |
| 18.7325 | 323.10 | 0.1151 | 4.73708 | 7.91 |
| 19.1220 | 297.91 | 0.0768 | 4.64147 | 7.29 |
| 21.1218 | 319.88 | 0.0895 | 4.20631 | 7.83 |
| 21.5911 | 436.48 | 0.0895 | 4.11595 | 10.68 |
| 22.0424 | 90.86 | 0.1023 | 4.03268 | 2.22 |
| 22.5001 | 288.81 | 0.1023 | 3.95168 | 7.07 |
| 23.7460 | 284.31 | 0.1023 | 3.74709 | 6.96 |
| 24.0248 | 385.20 | 0.0640 | 3.70424 | 9.42 |
| 24.6366 | 334.03 | 0.1023 | 3.61361 | 8.17 |
| 24.8442 | 412.87 | 0.1279 | 3.58389 | 10.10 |
| 25.7707 | 149.10 | 0.1023 | 3.45710 | 3.65 |
| 26.7744 | 232.64 | 0.1535 | 3.32974 | 5.69 |
| 27.6541 | 137.58 | 0.1279 | 3.22579 | 3.37 |
| 27.9653 | 162.60 | 0.1023 | 3.19059 | 3.98 |
| 28.6897 | 275.24 | 0.1151 | 3.11166 | 6.73 |
| 28.9895 | 364.49 | 0.1535 | 3.08016 | 8.92 |
| 29.5426 | 131.98 | 0.0768 | 3.02374 | 3.23 |
| 30.5432 | 165.07 | 0.1535 | 2.92693 | 4.04 |
| 31.5456 | 286.86 | 0.2047 | 2.83617 | 7.02 |
| 32.1909 | 220.96 | 0.1535 | 2.78077 | 5.41 |
| 32.6640 | 188.21 | 0.1535 | 2.74157 | 4.61 |
| 33.0837 | 148.40 | 0.1791 | 2.70774 | 3.63 |
| 33.7583 | 278.62 | 0.1279 | 2.65517 | 6.82 |

HPLC analysis for solid chemical purity indicated solids from batch 1 had a purity of 97.8 %; solids from batch 2 had a purity of 97.5%; and solids from batch 3 has a purity of 97.5%. The final filtered mother liquor had a concentration of 5.6 mg/mL. FIG. 17C, D and E shows HPLC chromatogram of the isolated solids of batch 1, 2 and 3 respectively. KF analysis indicated the solids had a water content of 2.7 wt. %. GC analysis indicated residual solvent contents of 2847 ppm methanol; and 27 ppm TBME. The ¹H NMR spectrum showed ethanolamine (S 3.37; 2.58 ppm) present at a content of 9.78 wt. % (ca. 1 mole equivalent), indicating that the ethanolamine salt is a mono-ethanolamine salt (FIG. 17F). No residual methanol or TBME was detected due to signal over-lap. PLM analysis indicated the isolated solids had a rod-like morphology and were birefringent (FIG. 17G). TG-DSC analysis indicated two weight losses. The first weight loss of 2.3 wt. % was observed between 20-170° C. The second weight loss of 10.1 wt. % was observed between 170 −228° C., and would theoretically be equal to 1 equivalent of ethanolamine (FIG. 17H). In the DSC thermogram, three endothermic events were observed (FIG. 17I). The first endothermic event had an onset temperature of 198° C., and a peak temperature of 204° C., and corresponds to the potential loss of ethanolamine. The second endothermic event had an onset temperature of 271° C., and a peak temperature of 279° C. The third endothermic event had an onset temperature of 291° C., and a peak temperature of 296° C. DSC analysis showed two endothermic events in the region of 20-300° C. The first event had an onset temperature of 200° C., and a peak temperature of 201° C. The second endothermic event had an onset temperature of 276° C., and a peak temperature of 286° C. FT-IR (FIG. 17J) and Raman spectra (FIG. 17K) were recorded for reference. In the DVS analysis the ethanolamine salt had a water uptake of 3.0 wt. % at 80% RH, and 25° C., indicating the material was slightly hygroscopic. FIG. 17L shows DVS mass plot of the isolated solids ethanolamine salt. FIG. 17M shows DVS isotherm plot of the isolated solids ethanolamine salt. XRPD pattern of the ethanolamine salt was retained post-DVS (FIG. 17N). Hot stage microscopy was carried out between 25-300° C., and showed no changes. However, at the end of the experiment the solids on the microscope slide had discolored (FIG. 17O).

VT-XRPD was carried out according to the temperature program of Table 17B. Diffractograms consistent with ethanolamine salt were observed between 25° C. and 190° C. At 210° C., the XRPD diffractogram was consistent with poorly crystalline BBI-3808 free acid of Pattern 1. Free acid (HBI-3808) of Pattern 1 was observed between 210-240° C. At temperatures of 270° C. and above, the material was observed to be amorphous (FIG. 17P).

TABLE 17B
Temperature program for the VT-XRPD of the ethanolamine salt

| Temperature / ° C. | Heating Rate / ° C./min |
|---|---|
| 25 | n/a |
| 160 | 10 |
| 170 | 2 |
| 190 | 2 |
| 210 | 2 |
| 230 | 2 |
| 240 | 2 |
| 270 | 2 |
| 280 | 2 |
| 290 | 2 |
| 300 | 2 |
| 25 | n/a |

VH-XRPD analysis (FIG. 17Q), carried out according to the humidity program of Table 17C, showed no changes in XRPD Pattern between 0-90% RH.

TABLE 17C
Humidity program for the VH-XRPD of ehtanolamine salt

| Relative Humidity / | | Temperature / | |
|---|---|---|---|
| % RH | Time / min | ° C. | Step |
| 40 | 5 | 30 | Desorption 1 |
| 30 | 5, 60 | | |
| 20 | 5, 60 | | |
| 10 | 5, 60 | | |
| 0 | 5, 60, 840 | | |
| 40 | 5, 60 | | Sorption 1 |
| 50 | 5, 60 | | |
| 60 | 5, 60 | | |
| 70 | 5, 60 | | |
| 80 | 5, 60 | | |
| 90 | 5, 60, 120 | | |
| 40 | 5 | | Desorption 2 |

Salt disproportionation studies of ethanolamine salt. The recovered solids from salt disproportionation studies had a XRPD diffractogram (FIG. 17R) consistent with ethanolamine salt Pattern, indicating that no salt disproportionation had occurred. The pH of the filtered mother liquor was 8.65.

Hydration studies of ethanolamine salt. Hydration studies in methanol/water at a_{w}0.1, 0.5 and 0.9 indicated that the solids isolated at the end from all solvent systems had XRPD Patterns consistent with ethanolamine salt (FIG. 17S).

pH dependent solubility studies of ethanolamine salt of Pattern 1. pH solubility studies carried out showed, the concentration in the mother liquor was <0.001 mg/mL at pH 1.2 -4.0. At pH 6.8, the concentration was 0.5 mg/mL. At pH 8.0, the concentration was 17.7 mg/mL.

In FaSSGF the final pH was 8.62, and the concentration was 57.2 mg/mL. Amorphous solids were isolated from buffers pH 1.2 hydrochloride; pH 6.8 phosphate; and pH 8.0 phosphate. Solids with XRPD diffractograms consistent with HBI-3808 free acid of Pattern 1 were recovered from pH 3.0 and pH 4.0 acetate buffers, indicating disproportionation is occurring at these pH levels. Solids recovered from FaSSGF had a XRPD Pattern consistent with ethanolamine salt (FIG. 17T). Starting concentration of HBI- 3808 ethanolamine salt of Pattern 1, for the solubility studies were 100 mg/mL. Summary of the results from the pH dependent solubility studies with the ethanolamine salt is shown in Table 17D.

TABLE 17D
Summary of the results from the pH dependent solubility studies with the ethanolamine salt.

| | 1 h | | 3 h | | 22 h | | 24 h | HPLC | |
|---|---|---|---|---|---|---|---|---|---|
| Target | Measured | Adjusted | Measured | Adjusted | Measured | Adjusted | Measured | Conc./ | |
| pH | pH | pH | pH | pH | pH | pH | pH | mg/mL * | XRPD |
| 1.2 | 3.90 | 1.22 | 1.41 | 1.22 | 1.41 | 1.17 | 1.52 | <0.001 | |
| 3.0 | 3.78 | 3.10 | 3.32 | 3.08 | 3.17 | 3.08 | 3.16 | <0.001 | 1** |
| 4.0 | 4.60 | 4.12 | 4.10 | | 4.16 | 4.05 | 4.18 | <0.001 | 1** |
| 6.8 | 8.43 | 6.86 | 7.01 | 6.86 | 6.89 | | 6.89 | 0.5 | n/a |
| 8.0 | 8.60 | 8.04 | 8.46 | 8.08 | 8.21 | 7.93 | 8.27 | 17.7 | n/a |
| FaSSGF | n/a | n/a | n/a | n/a | n/a | n/a | 8.62 | 57.2 | 1*** |
| | | | | | | | | | PC |

* Based on free acid
**Free acid
***Crystalline salt

A 13-week stability studies of ethanolamine salt. The input for this set of experiments was batch 2 material, which had a chemical purity of 97.5% area. Ethanolamine salt Pattern was maintained across all stability conditions for the duration of the 13-week study.

There was no significant decrease in chemical purity, by HPLC, after exposure to 40° C./75 % RH, 25° C./60% RH, or 60° C. HPLC indicated solids recovered from these conditions had chemical purities of 97.4% area, after 13 weeks.

FIG. 17U shows XRPD diffractogram of isolated solids from a 1-week stability study with ethanolamine salt. FIG. 17V shows HPLC chromatogram of solids after 1 week at 25° C./60% RH (ethanolamine salt as input). FIG. 17W shows HPLC chromatogram of solids after 1 week at 40° C./75% RH (ethanolamine salt as input). FIG. 17X shows HPLC chromatogram of solids after 1 week at 60° C. (ethanolamine salt as input). FIG. 17Y shows XRPD diffractogram of isolated solids from a 2-week stability study with ethanolamine salt. FIG. 17Z shows HPLC chromatogram of solids after 2 weeks at 25° C./60% RH (ethanolamine salt as input). FIG. 17AA shows HPLC chromatogram of solids after 2 weeks at 40° C./75% RH (ethanolamine salt as input). FIG. 17BB shows HPLC chromatogram of solids after 2 weeks at 60° C. (ethanolamine salt as input). FIG. 17CC shows XRPD diffractogram of isolated solids from a 4-week stability study with ethanolamine salt. FIG. 17DD shows HPLC chromatogram of solids after 4 weeks at 25° C./60% RH (ethanolamine salt as input). FIG. 17EE shows HPLC chromatogram of solids after 4 weeks at 40° C./75% RH (ethanolamine salt as input). FIG. 17FF shows HPLC chromatogram of solids after 4 weeks at 60° C. (ethanolamine salt as input).

FIG. 17GG shows XRPD diffractogram of isolated solids from an 8-week stability study with ethanolamine salt. FIG. 17HH shows HPLC chromatogram of solids after 8 weeks at 25° C./60 %RH (ethanolamine salt as input). FIG. 17II shows HPLC chromatogram of solids after 8 weeks at 40° C./75 RH (ethanolamine salt as input). FIG. 17JJ shows HPLC chromatogram of solids after 8 weeks at 60° C. (ethanolamine salt as input). FIG. 17KK shows XRPD diffractogram of isolated solids from a 13-week stability study with ethanolamine salt. FIG. 17LL shows HPLC chromatogram of solids after 13 weeks at 25° C./60 % RH (ethanolamine salt as input). FIG. 17MM shows HPLC chromatogram of solids after 13 weeks at 40° C./75 % RH (ethanolamine salt of Pattern 1 as input). FIG. 17NN shows HPLC chromatogram of solids after 13 weeks at 60° C. (ethanolamine salt as input). Table 17E presents summary of the results from a 13-week stability study of ethanolamine salt. Table 17F presents impurity tracking results from a 13-week stability study of ethanolamine salt.

TABLE 17E
Summary of the results from a 13-week stability study of the ethanolamine salt.

| | 1 Week | | 2 Weeks | | 4 Weeks | | 8 Weeks | | 13 Weeks | |
|---|---|---|---|---|---|---|---|---|---|---|
| Stability | | HPLC/% | | HPLC/% | | HPLC/% | | HPLC/% | | HPLC/% |
| Condition | XRPD | area | XRPD | area | XRPD | area | XRPD | area | XRPD | area |
| 25° C./60% RH | 1* | 97.3 | 1* | 97.3 | 1* | 97.5 | 1* | 97.0 | 1* | 97.3 |
| (open vial) | | | | | | | | | | |
| 40° C./75% RH | 1* | 97.3 | 1* | 97.3 | 1* | 97.5 | 1* | 96.9 | 1* | 97.4 |
| (open vial) | | | | | | | | | | |
| 60° C. | 1* | 97.5 | 1* | 97.3 | 1* | 97.5 | 1* | 97.0 | 1* | 97.4 |
| (closed vial) | | | | | | | | | | |

c.f.: input purity 97.8% area
HPLC results based on free acid
*crystalline salt

TABLE 17F
Impurity tracking results from a 13-week stability study of the
ethanolamine salt.

| | | | | | | | | Total |
|---|---|---|---|---|---|---|---|---|
| | | RRT | | | | | | Impu- |
| | | 0.801 | 0.970 | 1.000 ^{#} | 1.025 * | 1.767 | 1.935 | rities |
| 25° C./ | T = 0 | n/a | n/a | 97.82 | 2.18 | n/a | n/a | 2.18 |
| 60% RH | 1 week | 0.10 | 0.10 | 97.31 | 2.48 | n/a | n/a | 2.69 |
| (% area) | 2 | n/a | 0.12 | 97.29 | 2.39 | n/a | 0.20 | 2.71 |
| | weeks | | | | | | | |
| | 4 | n/a | 0.11 | 97.47 | 2.41 | n/a | n/a | 2.53 |
| | weeks | | | | | | | |
| | 8 | n/a | 0.10 | 96.99 | 2.51 | 0.29 | n/a | 3.01 |
| | weeks | | | | | | | |
| | 13 | 0.12 | n/a | 97.33 | 2.37 | n/a | 0.19 | 2.67 |
| | weeks | | | | | | | |
| 40° C./ | T = 0 | n/a | n/a | 97.82 | 2.18 | n/a | n/a | 2.18 |
| 75% RH | 1 week | 0.10 | 0.14 | 97.28 | 2.48 | n/a | n/a | 2.72 |
| (% area) | 2 | n/a | 0.12 | 97.26 | 2.40 | n/a | 0.22 | 2.74 |
| | weeks | | | | | | | |
| | 4 | n/a | 0.11 | 27.48 | 2.41 | n/a | n/a | 2.52 |
| | weeks | | | | | | | |
| | 8 | n/a | 0.21 | 96.88 | 2.51 | 0.40 | n/a | 3.12 |
| | weeks | | | | | | | |
| | 13 | n/a | n/a | 97.41 | 2.40 | n/a | 0.20 | 2.59 |
| | weeks | | | | | | | |
| 60° C. | T = 0 | n/a | n/a | 97.82 | 2.18 | n/a | n/a | 2.18 |
| (% area) | 1 week | 0.12 | n/a | 97.46 | 2.43 | n/a | n/a | 2.54 |
| | 2 | n/a | 0.12 | 97.27 | 2.41 | n/a | 0.20 | 2.73 |
| | weeks | | | | | | | |
| | 4 | n/a | 0.11 | 97.49 | 2.40 | n/a | n/a | 2.51 |
| | weeks | | | | | | | |
| | 8 | n/a | 0.12 | 97.04 | 2.49 | 0.35 | n/a | 2.96 |
| | weeks | | | | | | | |
| | 13 | n/a | n/a | 97.41 | 2.41 | n/a | 0.18 | 2.59 |
| | weeks | | | | | | | |

^{#} HBI-3808;
* Isomer of HBI-3808

### Example 18

Preparing sodium salt of HBI-3808 having a XRPD of Pattern 5, from sodium salt of HBI-3808 having a XRPD of Pattern 15

Sodium salt of pattern 5 (i.e. sodium salt of HBI-3808 having a XRPD of Pattern 5) exhibits an inverse solubility in water. Sodium salt of Pattern 5 was prepared using inverse solubility, with dissolution of HBI-3808 free acid in water at low temperature, and heating to reach super saturation, followed by anti-solvent addition. The solids isolated from this process were consistent with sodium salt of pattern 15, which can be converted to sodium salt of pattern 5 via drying under vacuum with exposure to elevated humidity (and the conversion proceeds via sodium salt of pattern 2 as an intermediate).

Method: 40 g of HBI-3808 free acid was weighed out and transferred to a 1 L vessel, equipped with a retreat curve impeller, a temperature probe baffle, and a FBRM probe. The HBI-3808 free acid was dissolved in 133 mL of water containing 1.2 equivalents of sodium hydroxide (3.80 g) at 10° C. After dissolution the experiment was heated to 25° C. over 30 minutes, and stirred at 25° C. for ca. 30 minutes. The experiment was stirred at 200 rpm. The experiment was seeded with 1 wt. % sodium salt Pattern 5. The seeds persisted. The experiment was stirred for 1 h at 25° C. 533 mL of acetone anti-solvent was then added using the following conditions, 0.5 vol./h (20 mL/h) for 7 h, then 1 vol./h (40 mL/h) for 4 h, and then 2 vol./h (80 mL/h) for 3 h. The final solvent system after anti-solvent addition was acetone:water 80:20% v:% v. After anti-solvent addition was complete the experiment was stirred at 25° C. for 1 h then cooled to 5° C. at 0.1° C./min, and held at 5° C. for ca. 4 h. The solids were isolated by Buchner filtration (90 mm, grade 1 paper) and washed with 2 volumes of pre-chilled acetone:water 80:20% v:% v. A sub-sample of the solids was analyzed by XRPD and PLM. The solids were dried at 40° C. under vacuum with exposure to humidity for 72 h. The solids were then exposed to 40° C./75% RH for ca. 3 days.

Results: The initial dissolution of the HBI-3808 free acid was slow, owing to the hydrophobic nature of the material, and took ca. 1.5 h. The total process volumes were 16.7. Focused beam reflectance measurement (FBRM) showed that during the anti-solvent addition there was a rapid increase in counts, at an approximate solvent system of acetone:water 12.5:87.5 % v: % v, after which there was no further significant increase in the total number of counts. FIG. 18A shows FBRM trend obtained during the process. The arrow 1 denotes the time point of seeding with sodium salt of HBI-3808 of XRPD Pattern 5, arrow 2 denotes the time point for start of addition of the anti-solvent, arrow 3 denotes the time point for end of addition of the anti-solvent, arrow 4 denotes the time point for start of cooling from 25° C., arrow 5 denotes time point when the experiment reaches 5° C. * denotes the time point where large increase in counts was observed, the solvent system at this point was acetone:water 12.5:87.5% v:% v. FIG. 18B shows non-weighted and square-weighted size distributions from FBRM. Concentration of HBI-3808 free acid in the mother liquor was 4.29 mg/mL, and in the wash liquor was 4.44 mg/mL. XRPD analysis of the damp isolated solids showed they were consistent with sodium salt of pattern 15 (FIG. 18C). After drying at 40° C. under vacuum with exposure to humidity for ca. 48 h the solids had a diffractogram which was predominately consistent with sodium salt of pattern 5, with some small peaks consistent with sodium salt of pattern 2. After exposure to 40° C./75% RH for 72 h, conversion to sodium salt of pattern 5 was observed. The isolated yield was 90%. HPLC analysis showed the solids had a chemical purity of 99.9% area, and were isolated in a theoretical yield of 94% (FIG. 18D). PLM analysis showed that the damp solids had a rod-like morphology and were birefringent. After drying the rod-like morphology persisted, with aggregation also observed (FIG. 18E). PSD analysis showed a d₁₀ of 5.8 μm, a d₅₀ of 22.0 μm and a d₉₀ of 60.0 μm (FIG. 18F). TG-DSC analysis showed a weight loss of 3.3 wt. % between 92-212° C., equal to loss of one equivalent of water. An endothermic event was observed with an onset temperature of 126° C., and a peak temperature of 157° C. (FIG. 18G). DSC analysis was carried out between −80° C. and 250° C. (FIGS. 18H-J). In the first heat, between 20° C. and 250° C., an endothermic event was observed with an onset temperature of 108° C., and a peak temperature of 180° C. (FIG. 18H). In the cooling step between 250° C. and −80° C., no thermal events were observed (FIG. 18I). In the final heating step between −80° C. and 250° C., no thermal events were observed (FIG. 18J). The ¹H NMR (FIG. 18K) and ¹³C DEPTQ (FIG. 18L) analysis was consistent with a sodium salt of HBI-3808. A residual acetone content of 0.03 wt. % was observed. Quantitative ¹H NMR showed the purity was 91.4%, with respect to the HBI-3808 free acid (FIG. 18M). GC analysis showed a residual acetone content of 281 ppm. CAD analysis showed the sodium content was 5.4% w/w, equal for 1.2 equivalents of sodium. IR analysis was recorded on these solids for reference for sodium salt of pattern 5 (FIG. 18N). The peak observed at 3479 cm⁻¹ is due to the presence of water in the damp solids. The peak observed at 1708 cm⁻¹ may be due to the presence of acetone in the damp solids. Raman analysis was recorded on these solids for reference for sodium salt of pattern 5 (FIG. 18O). The additional signal at ca. 1700 cm⁻¹ in the spectrum may be due to the presence of acetone in the damp solids. KF indicated a water content of 3.2% w/w, equal to 1 equivalent of water. The specific optical rotation was 26.1° at a wavelength of 589 nm, at 19° C., at 5 mg/mL in methanol. LC-MS a main peak with a retention time of 6.92 min, and a parent ion of 503.3 m/z, consistent with a [M−H]⁻ speciation for the HBI-3808 free acid. An additional signal was observed in the TIC spectrum, with a retention time of 7.38 min, for which the parent ion was 503.2 m/z, consistent with a [M−H]⁻ speciation for the HBI-3808 free acid. FIG. 18P shows TIC (top) and DAD (bottom) scan from LC-MS of solids isolated. FIG. 18Q and FIG. 18R show LC-MS spectrum for TIC signals with retention time 6.92 min (FIG. 18Q) and 7.38 min (FIG. 18R). UV spectrum of the isolated solids showed a signal (λmax) at 209 nm (FIG. 18S).

### Example 19

Preparing sodium salt of HBI-3808 having a XRPD of Pattern 5, from a slurry of sodium salt of HBI-3808 having a XRPD of Pattern 2

Sodium salt of HBI-3808 having a XRPD of pattern 5 was prepared from a slurry of sodium salt of HBI-3808 having a XRPD of Pattern 2 in water.

Method: 40 mg of sodium salt of HBI-3808 having a XRPD of Pattern 2, was weighed into a 2 mL push cap vial and a stirrer bar added. 0.2 mL of water was added to the vial and the experiment was stirred for ca. 24 h at 25° C. After 24 h the slurry was filtered by centrifuge filtration and the recovered solids were analyzed by XRPD. The pH of the filtered mother liquor was measured.

Results: The recovered solids had a XRPD diffractogram consistent with sodium salt of HBI-3808 having a XRPD of pattern 5 (FIG. 19). The pH of the filtered mother liquor was 9.77. The ¹H NMR spectrum of the isolated sodium salt of pattern 5 material did not display a carboxylic acid signal, indicating the material likely remained as a sodium salt of HBI-3808.

### Example 20

Preparing sodium salts of HBI-3808 from amorphous sodium salt of HBI-3808

Various polymorphs of the sodium salt of HBI-3808 was prepared from amorphous sodium salt of HBI-3808, according to the steps shown in FIG. 20. FIG. 20A shows steps for preparation of sodium salt of HBI-3808 having XRPD of pattern 1, 3, 5, 8, 11, 12, 13, and 14 from amorphous sodium salt of HBI-3808. FIG. 20B shows steps for preparation of sodium salt of HBI-3808 having XRPD of pattern 2, 5, 6, 7, 9, 10, 11, and 15 from amorphous sodium salt of HBI-3808.

“TC” in FIGS. 20A and B refers to temperature cycling between 40° C. to 5° C. “Evap” in FIGS. 20A and B refers to evaporation at 20° C., and ambient pressure. ASA in FIGS. 20A and B refer to anti-solvent addition. “TS” in FIGS. 20A and B refers to thermodynamic solubility. “Sodium salt pattern X” in FIGS. 20A and B to sodium salt of HBI-3808 having XRPD of pattern X, where X is 1, 2, 3, 4, 5, 6, 7, 8, 9, 10, 11, 12, 13, 14, 15 or 16.

### Example 21

Preparing sodium salts of HBI-3808 through reactive crystallization

Various polymorphs of the sodium salt of HBI-3808 was prepared through reactive crystallization. “Sodium salt pattern X” in FIG. 2I refers to sodium salt of HBI-3808 having XRPD of pattern X, where X is 1, 2, 3, 4, 5, 6, 7, 8, 9, 10, 11, 12, 13, 14, 15 or 16.

### Example 22

A Single-Dose Study of Intravenously and Orally Delivered HBI-3802 and HBI-3808 in New Zealand White Rabbits for Determination of their Respective Pharmacokinetic Profiles

Eight groups (n=3) of rabbits received single doses of HBI-3802 or the sodium salt (form 5) HBI-3808 either by intravenous dosing or by oral dosing. HBI-3802 was formulated in 20% PG +80% (20% Captisol in pH 7.4 PBS buffer), and HBI-3808 was formulated in 5% NMP+15% EtOH+10% Tween 80+70% (20% Captisol in pH 7.4 PBS buffer). In addition, normal or enteric coated capsules were used to orally deliver the sodium salt (form 5) of HBI-3808. To allow determination of oral absolute bioavailability, both HBI-3802 and HBI-3808 were delivered IV at a dose of 0.3 mg/kg and blood samples obtained at 0, 0.25, 0.5, 0.75, 1,2,4,8,12,24,48, and 72 hours after dosing for derivation of plasma used to determine plasma concentrations by LC-MS. The HBI-3802 0.3 mg/kg plasma concentrations were compared to plasma concentrations obtained at the same time points from rabbits receiving single oral doses of HBI-3202 at 1 or 5 mg/kg. The HBI-3808 0.3 mg/kg plasma concentrations were compared to plasma concentrations from groups of rabbits receiving oral gavage of 1 mg/kg HBI-3808 solution or 5 mg/kg HBI-3808 solution and from rabbits receiving 5 mg/kg of the sodium salt (form 5) of HBI-3808 in normal capsules or in enteric coated capsules.

HBI-3802 had low bioavailability (0.09%) when delivered orally, while the sodium salt of HBI-3808 had better bioavailability (17.9% and 27.7%) when the sodium salt of HBI-3808 was delivered at 1 and 5 mg/kg in 5% NMP+15% EtOH+10% tween 80+70% (20% Captisol in pH 7.4 PBS buffer), respectively. Both the Cmax and the AUC exhibited dose proportional increases with low variability.

When the sodium salt of HBI-3808 delivered in normal capsules or in enteric coated capsules, HBI-3808 5 mg/kg had 10.7% and 10.9% bioavailability, respectively. However, absorption onset was delayed until 8 hours with the enteric coated capsules. The Cmax and AUCs were similar to one another, but their respective values were ˜40%-50% of the values from the 5 mg/kg formulated dose.

The individual plasma concentration-time profiles after single-dose administrations of HBI-3802 or HBI-3808 in rabbits are summarized in FIGS. 22-34. Briefly, FIG. 22 and FIG. 23 show results for HBI-3802 after an IV dose of 0.3 mg/kg and a PO dose of 5 mg/kg, respectively. FIG. 24, FIG. 25, and FIG. 26 show results for HBI-3808 after IV doses of 0.3, 1, and 5 mg/kg, respectively. FIG. 27 and FIG. 28 show results for HBI-3808 after PO doses of 1 mg/kg and 5 mg/kg, respectively. FIG. 29 and FIG. 30 show results for HBI-3808 after a PO Dose of 5 mg/kg administered by normal capsule (FIG. 29) and enteric coated capsule (FIG. 30), respectively.

### Example 23

Induction of Differentiation of hESCs to CMs by HBI-3808

In order to test the effect of HBI-3808 on human embryonic stem cells (hESCs) on stimulating differentiation to cardiomyocytes (CMs), hESCs were incubated in the presence of media (see legend, FIG. 31) or 100 ng/ml HBI-3808. As can be seen in FIG. 31, HBI-3808 successfully stimulated hESCs to differentiate into viable, beating CMs by day 13. Moreover, stimulation of hESCs by HBI-3808 produced detectable modulation of cardiac-associated genes. As can be seen in FIG. 32, on day 1, Serine Protease 1 and FGF9 were up-regulated. On day 3, synaptotagmin-8, potassium voltage gated channel subfamily Q member 5, synaptotagmin like 4 and tubulin alpha like 3 were upregulated. On day 6, collagen type V alpha 1 chain, collagen type III alpha 1 chain, proponin Il, slow skeletal type potassium voltage-gated channel subfamily A, member 4 were upregulated. On day 9, troponin I1, slow skeletal type myosin heavy chain 6 and myosin light chain 3 were upregulated. And on day 13, when the CMs began beating, IGFBP5, collagen type III alpha 1 chain, natriuretic peptide receptor 3 and ISL LIM homeobox 1 were all upregulated. In FIG. 33, it is shown that media harvested from HBI-3808-treated CMs is enriched in VEGF, which suggests that there is an induction of angiogenesis in infarcted tissue as an alternative regenerative mechanism. As can be seen in FIG. 34, HBI-3808 induces angiogenesis in supernatant of CM cell suspensions. This suggests that HBI-3808 not only stimulates differentiation of SCs in CMs, but also stimulates growth of blood vessels necessary for supplying blood and nutrients and maintaining the prolonged survival of CMs.

### Example 24

Pre-Clinical Study-Adult Rat Model

Effects of HBI-3808 (Na⁺ salt) On Cardiac Function Post Myocardial Infarction

Introduction: Ischemic cardiomyopathy is a leading cause of morbidity and mortality worldwide. (Collaborators GBDCoD, “Global, regional, and national age-sex-specific mortality for 282 causes of death in 195 countries and territories, 1980-2017: a systematic analysis for the Global Burden of Disease Study 2017,” Lancet, 2018; 392: pp. 1736-88; Roth GA, Abate D, Abate KH, Abay SM, Abbafati C, Abbasi N, et al. “Global, regional, and national age-sex-specific mortality for 282 causes of death in 195 countries and territories 1980-2017: a systematic analysis for the Global Burden of Disease Study 2017,” The Lancet, 2018; 392: pp. 1736-88.) Current therapies, including medication, percutaneous coronary intervention (stents and balloon dilatation), and bypass surgery, which all aim to restore blood supply into the ischemic myocardium, are effective in most patients. However, despite great improvements, it is inevitable that myocardial infarction (MI) leads to permanent loss of cardiac tissue and ultimately heart failure (Roger V L, “Epidemiology of heart failure,” Circ. Res. 2013; 113: pp. 646-59) and mortality and morbidity are high. New therapies are urgently needed.

In a previous study, it was demonstrated that “Oral administration of HBI-3800 at the optimal dose of 0.5 mg/day beginning 6 days following a MI after permanent occlusion of the left coronary artery (LAD) of rats prevents ventricular dilation and cardiac dysfunction.” (Cheng L, Chen H, Yao X, Qi G, Liu H, Lee K, Lee K, Zhang J, Chen S, Lin X, Zhao W, Li J, Li M. “A plant-derived remedy for repair of infarcted heart,” PLoS One, 2009;4(2):e4461. doi: 10.1371/journal.pone.0004461. Epub 2009 Feb 14. PMID: 19221596; PMCID: PMC2637970).

HBI-3808, HBI-3802 aglycone, has been recently developed, which the Sponsor believes represents the active pharmacophore. The preliminary data demonstrated its beneficial effect in cultured cardiomyocytes. Therefore, we hypothesized that HBI-3808 have better beneficial effect on cardiac function restoration than the previous product of HBI-3802 in animal model post MI.

To test this hypothesis, we used the same animal model and evaluated effectiveness of new chemical HBI-3808 on cardiac protection in comparison with HBI-3802 as well as dose response of new HBI-3808 for prevention of ventricular dysfunction post myocardial infarction.

Research Aim: Investigate the pharmacologic activity and efficacious dose range of HBI-3808 on preservation of cardiac function post myocardial infarction.

Animal Model and Myocardial Infarction: Adult rats (Sprague Dawley (SD), body weight, -250 g) were used to generate a model with MI by LAD ligation of the heart as described in the previous study (Chongyu Zhang, Meng-Hsuan Hsieh, Song-Yi Wu, Shu-Hong Li, Jun Wu, Shi-Ming Liu, Hao-Ji Wei, Richard D. Weisel, Hsing-Wen Sung, Ren-Ke Li, “A self-doping conductive polymer hydrogel that can restore electrical impulse propagation at myocardial infarct to prevent cardiac arrhythmia and preserve ventricular function,” Biomaterials, Volume 231, 2020, 119672, ISSN 0142-9612). In brief, each rat was pre-medicated with 5% isoflurane in a chamber.

The rat was intubated, and the tube was then connected to a Harvard ventilator. Positive pressure ventilation was maintained with the ventilator and respiratory rate was set at 60/minute with tidal volume at 5 mL/respiration. The rat was ventilated with a mixture of room air, oxygen, and 2.5% isoflurane. The rat's chest was then shaved, and the skin incision area was cleaned with a povidone-iodine solution.

The heart was then exposed through a left anterolateral thoracotomy incision. A 7-0 polypropylene suture was used to ligate LAD at 2-3 mm below the anterior-inferior edge of the left atrium. Sham animals was treated identically except that the suture was passed through the myocardium beneath the LAD without ligation. The incision was then closed in layers using 3-0 Vicryl suture and 3-0 silk for the skin. Post-surgery treatment followed per UHN ARC requirement with antibiotics (Duplocillin LA, 15,000 IU/kg, I.M.) and analgesics (Buprenorphrine, 0.03 mg/Kg, SC), and the animals recovered from anesthesia in a warmed environment, under continuous monitoring and care for 1-2 hours.

Animal Pre-selection and Research Groups: On day 6 post myocardial infarction, cardiac function of the infarcted animals was evaluated using 2-dimensional echocardiography (Echo).

The percentage fraction shortening (FS) was evaluated under Echo and the rats with FS between 20 to 30% were selected for the study to minimize group variation within the group.

The selected rats having similar FS were randomly divided into 6 groups: (1) Negative control group (treated with 40% SBCD); (2) Positive control group (with HBI-3802, 2 mg/kg /day); (3) Dose 1 (HBI-3808 2 mg/kg/day); (4) Dose 2 (HBI-3808 0.64 mg/kg day); (5) Dose 3 (HBI-3808 0.2 mg/kg/day); and (6) Sham control (No myocardial infarction and treated with 40% SBCD).

Treatments: The rats were treated by gastric gavage daily starting 6 days after MI for 2 weeks (Turner PV, Vaughn E, Sunohara-Neilson J, Ovari J, Leri F, “Oral gavage in rats: animal welfare evaluation,” J. Am. Assoc. Lab. Anim. Sci., 2012 Jan; 51(1): pp. 25-30. PMID: 22330864; PMCID: PMC3276962) as described in Table 18. The same volume of vehicle (1 mL/rat/day) was given to the rats in sham control group. Ten rats were studied in each group (N=10/group). The schematic plan is depicted in FIG. 35.

TABLE 18
Rats treated by gastric gavage

| Group | Treatment per day | Number of Rats |
|---|---|---|
| 1 | Vehicle control (40% SB-CD) | 9 |
| 2 | 2 mg/kg/day HBI-3802 | 10 |
| 3 | 2 mg/kg/day HBI-3808 | 10 |
| 4 | 0.64 mg/kg/day HBI-3808 | 10 |
| 5 | 0.2 mg/kg/day HBI-3808 | 9 |
| 6 | Sham (40% SB-CD) | 10 |

Preparation of HBI-3802 and HBI-3808: The following method was used to prepare HBI-3802 or HBI-3808 free acid (aglycone) at a concentration of 0.5 mg/mL. SB-CD solution was prepared by dissolving 3.2 grams of SB-CD in 8 mL phosphate buffer (pH 6.8) to get 40% w/v SB-CD solution. The SB-CD solution was used to dissolve the compounds. Four mg of HBI-3802 or 3808 were weighed and 8 mL of the SB-CD solution was added to generate the solutions, which was adjusted pH using 0.2 M NaOH solution to >pH 6.8. The reagent solutions were stirred at ambient temperature for 10 minutes and then sonicated at ambient temperature for 10 minutes. The final solution is a clear liquid. The dose level 2 of HBI-3808 (0.16 mg/mL) was prepared by diluting 2 mL of 0.5 mg/mL HBI-3808 with 4.25 mL SB-CD solution. The dose level 3 of HBI-3808 (0.05 mg/mL) was prepared by diluting 2 mL of 0.16 mg/mL HBI-3808 with 4.4 mL SB-CD solution.

Oral Treatment: Six days after permanent LAD ligation, the selected rats received the first daily treatment. HBI-3808 (at 1 of the 3 doses) or 3802 at 2 mg/kg/day (1 ml/rat/day) delivered by gastric gavage daily for 14 days (2). 40% SB-CD alone (1 ml/rat/day) was similarly administered to rats in the control and sham groups.

Pharmacokinetic Analyses: On the final day of treatment, plasma samples were collected from the tail vein at 30 minutes and 2 hours after dosing for measurement of 3802 and 3808 levels. The details of the analytic method and the results will be reported elsewhere.

Measurement of Cardiac Function: Cardiac function was evaluated by echocardiography prior to LAD ligation, prior to treatment (6 days post myocardial infarction), and at 7- and 21-days post first treatment (13 and 27 days post myocardial infarction). The end-point cardiac function was also evaluated using a pressure-volume catheter on Day 21 after first treatment (27 days post myocardial infarction).

Echocardiography: Each rat was pre-medicated with isoflurane in a chamber, and anesthesia was maintained during measurement with 2.5% isoflurane delivered using a nose cone. Echocardiographic examinations were performed using a GE Vivid-7 System with a 10S transducer. Depth was set at 2 cm. Short-axis views were obtained using the parasternal approach.

Left ventricular (LV) dimensions [end-diastolic dimension (LVEDD) and end-systolic dimension (LVESD)] from M mode at short axis views of the mid-level LV. Fractional shortening (% FS) was calculated using the equation: [(LVEDD−LVESD)/LVEDD]×100. Ejection fraction (% EF) was calculated using the equation: [(LVEDD³−LVESD³)/LVEDD³]×100.

Pressure-Volume Measurement: End-point functional and morphological assessments were performed at 21 days after first treatment (27 days post myocardial infarction). Each rat was anesthetized as described above (Myocardial Infarction), and ventilated. The sternotomy was performed, and a calibrated Millar conductance pressure-volume catheter (Millar Instruments, USA) was inserted into the LV cavity through the apex. Pressure and volume data were collected with the Millar software. Parallel conductance was evaluated after the injection of hypertonic saline solution into the right jugular vein, with the volume measurements corrected for the parallel conductance. To analyze the LV pressure and volume relationship, pressure and volume data were acquired, and real-time pressure-volume loops were constructed. Stroke work (SW) and dP/dt max (indices of systolic function) and dP/dt min and Tau_w (index of diastolic function) as well as LV volumes were compared between the groups.

Morphological Studies: After cardiac functional measurements, the animals were euthanized. The hearts were rapidly excised, fixed with 10% formaldehyde for 7 days, sliced into 2 mm thick slices, and photographed.

Cross-sectional areas of the left ventricular free wall (LVFW) and scar tissue were measured using computed planimetry. Surface areas of the epicardial LVFW and scar tissue were measured as the sum of the epicardial length multiplied by the slice thickness (2 mm). The surface area percentage of scar tissue in the LVFW was calculated as follows: [(epicardial scar area)/(epicardial LVFW area)×100]. The thickness of scar area was presented as an average of wall thickness measured at the middle and at each edge of the scar from the slice having the thinnest scar.

Statistical Analyses: All data were expressed as mean±SD. Between-group comparisons were made using one-way analysis of variance (ANOVA). When the F ratio was significant, differences were specified by Fisher's multiple range tests. Differences were deemed significant when p<0.05.

### Results:

General Information: The body weight of the rats was measured prior to LAD ligation, prior to treatment, and 7- and 21-days post first treatment. There were no significant differences in body weight among groups (FIG. 36) during the study. One rat died after 1 day after LAD ligation in each of the vehicle control and 0.2 mg/kg/day HBI-3808 group.

Blood Analysis: Two plasma samples were collected in each rat at 14 days after oral treatment with 30- and 120-minutes interval after dosing. There are 58 survival rats and 116 plasma samples in total were collected. The plasma samples were analyzed at WuXi AppTec for PK assessment.

Cardiac Function Evaluated using ECHO: Cardiac function (FIG. 39A: LV-end of systolic dimension; 39B: LV-end of diastolic dimension was evaluated by Echo before LAD ligation (day 0), immediately before treatment (day 6), and 7 and 21 days after treatment (13 and 27 days post myocardial infarction). Changes in cardiac function during the course the study are presented in (FIG. 37A, 37B, 37C, FIG. 38A, 38B. 38C)

Ejection fraction (EF) and fractional shortening (FS) decreased similarly in all ligated groups prior to treatment (FIG. 37A-37C, FIG. 38A-38C). In analysis EF (FIG. 37A-37C) we found that the treatment on day 6 post myocardial infarction made significant differences among the groups. The EF in groups of 2 mg/kg of HBI-3802 and 2 mg/kg of HBI-3808 increased significantly after treatment in comparison with groups of 0.64 mg/kg and 0.2 mg/kg of HBI-3808 and the control group (FIG. 37A, 37B). The treatment also improved EF in 0.64 mg/kg HBI-3808 group compared to 0.2 mg/kg HBI-3808, which also has better cardiac function than the control group over the 21 days of study (FIG. 37A, 37B, p<0.02 and 0.01 respectively). There was an improvement in cardiac function in 2 mg/kg HBI-3808 compared with 2 mg/kg HBI-3802 group at 21 days after treatment (FIG. 38A-C, p<0.01). FIG. 3C (FIG. 37C) demonstrated difference of cardiac function between end point and pre-treatment (Days 27 vs 6 post infarction) phase of the study. The data showed that cardiac function is progressively decreasing in control group. The rats treated with 0.2 mg/kg of HBI-3808 prevented further deterioration of cardiac dysfunction compared with Day 6 post MI. In contrast vehicle treated controls showed a significant deterioration in EF between Day 6 and Day 21. The rats treated with 0.16 mg of HBI-3808 showed improvement in cardiac function compared with Day 6, which was significantly illustrated in the group of 2 mg/kg of HBI-3808. The rats treated with 2 mg/kg of HBI-3802 also showed improvement in cardiac function, however, it was not as effective as observed in the group of 2 mg/kg of HBI-3808 group.

In analysis of FS (FIG. 38A, 38B, 38C) we found that 2 mg/kg HBI-3802 and 2 mg/kg HBI-3808 treatment increased FS significantly in comparison with groups of 0.64 mg/kg and 0.2 mg/kg HBI-3808 and the control group (FIG. 38A). In contrast vehicle treated controls showed a significant deterioration in EF between Day 6 and Day 21. The treatment also improved cardiac function in 0.64 mg/kg HBI-3808 group compared to 0.2 mg/kg HBI-3808, which showed better function compared with the control group over the 21 days of study (FIG. 38A-38C, p<0.02 and 0.01 respectively). There was an improvement in cardiac function in 2 mg/kg HBI-3808 compared with 2 mg/kg HBI-3802 group at 21 days after treatment (FIG. 38A-38C, p<0.01). FIG. 38C also confirmed observation of the EF data as described in FIG. 37C.

Left ventricular end systolic dimension (LVESD) was smaller in the 2 mg/kg HBI-3808 group than in the 2 mg/kg HBI-3802 and 0.64 mg/kg, 0.2 mg/kg HBI-3808 groups as well as in the control group (FIG. 39A, p<0.01). In addition, 0.2 mg/kg HBI-3808 group had smaller LVESD compared with control group at 21 days after treatment (FIG. 39A p<0.01). Most importantly 2 mg/kg HBI-3802, 2 mg/kg HBI-3808 and 0.64 mg/kg HBI-3808 actually showed a reversal in systolic dilation evident between Day 6 and Day 21 in vehicle control treated animals.

Left ventricular end diastolic dimension (LVEDD) was smaller in the 2 mg/kg HBI-3808 group than in the 0.64 mg/kg, 0.2 mg/kg and control groups (FIG. 39B, p<0.01). In addition, 2 mg/kg HBI-3808 group had smaller LVEDD compared with control group at 21 days after treatment (FIG. 39B, p<0.01). The 2 mg/kg HBI-3808 and HBI-3802 groups showed no diastolic dilation between Day 6 and 21 compared with all other treatment groups.

Cardiac Function Evaluated using Pressure Volume (PV) Catheter: Cardiac function and ventricular volumes were also measured using PV catheters at 21 days after treatment (27 days post myocardial infarction). FIG. 40A shows representative cardiac performance loops recorded for each of the 6 groups. Smaller end systolic pressure and larger LV volume represent the worse cardiac function. FIG. 40A-40C show that stroke work (SW, FIG. 40B) and maximal rate of rise of left ventricular pressure (dP/dt max, FIG. 40C) values (indices of systolic function) were significantly greater in the 2 mg/kg HBI-3808 group than in the 2 mg/kg HBI-3802 and in the 0.64 mg/kg, 0.2 mg/kg HBI-3808 groups as well as in the control group (p<0.01). In addition, 0.2 mg/kg HBI-3808 group had better cardiac systolic function compared with control group at 21 days after treatment (p<0.05). FIG. 41A-41B shows that minimum rate of pressure change in the left ventricle (dP/dt min, FIG. 41A) and isovolumic relaxation constant (Tau_w, FIG. 41B) values (index of diastolic function) were also improved in the 2 mg/kg HBI-3808 group more than in the 2 mg/kg group of HBI-3802, and 0.64 mg/kg and 0.2 mg/kg of HBI-3808 groups as well as in the control group (FIG. 41A, p<0.05 and 0.01 respectively). In addition, 0.2 mg/kg HBI-3808 group had better cardiac diastolic function compared with control group at 21 days after treatment (p<0.01).

FIG. 42A demonstrates that left ventricular end systolic volumes (ESV) was smaller in the 2 mg/kg HBI-3808 group compared with 2 mg/kg HBI-3802, and 0.64 mg/kg and 0.2 mg/kg HBI-3808 groups as well as control group (p<0.05 and 0.01 respectively), and significantly smaller in the 0.2 mg/kg group of HBI-3808 than in the control group (p<0.02). Left ventricular end diastolic volumes (EDV) was smaller in the 2 mg/kg HBI-3808 group compared with 0.64 mg/kg and 0.2 mg/kg of HBI-3808 groups as well as control group (FIG. 42B, p<0.01), and significantly smaller in the 0.64 mg/kg of HBI-3808 group than in the 0.2 mg/kg group (FIG. 42B, p<0.01). Diastolic volumes were in fact the same for the HBI-3808 and HBI-380 groups compared with non-infarcted sham animals.

Morphological Analysis: FIG. 43 shows the representative sections of hearts from the 6 groups after formalin fixation (arrows indicate infarct areas). Computed planimetric analysis was performed on all hearts at 21 days after first treatment. FIG. 44A-44B demonstrates that scar areas were larger in the control group than in the 0.2 mg/kg, 0.64 mg/kg and 0.2 mg/kg HBI-3808 groups (FIG. 44A, p<0.05 and 0.01 respectively). Additionally, 2 mg/kg of HBI-3808 group had smaller infarct areas than the 2 mg/kg HBI-3802 group (FIG. 44A, p<0.01). Scars were thinner in the control group than in the 0.2 mg/kg, 0.64 mg/kg and 2 mg/kg of HBI-3808 groups (B, p<0.05 and 0.01 respectively). Additionally, 2 mg/kg of HBI-3802 group had thinner scar than the 2 mg/kg HBI-3808 group (FIG. 44B, p<0.01).

Histological Evaluation: To explore possible mechanisms of action, heart tissue was evaluated of for cardiomyocyte structure, myocardial fibrosis, blood vessel density as well as cellular interaction by staining heart section with (1) H&E; (2) Masson's Trichrome; (3) Smooth muscle actin; (4) endothelial cells (Factor VIII); and (5) Connexin 43. Initial evaluation compared results from the HBI-3808 (2.0 mg/kg), and control groups (N=8/group). In addition, two hearts from sham group were used as positive controls.

### Conclusion:

At 6 days after myocardial infarction, cardiac function decreased -50% evaluated using echocardiography in ligated groups compared to sham group. Cardiac function in the control group continuously decreased over the 21-day duration of the study. Oral administration of HBI-3802 prevented ventricular dilation and cardiac dysfunction following initiation of treatment as we presented 10 years ago. The new chemical entity HBI-3808 demonstrated enhanced cardiac function restoration and had a dose response. HBI-3808 has greater efficacy compared with HBI-3802 at 2 mg/kg/day. The highest dose of HBI-3808 (2 mg//kg/day) improved both diastolic and systolic function, while a dose of 0.64 or 0.2 mg/kg/day produced a lower-magnitude improvement of cardiac function. At Day 21, end diastolic volumes for the 2 mg/kg HBI-3808 and HBI-3802 treated groups were equivalent to non-infarcted sham animals. The functional restoration was associated with smaller end systolic and diastolic volumes, smaller infarct areas and thicker ventricular walls in scar regions. The mechanism responsible for functional improvement might be associated with a reduction of adverse myocardial remodeling, which requires further analysis.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1. A sodium salt of HBI-3808, wherein HBI-3808 has the following chemical formula,

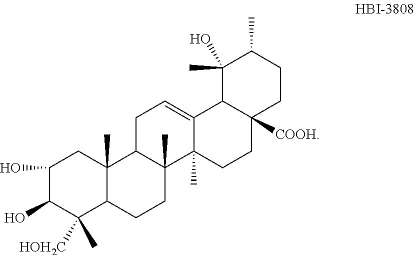

2. The sodium salt of HBI-3808 of claim 1, comprising HBI-3808 monosodium salt.

3. The sodium salt of HBI-3808 of claim 1, having:
a. an x-ray powder diffraction (XRPD) diffractogram of Pattern 1, wherein the XRPD diffractogram comprises 1 to 8 peaks (±0.2 °2θ) of Table 1;
b. an XRPD diffractogram of Pattern 2, wherein the XRPD diffractogram comprises 1 to 9 peaks (±0.2 °2θ) of Table 2A;
c. a thermogravimetry (TG)—differential scanning calorimetry (DSC) thermogram substantially matching that of FIG. 2G;
d. a DSC thermogram substantially matching that of FIG. 2H;
e. a dynamic vapor sorption (DVS) mass plot substantially matching the mass plot of FIG. 2K;
f. a DVS isotherm plot substantially matching the isotherm plot of FIG. 2L;
g. an FT-IR spectrum substantially matching the FT-IR spectrum of FIG. 2I;
h. a Raman spectrum substantially matching the Raman spectrum of FIG. 2J;
i. an XRPD diffractogram of Pattern 3, wherein the XRPD diffractogram comprises 1 to 8 peaks (±0.2°2θ) of Table 3;
j. an XRPD diffractogram of Pattern 4, wherein the XRPD diffractogram comprises 1 to 8 peaks (±0.2°2θ) of Table 4;
k. an XRPD diffractogram of Pattern 5, wherein the XRPD diffractogram comprises 1 to 10 peaks (±0.2 °2θ) of Table 5A;
l. a TG-DSC thermogram substantially matching that of FIG. 5G;
m. a DSC thermogram substantially matching that of FIG. 5H;
n. a DVS mass plot substantially matching the mass plot of FIG. 51;
o. a DVS isotherm plot substantially matching the isotherm plot of FIG. 5J;
p. an XRPD diffractogram of Pattern 6, wherein the XRPD diffractogram comprises 1 to 10 peaks (±0.2 °2θ) of Table 6;
q. an XRPD diffractogram of Pattern 7, wherein the XRPD diffractogram comprises 1 to 10 peaks (±0.2 °2θ) of Table 7;
r. an XRPD diffractogram of Pattern 8, wherein the XRPD diffractogram comprises 1 to 34 peaks (±0.2 °2θ) of Table 8;
s. an XRPD diffractogram of Pattern 9, wherein the XRPD diffractogram comprises 1 to 35 peaks (±0.2 °2θ) of Table 9;
t. an XRPD diffractogram of Pattern 10, wherein the XRPD diffractogram comprises 1 to 30 peaks (0.2°2θ) of Table 10;
u. an XRPD diffractogram of Pattern 11, wherein the XRPD diffractogram comprises 1 to 38 peaks (0.2°2θ) of Table 11;
v. an XRPD diffractogram of Pattern 12, wherein the XRPD diffractogram comprises 1 to 40 peaks (±0.2 °2θ) of Table 12;
w. an XRPD diffractogram of Pattern 13, wherein the XRPD diffractogram comprises 1 to 28 peaks (0.2°2θ) of Table 13;
x. an XRPD diffractogram of Pattern 14, wherein the XRPD diffractogram comprises 1 to 28 peaks (±0.2 °2θ) of Table 14;
y. an XRPD diffractogram of Pattern 15, wherein the XRPD diffractogram comprises 1 to 40 peaks (0.2 °2θ) of Table 15; or
z. an XRPD diffractogram of Pattern 16, wherein the XRPD diffractogram comprises 1 to 70, peaks (1 0.2 °2θ) of Table 16.

4. A method of making a sodium salt of HBI-3808, comprising:
a. contacting HBI-3808 with a first solvent to form a first slurry,
b. dissolving sodium hydroxide in a second solvent to form a first solution,
c. contacting the first slurry of a with the solution of b to form a mixture at a first temperature,
d. contacting the mixture of c with an anti-solvent to form a second slurry,
e. cooling the second slurry to a second temperature lower than the first temperature; and
f. isolating solids from the second slurry.

5. A method of making a sodium salt of HBI-3808, comprising:
a. contacting HBI-3808 with a first solvent at a first temperature to form a slurry;
b. contacting the slurry with sodium hydroxide and optionally additional solvent;
c. temperature cycling the slurry between the first temperature and a second temperature, which is lower than the first temperature; and
d. isolating solids from the slurry.

6. A method of making an HBI-3808 salt polymorph, comprising:
a. dissolving HBI-3808 sodium salt in a solvent to form a solution at a first temperature;
b. contacting the solution with a counter solvent;
c. optionally, cycling the solution between the first temperature and a second temperature lower than the first temperature; and
d. isolating solids from the solution.

7. A method of making a sodium salt of HBI-3808 of XRPD Pattern 5, comprising:
a. providing a sample of a sodium salt of HBI-3808 of XRPD Pattern 2, and
b. exposing the sodium salt of HBI-3808 of XRPD Pattern 2 to conditions comprising a combination of temperature and humidity for a time sufficient to convert the sodium salt of HBI-3808 of XRPD Pattern 2 to a sodium salt of HBI-3808 of XRPD Pattern 5.

8. An ethanolamine salt of HBI-3808.

9. The ethanolamine salt of HBI-3808 of claim 8, comprising HBI-3808 monoethanolamine salt.

10. A method of making an ethanolamine salt of HBI-3808, comprising:
a. contacting HBI-3808 with a solvent to form a slurry;
b. adding a volume of a solution comprising ethanolamine and solvent at a first temperature, the volume being sufficient to form a solution;
c. adding a counter solvent to the solution;
d. cooling the solution a second temperature lower than the first temperature for a time sufficient to precipitate solids from the solution; and
e. isolating the solids from the solution.

11. A method treating myocardial infarction in a subject in need of thereof, comprising administering an effective amount of a composition comprising HBI-3808 monosodium salt or HBI-3808 ethanolamine salt to the subject.

12. The method of claim 11, wherein the composition comprises less than 0.1% of N-methylpyrrolidinone (NP), is substantially free of NP, or is free of NMP.

13. The method of claim 11, wherein the composition is at least as bioavailable as an equimolar amount of HBI-3808 free acid.

14. A method of improving injection fraction, improving stroke work, increasing the area within pressure-volume loops, enhancing cardiogenic differentiation efficiency of endogenous mesenchymal stem cells (MSCs), increasing expression of MSC-specific biomarkers, facilitating transplantation and differentiation of MSCs into infarcted cardiac tissue, stimulating myocardial regeneration in infarcted cardiac tissue, stimulating stem cell differentiation into functional cardiomyocytes, replacing and remodeling the myocardium with new functional tissue, limiting infarct size, preventing or treating cardiomyocyte death, and preventing or treating heart failure in a subject in need of thereof, comprising administering an effective amount of a composition comprising HBI-3808 sodium salt or HBI-3808 ethanolamine salt to the subject.

15. A method of stimulating differentiation of human mesenchymal stem cells, human embryonic stem cells, or both into cardiomyocytes, comprising contacting the human mesenchymal stem cells, human embryonic stem cells, or both with HBI-3808, HBI-3808 sodium salt or HBI-3808 ethanolamine salt.

16. The method of claim 15, where the human mesenchymal stem cells or human embryonic stem cells are suspended in a growth medium.

17. The method of claim 15, wherein the human mesenchymal stem cells are autologous mesenchymal stem cells.

18. The method of claim 15, wherein the method comprises administering the HBI-3808, HBI-3808 sodium salt, or HBI-3808 ethanolamine salt to a human cardiac infarct patient and the human mesenchymal stem cells are within the patient.

19. The method of any one of claim 15, wherein HBI-3808, HBI-3808 sodium salt or HBI-3808 ethanolamine salt is present at a concentration of about 0.1 to about 10,000 ng/ml.
